SEQUENCE LISTING (276 sequences)

SEQ ID NO: 1 - DNA, 1641 bp, Streptococcus agalactiae
gtgtttatgatgaaaaaaggacaagtaaatgatactaagcaatcttactctctacgtaaatataaatttggtttagcatcagtaattttagggtcattcataatggtcacaagtcctgtttttgcggatcaaactacatcggttcaagttaataatcagacaggcactagtgtggatgctaataattcttccaatgagacaagtgcgtcaagtgtgattacttccaataatgatagtgttcaagcgtctgataaagttgtaaatagtcaaaatacggcaacaaaggacattactactcctttagtagagacaaagccaatggtggaaaaaacattacctgaacaagggaattatgtttatagcaaagaaaccgaggtgaaaaatacaccttcaaaatcagccccagtagctttctatgcaaagaaaggtgataaagttttctatgaccaagtatttaataaagataatgtgaaatggatttcatataagtcttttggtggcgtacgtcgatacgcagctattgagtcactagatccatcaggaggttcagagactaaagcacctactcctgtaacaaattcaggaagcaataatcaagagaaaatagcaacgcaaggaaattatacattttcacataaagtagaagtaaaaaatgaagctaaggtagcgagtccaactcaatttacattggacaaaggagacagaattttttacgaccaaatactaactattgaaggaaatcagtggttatcttataaatcattcaatggtgttcgtcgttttgttttgctaggtaaagcatcttcagtagaaaaaactgaagataaagaaaaagtgtctcctcaaccacaagcccgtattactaaaactggtagactgactatttctaacgaaacaactacaggttttgatattttaattacgaatattaaagatgataacggtatcgctgctgttaaggtaccggtttggactgaacaaggagggcaagatgatattaaatggtatacagctgtaactactggggatggcaactacaaagtagctgtatcatttgctgaccataagaatgagaagggtctttataatattcatttatactaccaagaagctagtgggacacttgtaggtgtaacaggaactaaagtgacagtagctggaactaattcttctcaagaacctattgaaaatggtttaccaaagactggtgtttataatattatcggaagtactgaagtaaaaaatgaagctaaaatatcaagtcagacccaatttactttagaaaaaggtgacaaaataaattatgatcaagtattgacagcagatggttaccagtggatttcttacaaatcttatagtggtgttcgtcgctatattcctgtgaaaaagctaactacaagtagtgaaaaagcgaaagatgaggcgactaaaccgactagttatcccaacttacctaaaacaggtacctatacatttactaaaactgtagatgtgaaaagtcaacctaaagtatcaagtccagtggaatttaattttcaaaagggtgaaaaaatacattatgatcaagtgttagtagtagatggtcatcagtggatttcatacaagagttattccggtattcgtcgctatattgaaatttaa

SEQ ID NO: 2 - protein, 546 aa, Streptococcus agalactiae
MFMMKKGQVNDTKQSYSLRKYKFGLASVILGSFIMVTSPVFADQTTSVQVNNQTGTSVDANNSSNETSASSVITSNNDSVQASDKVVNSQNTATKDITTPLVETKPMVEKTLPEQGNYVYSKETEVKNTPSKSAPVAFYAKKGDKVFYDQVFNKDNVKWISYKSFGGVRRYAAIESLDPSGGSETKAPTPVTNSGSNNQEKIATQGNYTFSHKVEVKNEAKVASPTQFTLDKGDRIFYDQILTIEGNQWLSYKSFNGVRRFVLLGKASSVEKTEDKEKVSPQPQARITKTGRLTISNETTTGFDILITNIKDDNGIAAVKVPVWTEQGGQDDIKWYTAVTTGDGNYKVAVSFADHKNEKGLYNIHLYYQEASGTLVGVTGTKVTVAGTNSSQEPIENGLPKTGVYNIIGSTEVKNEAKISSQTQFTLEKGDKINYDQVLTADGYQWISYKSYSGVRRYIPVKKLTTSSEKAKDEATKPTSYPNLPKTGTYTFTKTVDVKSQPKVSSPVEFNFQKGEKIHYDQVLVVDGHQWISYKSYSGIRRYIEI

SEQ ID NO: 3 - DNA, 822 bp, Streptococcus agalactiae
atgatattgagacgtcgaactattgttttatggcaactgggtatcgccatttctctcattcttagtattctagccttaaatctttatttccatagtactcccttgcaaaccaatgcagctttacggaaccttgctccttcattaaaccatctttttgggacagatggtttaggtagggatatgtttgtcagaacgattaaaggactttatttctctctacaagtcggcttattaggtgcccttatgggggtcattctggcgacagtttttggagtgcttgcaggtttaggaaatagcattattgataaaataatagcatggttagttgatttgtttattggtatgcctcatttgatttttatgattctcatttcttttgttgttgggaaaggtgctcaaggggtcatcattgcaacggctgttacacattggccttctttagcaaggcttatccgcaatgaagtctatcatctaaagaataaagaatttgtccaactttctaaaagtatgggaaaaacgccttattatattgtgaggcatcatatcctgcctttgattgcttctcaaattttcattggttttatcctcttatttccacatgtcatcctacatgaagcatcaatgactttcttaggatttgggctctctgccgaacaaccttcggttggtatcattctgtcagaggcagctaagcatatctctcttggaaattggtggttggttatctttccaggactttatcttattttggttgtcaatgcatttgatactatcggagaatctttaaagaaactcttttaccctcaaactgatcatttttag

SEQ ID NO: 4 - protein, 273 aa, Streptococcus agalactiae
MILRRRTIVLWQLGIAISLILSILALNLYFHSTPLQTNAALRNLAPSLNHLFGTDGLGRDMFVRTIKGLYFSLQVGLLGALMGVILATVFGVLAGLGNSIIDKIIAWLVDLFIGMPHLIFMILISFVVGKGAQGVIIATAVTHWPSLARLIRNEVYHLKNKEFVQLSKSMGKTPYYIVRHHILPLIASQIFIGFILLFPHVILHEASMTFLGFGLSAEQPSVGIILSEAAKHISLGNWWLVIFPGLYLILVVNAFDTIGESLKKLFYPQTDHF

SEQ ID NO: 5 - DNA, 804 bp, Streptococcus agalactiae
atgacagaaacattattaagcattaaagacctctccatcaccttcactcaatacggaagatttttaaaaccatttcaatcaacaccgatacaagcgctgaatttagaaattaaaaaaggtgagttattagctattataggtgctagtggttcggggaagagtttattagcacatgctattatggatattcttcctaaaaatgcatctgtaacaggagatatgatttatcgtggtcaatcactaaattctaaacgcattaaacagttgcgaggaaaagatattacgttgattccacaatcagttaattatttagatccatctatgaaagtcaaacatcaggtgcgcttaggtatctcagaaaattcaaaggctactcaagaaggattgtttcaacagtttggtttaaaagaaagtgatggtgacttggatcctttccaactttctggcggaatgctccgacgtgttttgtttacaacgtgtattagtgataaggtttctttgattattgcggatgagcccacccctggattacatccagatgctctgcaaatggttttagaccaactacgctcctttgcagataaaggaataagcgttatatttatcactcatgatattgtagcagctagtcaaattgctgatcgtattactatttttaaagagggaaaagctattgaaacagctccagctagtttctttagcggaaatggagagcagttacaaacagaatttgctagaagtttatggcgctctctcccacagcaagaatttttgaaaggagttactcatgaccttagaggctaa

SEQ ID NO: 6 - protein, 267 aa, Streptococcus agalactiae
MTETLLSIKDLSITFTQYGRFLKPFQSTPIQALNLEIKKGELLAIIGASGSGKSLLAHAIMDILPKNASVTGDMIYRGQSLNSKRIKQLRGKDITLIPQSVNYLDPSMKVKHQVRLGISENSKATQEGLFQQFGLKESDGDLDPFQLSGGMLRRVLFTTCISDKVSLIIADEPTPGLHPDALQMVLDQLRSFADKGISVIFITHDIVAASQIADRITIFKEGKAIETAPASFFSGNGEQLQTEFARSLWRSLPQQEFLKGVTHDLRG

SEQ ID NO: 7 - DNA, 495 bp, Streptococcus agalactiae
gtccatctggggtggttcccgattggtatttcttctccgataggtactttgagtcaagatattacgttagctgatcgtattaagcaccttattttacctgttttcacggtaagtattctaggcattgccaatgtaactcttcatactagaactaaaatgatgtcggtactttctagtgaatatgtcttatttgccagagcgcgtggggaaacggaatggcaaatttttaaaaatcattgtcttagaaatgctatcgtaccagctattacactgcatttttcctattttggagaattgtttggaggatccgttcttgctgagcaagttttctcatatccaggactagggtctaccctaactgaagcaggacttaaaagtgatacaccgctacttctagctattgtgatgatagggacattatttgtttttgcgggcaatcttattgcggatattttaaatagcataatcaatccacagttaaggagaaaagtatga

SEQ ID NO: 8 - protein, 164 aa, Streptococcus agalactiae
VHLGWFPIGISSPIGTLSQDITLADRIKHLILPVFTVSILGIANVTLHTRTKMMSVLSSEYVLFARARGETEWQIFKNHCLRNAIVPAITLHFSYFGELFGGSVLAEQVFSYPGLGSTLTEAGLKSDTPLLLAIVMIGTLFVFAGNLIADILNSIINPQLRRKV

SEQ ID NO: 9 - DNA, 579 bp, Streptococcus agalactiae
ttgcggacaattacgttcaaacacaatgaaacgcgatcgtcaaaaagcgaaggtagggcggtaatgcttaaaagattatttactgaagatggggaattgacaaagattagtcgtcgtttcgtttggatgttagtggttatctattgtcttattattgtcaggatgtgttttgggcctcaaattatgattgagggggtatcaactccgaatgttcagcgcttcggaagaattgtagctcttttagtaccatttaattcttttcgtagtttagatcagctaactagctttaaagagattttttgggttattggtcaaaatgtagtgaatattttactgctgtttcctctcattatagggttactatccctaaagccaagtttacggaaatataaaagcgttatattacttgctttcttgatgtctcttttcatagagtgtactcaagttgttttagatattttaatagatgctaatcgggtttttgaaatcgacgatctatggacaaataccttaggcggtcctttcgccctatggagttatcgaaacataaaaggttggcttctaactattagaaaatga

SEQ ID NO: 10 - protein, 192 aa, Streptococcus agalactiae
MRTITFKHNETRSSKSEGRAVMLKRLFTEDGELTKISRRFVWMLVVIYCLIIVRMCFGPQIMIEGVSTPNVQRFGRIVALLVPFNSFRSLDQLTSFKEIFWVIGQNVVNILLLFPLIIGLLSLKPSLRKYKSVILLAFLMSLFIECTQVVLDILIDANRVFEIDDLWTNTLGGPFALWSYRNIKGWLLTIRK

SEQ ID NO: 11 - DNA, 1221 bp, Streptococcus agalactiae
ttgaaaaatttaaatcgttatgtagttgcggtttctggagtcgttttacatttaatgctaggatcaacttatgcttggagtgtgtttcgtaacccaattatctcagagactggttgggatatttcatcagtttcattcgcttttagtttggctattttttgtctaggaatgtctgcagcttttatgggacacttagtagagcgttttggtcctaggataatgggaatgatttctgctattttatatggagcagggaatgtgttaacaggcttagccattgaaactcagcagttatggttactgtatgttgcatacggtattttaggaggaatcggacttggttcaggttatattactccagtatcgactattattaaatggtttcctgataggaggggactagcaacaggattcgctattatgggatttggctttgcttctttagtaacaagtccgcttgcacaatccttactgattaggattggtgtgggtaaaacgttttatattttgggattagtatatttttttgtcatgatgattgcctcacaatttattaaacaaccacctcaggaaaaaataactattttgactcacgatggtaaaaagaatgctatgaattcacaaattatcactggattaaaagcaaacgtcgctataaaatcaaaaaccttttacatcatttggttgaccttgtttattaatatttcgtgtggcttaggtttaatatcagcagcttcaccaatggcacaagatttagcaggctattccgcagaatctgcagccttattagtaggggtactagggatatttaacggttttggacgtctgttatgggcaagtctctctgactacattggacgcccgttgacctttataatattatttattgtgaactttattatgacttctagtttatttttgtcattcaatgctattgtatttgcaatagcgatgtctattttaatgacttgttatggtgcaggtttttccttattacctgcttatctaagtgatatttttggaacaaaggaattagctactttacatggttatagtttaacagcatgggcaatagcaggtctgtttgggcccctattgttatcaaagacatattcatggggaaattcctatcaattgacattaatggtttttggttttttattcttattcggattattgttatctctatatttaagaaaattaacaactaaagttgtgtag

SEQ ID NO: 12 - protein, 406 aa, Streptococcus agalactiae
LKNLNRYVVAVSGVVLHLMLGSTYAWSVFRNPIISETGWDISSVSFAFSLAIFCLGMSAAFMGHLVERFGPRIMGMISAILYGAGNVLTGLAIETQQLWLLYVAYGILGGIGLGSGYITPVSTIIKWFPDRRGLATGFAIMGFGFASLVTSPLAQSLLIRIGVGKTFYILGLVYFFVMMIASQFIKQPPQEKITILTHDGKKNAMNSQIITGLKANVAIKSKTFYIIWLTLFINISCGLGLISAASPMAQDLAGYSAESAALLVGVLGIFNGFGRLLWASLSDYIGRPLTFIILFIVNFIMTSSLFLSFNAIVFAIAMSILMTCYGAGFSLLPAYLSDIFGTKELATLHGYSLTAWAIAGLFGPLLLSKTYSWGNSYQLTLMVFGFLFLFGLLLSLYLRKLTTKVV

SEQ ID NO: 13 - DNA, 303 bp, Streptococcus agalactiae
atggcagataaaaacagaacatttaaacttgtaggtgcaggatcttctagcacacaagaaaaaattgaaaagcctgctctttcgtttatgcaagatgcgtggcgtcgcttgaaaaaaaacaaattagcagtagtttcactctatttattagctcttttacttactttttcgttagcctcaaatttatttgtaactcagaaggatgctaatgggtttgattcgaaaaaagtaacgacatatcgcaacttaccacctaaattgagttcaaaccttcctttttggaatggtagcattaatccatca

SEQ ID NO: 14 - protein, 101 aa, Streptococcus agalactiae
MADKNRTFKLVGAGSSSTQEKIEKPALSFMQDAWRRLKKNKLAVVSLYLLALLLTFSLASNLFVTQKDANGFDSKKVTTYRNLPPKLSSNLPFWNGSINPS

SEQ ID NO: 15 - DNA, 678 bp, Streptococcus agalactiae
atgaaaatagtagtaccagtaatgcctcgcagtcttgaagaggctcaagaaatagatttatcaaaatttgatagtgttgatattattgaatggcgagctgatgccttaccaaaggatgacattattaatgtagctccagctatttttgagaaattcgcaggtcatgaaattatttttacttttcgtacaacgcgtgaaggtggtaatattgtcttatctgatgctgagtatgttgagttaatccagaaaattaattctatctacaatccagattatattgattttgagtatttttcacataaagaagtttttcaagaaatgctagaatttccaaatttagtcctgtcttatcacaattttcaagagacaccggagaatattatggagatattttcagaattaacagccctagcaccacgagttgtgaaaatcgcagtaatgccaaagaatgaacaagatgtcttagacgttatgaattacactcgcggtttcaagactattaatcctgatcaagtttatgcgacggtatctatgagtaaaattggacgtatttctcgttttgctggtgatgtaactggatctagttggacatttgcatatttagattcatctatcgcacccggacaaattactatttcagagatgaagcgtgtcaaagcattgcttgacgctgactga

SEQ ID NO: 16 - protein, 225 aa, Streptococcus agalactiae
MKIVVPVMPRSLEEAQEIDLSKFDSVDIIEWRADALPKDDIINVAPAIFEKFAGHEIIFTFRTTREGGNIVLSDAEYVELIQKINSIYNPDYIDFEYFSHKEVFQEMLEFPNLVLSYHNFQETPENIMEIFSELTALAPRVVKIAVMPKNEQDVLDVMNYTRGFKTINPDQVYATVSMSKIGRISRFAGDVTGSSWTFAYLDSSIAPGQITISEMKRVKALLDAD

SEQ ID NO: 17 - DNA, 333 bp, Streptococcus agalactiae
atgaaagacttatttgcaacaacagaagcatcatcaaggaaacaggaacaagatagaattgtcaattacataaaacaacatgttgagttaacaaatggtaatcaaataaaaaaaattgagtttatcgactttcaaaaaaatgagatgacaggtacatggggaatttctactaaaattaatgaacaattttcgattagtttttctgaagatagaattggtggtaaacttagagcattaggatatcaaccgaatgaaataggtttttcaaaggacatcaatagtaataatcaaaatgttaatgatattgaagtgatttatatgaagaaagaatag

SEQ ID NO: 18 - protein, 110 aa, Streptococcus agalactiae
MKDLFATTEASSRKQEQDRIVNYIKQHVELTNGNQIKKIEFIDFQKNEMTGTWGISTKINEQFSISFSEDRIGGKLRALGYQPNEIGFSKDINSNNQNVNDIEVIYMKKE

SEQ ID NO: 19 - DNA, 350 bp, Streptococcus agalactiae
atgaaaaaacgtatatggtatttgataataataatcacagtaattttaggaggactagccatgaaaaacttatttgcaacaacagaagcatcatcaaggaaacaggaacaagatagaattgtcaattacataaaacaacatgttgagttaacaaatggtaatcaaataaaaaaaattgagtttatcgactttcaaaaaaatgagatgacaggtacatggggaatttctactaaaattaatgaacaattttcgattagtttttctgaagatagaattggtggtaaacttagagcattaggatatcaaccgaatgaaataggtttttcaaaggacatcaatagtaataatca

SEQ ID NO: 20 - protein, 117 aa, Streptococcus agalactiae
MKKRIWYLIIIITVILGGLAMKNLFATTEASSRKQEQDRIVNYIKQHVELTNGNQIKKIEFIDFQKNEMTGTWGISTKINEQFSISFSEDRIGGKLRALGYQPNEIGFSKDINSNNQ

SEQ ID NO: 21 - DNA, 1350 bp, Streptococcus agalactiae
atgtcaaatcaatatgattatatcgttattggtggaggtagtgcaggcagtggtaccgctaatagggcagccatgtatggagcaaaagtcctgttaattgaaggtggacaagtaggtggaacttgtgttaacttaggttgtgtacctaagaaaatcatgtggtatggtgcacaagtttctgagacactccataagtatagttcaggttatggttttgaagccaataatcttagttttgattttactactctaaaagctaatcgcgatgcttacgtgcagcggtctagacagtcgtatgccgctaattttgagcgtaatggggtcgaaaagattgatggatttgctcgttttattgataaccatactattgaagtgaatggtcagcaatataaagctcctcacattactattgcaacaggtggacaccctctttaccctgatattattggaagtgaacttggtgagacttctgatgatttttttggatgggagaccttaccaaattctatattgattgttggggcgggctatatcgcggcagaacttgctggagtggttaatgaattaggcgttgaaacccatcttgcatttagaaaagaccatattctacgcggatttgatgacatggtaacaagtgaggttatggctgaaatggagaaatcaggtatctctttacatgctaaccatgtacctaaatctcttaaacgcgatgaaggtggcaagttgatttttgaagctgaaaatgggaaaacgcttgtcgttgatcgtgtaatatgggctatcggccgtggaccaaatgtagacatgggacttgaaaataccgatattgttttaaatgataaagattatatcaaaacagatgaatttgagaatacttctgtagatggcgtgtatgctattggagatgttaatgggaaaattgccttgacaccggtagcaattgcagcaggtcgtcgcttatcagaaagactttttaatcataaagataacgaaaaattagattaccataatgtaccttcagttatttttactcaccctgtaattgggacggtaggactttcagaagcagcagctatcgagcaatttggaaaagataatatcaaagtctatacatcaacttttacctctatgtatacggctgttaccagtaatcgccaagcagttaagatgaagctcataaccctaggaaaagaggaaaaagttattgggcttcatggtgttggttatggtattgatgaaatgattcaaggtttttcagttgctatcaaaatgggggctactaaagcagactttgatgatactgttgctattcacccaactggatctgaggaatttgttacaatgcgctaa

SEQ ID NO: 22 - protein, 449 aa, Streptococcus agalactiae
MSNQYDYIVIGGGSAGSGTANRAAMYGAKVLLIEGGQVGGTCVNLGCVPKKIMWYGAQVSETLHKYSSGYGFEANNLSFDFTTLKANRDAYVQRSRQSYAANFERNGVEKIDGFARFIDNHTIEVNGQQYKAPHITIATGGHPLYPDIIGSELGETSDDFFGWETLPNSILIVGAGYIAAELAGVVNELGVETHLAFRKDHILRGFDDMVTSEVMAEMEKSGISLHANHVPKSLKRDEGGKLIFEAENGKTLVVDRVIWAIGRGPNVDMGLENTDIVLNDKDYIKTDEFENTSVDGVYAIGDVNGKIALTPVAIAAGRRLSERLFNHKDNEKLDYHNVPSVIFTHPVIGTVGLSEAAAIEQFGKDNIKVYTSTFTSMYTAVTSNRQAVKMKLITLGKEEKVIGLHGVGYGIDEMIQGFSVAIKMGATKADFDDTVAIHPTGSEEFVTMR

SEQ ID NO: 23 - DNA, 3168 bp, Streptococcus agalactiae
atgacaaaaaaacatcttaaaacgcttgccttggcacttactacagtatcagtagtgacatacagccaggaggtatatggattagaaagagaggaatcggtcaaacaagaacaaacccagtcagcttcagaagatgattggttcgaagaagataatgagaggaaaacaaatgtttctaaagagaattctactgttgatgaaacagttagtgatttattttctgatggaaatagtaataactctagttctaaaaccgagtcagtggtaagtgaccctaaacaagtccccaaagcaaaaccagaggttacacaagaagcaagcaattctagtaatgatgctagcaaagtagaagtaccaaaacaggatacagcttcaaaaaaggaaactctagaaacatcaacttgggaggcaaaagatttcgtaactagaggggatactttagtaggtttttcaaaatctggaattaataagttatctcaaacatcacacttggttttaccaagtcatgcagcagatggaactcaattgacacaagtagctagctttgcttttactccagataaaaagacggccattgcagaatatacaagtaggctaggagaaaatgggaaaccgagtcgtttagatattgatcagaaggaaattattgatgagggagaaatatttaatgcttaccagttgactaagcttactattccaaatggttataagtctattggtcaagatgcttttgtggacaataagaatattgctgaggttaaccttcctgagagtctcgagactatttcagactatgcttttgctcacatgtctttaaaacaagtaaagttaccagataacctaaaggtcattggagaattagctttttttgataatcagattggtggtaagctttacttgccacgtcacttgataaaattagcagaacgcgctttcaaatctaatcgtattcaaacagttgaatttttgggaagtaagcttaaggttataggagaagcaagttttcaagataataatctgaggaatgttatgcttccggatggacttgaaaaaatagaatcagaagcttttacaggaaatccaggagatgaacattacaacaatcaggttgtattgcgcacaaggacaggccaaaatccacatcaacttgcgactgagaatacttacgtcaatccggacaaatcattgtggcgtgcaacacctgatatggattataccaaatggttagaggaagattttacctatcaaaaaaatagtgttacaggtttttcaaataaaggcttacaaaaggtaagacgtaataaaaacttagaaattccaaaacaacacaatggtattactattactgaaattggtgataacgcttttcgcaatgttgattttcaaagtaaaactttacgtaaatatgatttggaagaaataaagctcccctcaactattcggaaaataggtgcttttgcttttcaatctaataacttgaaatcctttgaagcaagtgaagatttagaagagattaaagagggagcctttatgaataatcgtattggaactctagacttgaaagacaaacttatcaaaataggtgatgctgctttccatattaatcatatttatgccattgttcttccagaatctgtacaagaaataggacgttcagcttttcgacaaaatggtgcgcttcaccttatgtttatcggaaataaggttaaaacaattggtgaaatggcttttttatccaataaactggaaagtgtaaatctctctgagcaaaaacaattaaagacaattgaggtccaagctttttcggataatgcccttagtgaagtagtcttaccgccaaatttacagactattcgtgaagaggctttcaaaaggaatcatttgaaagaagtgaagggttcatctacattatctcagattacttttaatgcttttgatcaaaatgatggggacaaacgctttggtaagaaagtggttgttaggacacataataattctcatatgttagcagatggtgagcgttttatcattgatccagataagctatcttctacaatggtagaccttgaaaaggttttaaaaataatcgaaggtttagattactctacattacgtcagactactcaaactcagtttagagaaatgactactgcaggtaaagcgttgttatcaaaatctaacctccgacaaggagaaaaacaaaaattccttcaagaagcacaatttttccttggtcgcgttgatttggataaagccatagctaaagctgagaaggctttagtgaccaagaaggcaacaaagaatggtcatttgcttgagaggagtattaacaaagcggtattagcttataataatagtgctattaaaaaagctaatgttaagcgcttggaaaaagagttagacttgctgacagatttagtcgagggaaaaggaccattagcgcaagctacaatggtacaaggagtttatttattaaagacgcctttaccattgccagaatattatatcggattgaacgtttattttgacaagtctggaaaattgatttatgcacttgatatgagtgatactattggcgagggacaaaaagatgcatatggtaatcctatattaaatgttgacgaggataatgaaggttatcataccttggcagttgccactttagctgattatgaaggtctttatattaaagatattttaaatagttcccttgataagattaaagcaatacgccagattcctttggcaaaatatcatagattaggaattttccaagctatccgaaatgcagcggcagaagcagaccgattgcttcctaagacacctaaggggtacctaaatgaagtcccaaattatcgtaaaaaacaaatggagaaaaatttaaaaccagttgattataaaacgccgatttttaataaggctttacctaatgaaaaggtagacggtgatagagcggctaaaggtcataatataaatgcggagactaataattctgtagctgtaacaccaataaggtccgagcagcaattacataagtcacagtctgatgtaaatttacctcaaacaagttctaaaaataattttatatacgagattctaggatacgttagtttatgtttgcttttcctagtaactgctgggaaaaaaggaaaacgagcaagaaaataa

SEQ ID NO: 24 - protein, 1055 aa, Streptococcus agalactiae
MTKKHLKTLALALTTVSVVTYSQEVYGLEREESVKQEQTQSASEDDWFEEDNERKTNVSKENSTVDETVSDLFSDGNSNNSSSKTESVVSDPKQVPKAKPEVTQEASNSSNDASKVEVPKQDTASKKETLETSTWEAKDFVTRGDTLVGFSKSGINKLSQTSHLVLPSHAADGTQLTQVASFAFTPDKKTAIAEYTSRLGENGKPSRLDIDQKEIIDEGEIFNAYQLTKLTIPNGYKSIGQDAFVDNKNIAEVNLPESLETISDYAFAHMSLKQVKLPDNLKVIGELAFFDNQIGGKLYLPRHLIKLAERAFKSNRIQTVEFLGSKLKVIGEASFQDNNLRNVMLPDGLEKIESEAFTGNPGDEHYNNQVVLRTRTGQNPHQLATENTYVNPDKSLWRATPDMDYTKWLEEDFTYQKNSVTGFSNKGLQKVRRNKNLEIPKQHNGITITEIGDNAFRNVDFQSKTLRKYDLEEIKLPSTIRKIGAFAFQSNNLKSFEASEDLEEIKEGAFMNNRIGTLDLKDKLIKIGDAAFHINHIYAIVLPESVQEIGRSAFRQNGALHLMFIGNKVKTIGEMAFLSNKLESVNLSEQKQLKTIEVQAFSDNALSEVVLPPNLQTIREEAFKRNHLKEVKGSSTLSQITFNAFDQNDGDKRFGKKVVVRTHNNSHMLADGERFIIDPDKLSSTMVDLEKVLKIIEGLDYSTLRQTTQTQFREMTTAGKALLSKSNLRQGEKQKFLQEAQFFLGRVDLDKAIAKAEKALVTKKATKNGHLLERSINKAVLAYNNSAIKKANVKRLEKELDLLTDLVEGKGPLAQATMVQGVYLLKTPLPLPEYYIGLNVYFDKSGKLIYALDMSDTIGEGQKDAYGNPILNVDEDNEGYHTLAVATLADYEGLYIKDILNSSLDKIKAIRQIPLAKYHRLGIFQAIRNAAAEADRLLPKTPKGYLNEVPNYRKKQMEKNLKPVDYKTPIFNKALPNEKVDGDRAAKGHNINAETNNSVAVTPIRSEQQLHKSQSDVNLPQTSSKNNFIYEILGYVSLCLLFLVTAGKKGKRARK

SEQ ID NO: 25 - DNA, 153 bp, Streptococcus agalactiae
gcaggatacatcatgcacaagcacgaggctatcgtgtcatgctggggtcaacccaggaagacatgtcggcacaagctgaagatttctttacagtctgtacacaataaagagacgggtaagagcgcttttaatgacaaagaacgactagcaatt

SEQ ID NO: 26 - protein, 51 aa, Streptococcus agalactiae
AGYIMHKHEAIVSCWGQPRKTCRHKLKISLQSVHNKETGKSAFNDKERLAI

SEQ ID NO: 27 - DNA, 1095 bp, Streptococcus agalactiae
gtgtcatttatgcaaagaaaatcctatttaaaatccatgagtgttcttactttaacagcttgtcttatatcaggatatgtggttaaagatattgctatgttacatgcagtatctgccagtgagaagaaagcaaataatgtcagtccgagagaaaatctctacagggctgtcaatgataattggctagccaatacaaaactcaaacaagggcagactagtgttaatagtttttcagaaattgaggataaattaaagcaactgttagtgtctgatatggctaaaatggcctcaggaaagattgaaacaaccaatgatgaacagaaaaaaatggttgcatactataaacaaggtatggactttaaaacaagagataaaaatggtctcaaacctctaaaaccagttttacaaaaacttgaagcagtctcttcaatgaaagactttcaaagtttggcccatgattttgtgatgagtggttttgttttaccatttggtttgactgtggaaaccaatgctcgagataatagccaaaagcaattggtgcttcgtcaagcacccgcattacttgaatcacctgaccaatataagaagggcaataaagaaggtgaggctaaattatcagcttaccgtacttcagcaatggctttgcttaaacaagctggaaaaagtaacattgaagatagaaaactagttaaacaagctatagcatttgatagactcttatcagaaaaaacgcaagttgatcaaagtaaaatcacagctgaaagtgagacagctgcggggcgatataaccctgaaagtatggaaacggttcacaattacgccaaggaatttgactttaaagaattgattgaaaaactagttgggccaacgaataaggcagtcaatgtagaagataaaacttattttaaacaggttaatgatgttataaatagtaaacaattagccaatatgaaagcatggatgatgatttctatgctagttgatcaatcagattttctaggagaacaaaatcgtcaagcagcgagtgcttttaagaatgttgcgtctggtttgactcagattgaatcgaaagaaaaaatgcttacacccaattag

SEQ ID NO: 28 - protein, 364 aa, Streptococcus agalactiae
MSFMQRKSYLKSMSVLTLTACLISGYVVKDIAMLHAVSASEKKANNVSPRENLYRAVNDNWLANTKLKQGQTSVNSFSEIEDKLKQLLVSDMAKMASGKIETTNDEQKKMVAYYKQGMDFKTRDKNGLKPLKPVLQKLEAVSSMKDFQSLAHDFVMSGFVLPFGLTVETNARDNSQKQLVLRQAPALLESPDQYKKGNKEGEAKLSAYRTSAMALLKQAGKSNIEDRKLVKQAIAFDRLLSEKTQVDQSKITAESETAAGRYNPESMETVHNYAKEFDFKELIEKLVGPTNKAVNVEDKTYFKQVNDVINSKQLANMKAWMMISMLVDQSDFLGEQNRQAASAFKNVASGLTQIESKEKMLTPN

SEQ ID NO: 29 - DNA, 174 bp, Streptococcus agalactiae
atggaaatgcctaaaagaaatgaattactcaataaagaaattaaaatgagtattgataaacttagatataaagaaccagagagtgaacatgacaagcgacctactttttatttggtagtacttatacttgttactgtagcagttatattgtcgttatttaaatattttttatag

SEQ ID NO: 30 - protein, 57 aa, Streptococcus agalactiae
MEMPKRNELLNKEIKMSIDKLRYKEPESEHDKRPTFYLVVLILVTVAVILSLFKYFL

SEQ ID NO: 31 - DNA, 140 bp, Streptococcus agalactiae
atgcaggtatttttaaatattgtcaataaattctttgatccagttattcatatgggttcgggagttgtgatgctaattgtcatgacaggtttagccatgatatttggagtgaagttttctaaagcacttgaaggtggtat

SEQ ID NO: 32 - protein, 46 aa, Streptococcus agalactiae
MQVFLNIVNKFFDPVIHMGSGVVMLIVMTGLAMIFGVKFSKALEGG

SEQ ID NO: 33 - DNA, 110 bp, Streptococcus agalactiae
atgaaaaagaaaacattcagtgcttataactttttaacggctcttatcctttgtcttttgacagtgctttttatctttccattttattggattatgacaggagcttttaa

SEQ ID NO: 34 - protein, 36 aa, Streptococcus agalactiae
MKKKTFSAYNFLTALILCLLTVLFIFPFYWIMTGAF

SEQ ID NO: 35 - DNA, 744 bp, Streptococcus agalactiae
atgactgagaactggttacatactaaagatggttcagatatttattatcgtgtcgttggtcaaggtcaaccgattgtttttttacatggcaatagcttaagtagtcgctattttgataagcaaatagcatatttttctaagtattaccaagttattgttatggatagtagagggcatggcaaaagtcatgcaaagctaaataccattagtttcaggcaaatagcagttgacttaaaggatatcttagttcatttagagattgataaagttatattggtaggccatagcgatggtgctaatttagctttagtttttcaaacgatgtttccagatatggttagagggcttttgcttaattcagggaacctgactattcatggtcagcgatggtgggatattcttttagtaaggattgcctataaattccttcactatttagggaaactctttccgtatatgaggcaaaaagctcaagttatttcgcttatgttggaggatttgaagattagtccagctgatttacagcatgtgtcaactcctgtaatggttttggttggaaataaggacataattaagttaaatcattctaagaaacttgcttcttattttccaaggggggagttttattctttagttggctttgggcatcacattattaagcaagattcccatgtttttaatattattgcaaaaaagtttatcaacgatacgttgaaaggagaaattgttgaaaaagctaattga

SEQ ID NO: 36 - protein, 247 aa, Streptococcus agalactiae
MTENWLHTKDGSDIYYRVVGQGQPIVFLHGNSLSSRYFDKQIAYFSKYYQVIVMDSRGHGKSHAKLNTISFRQIAVDLKDILVHLEIDKVILVGHSDGANLALVFQTMFPDMVRGLLLNSGNLTIHGQRWWDILLVRIAYKFLHYLGKLFPYMRQKAQVISLMLEDLKISPADLQHVSTPVMVLVGNKDIIKLNHSKKLASYFPRGEFYSLVGFGHHIIKQDSHVFNIIAKKFINDTLKGEIVEKAN

SEQ ID NO: 37 - DNA, 405 bp, Streptococcus agalactiae
atggtagcaaaagagttaggtaaaaatagctttactatcccaactatttgttctaattgctccgcaggtactgccattgcagttgtatataatgatgaccattctttcttaagatacggctatcccgagtctccacttcatatttttatcaatacacggatcattgcacaggcaccaagcaaatatttttgggctggtattggggacggtatttcaaaagcccctgaagtagaacgtgctaccttagaggctaagaccaataaactaccacatactgcagtgttaggacaagcagtcgctctgtcttcaaaggaagctttttatcaatttggtgaacaaggtctaaaagacgttgaagctaatttagcttcgcgtgcagttgaagaaattgcgcttgatatctta

SEQ ID NO: 38 - protein, 135 aa, Streptococcus agalactiae
MVAKELGKNSFTIPTICSNCSAGTAIAVVYNDDHSFLRYGYPESPLHIFINTRIIAQAPSKYFWAGIGDGISKAPEVERATLEAKTNKLPHTAVLGQAVALSSKEAFYQFGEQGLKDVEANLASRAVEEIALDIL

SEQ ID NO: 39 - DNA, 921 bp, Streptococcus agalactiae
ttgagggaaacttactggaaaatttcaagcgattgcgataaaataaatcttgcagagttttctagagaaaggaggtcagatttattggagtggcaagatctagcgcagttacctgtatctatttttaaagactatgttacagatgctcaagacgcggaaaaaccttttatatggacagaagtatttttaagggagattaatcgctcaaatcaagaaattattttgcatatttggccgatgactaagacagtcattctggggatgttagatcgagaattaccacatttagaattagctaaaaaagaaatcatcagtcgtggttatgaaccagttgttcggaattttggaggtctcgcagttgtagctgatgaaggaattttaaatttttcattggttattccagatgtttttgagagaaaattgtctatctcagatgggtatcttataatggtcgattttattagaagtatattttcggatttttatcaacctattgagcactttgaagtagagacctcctattgtcctggtaagtttgatcttagtataaatggcaaaaaatttgctggcttggctcagcgccgtataaagaatggtattgcggtatcaatttaccttagcgtttgtggcgatcaaaaagggcggagtcaaatgatttcagatttttataagattggtctaggtgatacgggtagtccaattgcttatccaaatgtagatcctgaaattatggctaatctatctgatctattagattgtcctatgacagtagaagatgttattgatcgtatgttgattagccttaaacaagtaggttttaatgatcgtttactgatgattagacccgatttagttgcagagtttgatagatttcaggctaagtctatggctaataaggggatggtgagcagagatgaataa

SEQ ID NO: 40 - protein, 306 aa, Streptococcus agalactiae
MRETYWKISSDCDKINLAEFSRERRSDLLEWQDLAQLPVSIFKDYVTDAQDAEKPFIWTEVFLREINRSNQEIILHIWPMTKTVILGMLDRELPHLELAKKEIISRGYEPVVRNFGGLAVVADEGILNFSLVIPDVFERKLSISDGYLIMVDFIRSIFSDFYQPIEHFEVETSYCPGKFDLSINGKKFAGLAQRRIKNGIAVSIYLSVCGDQKGRSQMISDFYKIGLGDTGSPIAYPNVDPEIMANLSDLLDCPMTVEDVIDRMLISLKQVGFNDRLLMIRPDLVAEFDRFQAKSMANKGMVSRDE

SEQ ID NO: 41 - DNA, 867 bp, Streptococcus agalactiae
ttggaaggtttacttattgcattgattcccatgtttgcgtgggaaagtattggatttgttagtaataaaattggagggcgtccaaatcaacaaacatttggaatgactttaggagcattgctatttgcgattatcgtatggttatttaaacagccagagatgactgcctcattgtggatttttggtatcttaggtggtatcctatggtcagtcggccaaaatggtcaatttcaagcaatgaaatatatgggagtctctgttgctaatccactgtcaagtggtgcacaattagtaggtggaagcctagttggtgctttagtctttcatgaatggactaagccaatccaatttattttaggattgacagcgttgacattattagttatcggcttctatttctcaagtaaacgtgatgtttcagaacaagctttggcaacacatcaagagttttcaaaaggatttgctacaattgcttattcaactgtaggttacatctcgtacgcagttttatttaacaacattatgaagttcgacgctatggccgtcattttacccatggctgttggaatgtgtctaggtgcaatttgtttcatgaagtttcgtgttaactttgaggctgttgttgttaaaaatatgattacaggtctcatgtggggcgttggtaatgtcttcatgttattggcagcagctaaagcagggctagcaattgcttttagtttttctcaacttggagtaattatctctattattggtggtattttatttttaggtgagacaaaaacgaagaaagagcagaaatgggttgtcatgggtatcctttgttttgttatgggtgctatattacttggtattgttaaatcttattaa

SEQ ID NO: 42 - protein, 288 aa, Streptococcus agalactiae
MEGLLIALIPMFAWESIGFVSNKIGGRPNQQTFGMTLGALLFAIIVWLFKQPEMTASLWIFGILGGILWSVGQNGQFQAMKYMGVSVANPLSSGAQLVGGSLVGALVFHEWTKPIQFILGLTALTLLVIGFYFSSKRDVSEQALATHQEFSKGFATIAYSTVGYISYAVLFNNIMKFDAMAVILPMAVGMCLGAICFMKFRVNFEAVVVKNMITGLMWGVGNVFMLLAAAKAGLAIAFSFSQLGVIISIIGGILFLGETKTKKEQKWVVMGILCFVMGAILLGIVKSY

SEQ ID NO: 43 - DNA, 960 bp, Streptococcus agalactiae
atgacaacttactacgaagctataaactggaacgaaattgaagatgttattgataaatcaacttgggaaaaactaaccgaacaattttggctcgatacacgtatccctttatcaaatgacttagacgattggcgcaaactttccgctcaagaaaaagatcttgttggcaaggtttttggaggcttaaccctacttgataccatgcaatcagaaactggtgttgaagctattcgtgccgatgttcgcacgcctcacgaagaagctgtcttaaacaatattcaattcatggaatctgttcacgctaaatcttattcttcaattttctcaactttaaatactaaatcagaaattgaagaaattttcgagtggactaataataatgagttccttcaagaaaaagcacgtattatcaatgacatttatgctaatggaaatgcccttcaaaaaaaggtggcttccacctacctcgaaactttccttttttattctggctttttcacacctctttactatttgggaaataataagttagcaaatgttgctgaaatcattaaattaattattcgtgatgaatctgtacatggtacttatatcggttacaaattccagcttggttttaacgaattaccagaagatgagcaagagaattttcgtgattggatgtatgacctcctttatcagctgtatgaaaacgaagaaaaatacaccaagacactttatgatggcgtaggatggactgaagaagttatgacctttttacgctacaatgctaataaagctcttatgaatttaggacaagatcctttattcccagatacagcaaatgatgtcaacccaattgttatgaatggtatttcaacaggaacatcaaaccatgacttcttctctcaagtaggtaatggttacctacttggtagcgttgaagctatgcatgatgatgactataactatggattataa

SEQ ID NO: 44 - protein, 319 aa, Streptococcus agalactiae
MTTYYEAINWNEIEDVIDKSTWEKLTEQFWLDTRIPLSNDLDDWRKLSAQEKDLVGKVFGGLTLLDTMQSETGVEAIRADVRTPHEEAVLNNIQFMESVHAKSYSSIFSTLNTKSEIEEIFEWTNNNEFLQEKARIINDIYANGNALQKKVASTYLETFLFYSGFFTPLYYLGNNKLANVAEIIKLIIRDESVHGTYIGYKFQLGFNELPEDEQENFRDWMYDLLYQLYENEEKYTKTLYDGVGWTEEVMTFLRYNANKALMNLGQDPLFPDTANDVNPIVMNGISTGTSNHDFFSQVGNGYLLGSVEAMHDDDYNYGL

SEQ ID NO: 45 - DNA, 311 bp, Streptococcus agalactiae
atgaattggtcacgtatctgggaactcgtaaaaattaatatcctttattcaaaccctcagactctatcggcactaagaaaaaagcaagaaaagcatcctaaaaaagaattttcagcttataaatccatgtttagaaatcagttatttcagattttgctcttttcaataatttatgtatttctctttgtatcacttgattttaaagaatatccgggctatttcacgttctacattggtatctttacactagtatccattatctactcttttattgcgatgtacagtgttttctatgagagtgacgatgttaa

SEQ ID NO: 46 - protein, 103 aa, Streptococcus agalactiae
MNWSRIWELVKINILYSNPQTLSALRKKQEKHPKKEFSAYKSMFRNQLFQILLFSIIYVFLFVSLDFKEYPGYFTFYIGIFTLVSIIYSFIAMYSVFYESDDV

SEQ ID NO: 47 - DNA, 312 bp, Streptococcus agalactiae
taatcttttagtcaacggagcaacaggaaaattgcaggctatgcgacagatattccaccacataatttagcagaagtcattgatgctgtcgtgtacatgattgatcaccctaaagctaaattagataaattaatggaatttctacctggtccagattttccaactggcgctatcattcaaggaaaagatgaaattcgtaaggcatatgagactggtaaggggagagtagcggttcgctcgcgaactgctattgaaaccttaaaaggtggtaagaaacaaattattgttactgaaattccttatgaagttaat

SEQ ID NO: 48 - protein, 103 aa, Streptococcus agalactiae
SFSQRSNRKIAGYATDIPPHNLAEVIDAVVYMIDHPKAKLDKLMEFLPGPDFPTGAIIQGKDEIRKAYETGKGRVAVRSRTAIETLKGGKKQIIVTEIPYEVN

SEQ ID NO: 49 - DNA, 654 bp, Streptococcus agalactiae
atgggacgtaagtgggccaatattgttgccaaaaagactgctaaagatggtgctaactcaaaagtatacgctaaattcggtgttgaaatatatgttgctgcaaagcaaggtgaaccagaccccgagtcaaactcagctctaaaattcgttttggaccgtgctaagcaagcacaagttccaaagcatgttattgataaagcgattgataaagccaaaggaaacacagatgaaactttcgtagagggacgctatgaaggttttggtccaaatggttcaatgattattgtggatactttgacatcaaatgttaaccgtacggcagcaaatgtacgtactgcttacggtaagaacggtggcaatatgggagcttcaggatcggtatcctacttatttgataaaaaaggtgtcatcgtttttgctggtgatgatgctgacactgtcttcgaacaattacttgaagcggatgtagacgtagatgatgttgaagcagaagagggaacaataacagtttataccgccccaacagatcttcataaaggtatccaagcacttcgcgataatggtgtagaagaattccaagttactgaacttgaaatgattcctcaatcagaagtagtattggaaggtgatgaccttgaaacttttgaaaagctt

SEQ ID NO: 50 - protein, 218 aa, Streptococcus agalactiae
MGRKWANIVAKKTAKDGANSKVYAKFGVEIYVAAKQGEPDPESNSALKFVLDRAKQAQVPKHVIDKAIDKAKGNTDETFVEGRYEGFGPNGSMIIVDTLTSNVNRTAANVRTAYGKNGGNMGASGSVSYLFDKKGVIVFAGDDADTVFEQLLEADVDVDDVEAEEGTITVYTAPTDLHKGIQALRDNGVEEFQVTELEMIPQSEVVLEGDDLETFEKL

SEQ ID NO: 51 - DNA, 135 bp, Streptococcus agalactiae
ttggagaaatatttgaagaacccgattacatggattggattagttcttgtggttacgtggtttttaactaaaagtagtgaatttttgatttttggtgtgtgtgtcttgttgttagtatttgctagtcaaagtgat

SEQ ID NO: 52 - protein, 45 aa, Streptococcus agalactiae
MEKYLKNPITWIGLVLVVTWFLTKSSEFLIFGVCVLLLVFASQSD

SEQ ID NO: 53 - DNA, 318 bp, Streptococcus agalactiae
atgacacaatcagatgcatatctctcgttgaacgcgaagacacgctttagagatcgcacaggtaattatcattttacttcggataaagaggctgttgaacaatatatgatagaacatgttgaacctaatacgatggtgttcacatcactaattgaaaagctagattatttggtttctaataactactatgaatcggaccttctaaaacaatataaccttgagtttatttgccaaatttttgagcatgcatacgctaagaaatttgcttttctaaattttatgggggctttaaaattttataatgcttatgctcttaat

SEQ ID NO: 54 - protein, 106 aa, Streptococcus agalactiae
MTQSDAYLSLNAKTRFRDRTGNYHFTSDKEAVEQYMIEHVEPNTMVFTSLIEKLDYLVSNNYYESDLLKQYNLEFICQIFEHAYAKKFAFLNFMGALKFYNAYALN

SEQ ID NO: 55 - DNA, 2451 bp, Streptococcus agalactiae
atggtatttatggcaaataagaaaaaaacaaaaggaaagaaaaccagaagacctactaaggcagaaatagagcgtcaaagagctattcaaaggatgattactgctcttgttttaacaattattctcttctttggtattatcagattaggtatttttggtattacagtctataacgtcatccgttttatggtaggtagcttggcttacttatttattgcggcaactttaatctacctttatttctttaaatggttgcgaaagaaagatagcttagtagcaggttttttgatagcttctttaggattattgattgagtggcatgcttaccttttctcaatgcctattttgaaagataaagaaattttgcgttcaactgctcgattaattgtgtctgatttaatgcaatttaaaatcactgtttttgccggtggaggtatgttgggtgctttgatttacaagccaattgcttttctcttttctaatattggtgcctatatgattggtgttctcttcatcattttgggtctctttttaatgagttctctggaagtttatgacatcgtcgaatttattagagcttttaaaaataaagtggcagagaagcacgagcaaaataaaaaggagcgttttgctaagcgagagatgaaaaaagcaatcgctgaacaagagcgcatagagcgtcaaaaagctgaagaagaagcttatttagcttcggttaatgtagaccctgaaacgggtgagattctagaggatcaagctgaggacaatttggatgatgcgctaccacctgaggtaagtgaaacatcaactccggtatttgagccagagatccttgcttatgagacatcgcctcaaaatgatcctttaccagtagagccgacaatttatttagaagactatgattcgccgattcctaatatgagagaaaatgatgaggaaatggtttatgatttagatgatgatgtagatgatagtgatatagaaaatgtcgactttacacctaaaacgacactggtttataaattaccaacgatagatttatttgcaccagataagcctaaaaatcaatccaaagaaaaggatttagtccgaaagaatatcagagttttagaagaaacatttagaagttttggtatcgatgtaaaagtagaacgtgctgaaattggaccatcagttactaaatatgaaattaaaccagcagttggagttcgtgtgaatcgtatttcaaatctatctgacgacctagctcttgctcttgcagcaaaagatgtgcgtatagaagcaccaattcctggaaaatcattaataggtattgaagttcctaactcagaaattgcaacggtttctttccgcgaactttgggaacaatctgatgccaatcctgaaaaccttttagaagtaccactaggaaaagctgttaacggcaatgctcgcagttttaacttagctagaatgccgcatcttttggtagctggttcaactggttcaggtaaatctgtggcagttaatggaattatttcaagtattttgatgaaggcacgtccagatcaagttaagtttatgatgattgatcccaaaatggttgaattatctgtttataatgatattccacatttattaatccctgttgtaaccaatccgcgtaaagcaagtaaggcactccaaaaagttgttgatgaaatggaaaatcgatacgagttatttagcaaaattggtgtgcgtaatatagcaggttataatacaaaggttgaagagtttaatgcttcctctgagcaaaaacaaatgcctttgcctttaatcgttgtcattgtagatgaattggctgacttgatgatggttgctagtaaagaagttgaagatgctattattcgtttggggcaaaaagcacgtgctgcaggtatccatatgattcttgcaactcaacgtccatccgtagatgttatttctggtttgattaaagcaaatgttccgtcgcgtattgcatttgctgtttcaagtggtactgatagccgtacgatccttgatgaaaatggtgctgaaaagctcttgggacggggtgacatgctctttaagcctattgatgagaatcatccagtacgactacaaggttcctttatttcagatgatgatgttgaaaggatcgttggttttatcaaagaccaagccgaggctgactatgatgatgcctttgatcctggagaagtatctgaaacagataacggctctggtggtggcggcggagtacctgaaagtgatcctctttttgaagaagccaagggactcgttttagagacgcaaaaagcaagtgcctcaatgattcaacgccgattgtctgttggtttcaatagagcaacaagactaatggaagaattagaagcagcgggggttattggtccagcagaaggaaccaagccacgaaaagttttaatgactccaactccgagtgaataa

SEQ ID NO: 56 - protein, 816 aa, Streptococcus agalactiae
MVFMANKKKTKGKKTRRPTKAEIERQRAIQRMITALVLTIILFFGIIRLGIFGITVYNVIRFMVGSLAYLFIAATLIYLYFFKWLRKKDSLVAGFLIASLGLLIEWHAYLFSMPILKDKEILRSTARLIVSDLMQFKITVFAGGGMLGALIYKPIAFLFSNIGAYMIGVLFIILGLFLMSSLEVYDIVEFIRAFKNKVAEKHEQNKKERFAKREMKKAIAEQERIERQKAEEEAYLASVNVDPETGEILEDQAEDNLDDALPPEVSETSTPVFEPEILAYETSPQNDPLPVEPTIYLEDYDSPIPNMRENDEEMVYDLDDDVDDSDIENVDFTPKTTLVYKLPTIDLFAPDKPKNQSKEKDLVRKNIRVLEETFRSFGIDVKVERAEIGPSVTKYEIKPAVGVRVNRISNLSDDLALALAAKDVRIEAPIPGKSLIGIEVPNSEIATVSFRELWEQSDANPENLLEVPLGKAVNGNARSFNLARMPHLLVAGSTGSGKSVAVNGIISSILMKARPDQVKFMMIDPKMVELSVYNDIPHLLIPVVTNPRKASKALQKVVDEMENRYELFSKIGVRNIAGYNTKVEEFNASSEQKQMPLPLIVVIVDELADLMMVASKEVEDAIIRLGQKARAAGIHMILATQRPSVDVISGLIKANVPSRIAFAVSSGTDSRTILDENGAEKLLGRGDMLFKPIDENHPVRLQGSFISDDDVERIVGFIKDQAEADYDDAFDPGEVSETDNGSGGGGGVPESDPLFEEAKGLVLETQKASASMIQRRLSVGFNRATRLMEELEAAGVIGPAEGTKPRKVLMTPTPSE

SEQ ID NO: 57 - DNA, 669 bp, Streptococcus agalactiae
atgtcacaagagcaaggaaaaatttatattgtagaagatgatatgacgattgtgtcacttttaaaagatcatttatcagctagctatcatgtctctagtgtcagcaattttcgtgatgtgaaacaagaaattatcgcatttcaacccgatttgatactaatggatattacgttaccctattttaatggtttttactggactgcagaattgcgtaagtttttaacaattcctattattttcatttcatctagtaatgatgaaatggatatggttatggcattaaatatggggggtgatgactttatttcaaaaccattctctctagctgtattagatgctaagctaactgctattttaaggagaagtcaacaatttatccaacaggaattaacttttgggggatttacgttgacaagagaagggttattgtctagccaagataaagaggttattttatcgccaacagaaaataaaatcctatctatcttgctcatgcatcctaaacaagtagtctcaaaagagtctctattagagaaactttgggaaaatgatagttttattgatcaaaatacacttaatgttaatatgacacgcttacgtaaaaaaattgtcccaataggttttgattacattcatacagtgagaggagttgggtatttactacaatga

SEQ ID NO: 58 - protein, 222 aa, Streptococcus agalactiae
MSQEQGKIYIVEDDMTIVSLLKDHLSASYHVSSVSNFRDVKQEIIAFQPDLILMDITLPYFNGFYWTAELRKFLTIPIIFISSSNDEMDMVMALNMGGDDFISKPFSLAVLDAKLTAILRRSQQFIQQELTFGGFTLTREGLLSSQDKEVILSPTENKILSILLMHPKQVVSKESLLEKLWENDSFIDQNTLNVNMTRLRKKIVPIGFDYIHTVRGVGYLLQ

SEQ ID NO: 59 - DNA, 1341 bp, Streptococcus agalactiae
atgtatcaaactcagacaaataaggaaaaatttgttttatttttgaaattatttatcccagtattgatttatcaatttgctaatttttcagctacttttattgattcggttatgactggacagtatagtcagctacatttggcaggtgtgtcaactgctagtaatttatggactccgtttttcgctttattagtaggtatgatttcagcattagtaccagtagttggtcaacatttgggtagaggaaataaagaacaaattcgcacagaatttcatcaatttctatatttaggtttgatactgtccttaatattatttttaatcatgcaatttattgctcaacctgtcttggggagtttgggtttagaagatgaagttctagcagttggtcgtggttatttaaattatatgttgattggaatcatgccgctggtgttgtttagcatttgccgttcattctttgatgcattggggttaacaaggttatctatgtatctgatgcttttaattctaccctttaattcattttttaattatatgcttatctacggtaaatttggtatgcctagactaggaggtgcgggggcaggtcttggaacttctttaacttattgggctatttttattggtattattattgtgatgtcacttcatcctcaaattaaaacatatcatatatggactctggaaagaataaaagctcctttgattattgaagatattcgattgggattaccgattggtttacaaatttttgcagaagttgcaatttttgcagtagtaggcttattcatggcaaaattttcttcaatcattattgcagcacatcaggctgctatgaatttttcatcattaatgtatgcatttcctttaagtatttccactgctctagctattacaatatcgtttgaagtaggggcagagcgctttcaggacgcaaccacttatagtaggataggacgcttaacagcggtagggattacatcaggaaccttactatttttatttctatttcgtgagaatgtagcagcaatgtataatagtgcccctcactttgtcgctattacagctcaattcctaacttatagtctctttttccagtttgcagatgcttatgcagctcctgtacaggggattttacgaggctataaggatacaacaaaaccatttatgatcggtgcgggctcttattggttatgtgctttgccattagcggttatcttagaaaaaaatagccagttaggtccgtttgcctattggattggtttaatcacaggtatttttgtttgtggtctatttctaaaccaacgtctgcaaaagattaagaagttgtattattaa

SEQ ID NO: 60 - protein, 446 aa, Streptococcus agalactiae
MYQTQTNKEKFVLFLKLFIPVLIYQFANFSATFIDSVMTGQYSQLHLAGVSTASNLWTPFFALLVGMISALVPVVGQHLGRGNKEQIRTEFHQFLYLGLILSLILFLIMQFIAQPVLGSLGLEDEVLAVGRGYLNYMLIGIMPLVLFSICRSFFDALGLTRLSMYLMLLILPFNSFFNYMLIYGKFGMPRLGGAGAGLGTSLTYWAIFIGIIIVMSLHPQIKTYHIWTLERIKAPLIIEDIRLGLPIGLQIFAEVAIFAVVGLFMAKFSSIIIAAHQAAMNFSSLMYAFPLSISTALAITISFEVGAERFQDATTYSRIGRLTAVGITSGTLLFLFLFRENVAAMYNSAPHFVAITAQFLTYSLFFQFADAYAAPVQGILRGYKDTTKPFMIGAGSYWLCALPLAVILEKNSQLGPFAYWIGLITGIFVCGLFLNQRLQKIKKLYY

SEQ ID NO: 61 - DNA, 1029 bp, Streptococcus agalactiae
ttgctagtttcttctctagtttcttgttcattttttcttgtcatttcgtcgttgtcttcatcaacacgaaataagtctataaacttatcaaataatttcatagacttattatatcaattttcaataaaatgctataataaaaccatgtcattttcattaaaaattagaaatccatacggtgaacataccgttaaagaactccttgaagattattttttgattccacgtaagattagacattttttgcgtgttaaaaaacatgtacttataaacaatgaattcattaattggcaaactgtcgtccaagaaaacgatactattaccttaatctttgatgatgaggattaccctactaaaaaaattcctctgggcagagcagagcttattgattgtctttatgaggatgaacatcttattatcgttaataaacctgaaggtatgaaaactcacggtaaccaaccaaatgaaatagcactgttaaatcatgtatctgcctattctggacaaacatgctatgttgttcatcgcctagatatggagaccagtggagctgttttatttgctaaaaatccatttatacttccccttatcaatcaacgcttagaacgaaaagaaatttggcgtgaatattgggctttagttgaaggaaaattttcacctaagcatcaagttttgagagacaaaattggacggaaccgtcatgacagacgtaaacgaatcattgattctaaaaacggtcaacatgctatgacaatcattgacgttttgaagtatatccaaaatagtagtctcataaaatgccgactggaaaccggaagaacccatcaaattcgcattcacttatctcatcacggacatcctttaataggagatcccctctacaacccttcttctaataatgaaaggttaatgctacacgctcaccgattgactctatcccatccattaacttgcgaaactattagcgtagaggccccttcatctactttcgagaaggttttaaacaattataaaaaaggagttggataa

SEQ ID NO: 62 - protein, 342 aa, Streptococcus agalactiae
MLVSSLVSCSFFLVISSLSSSTRNKSINLSNNFIDLLYQFSIKCYNKTMSFSLKIRNPYGEHTVKELLEDYFLIPRKIRHFLRVKKHVLINNEFINWQTVVQENDTITLIFDDEDYPTKKIPLGRAELIDCLYEDEHLIIVNKPEGMKTHGNQPNEIALLNHVSAYSGQTCYVVHRLDMETSGAVLFAKNPFILPLINQRLERKEIWREYWALVEGKFSPKHQVLRDKIGRNRHDRRKRIIDSKNGQHAMTIIDVLKYIQNSSLIKCRLETGRTHQIRIHLSHHGHPLIGDPLYNPSSNNERLMLHAHRLTLSHPLTCETISVEAPSSTFEKVLNNYKKGVG

SEQ ID NO: 63 - DNA, 2052 bp, Streptococcus agalactiae
gaactaaatgcaactcaacctaataatagaactacctatattatacccgaaagcagtcattccattgcagaacaacagagattcctgatagaatcaaagggttcttcggttgcattacttaatagcgatgaatttagaaagacagcgggagaggatagaggttttgaaagggataagttgaggtctttggatatcattcctaagggagatttatcgacaagtaatgtcataggtaatacggacattgctagtcagatatcgttgggctttaaaaagaatgcgatgcaggaacaccatcttactaaaacattctctcaaaaggatggaaagttatcgtctgttatagaggggatgcttgctattggcaaagagaaagtagagaaagaaataaaatatagtggtaatttatggcaaaaattaaaagctaaggcacactgccttgtttgctgtgttgataatttgaattttgaagatataaaatcttattttcaatattattgtcatctaaaccatcagctcaaattacctaaaggtgctatactttctgctaaaacagaagtatataggggaggagattttgggagaaaaaataaagataatgtgtttggttaccgtatcccctcattattgaaaacccaaaaaggaactttacttgcgggagctgatgaaagaattgagcaagcttgtgattggggaaacataggaatggttattcgccgtagtgaggatgatggtgtcacttggggaaaaagagaaactattgtcaatctccgtaataaccctagagttccgctagttactagtggtgactatagtggctcacctattaatatggatatggcattagttcaagatactagctccaagacgaaacgtattttttcaatatatgatatgtttccagaaggaagaggcgttattagtattgctaacacacctgaaaaagaatatacccaaatcggaggacagtcttatcttaatttatataataatggaaagaaatcgaaggtttttactatccgtgacaaaggtattgtatataattttaaagggaaaaagactgattatcatgttataacagaaactactaaaagtgaccattcaaatctaggggatatttataagggaaaacagctacttggaaatatatattttacaaaacataaaacgtcaccatttcgtttagcaaaatcaagctatgtgtggatgtcatatagcgatgatgatggtaggacatggtcatcacctagagatataacagcaagtcttcgtcagaaaggcatgaaatttttgggaataggacctggaaaaggtatagttttaaaatgggggccacacgctggtcgtattattattcctgcctattctacgaattggaaatctcatctaagaggttcacaatcttcacgcctaatttattcagacgaccatggaaaaacgtggcatactggaaaagcagttaatgataaccgtatactttctaatggtgaaaaaattcactccttaacaatggataataaaaaagaacaaaatacagaatccgtacccgttcaattgaaaaatggggacattaagttatttatgaggaatctaactggtaacctagaagtagccacaagtaaagacggcggggagacttggcaaaaccatgttaaacgatataaggaaattcatgatgcttacgtccaactatcagctattcgctttgagcatgacaaaaaagagtatattttattagtgaatgctaatgggccagggaagaagtgccaagatggatatgcacgtctagcgcaagttaatcgaaatggtagttttaagtggttatatcaccatcacattcaagatggttcgtttgcttacaactctgttcaacaacttaataatgatcaatttggtgtcctttatgaacatagagaaaaacatcaaaatagttttactttaaattacaaagtttttaattggagttttcttagtcaaaatacagagaagcaaggcactttatgggagaaaatggcagcaaattggcatgttttgtttaaattttatttatga

SEQ ID NO: 64 - protein, 683 aa, Streptococcus agalactiae
ELNATQPNNRTTYIIPESSHSIAEQQRFLIESKGSSVALLNSDEFRKTAGEDRGFERDKLRSLDIIPKGDLSTSNVIGNTDIASQISLGFKKNAMQEHHLTKTFSQKDGKLSSVIEGMLAIGKEKVEKEIKYSGNLWQKLKAKAHCLVCCVDNLNFEDIKSYFQYYCHLNHQLKLPKGAILSAKTEVYRGGDFGRKNKDNVFGYRIPSLLKTQKGTLLAGADERIEQACDWGNIGMVIRRSEDDGVTWGKRETIVNLRNNPRVPLVTSGDYSGSPINMDMALVQDTSSKTKRIFSIYDMFPEGRGVISIANTPEKEYTQIGGQSYLNLYNNGKKSKVFTIRDKGIVYNFKGKKTDYHVITETTKSDHSNLGDIYKGKQLLGNIYFTKHKTSPFRLAKSSYVWMSYSDDDGRTWSSPRDITASLRQKGMKFLGIGPGKGIVLKWGPHAGRIIIPAYSTNWKSHLRGSQSSRLIYSDDHGKTWHTGKAVNDNRILSNGEKIHSLTMDNKKEQNTESVPVQLKNGDIKLFMRNLTGNLEVATSKDGGETWQNHVKRYKEIHDAYVQLSAIRFEHDKKEYILLVNANGPGKKCQDGYARLAQVNRNGSFKWLYHHHIQDGSFAYNSVQQLNNDQFGVLYEHREKHQNSFTLNYKVFNWSFLSQNTEKQGTLWEKMAANWHVLFKFYL

SEQ ID NO: 65 - DNA, 1188 bp, Streptococcus agalactiae
atgcctaaattaatcgtatctttcctctgcattttattatccctgacttgtgtaaactctgtgcaagctgaagaacataaagatattatgcaaattacccgagaagccggatatgatgttaaagatattaataaacctaaagcgtctatcgttattgacaataaaggtcatattttgtgggaagataacgccgatttagaacgtgatcccgctagcatgtctaaaatgtttactttatatttactatttgaagacttagctaaaggaaaaacaaacctcaacaccacagtgactgcaacagaaacagaccaagccataagtaagatttatgaaattagtaataacaatattcatgctggggttgcttatcctattcgtgaactgattactatgacggctgtcccgtcatctaatgtagcaactattatgattgctaaccacttatcacaaaacaatcctgacgcctttattaaacgaatcaatgaaaccgccaagaaactcggtatgacaaaaactcacttttataaccccagtggggcggtagcgagtgcttttaatggactttactccccaaaagaatacgataacaatgctactaacgttacgactgcacgtgatctatcaattttaacctatcatttccttaaaaaataccctgatatactgaactatacaaaatatcctgaagtcaaggccatggtcggaactccttatgaagaaacatttacaacttataactactctacccccggcgctaaatttggattagaaggagtagatggcttaaaaactggttctagccctagcgctgcttttaatgccttagttacagctaaacgccagaatactcgcttgataactgtggttttaggagttggcgattggtcagaccaagacggagagtactatcgtcatccgtttgtcaacgctcttgtagaaaaaggttttaaagacgctaaaaatatttcttctaaaactcctgtattaaaagccgttaaacctaaaaaagaagttactaaaaccaaaactaaatctattcaagaacagcctcaaacaaaagaacagtggtggacaaaaacagatcaatttatccaatcacattttgtatctattttaattgttctgggcaccatcgctagcctttgtcttttagctgggatagtattacttataaagcgctctagataa

SEQ ID NO: 66 - protein, 395 aa, Streptococcus agalactiae
MPKLIVSFLCILLSLTCVNSVQAEEHKDIMQITREAGYDVKDINKPKASIVIDNKGHILWEDNADLERDPASMSKMFTLYLLFEDLAKGKTNLNTTVTATETDQAISKIYEISNNNIHAGVAYPIRELITMTAVPSSNVATIMIANHLSQNNPDAFIKRINETAKKLGMTKTHFYNPSGAVASAFNGLYSPKEYDNNATNVTTARDLSILTYHFLKKYPDILNYTKYPEVKAMVGTPYEETFTTYNYSTPGAKFGLEGVDGLKTGSSPSAAFNALVTAKRQNTRLITVVLGVGDWSDQDGEYYRHPFVNALVEKGFKDAKNISSKTPVLKAVKPKKEVTKTKTKSIQEQPQTKEQWWTKTDQFIQSHFVSILIVLGTIASLCLLAGIVLLIKRSR

SEQ ID NO: 67 - DNA, 984 bp, Streptococcus agalactiae
atgactgaaaaatattataattgggcaacgcttggaaccggcgttattgccaacgaattagcccaagcactggaagcacgtggacaaaaattatattctgtagctaatagaacttacgacaaaggacttgaatttgctaacaaatatggtatccaaaaagtttatgatcacatagatcaagtatttgaagaccctgaagtggatatcatttatatctctactccccacaatactcacatctcatttttacgaaaggctttagcaaatggtaagcacgttctttgcgaaaaatctattactttaaatagtactgagcttaaagaagccatagatttagccgaaactaaccatgttgtcttagctgaagccatgactatttttcatatgccaatttaccgccaattaaaaacattagttgatagtggaaaattaggaccgttaaaaatgattcaaatgaatttcggaagttataaagaatatgatatgactaaccgttttttcagtcgtgacctagcaggcggtgctttgctggacattggtgtttatgcactttcttgtattcgctggtttatgtcagaagcacctcacaacattacctctcaagttacatttgcaccaacaggggttgatgaacaagttggtatcctactaaccaacccagcaaatgagatggcgactgtcagccttagtttacatgcaaaacaacctaaacgagcaactatcgcttacgataaaggctacattgaactttttgaatatccgcgaggacaaaaggcagttattacttatactgaggatgggcatcaagatattatcgaagctggcaaaactgaaaatgctctccaatatgaggtagctgatatggaagaagccatttcaggaaaaactaaccacatgtacttaaactataccaaagatgttatggatatcatgacacagctacgtcaagaatggggatttacctacccagaagaagaaaaatga

SEQ ID NO: 68 - protein, 327 aa, Streptococcus agalactiae
MTEKYYNWATLGTGVIANELAQALEARGQKLYSVANRTYDKGLEFANKYGIQKVYDHIDQVFEDPEVDIIYISTPHNTHISFLRKALANGKHVLCEKSITLNSTELKEAIDLAETNHVVLAEAMTIFHMPIYRQLKTLVDSGKLGPLKMIQMNFGSYKEYDMTNRFFSRDLAGGALLDIGVYALSCIRWFMSEAPHNITSQVTFAPTGVDEQVGILLTNPANEMATVSLSLHAKQPKRATIAYDKGYIELFEYPRGQKAVITYTEDGHQDIIEAGKTENALQYEVADMEEAISGKTNHMYLNYTKDVMDIMTQLRQEWGFTYPEEEK

SEQ ID NO: 69 - DNA, 96 bp, Streptococcus agalactiae
gtgtattctcctgttaaatcttctaaaggaaaagtgatattgttaaaaagtgattttctaaagagcttcatagaaaggagaggaaatatttgtttt

SEQ ID NO: 70 - protein, 32 aa, Streptococcus agalactiae
MYSPVKSSKGKVILLKSDFLKSFIERRGNICF

SEQ ID NO: 71 - DNA, 429 bp, Streptococcus agalactiae
aaatactgtatcattgcaacctcaaatgcaggttttggaaacgaagcatttacaggtgacagcgataaagacttgaaaattatggaacgaatttctccatatttccgtccagaatttctaaatcgtttcaatggtgttattgaattctctcacctaagcaaagatgacttaagcgaaattgtagatttgatgcttgatgaagttaaccaaacaattggcaaaaaaggaattgaccttgtggtagatgaaaatgttaaatcacacttaattgaactgggttatgacgaagcaatgggagtacgtccattgcgccgtgtcatcgagcaagaaattcgagatcgcatcacagactactatctcgatcatacagacgttaaacacctaaaagctaatttgcaagatggccaaatcgtcatttctgaaagataa

SEQ ID NO: 72 - protein, 142 aa, Streptococcus agalactiae
KYCIIATSNAGFGNEAFTGDSDKDLKIMERISPYFRPEFLNRFNGVIEFSHLSKDDLSEIVDLMLDEVNQTIGKKGIDLVVDENVKSHLIELGYDEAMGVRPLRRVIEQEIRDRITDYYLDHTDVKHLKANLQDGQIVISER

SEQ ID NO: 73 - DNA, 699 bp, Streptococcus agalactiae
atgtcaatgaatttttcatttttaccacaatattggtcctattttaattatggtgtgatggtaaccattatgatttcaacatgtgttgttttttttggaactattataggcgtgttaattgctttagtaaagcgtactaatttacattttctcacaatattagctaatttctatgtatgggtatttcgtgggacaccgatggtagttcaaattatgattgctttcgcatggatgcattttaacaatttaccaacaattagctttggtgttttagatttagattttacacgacttttacctggtatcattatcatttccttaaatagtggtgcctatatttcggaaattgtacgtgcagggattgaggctgtaccatctggacaaatagaagcagcttactcgttggggattcgacctaaaaatacacttcgctatgttatcttaccccaagcttttaaaaatattttacctgctctagggaatgaatttattacaattattaaagatagtgctctccttcaaactattggtgtcatggaattatggaacggagcacaatcagttgtaacggctacttactcaccagttgcaccgttattatttgcagcattttactatttaatgttgacaacgattctctcagctttgttaaaacaaatggagaaatatcttgggaaaggggtaaaaatagatggttga

SEQ ID NO: 74 - protein, 232 aa, Streptococcus agalactiae
MSMNFSFLPQYWSYFNYGVMVTIMISTCVVFFGTIIGVLIALVKRTNLHFLTILANFYVWVFRGTPMVVQIMIAFAWMHFNNLPTISFGVLDLDFTRLLPGIIIISLNSGAYISEIVRAGIEAVPSGQIEAAYSLGIRPKNTLRYVILPQAFKNILPALGNEFITIIKDSALLQTIGVMELWNGAQSVVTATYSPVAPLLFAAFYYLMLTTILSALLKQMEKYLGKGVKIDG

SEQ ID NO: 75 - DNA, 678 bp, Streptococcus agalactiae
atgaaagacctattacgaaatagtctagagcaaagtggaaatttaagttttcaagatatgattttacatattcttgtagcagctttattgagtgtagttatttatgtttcctatgcttatacgcatagtggaactgcctatagtaaaaagtttaatgtttcattaatgacattgacggtcttgactgcaacagtaatgaccgttattggtaataatgtagccttgtcattgggtatggtcggtgccttgtcagttgttcgttttaggacagccataaaagattcaagagatacagtttatattttttggaccatagttgttggtatctgttgtggtgtcggtgactatgtggtagctgcattaggaagtagcgttatctttatcttattatgggttatgggacgtgttaaaaacgagaatcgtatgttattgattgtgaagtgcgatagaacactagaagttgatttagaaggaattttcttccaatattttgacggaaaagctgttcagcgtgttaaaaattcaacaactaatactattgaaatgattttcgaaatctctagaaaagattacgataagcaactccatgtagataatcagttaactgaaaaagtgtaccaattgggaaatattgattatttcaacattgttagccaaagcgacgaaatcaatgggtag

SEQ ID NO: 76 - protein, 225 aa, Streptococcus agalactiae
MKDLLRNSLEQSGNLSFQDMILHILVAALLSVVIYVSYAYTHSGTAYSKKFNVSLMTLTVLTATVMTVIGNNVALSLGMVGALSVVRFRTAIKDSRDTVYIFWTIVVGICCGVGDYVVAALGSSVIFILLWVMGRVKNENRMLLIVKCDRTLEVDLEGIFFQYFDGKAVQRVKNSTTNTIEMIFEISRKDYDKQLHVDNQLTEKVYQLGNIDYFNIVSQSDEING

SEQ ID NO: 77 - DNA, 499 bp, Streptococcus agalactiae
aaaaattcattttagattcattttacgactatatactcagaagtaccaaacctaatccaaggtttgaaaaaagaaagaaggaagtcagtatgacaaactataaaaacaactttaaagatgaggctatacgtgttgaagagacaacaaaagaatcattttacgatgttgatattgccttgttttcagctggtggatctatttcagcaaagttcgctccttatgcagtaaagtctggagcagttgtagtagataacacgtcatattttcgtcagaatcctgatgttccactagttgttcctgaagtaaatgctcatgccatgattggtcataatggtatcatagcttgtcccaattgttctactattcaaatgatgattgctttagagcccattcgtcaaaaatgggggatagagcgtgttatagtttccacctatcaagctgtttcgggttcaggtgcacgtgctgttgaagaaactaaggaacagttgagacaagtttt

SEQ ID NO: 78 - protein, 165 aa, Streptococcus agalactiae
KFILDSFYDYILRSTKPNPRFEKRKKEVSMTNYKNNFKDEAIRVEETTKESFYDVDIALFSAGGSISAKFAPYAVKSGAVVVDNTSYFRQNPDVPLVVPEVNAHAMIGHNGIIACPNCSTIQMMIALEPIRQKWGIERVIVSTYQAVSGSGARAVEETKEQLRQV

SEQ ID NO: 79 - DNA, 456 bp, Streptococcus agalactiae
atgacaaatgaattgataatgcaagcttttgagtggtatttacctagtgatgggaatcactggaagaaattagaggagtctatatcagaccttaaaaaacttggaattagtaaaatctggttaccaccagcatttaagggaactagcagtgatgatgtaggatatggtgtttatgatctctttgatttaggagaatttgaccagaatggaacaattagaacaaaatatggtaggaaagaagagtatctaaagcttattaagtcgttaaaggcaaatggcattaaaccgtttgcagatatcgttcttaaccataaagccaatggtgatcataaagaaaaatttcaagtcatcaaagtcaatcctgaaaatcgtcaagaagcattaagtgaaccctatgagattgaaggatggacgggatttgatttcccaggtagacagggtgagtacaatgatttt

SEQ ID NO: 80 - protein, 152 aa, Streptococcus agalactiae
MTNELIMQAFEWYLPSDGNHWKKLEESISDLKKLGISKIWLPPAFKGTSSDDVGYGVYDLFDLGEFDQNGTIRTKYGRKEEYLKLIKSLKANGIKPFADIVLNHKANGDHKEKFQVIKVNPENRQEALSEPYEIEGWTGFDFPGRQGEYNDF

SEQ ID NO: 81 - DNA, 516 bp, Streptococcus agalactiae
atggaggttcttatgaagaaagtgttagtaagtagtcttttggttttagggattacgataacgttacaaccagtagttgaggctaaggggccaaaagtagcttatacacaagagggaatgactgctctttcggacacaaataaagataaagtcactactatttctattgacgagattcaaaaaagcttagaaggtaagaagccgattactgttagttttgatattgatgatacactgcttttcagtagtcaatattttcaatatggtaaagaatatgtaactcctggatcgtttgattttcttcataaacaaaaattctgggatcttgttgcaaaacgaggagatcaagattccattcccaaagaatatgctaaaaaattaattgctatgcatcaaaaacgaggagataaaattgtttttataacaggtaggacaagagggtcaatgtataaggagggcgaggttgataaaacagctaaagccttagctaaagattttaaatttgtaccatctgat

SEQ ID NO: 82 - protein, 172 aa, Streptococcus agalactiae
MEVLMKKVLVSSLLVLGITITLQPVVEAKGPKVAYTQEGMTALSDTNKDKVTTISIDEIQKSLEGKKPITVSFDIDDTLLFSSQYFQYGKEYVTPGSFDFLHKQKFWDLVAKRGDQDSIPKEYAKKLIAMHQKRGDKIVFITGRTRGSMYKEGEVDKTAKALAKDFKFVPSD

SEQ ID NO: 83 - DNA, 516 bp, Streptococcus agalactiae
atgcttaaaagattatttactgaagatggggaattgacaaagattagtcgtcgtttcgtttggatgttagtggttatctattgtcttattattgtcaggatgtgttttgggcctcaaattatgattgagggggtatcaactccgaatgttcagcgcttcggaagaattgtagctcttttagtaccatttaattcttttcgtagtttagatcagctaactagctttaaagagattctttgggttattggtcaaaatgtagtgaatattttactgctgtttcctctcattatagggttactatccctaaagccaagtttacggaaatataaaagcgttatattacttgctttcttgatgtctcttttcatagagtgtactcaagttgttttagatattttaatagatgctaatcgggtttttgaaatcgacgatctatggacaaataccttaggcggtcctttcgccctatggagttatcgaaacataaaaggttggcttctaactattagaaaatga

SEQ ID NO: 84 - protein, 171 aa, Streptococcus agalactiae
MLKRLFTEDGELTKISRRFVWMLVVIYCLIIVRMCFGPQIMIEGVSTPNVQRFGRIVALLVPFNSFRSLDQLTSFKEILWVIGQNVVNILLLFPLIIGLLSLKPSLRKYKSVILLAFLMSLFIECTQVVLDILIDANRVFEIDDLWTNTLGGPFALWSYRNIKGWLLTIRK

SEQ ID NO: 85 - DNA, 627 bp, Streptococcus agalactiae
atgaaaaagcttacttttatttgggatttagatgggacattaatagattcgtatgtaccaattatggaagctcttgaagaaacctatcgtcattttggcttaatatttgataaagaattaatccatgaatatattttacaggaatcagtggggcaattattggtaaacctttcagaggaagagcaaatacctcatgaaaaactgaaagcatattttacaaaagaacaagaaagtcgagattctaaaatacatttaatgccatatgcaaaagagattttagaatggaccaaagaacaagatattcccaattttatgtatacacataaaggagcaagtacgcattcagtgttggaaaccttgcagatctctcattattttgatgaaattttaactggtgtttcgggattcgagcgaaaaccacatccacaagggattaattatttagttaaacgatattctttagataaatcaatgacttattacataggagatcgtccactagatttggaggttgctcaaaatgctggtataaaatccataaacttaaggttagagaattccaaagaaaactataatatttcaagtctcaaagatataatatcacttgatttcactcgtttggattaa

SEQ ID NO: 86 - protein, 208 aa, Streptococcus agalactiae
MKKLTFIWDLDGTLIDSYVPIMEALEETYRHFGLIFDKELIHEYILQESVGQLLVNLSEEEQIPHEKLKAYFTKEQESRDSKIHLMPYAKEILEWTKEQDIPNFMYTHKGASTHSVLETLQISHYFDEILTGVSGFERKPHPQGINYLVKRYSLDKSMTYYIGDRPLDLEVAQNAGIKSINLRLENSKENYNISSLKDIISLDFTRLD

SEQ ID NO: 87 - DNA, 1356 bp, Streptococcus agalactiae
atggaaaaagaaaaaaaattaggtcttttaccactaacaatgcttgtcattggctctcttatcggtggcggaatctttgatttaatgcaaaatatgagttccagagccggtttggtaccaatgcttattgcttgggtaattactgctatcgggatgggaactttcgttttaagttttcaaaatttatctgaaaaaaggccggacctaacagctggaatctttagttacgctaaagaggggtttggaaactttatgggatttaactctgcatggggttattggttatcagcttggcttggaaatgttgcctacgctgcactcttattcagttcactcggttatttctttaaattctttggtaatggaaataatatcatctcaattattggagcaagtatagttatttgggttgtccatttcttaattttaagaggtgttaatacagctgcatttattaataccgtagttacctttgcaaaattagtacctgttattattttcttaatttcagcgttattagctttcaaatttaacatttttagtcttgatatctggggaaatggattacatcaatcaattttcaaccaagtcaattcaactatgaaaaccgctgtttgggtatttattggtattgagggcgccgttgtcttctcaggtcgtgctaaaaaacactctgatattggtaaagcaagtatcctagcattattcactatgatttcactttatgtattgatttctgttttatcacttggtatcatgtcacgtccagaacttgcaaacttaaaaacaccagctatggcttacgttctagaaaaagctgttggtcactggggtgctatcttagttaaccttggtgttatcatttcagtatttggcgctattcttgcttggactttatttgcagcagaattaccatatcaagctgctaaagaaggtgcttttcctaaattttttgcaaaagaaaataaaaacaaagctccaatcaactcactcttagtcactaatctttgtgtacaagcattcttaatcacgttcttattcacacaaagtgcttatcgttttggtttcgcattagcatcatctgctatcttaattccttatgcttttacagcactatatcaattacaattcacactccgtgaggataagtcaactccaggacatcaaaagaatttaattatcggtatcctcgctacaatctatgctgtttaccttatctacgctggtggttttgattacttacttttgacaatgattgcttatactctaggtatgattctctatattaaaatgagaaaagatgacaagcttggcgtaatcatggtcatagctgtttccagtgtgaaattgttatcc

SEQ ID NO: 88 - protein, 452 aa, Streptococcus agalactiae
MEKEKKLGLLPLTMLVIGSLIGGGIFDLMQNMSSRAGLVPMLIAWVITAIGMGTFVLSFQNLSEKRPDLTAGIFSYAKEGFGNFMGFNSAWGYWLSAWLGNVAYAALLFSSLGYFFKFFGNGNNIISIIGASIVIWVVHFLILRGVNTAAFINTVVTFAKLVPVIIFLISALLAFKFNIFSLDIWGNGLHQSIFNQVNSTMKTAVWVFIGIEGAVVFSGRAKKHSDIGKASILALFTMISLYVLISVLSLGIMSRPELANLKTPAMAYVLEKAVGHWGAILVNLGVIISVFGAILAWTLFAAELPYQAAKEGAFPKFFAKENKNKAPINSLLVTNLCVQAFLITFLFTQSAYRFGFALASSAILIPYAFTALYQLQFTLREDKSTPGHQKNLIIGILATIYAVYLIYAGGFDYLLLTMIAYTLGMILYIKMRKDDKLGVIMVIAVSSVKLLS

SEQ ID NO: 89 - DNA, 1134 bp, Streptococcus agalactiae
atgaaatttgaaaaacggcaggtctattatgttgtcataacatttgctatttgctatgctatacaggcttattggggagctgtttctaatattttaactacgcttcataaggcaatatttccttttttgatgggagctggaattgcctatattattaatattgtaatgtcagtctatgagcgattatatataaagctttttaaaggatctagactattaatggcaatcaagcgtagtgtttctatgattttatcctatgcaacttttattggtttaattgtctggctattttcaattgtcattccagatttgatttctagtttgagttctttattggttattgataccggagcacttgctaaattggttaataatctcaatgaaaataaacaaatttctgaggctttaaattatatgggaacagataaagacttagtttctactttaagtggttatagccagcagattttgaagcaagttttatctgttttaacaaatttactaacctcagtttcctctattgcggcaacacttctgaatgtttttgttagttttattttttcaatttacgttttggcaaacaaggagcagttgggacgtcaatttaatttgttaattgatacctatttaggttcaacaggcaaaacattccattacgttcgtcatatccttcatcaacgtttccatggtttttttgtaagccaaactttagaagctatgattttaggaagtttgacggttattggtatgttgatcttccaatttccttatgctttaacagttggggttttagttgcttttacagctctaataccggttgtgggagcctacattggtgttacaatcggtttcatcttaattgctactgaatcgcttactgaagcattcttgtttgttcttttcttgatccttttacaacaatttgagggaaatgtcatttatccgaaagttgtcggtggatcgattggactgccttctatgtgggttttaatggctattactatcggaggtgctttatgggggatcttaggcatgttacttgctgttcctgttgcagctactatctatcagattgtaaaagatcatattatcaagcgacaaacgcttagaaatcgtgcacgaacctatcgttaa

SEQ ID NO: 90 - protein, 377 aa, Streptococcus agalactiae
MKFEKRQVYYVVITFAICYAIQAYWGAVSNILTTLHKAIFPFLMGAGIAYIINIVMSVYERLYIKLFKGSRLLMAIKRSVSMILSYATFIGLIVWLFSIVIPDLISSLSSLLVIDTGALAKLVNNLNENKQISEALNYMGTDKDLVSTLSGYSQQILKQVLSVLTNLLTSVSSIAATLLNVFVSFIFSIYVLANKEQLGRQFNLLIDTYLGSTGKTFHYVRHILHQRFHGFFVSQTLEAMILGSLTVIGMLIFQFPYALTVGVLVAFTALIPVVGAYIGVTIGFILIATESLTEAFLFVLFLILLQQFEGNVIYPKVVGGSIGLPSMWVLMAITIGGALWGILGMLLAVPVAATIYQIVKDHIIKRQTLRNRARTYR

SEQ ID NO: 91 - DNA, 1386 bp, Streptococcus agalactiae
gtgattacaattaaaaaggaatctgttatcaaactattgaagtatgcttttggcattataatgggatttattatcttagctattgtaataggtgggctcctatttgcatactacgttagtcgttctccgaaattaaccgatcaagctttaaaatccgttaactctagtttggtttatgatggtaataataaacttattgccgatttaggctcagaaaagcgtgaaagtgttagtgcggatagcattccactaaatttggttaacgctatcacttctatagaagataaacgtttctttaaacatagaggtgtcgatatttatcgtattttaggtgcagcttggcataaccttgttagtagtaatacgcaaggtggttcaacccttgatcaacagttgattaaactggcttacttttctaccaataaatctgaccaaacgttaaaacgtaaatcacaggaagtttggcttgcgcttcaaatggagcgtaaatacaccaaagaagaaattcttactttctatattaataaagtttatatgggaaatgggaattatggtatgagaacaacagctaaatcatactttggtaaagacctaaaggaattatctattgcacaacttgctttgctcgctggtattcctcaagcacctacacaatatgacccttataaaaacccagaatctgctcaaacaagacgtaataccgttcttcagcagatgtatcaagataaaaacatttctaaaaaggaatacgaccaagctgttgcaactccagtaactgatggcttaaaagaattaaagcaaaaatctacttatccaaaatatatggataactacttaaaacaagttattagtgaagttaaacaaaaaactggtaaagatatctttactgctgggctaaaagtgtatactaatatcaacactgatgcacaaaaacaactatatgacatctacaacagtgatacttacatcgcttatccaaacaatgaattacaaatagcatctaccatcatggatgcgactaatggtaaagtcattgcacaattaggcgggcgtcatcagaatgaaaatatttcatttgggacaaatcaatctgtcttaacagaccgcgattggggttctacaatgaaacctatctcagcttatgcacctgctattgatagtggtgtctataattcaacaggtcaatcattaaacgactcagtttactactggcctggtacttctactcaactatatgactgggatcgtcaatatatgggttggatgagtatgcagaccgctattcaacaatcacgtaacgtccctgctgtcagagcacttgaagccgctggattagacgaagcaaaatctttccttgaaaaattaggcatatactatccagaaatg

SEQ ID NO: 92 - protein, 462 aa, Streptococcus agalactiae
MITIKKESVIKLLKYAFGIIMGFIILAIVIGGLLFAYYVSRSPKLTDQALKSVNSSLVYDGNNKLIADLGSEKRESVSADSIPLNLVNAITSIEDKRFFKHRGVDIYRILGAAWHNLVSSNTQGGSTLDQQLIKLAYFSTNKSDQTLKRKSQEVWLALQMERKYTKEEILTFYINKVYMGNGNYGMRTTAKSYFGKDLKELSIAQLALLAGIPQAPTQYDPYKNPESAQTRRNTVLQQMYQDKNISKKEYDQAVATPVTDGLKELKQKSTYPKYMDNYLKQVISEVKQKTGKDIFTAGLKVYTNINTDAQKQLYDIYNSDTYIAYPNNELQIASTIMDATNGKVIAQLGGRHQNENISFGTNQSVLTDRDWGSTMKPISAYAPAIDSGVYNSTGQSLNDSVYYWPGTSTQLYDWDRQYMGWMSMQTAIQQSRNVPAVRALEAAGLDEAKSFLEKLGIYYPEM

SEQ ID NO: 93 - DNA, 336 bp, Streptococcus agalactiae
atggctaatgtatatgatttagcaaatgaattagaacgtgctgttcgtgctttaccagaataccaagcagttttaactgcaaaagcagctattgaaaatgatgcggatgcacaagtgctttggcaagactttttggctacccaatcaaaagttcaagaaatgatgcaatctggccaaatgccaagtcaagaagaacaagatgaaatgtctaaacttggggaaaaaattgaatccaatgaccttttaaaagtttattttgaccaacaacaacggttgtctgtctatatgtctgatatcgaaaaaattgtctttgcacccatgcaggacttgatgtaa

SEQ ID NO: 94 - protein, 111 aa, Streptococcus agalactiae
MANVYDLANELERAVRALPEYQAVLTAKAAIENDADAQVLWQDFLATQSKVQEMMQSGQMPSQEEQDEMSKLGEKIESNDLLKVYFDQQQRLSVYMSDIEKIVFAPMQDLM

SEQ ID NO: 95 - DNA, 230 bp, Streptococcus agalactiae
atggcagaaatcacagctaaacttgtaaaagaattgcgtgaaaaatcaggtgcaggcgttatggacgctaaaaaagcattagtagaaactgatggtgaccttgataaagcgattgaattacttcgcgaaaaaggtatggctaaagcagctaaaaaagcagaccgtgttgctgctgaaggtttaacaggtgtttatgttgatggtaacgttgcagcagttattgaagttaa

SEQ ID NO: 96 - protein, 76 aa, Streptococcus agalactiae
MAEITAKLVKELREKSGAGVMDAKKALVETDGDLDKAIELLREKGMAKAAKKADRVAAEGLTGVYVDGNVAAVIEV

SEQ ID NO: 97 - DNA, 134 bp, Streptococcus agalactiae
atgataaaaaacctgttattaacaggttttttatcatttaatgacggaaaactggacacaaattatttttcttgtataattaaatatattatttcttatcaggaggttatgatgacattagagaaacgatttaa

SEQ ID NO: 98 - protein, 44 aa, Streptococcus agalactiae
MIKNLLLTGFLSFNDGKLDTNYFSCIIKYIISYQEVMMTLEKRF

SEQ ID NO: 99 - DNA, 94 bp, Streptococcus agalactiae
atgaaaaataataaaaataatggttttctgaaaaattcctttatttacatattattgattattgcggttattacaacctttcaatactatttaa

SEQ ID NO: 100 - protein, 31 aa, Streptococcus agalactiae
MKNNKNNGFLKNSFIYILLIIAVITTFQYYL

SEQ ID NO: 101 - DNA, 158 bp, Streptococcus agalactiae
atgttagatattatcttatccggaatttcgcaaggattactttggtcaattatggcaattggcgtgtttatcacttttcgtatcttagacatagccgatctctctgcagaaggggctttccctatgggggctgcagtttgcgccttatgtatcgttaa

SEQ ID NO: 102 - protein, 52 aa, Streptococcus agalactiae
MLDIILSGISQGLLWSIMAIGVFITFRILDIADLSAEGAFPMGAAVCALCIV

SEQ ID NO: 103 - DNA, 161 bp, Streptococcus agalactiae
atggaaatgcctaaaagaaatgaattactcaataaagaaattaaaatgagtattgataaacttagatataaagaaccagagagtgaacatgacaagcgacctactttttatttggtagtacttatacttgttactgtagcagttatattgtcgttatttaa

SEQ ID NO: 104 - protein, 53 aa, Streptococcus agalactiae
MEMPKRNELLNKEIKMSIDKLRYKEPESEHDKRPTFYLVVLILVTVAVILSLF

SEQ ID NO: 105 - DNA, 179 bp, Streptococcus agalactiae
gtggtaagtaaattgagtttaacaacgatttttgcattgctattttcatcaatgctaatttacgcaacacctcttatctttacaagtattgggggaaccttctctgaacgtggtggtatcgtcaacgttggtttagaaggaattatggtaattggagctttctcaggcgttgtatttaa

SEQ ID NO: 106 - protein, 59 aa, Streptococcus agalactiae
MVSKLSLTTIFALLFSSMLIYATPLIFTSIGGTFSERGGIVNVGLEGIMVIGAFSGVVF

SEQ ID NO: 107 - DNA, 558 bp, Streptococcus agalactiae
atgagaattattgcaataactgaaaaggttataaaagaactgtttcgtgataaaagaacacttgctatgatgtttttagcacctattttaattatgtttttgatgaatgttatgttttctgcgaatagtaatacaaaagttaagattggaactattaacgttaacacgaaggtcgtttcaaatttagataatattaagcatattcaagtgagatcatttaaatttaactcatctgctaaaaaagcactcaaatcaaataaaattgatgctcttatttcggaggacaataaatcttatactgtcttctatgcgaatacagattcttcaaagacgactttaacaagacaagcttttaaaaccgctgttaatacaatgaacagtaaggaactgatttcgcaagttaaaattttagctaataagaatccgaaactagcacaatccttacaaactcgctccaaatatatcaaagaaaaatataattacggaaataaaaatacaggcttttttgcaaaaatgataccaatactaatgggatttatggtcttcttcttggttttt

SEQ ID NO: 108 - protein, 186 aa, Streptococcus agalactiae
MRIIAITEKVIKELFRDKRTLAMMFLAPILIMFLMNVMFSANSNTKVKIGTINVNTKVVSNLDNIKHIQVRSFKFNSSAKKALKSNKIDALISEDNKSYTVFYANTDSSKTTLTRQAFKTAVNTMNSKELISQVKILANKNPKLAQSLQTRSKYIKEKYNYGNKNTGFFAKMIPILMGFMVFFLVF

SEQ ID NO: 109 - DNA, 100 bp, Streptococcus agalactiae
gtgattatcgttatgagtaaacatcaagaaattttggagtacctagaaaatttagctgttggtaagagggttagtgtacgcagtatttcaaatcatttaa

SEQ ID NO: 110 - protein, 33 aa, Streptococcus agalactiae
MIIVMSKHQEILEYLENLAVGKRVSVRSISNHL

SEQ ID NO: 111 - DNA, 326 bp, Streptococcus agalactiae
atgtatagagaaattaccgctgtcgaacacgatcgctttgtgagcgaatccaaccaaacaaacctacttcaatctcttaattggcccaaagtaaaagacaactggggtagtcaattacttggcttttttgacggtgaaacccaaattgccagcgctagtattctcatcaaatcacttcctcttggcttctccatgctgtatattccgcgtggaccaatcatggattactccaatctagatattgtaactaaggtccttaaggaccttaaagcttttggcaaaaaacaaagagctctctttatcaagtgtgatcctctcatctattt

SEQ ID NO: 112 - protein, 108 aa, Streptococcus agalactiae
MYREITAVEHDRFVSESNQTNLLQSLNWPKVKDNWGSQLLGFFDGETQIASASILIKSLPLGFSMLYIPRGPIMDYSNLDIVTKVLKDLKAFGKKQRALFIKCDPLIY

SEQ ID NO: 113 - DNA, 215 bp, Streptococcus agalactiae
atggacaagaaaaaaatcttagtaacgggtattgtgcctaaagaaggtctaagaaagcttatggaccgatttgatgttacttattcagaagatcgcccattttcacgtgactatgtgttagagcatttatctgaatatgacggatggttactcatgggacaaaaaggtgataaagagatgattgatgcaggtgaaaacttacaaattatttcttt

SEQ ID NO: 114 - protein, 71 aa, Streptococcus agalactiae
MDKKKILVTGIVPKEGLRKLMDRFDVTYSEDRPFSRDYVLEHLSEYDGWLLMGQKGDKEMIDAGENLQIIS

SEQ ID NO: 115 - DNA, 459 bp, Streptococcus agalactiae
atttcgaaagatgactaccaaaatattagttttggacaggatccagaagttgttgattatgctggtctgtttgaaaaacgccgtccagttttagaaaaagcagttaaaaatttcttgcaagaagagagagctacgagaatgctatctgatttcttgcaagaagaaaaatgggtaactgattttgctgaatttatggcgatcaaagaacattttggtaataaggcgcttcaagaatgggatgacaaggctattatacgccgcgaagaagaagccttagcaggatatcgtcaaaagcttagtgaagtgataaaatatcatgaagtaacgcaatatttcttttacaaacaatggtttgagttaaaagaatatgctaatgataaagggattcaaattatcggtgatatgccaatctacgtttctgccgatagtgtagaagtttggacaatgcctgaactgttt

SEQ ID NO: 116 - protein, 153 aa, Streptococcus agalactiae
ISKDDYQNISFGQDPEVVDYAGLFEKRRPVLEKAVKNFLQEERATRMLSDFLQEEKWVTDFAEFMAIKEHFGNKALQEWDDKAIIRREEEALAGYRQKLSEVIKYHEVTQYFFYKQWFELKEYANDKGIQIIGDMPIYVSADSVEVWTMPELF

SEQ ID NO: 117 - DNA, 1143 bp, Streptococcus agalactiae
atggcaaaacagaaaaataactggcgccgtgttggagttggtgtccttacacttgcttcagttgcgactcttgctgcatgtggaagtaaatcagcttcccaggattctaatggagcgattaattgggctattccaacagaaatcaatacactagatttatctaaagttacagacacttactcaaatctagctattggtaactctagtagtaatttccttcgcttagataaagatggaaagacaagaccagacttggctactaaagttgatgtttcaaaagatggcttaacttatacagctacattacgtaaaggcttgaagtggtcagatggcagtaaacttactgcaaaggattttgtttattcatggcaacgtttagttgatcctaaaacagcttcacaatatgcttaccttgctgttgaagggcatgtgcttaatgccgataaaatcaacgaaggacaagagaaagacttgaataagctaggtgttaaggcagaaggcgatgacaaagttgttattactttatctagtccgtctccgcaattcatctactaccttgcattcactaacttcatgccacaaaaacaagaagttgttgaaaaatatggaaaagattacgcaactacttcaaaaaatacagtttactcaggaccatatactgttgaaggttggaatggttcgaatggtactttcacgctgaagaaaaacaaaaattattgggacgctaaaaatgtaaaaacaaaagaagttcgcatccagactgttaaaaaaccagataccgccgttcaaatgtataaacgtggtgagttagatgcagctaatatctcaaatacttctgctatttatcaagctaataaaaataataaagatgtcacagatgttctagaagcgaccactgcctatatggaatataatactactggttctgtgaaagggcttgataatgttaagattcgtcgcgccttaaacttagcaactaaccgtaaaggagttgttcaagcagccgttgatacaggctcaaaaccggcaattgcttttgcacctactggtttagccaaaacaccagatggaactgatttggcaaaatatgttgccccaggttatgaatataataaaactgaagcagcaaaactctttagacta

SEQ ID NO: 118 - protein, 381 aa, Streptococcus agalactiae
MAKQKNNWRRVGVGVLTLASVATLAACGSKSASQDSNGAINWAIPTEINTLDLSKVTDTYSNLAIGNSSSNFLRLDKDGKTRPDLATKVDVSKDGLTYTATLRKGLKWSDGSKLTAKDFVYSWQRLVDPKTASQYAYLAVEGHVLNADKINEGQEKDLNKLGVKAEGDDKVVITLSSPSPQFIYYLAFTNFMPQKQEVVEKYGKDYATTSKNTVYSGPYTVEGWNGSNGTFTLKKNKNYWDAKNVKTKEVRIQTVKKPDTAVQMYKRGELDAANISNTSAIYQANKNNKDVTDVLEATTAYMEYNTTGSVKGLDNVKIRRALNLATNRKGVVQAAVDTGSKPAIAFAPTGLAKTPDGTDLAKYVAPGYEYNKTEAAKLFRL

SEQ ID NO: 119 - DNA, 234 bp, Streptococcus agalactiae
ttgagagtttatgaaaataaagaagagttgaaaaaagaaataagtaaaacatttgagaaatacattatggaatttaataatattccagagaatctaaaagataaaagaattgatgaagttgatagaactccagcagaaaacctttcttatcaggttggctggaccaacttggttcttaaatgggaagaagatgaaagaaagggacttcaagtaaaaacaccatcggataaattt

SEQ ID NO: 120 - protein, 78 aa, Streptococcus agalactiae
MRVYENKEELKKEISKTFEKYIMEFNNIPENLKDKRIDEVDRTPAENLSYQVGWTNLVLKWEEDERKGLQVKTPSDKF

SEQ ID NO: 121 - DNA, 150 bp, Streptococcus agalactiae
atgtcaaagtttgatagtcagaaaataattactccgattatgaagtttgtcaatatgcgagggattattgcactcaaagatggcatgctagcaattttaccactaacagttgttgggagtctctttttaatattagggcagcttccattt

SEQ ID NO: 122 - protein, 50 aa, Streptococcus agalactiae
MSKFDSQKIITPIMKFVNMRGIIALKDGMLAILPLTVVGSLFLILGQLPF

SEQ ID NO: 123 - DNA, 535 bp, Streptococcus agalactiae
gagaccacttcatcagttaaaccagcaggaattgaccgtatcaatcatacctcaacacccccgaagaaaactacccccaacattgcaacgacgcatagcttcaaagatcgttgtgatactttagaaagaattcacaatgaagacattgatgtttgttctggattcatttgtggtatgggagagagcgatgaggggctcatcacattagctttcagactaaaagaactgaacccctattctatccctgtcaattttttacttgctgttgaaggaacacctcttggaaaatataactatttgactcccattaaatgcttaaaaattatggccatgttgcgttttgtttttcctttcaaggaattaagattaagtgctggacgggaggtccattttgagaattttgaatcattagtcaccttacttgttgactcaacttttttgggaaattacctaacagaggggggtcgcaatcaacataccgatattgaattcttggaaaaattacaactaaatcatactaaaaaggaattaattt

SEQ ID NO: 124 - protein, 178 aa, Streptococcus agalactiae
ETTSSVKPAGIDRINHTSTPPKKTTPNIATTHSFKDRCDTLERIHNEDIDVCSGFICGMGESDEGLITLAFRLKELNPYSIPVNFLLAVEGTPLGKYNYLTPIKCLKIMAMLRFVFPFKELRLSAGREVHFENFESLVTLLVDSTFLGNYLTEGGRNQHTDIEFLEKLQLNHTKKELI

SEQ ID NO: 125 - DNA, 563 bp, Streptococcus agalactiae
atgccggtttggactgcacagtctattccaaaggcatttttagaaaagcataatactaaggaaggcacctgggcaaaactaaccattctaagtggttctttagtattttaccagttatctcctgatggagaggaaatctcgcggcatatttttgatgctagtagtgatattccttttgttgatccacaagtctggcataaagtttcgccgaatagtccagacttaagttgctatctaactttttactgccaaaaagaagattacttccataaaaaatatggtctcacgcgcacacattctgaggttatcgccagtgcacctctcttatctgagaagagtaatatattagaccttgggtgtggtcaagggcgaaactcactttatttatcgctgctgggacatcaagtgacttctgtcgattcaaacggacagagccttgtagctttagaaaatatggcattagaagaagagcttccttacaatataaaaaggtatgatattaatactactgctattgaagggcactatgattttattttatcaactgtggtatttatgttttt

SEQ ID NO: 126 - protein, 187 aa, Streptococcus agalactiae
MPVWTAQSIPKAFLEKHNTKEGTWAKLTILSGSLVFYQLSPDGEEISRHIFDASSDIPFVDPQVWHKVSPNSPDLSCYLTFYCQKEDYFHKKYGLTRTHSEVIASAPLLSEKSNILDLGCGQGRNSLYLSLLGHQVTSVDSNGQSLVALENMALEEELPYNIKRYDINTTAIEGHYDFILSTVVFMF

SEQ ID NO: 127 - DNA, 417 bp, Streptococcus agalactiae
atgacaaagcaaataattgccatttgggctgaagatgaagaccatttgattggagttaatggcggtttaccatggaggcttcctaaagagttacatcacttcaaagaaacgaccatggggcaggctttgcttatgggacgaaagacctttgatggaatgaaccgtcgtgttttacctggtagagagacaatcatcttaacaaaagatgaacaattccaagcagatggagtgacagtcctaaatagtgttgaacaagttataaaatggtttcaggaacataataagaccttatttattgtaggtggtgcaagtatttataaagcatttctgccttattgtgaagcaatcataaaaactaaagttcatggaaaattcaaaggtgatacctattttcctgatgttaatctatctgagttt

SEQ ID NO: 128 - protein, 139 aa, Streptococcus agalactiae
MTKQIIAIWAEDEDHLIGVNGGLPWRLPKELHHFKETTMGQALLMGRKTFDGMNRRVLPGRETIILTKDEQFQADGVTVLNSVEQVIKWFQEHNKTLFIVGGASIYKAFLPYCEAIIKTKVHGKFKGDTYFPDVNLSEF

SEQ ID NO: 129 - DNA, 543 bp, Streptococcus agalactiae
ttgtggccaaactgtgccccgcttattaatagcactttgttcaccattgaagatatcttaacatcaggtgctcatagcaaccctattttaatgggggttatacttggcgggacaattgtagtagtggcgacagcaccactttcttctatggcattgacagctatgctaggattaaccggaatgcctatggctataggagccttgtctgtctttggttcgtcatttatgaatggtgtacttttccataaattaaaacttggaagtcgtaaagataatatagcttttgctgttgagcctctaactcaagctgacgtgacttcagctaaccctattccaatctatgtcactaattttgttggtggtgcagcttgtggtattttaattgccttgatgaaattagttaatgatactcctggaacagcgacaccaattgcaggatttgctgtcatgtttgcctataacccaatgataaaagtactaataaccgctctaggttgtattatcctatctttactagcaggctattttggaggcattgttttt

SEQ ID NO: 130 - protein, 181 aa, Streptococcus agalactiae
MWPNCAPLINSTLFTIEDILTSGAHSNPILMGVILGGTIVVVATAPLSSMALTAMLGLTGMPMAIGALSVFGSSFMNGVLFHKLKLGSRKDNIAFAVEPLTQADVTSANPIPIYVTNFVGGAACGILIALMKLVNDTPGTATPIAGFAVMFAYNPMIKVLITALGCIILSLLAGYFGGIVF

SEQ ID NO: 131 - DNA, 172 bp, Streptococcus agalactiae
atgtttttaagtataatggcaggtgtcatagcatttgtcctgacagttattgccattccacgcttcattaagttttaccaattgaagaaaattggcgggcaacaaatgcatgaagatgtcaaacaacatctagccaaagcaggtacgccgacaatgggaggaacggtatttt

SEQ ID NO: 132 - protein, 57 aa, Streptococcus agalactiae
MFLSIMAGVIAFVLTVIAIPRFIKFYQLKKIGGQQMHEDVKQHLAKAGTPTMGGTVF

SEQ ID NO: 133 - DNA, 113 bp, Streptococcus agalactiae
atgaaaccatatttatcttttattggtagaacgttattatacttcggtattttattgttactaatttacttttttgcataccttggtcgcggacaaggcagttttatttataa

SEQ ID NO: 134 - protein, 37 aa, Streptococcus agalactiae
MKPYLSFIGRTLLYFGILLLLIYFFAYLGRGQGSFIY

SEQ ID NO: 135 - DNA, 651 bp, Streptococcus agalactiae
atgtcatattttagaaattactggtatcgttttggagcaattttatttattattttagcagtaatattgcttgtttttagacctgactggtcaatgcttcactatctattgtatttttactttatggcacttctagcgcatcaatttgaagaatatcagtttcccggtggggcatcacctatcattaactatgttgtttatgatgaagaagagctgatggattgttttccaggcaatactcagtctattatgttggttaatactattgcttggttgctttacattgctagtattgcttttcctcaagcttattggcttggattaggagtcatgttctttagtctaacgcagctcttgggtcatggttttcagatgaatattaaacttaaaacttggtataatcctggtctagcaacgacagtatttctcctagtaccaatagcttgcgcatacatctatcaagctagtgcagaaggaatgctcacttggggagattggctaggtggttttatcatgttgattgtctgtgtactaactagcattattgcacctgtacagctattgaaggataaggagaccaattatattattagtccttggcaaatggaccgttttcataaggtcgttaattttgtaaggataaaaaaataa

SEQ ID NO: 136 - protein, 216 aa, Streptococcus agalactiae
MSYFRNYWYRFGAILFIILAVILLVFRPDWSMLHYLLYFYFMALLAHQFEEYQFPGGASPIINYVVYDEEELMDCFPGNTQSIMLVNTIAWLLYIASIAFPQAYWLGLGVMFFSLTQLLGHGFQMNIKLKTWYNPGLATTVFLLVPIACAYIYQASAEGMLTWGDWLGGFIMLIVCVLTSIIAPVQLLKDKETNYIISPWQMDRFHKVVNFVRIKK

SEQ ID NO: 137 - DNA, 75 bp, Streptococcus agalactiae
atgccacttacagcacttgaaattaaagataaaacattttcatcaaaatttcgcggttatagcgaagaagaagtt

SEQ ID NO: 138 - protein, 25 aa, Streptococcus agalactiae
MPLTALEIKDKTFSSKFRGYSEEEV

SEQ ID NO: 139 - DNA, 377 bp, Streptococcus agalactiae
atgtcactttttcaagaaaaaattgcttacaattgcgctaaaaaggaagcgctttataaagagagtttaggacgctacgccttgagatcaatgctagcaggggcttatttgacaatgagtactgctgccggtatcgtcgcagctgatactattggtaaaatttctcctgctctatcaggttttgtatttgctttcatctttagttttggacttatttatgttttaatatttaatggtgaattggcgacatctaatatgctttatctcactgcaggagcctataataaaaatatctcttggaaaaaagccataacaattttaatttattgtacttttttcaacctcgttggtgcttgtatattagcttggttgtttaa

SEQ ID NO: 140 - protein, 125 aa, Streptococcus agalactiae
MSLFQEKIAYNCAKKEALYKESLGRYALRSMLAGAYLTMSTAAGIVAADTIGKISPALSGFVFAFIFSFGLIYVLIFNGELATSNMLYLTAGAYNKNISWKKAITILIYCTFFNLVGACILAWLF

SEQ ID NO: 141 - DNA, 419 bp, Streptococcus agalactiae
aagttacaagcgactgaagttaagagcgttccggtagcacaaccagcttcaacaacaaatgcagtagctgcacatcctgaaaatgcagggctccaacctcatgttgcagcttataaagaaaaagtagcgtcaacttatggagttaatgaattcagtacataccgtgcgggagatccaggtgatcatggtaaaggtttagcagttgactttattgtaggtaaaaaccaagcacttggtaatgaagttgcacagtactctacacaaaatatggcagcaaataacatttcatatgttatctggcaacaaaagttttattcaaatacaaatagtatttatggacctgctaatacttggaatgcaatgccagatcgtggtggcgttactgccaaccactatgaccacgttcacgtatcatttaa

SEQ ID NO: 142 - protein, 139 aa, Streptococcus agalactiae
KLQATEVKSVPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSF

SEQ ID NO: 143 - DNA, 693 bp, Streptococcus agalactiae
atgattccagtagttattgaacaaacaagtcgtggtgaacgttcttatgatatttactcacgtcttttaaaagatcgtattattatgttgacaggccaagttgaggataatatggccaatagtatcattgcacagttattgtttctcgatgcacaagataatacaaaggatatttacctttatgtcaatacaccaggtggttcagtatcggctggacttgctattgtggacaccatgaacttcattaaatcggacgtacagacgattgttatggggatggctgcttcgatgggaaccattattgcttcaagtggtgctaaaggaaaacgttttatgttaccgaatgcagaatatatgatccaccaaccaatgggcggaacaggcggaggtacacagcaatctgatatggctatcgctgctgagcatcttttaaaaacgcgtcatactttagaaaaaatcttagctgataattctggtcaatctattgaaaaagtccatgatgatgcagagcgtgatcgttggatgagtgctcaagaacacttgattatggctttattgatgctattatggaaaataataatttacaataatagatttaaaagagttgagtttaccaactctttttttatttgttggaattatgttataatcttagtaattacagatatgacgcagaaaggaaaaaattattga

SEQ ID NO: 144 - protein, 230 aa, Streptococcus agalactiae
MIPVVIEQTSRGERSYDIYSRLLKDRIIMLTGQVEDNMANSIIAQLLFLDAQDNTKDIYLYVNTPGGSVSAGLAIVDTMNFIKSDVQTIVMGMAASMGTIIASSGAKGKRFMLPNAEYMIHQPMGGTGGGTQQSDMAIAAEHLLKTRHTLEKILADNSGQSIEKVHDDAERDRWMSAQEHLIMALLMLLWKIIIYNNRFKRVEFTNSFFICWNYVIILVITDMTQKGKNY

SEQ ID NO: 145 - DNA, 459 bp, Streptococcus agalactiae
atgaaaccaaaaattattggtgtacttggtctaggaatatttggacaaacactcgcacaagaactaagtaactttgaacaagatgttattgctattgacagcaatcctgaaaatgtacaagctgtcgccgaagttgttacaaaagcagctatcggagacattactgatttagctttcctaaaacacatcgggatcagtgactgtgatactgttattattgctacaggaaacagtttagagagctcagtattggccgtaatgcactgtaaaaagttaggcgtcccacaagttattgctaaagctcgaaaccttgtatacgaagaagtactttatgaaattggtgctgatttggttatctctccggagcgagaatctgggcaaaatgttgctgcaaacctcatgagaaataaaattacagatgtcttccagattgaatctgatatttctgtcattgaattt

SEQ ID NO: 146 - protein, 153 aa, Streptococcus agalactiae
MKPKIIGVLGLGIFGQTLAQELSNFEQDVIAIDSNPENVQAVAEVVTKAAIGDITDLAFLKHIGISDCDTVIIATGNSLESSVLAVMHCKKLGVPQVIAKARNLVYEEVLYEIGADLVISPERESGQNVAANLMRNKITDVFQIESDISVIEF

SEQ ID NO: 147 - DNA, 330 bp, Streptococcus agalactiae
gtgcgttatagtaaagagattattcagttagctataccagctatgattgaaaatatcttacaaatgctcatgggagtagttgataattatctagtggctcagttaggtgttgtagcagtatcaggtgtttcagttgctaataatataattactatttatcaagctatttttatagctttaggggcgagtatagcaagtctattggccaagtcgttagcaggtagtgagaaggatgatgcaatttcagtatgttctcaagccatttttctaacatcactgataggggcagtattaggaattatctcgattgtttttggacaaactttcttt

SEQ ID NO: 148 - protein, 110 aa, Streptococcus agalactiae
MRYSKEIIQLAIPAMIENILQMLMGVVDNYLVAQLGVVAVSGVSVANNIITIYQAIFIALGASIASLLAKSLAGSEKDDAISVCSQAIFLTSLIGAVLGIISIVFGQTFF

SEQ ID NO: 149 - DNA, 240 bp, Streptococcus agalactiae
ttgattaacaagtattcgtgctttttgaagaggattctccataataatactcctttaatagttatcgtgagaagtattttaaagaaaaaccgccaaggtagagcgacatttctgcctttaactacaataaaaccaagagaattagcacaacattatctctcaaaattacaaagttctcaagggtttttaggaatagctagtgaattggtaacctatgatcaacgcttgtcaaacattttt

SEQ ID NO: 150 - protein, 80 aa, Streptococcus agalactiae
MINKYSCFLKRILHNNTPLIVIVRSILKKNRQGRATFLPLTTIKPRELAQHYLSKLQSSQGFLGIASELVTYDQRLSNIF

SEQ ID NO: 151 - DNA, 649 bp, Streptococcus agalactiae
ttgttgactcacaaaaatatattattaaccattatatttggattatttatgattatattatcagcatgtggtatgtctaataaggaaatggctggtattgataattgggaacattatcaaaaggaaaagaaaattactattggatttgataatacttttgttcctatgggatttgaaagtcgttctggtgactataccggctttgatattgatttagctaatgctgtttttaaagaatacggtatttcagtgaaatggcagcctattaactgggatatgaaagaaactgaacttaataatggtaatatagaccttatttggaatggttattcaaaaacggcagaacgtgctaaaaaagtcgcttttacaaacccatatatgaataatcatcaagtaattgttactaaaacttcatcacatattaatagtattaaggatatgaaggggaaaaaactaggagcccagtcgggttcatctggttttgatgcttttaacgctaaacctgatattttaaaaaagtttgtaaaaggaaaagaagcagttcaatacgatactttcactcaggctttgattgatttaaaaaataaccgtattgatggtcttttgattgatgaagtttatgctaactattatttaaagcaagaaggaa

SEQ ID NO: 152 - protein, 216 aa, Streptococcus agalactiae
MLTHKNILLTIIFGLFMIILSACGMSNKEMAGIDNWEHYQKEKKITIGFDNTFVPMGFESRSGDYTGFDIDLANAVFKEYGISVKWQPINWDMKETELNNGNIDLIWNGYSKTAERAKKVAFTNPYMNNHQVIVTKTSSHINSIKDMKGKKLGAQSGSSGFDAFNAKPDILKKFVKGKEAVQYDTFTQALIDLKNNRIDGLLIDEVYANYYLKQEG

SEQ ID NO: 153 - DNA, 123 bp, Streptococcus agalactiae
atgaaaatttggaaaaaaataaccttaatgttttctgcaattattttaacaacagtaattgcattgggagtctatgttgcctcagcttataatttttcgactaatgaattgtctaagactttt

SEQ ID NO: 154 - protein, 41 aa, Streptococcus agalactiae
MKIWKKITLMFSAIILTTVIALGVYVASAYNFSTNELSKTF

SEQ ID NO: 155 - DNA, 687 bp, Streptococcus agalactiae
atgaaaaaacaaagactattactgctttttggaggcttattaataatgataatgatgacagcatgtaaggattcaaaaatcccagaaaaccgcacgaaaaaggaataccaggcagaacagaattttaagtcatactttaaatatatatcagataaaaataactatttagataatataaaagtttattacttttctataagtatttctaaagatgtacaagataaagtcagtgaaacaacaacttgttcatatagactagaaaagcaaaagaatcaagagttcattggtaattttgaacatgaagttagtgaatctagtcaatattcaaccgaagttaaaaatcaaatacagtatccaatccagtataaagataattcaattcgttttactgaaaaaacaccgtcagaacgttatgatgagtttgtttttagttcatttgattcttcattattaaaaaaatataaaatatatgattacttactaaaacatcccgaaactgaattaaaaggtgtttcctataagattcctataaattctgaaattgtagccccttttataaatcaattaaatataaaaaatcctaaaaaatcatctatttcggttacaaaaacggaaagtaaagaatattattatacaatcagtattgatactgattctgagatatattctatattcgaaggtattcat

SEQ ID NO: 156 - protein, 229 aa, Streptococcus agalactiae
MKKQRLLLLFGGLLIMIMMTACKDSKIPENRTKKEYQAEQNFKSYFKYISDKNNYLDNIKVYYFSISISKDVQDKVSETTTCSYRLEKQKNQEFIGNFEHEVSESSQYSTEVKNQIQYPIQYKDNSIRFTEKTPSERYDEFVFSSFDSSLLKKYKIYDYLLKHPETELKGVSYKIPINSEIVAPFINQLNIKNPKKSSISVTKTESKEYYYTISIDTDSEIYSIFEGIH

SEQ ID NO: 157 - DNA, 272 bp, Streptococcus agalactiae
atgacatttgacaccattgatcaattagcggttaatacagtccgcacgctttctattgatgctatccaagcagcaaattctgggcacccaggtcttcctatgggagctgcgcctatggcttatgtgctttggaataaattcttaaatgtaaacccaaaaacaagtcgcaattggacaaaccgtgaccgttttgtactttcagctgggcatggttcagctcttctttatagcctacttcatttagctggctatgatttatcaattgatgattt

SEQ ID NO: 158 - protein, 90 aa, Streptococcus agalactiae
MTFDTIDQLAVNTVRTLSIDAIQAANSGHPGLPMGAAPMAYVLWNKFLNVNPKTSRNWTNRDRFVLSAGHGSALLYSLLHLAGYDLSIDD

SEQ ID NO: 159 - DNA, 197 bp, Streptococcus agalactiae
atgagaacactatttagaatgatatttgctattccaaagtttatctttagattgatttggaatatcatttggggaatattcaagacagttcttgttattgcgattattttatttggcttgtattactatgcgaatcacagtcaatcagaatttgctaatcaacttagtgacattattcagacaggaaaaacattttt

SEQ ID NO: 160 - protein, 65 aa, Streptococcus agalactiae
MRTLFRMIFAIPKFIFRLIWNIIWGIFKTVLVIAIILFGLYYYANHSQSEFANQLSDIIQTGKTF

SEQ ID NO: 161 - DNA, 153 bp, Streptococcus agalactiae
atgtcaaaaaaaataatattaggaattttatctcttttatctgtcgttactttggtggcgtgtggttcatcagacaaacagctacaagataaagttgagaaaaaagggaagttagttttagcggtgagtccagattatgctccctttgagttt

SEQ ID NO: 162 - protein, 51 aa, Streptococcus agalactiae
MSKKIILGILSLLSVVTLVACGSSDKQLQDKVEKKGKLVLAVSPDYAPFEF

SEQ ID NO: 163 - DNA, 138 bp, Streptococcus agalactiae
atgaaaaatcaaagactattactgctttttggaggcttattaataatgataatgatgacagcatgtaaggattcaaaaatcccagaaaaccgcacgaaaaaggaataccaggcagaacagaattttaagtcatacttt

SEQ ID NO: 164 - protein, 46 aa, Streptococcus agalactiae
MKNQRLLLLFGGLLIMIMMTACKDSKIPENRTKKEYQAEQNFKSYF

SEQ ID NO: 165 - DNA, 423 bp, Streptococcus agalactiae
atgattggaaaattatattatagctatagaaagtcacgcttattaagaagtattttatggcttattttaattgttggtgtatatatgttaggacaacgtgttttattatccactgttcctttatcacatcaagagataaaactagcagtagatcaacatttactcaataacttttcagcagtaagtggtgggagttttaataaattaaatgttttcacactggggttgagtccatggatgtcaagtatgattatttggagattcgtttccttattttcgtgggcaaaaaatgcaacgaagcgaaaagcagaagtagctcaatatactttaatgcttactatctcagttatacaagcatatggtgtttcaggaaatcaatttataaaaagctctttattaggttcttatagtgatattgttttt

SEQ ID NO: 166 - protein, 141 aa, Streptococcus agalactiae
MIGKLYYSYRKSRLLRSILWLILIVGVYMLGQRVLLSTVPLSHQEIKLAVDQHLLNNFSAVSGGSFNKLNVFTLGLSPWMSSMIIWRFVSLFSWAKNATKRKAEVAQYTLMLTISVIQAYGVSGNQFIKSSLLGSYSDIVF

SEQ ID NO: 167 - DNA, 348 bp, Streptococcus agalactiae
atgaaaggtctattggattttttagttaatattgccagaacgccagctattttagtcgccttgatagccattatcggtttagtactgcagaaaaaaggtgttcctgatattgtaaaaggtggaataaaaacatttgttggcttcttagtggtttctgaaggtgcagggatagtccaaaattccttgaatccatttggaaaaatgtttgaacatgcttttcatttggtgggggtagttcctaataatgaagccattgtagcagtagctcttacgaagtatggctcagcaactgctttgattatgttagcgggaatgatttttaatattttaattgctcgttttacaaaa

SEQ ID NO: 168 - protein, 116 aa, Streptococcus agalactiae
MKGLLDFLVNIARTPAILVALIAIIGLVLQKKGVPDIVKGGIKTFVGFLVVSEGAGIVQNSLNPFGKMFEHAFHLVGVVPNNEAIVAVALTKYGSATALIMLAGMIFNILIARFTK

SEQ ID NO: 169 - DNA, 464 bp, Streptococcus agalactiae
ttggttggtaagccccaattactatttttagatgaacctacttccggaatggatacttccacacgtcaacgattttggaagctggttgcgacactaaaaaaagaaggtgacacaattgtctattctagtcattatatcgaagaggtagaacatacagctgataggattttagtacttcataaaggaaagttattacgcgatacaaccccctttgccatgaagcaagaaaaaaccgaaaagttattcaccgttccgcttagttatcaaaaattattacctacctatttgattacagagtgtgaagccaagagtgatagtataacgtttgttactggggaggctgaaactgtatggaaaatactggcagataatggttgtcctattgaagctattgagatgaccaatagaactttgttaaatcgtatttttgagactactaaggaggtaaaacatgagaatcttta

SEQ ID NO: 170 - protein, 154 aa, Streptococcus agalactiae
MVGKPQLLFLDEPTSGMDTSTRQRFWKLVATLKKEGDTIVYSSHYIEEVEHTADRILVLHKGKLLRDTTPFAMKQEKTEKLFTVPLSYQKLLPTYLITECEAKSDSITFVTGEAETVWKILADNGCPIEAIEMTNRTLLNRIFETTKEVKHENL

SEQ ID NO: 171 - DNA, 360 bp, Streptococcus agalactiae
ttgaaaaaatccaagagaagccgtaaggcagtgacaacaagtggtgagaagactttacttgaggatttggcaaaaatgaatttcctagacgaagtcattaatgttatggttttatataccttgaataagacaaaatctgctaacttaaataaggcctatatcatgaaagttgctaatgattttgcctttcagaatgttatgacggccgaagatgctgtgcttaaaattcgtgatttttcagatcaaaaagtaaggactaaaacagaaacgaagaagaaacaatcgaatgttcctgaatggagtaatcctgattataaagatgaggttagcccagaaaaagaaattgaattagaacagttt

SEQ ID NO: 172 - protein, 120 aa, Streptococcus agalactiae
MKKSKRSRKAVTTSGEKTLLEDLAKMNFLDEVINVMVLYTLNKTKSANLNKAYIMKVANDFAFQNVMTAEDAVLKIRDFSDQKVRTKTETKKKQSNVPEWSNPDYKDEVSPEKEIELEQF

SEQ ID NO: 173 - DNA, 216 bp, Streptococcus agalactiae
atgacgaatcatattactaaactgatagaaaatagcggaaaaaaattgacagaaattagcgaagctacagatatagcctatcctacactttctggatacaatcaaggaatccgcaaacctaaaaaagataatgctgaaaaattggcaaaatactttaatgtttccgtcgcttacattatgggacttgatagcaacccacatgctccatcaaatctt

SEQ ID NO: 174 - protein, 72 aa, Streptococcus agalactiae
MTNHITKLIENSGKKLTEISEATDIAYPTLSGYNQGIRKPKKDNAEKLAKYFNVSVAYIMGLDSNPHAPSNL

SEQ ID NO: 175 - DNA, 337 bp, Streptococcus agalactiae
ttgatgaaaaggaataaacatttaccgttaacagaaactacctattatattttattagctttgtttgaggaagcgcatggctatgctattatgaaaaaagttgaagaaatgagtggcggtgatgttagaatagccgcagggacaatgtacggtgccattgaaaatttacttaaacaaaaatggataaagtctatctcaagtgacgatagaagaagaaaagtttatattattactgagacaggaaaagaaatagtagaacttgaaacgaatcgattaagaaagttacttaatactgctaatcagttgggttttggaggagatggttatgataaagttt

SEQ ID NO: 176 - protein, 112 aa, Streptococcus agalactiae
MMKRNKHLPLTETTYYILLALFEEAHGYAIMKKVEEMSGGDVRIAAGTMYGAIENLLKQKWIKSISSDDRRRKVYIITETGKEIVELETNRLRKLLNTANQLGFGGDGYDKV

SEQ ID NO: 177 - DNA, 511 bp, Streptococcus agalactiae
cccattactggtgagttaatagctgagaaattaggagtaccaagagcagcactaaggtctgatttgcgggttttaagtatgctaggtatcatagatgcaaaacctaaggttggttatttttatttaggacagtatcatgcttcaatagggacaagtcattttgaaaagatgacagtttcagaaattatggggatccttctgacagttcatcaaaaagattcagtttatgatgttattgtacatatttttatggaagatgctggttgtgcttttatcttggatgatgatgattttctctgtggagtcgtgtcacgtaaagatttactaaaaaccagtattggcggaggagatctttctaaaatgccaataggaatggtgatgacacgtatgccacacgtgacaactgttttagaaaatgaaagtctttttgcggcagctgataaattagtgagcagaaaagtggatagtctccctgtcgttcgtcatgataagcaatatcccgaaaaattta

SEQ ID NO: 178 - protein, 170 aa, Streptococcus agalactiae
PITGELIAEKLGVPRAALRSDLRVLSMLGIIDAKPKVGYFYLGQYHASIGTSHFEKMTVSEIMGILLTVHQKDSVYDVIVHIFMEDAGCAFILDDDDFLCGVVSRKDLLKTSIGGGDLSKMPIGMVMTRMPHVTTVLENESLFAAADKLVSRKVDSLPVVRHDKQYPEKF

SEQ ID NO: 179 - DNA, 233 bp, Streptococcus agalactiae
ttggaagtcatcatgcaatttatttatagtattattggtattttattggtattaggaattgtgtatgcaatttctttcaatcgtaagagtgtttctctaagtttaattggaaaagctcttatcgttcaattcattattgcgctaatcttagtacgtatcccactaggccaacaagttgttagtgttgtttcaactggagttactaaagtaatcaactgtggtcaagctggttt

SEQ ID NO: 180 - protein, 77 aa, Streptococcus agalactiae
MEVIMQFIYSIIGILLVLGIVYAISFNRKSVSLSLIGKALIVQFIIALILVRIPLGQQVVSVVSTGVTKVINCGQAG

SEQ ID NO: 181 - DNA, 344 bp, Streptococcus agalactiae
caacctaataaagctttagaaagtgatgagattgatattaatgctttccagcattataattacttaaccaattggaataaagcaaataagaccaatcttgtttccgttgctgagacatactttacttcctttagattatactctggtactaagaacggtaaaggtaaataccaaacagtttctgaaattccaaataaagcaactattactatcccaaacgatgcagttaacgaaagtcgctctctctacttgttacaatcagcaggcttgctaaaattgaaagtatcaggtgatacattagcaacaatgtcagatgttgtttccaatcctaaatctttagattt

SEQ ID NO: 182 - protein, 114 aa, Streptococcus agalactiae
QPNKALESDEIDINAFQHYNYLTNWNKANKTNLVSVAETYFTSFRLYSGTKNGKGKYQTVSEIPNKATITIPNDAVNESRSLYLLQSAGLLKLKVSGDTLATMSDVVSNPKSLD

SEQ ID NO: 183 - DNA, 264 bp, Streptococcus agalactiae
atgaaatgtataataaataatataaataaaataaaaatgataattgagatttatcatagaaggaaaactattttgaaattaaataaaatcatattatctactgcagctcttactgctctctttttaggatataatagcgttactgcggatacatataataactatcagccacatagatcaaataatatggatttaactgaggaatataactataataaccagatagaacttcaggagcgtataaaaaacctaaatatacctttt

SEQ ID NO: 184 - protein, 88 aa, Streptococcus agalactiae
MKCIINNINKIKMIIEIYHRRKTILKLNKIILSTAALTALFLGYNSVTADTYNNYQPHRSNNMDLTEEYNYNNQIELQERIKNLNIPF

SEQ ID NO: 185 - DNA, 926 bp, Streptococcus agalactiae
ttgggtgattattatggtaagaaatattttggtgaggcagctaaaaaagacgtcgaacatatggctaagaaaatcattaatgtctataaaacacggttaaaaaacaacacttggttatcagaaaatacaaaagcaatggccattaagaaacttgataacatgagattaatgattggctatccagaagattatcctgatctttatcgtcagtaccaatttgatagtaaagcaagcttctttgaaaacaatgataactacagaaaattatcgaacaagaaaacatttgaagaatttaaccagtctaatcaacgtgaacattggcaaatgagtgccaatgctgtaaatgcttataatgatcctaataccaattccatagtctttccagcagcgatttttcaatcaccactgtacgataaaactaaaacagttagtcaaaattatggagctatcggagcaattattggtcatgaaatttcacactcatttgatattaatggtatgaaatatgacgagaaagggaatcttcacgattggtggactaaagaagatttaaatcattataagaaatcaacacaagctatgattgaccaatgggatggccttaaagcagatggcggtaaagttgatggtaaattaactttagcagaaaatattgcagataatggtggtgttatggcatctctagaagctcttaagactgaaaaaatccaaactataaagaattttttgaatcatgggcaagtatttggcgtcaaaaagcaaccaaagaacaaagtaagtcctcaattcagtcagatgttcatgcaccatatgaattgagagctaacatcccagtacgtaatttccaagaattttatgatgcctttggtgttaaaaaaggcgattcaatgtatctaaaaccagaaaaacgtttgacactttggtaa

SEQ ID NO: 186 - protein, 271 aa, Streptococcus agalactiae
MGDYYGKKYFGEAAKKDVEHMAKKIINVYKTRLKNNTWLSENTKAMAIKKLDNMRLMIGYPDYPDLYRQYQFDSKASFFENNDNYRKLSNKKTFEEFNQSNQREHWQMSANAVNAYNDPNTNSIVFPAAIFQSPLYDKTKTVSQNYGAIGAIIGHEISHSFDINGMKYDEKGNLHDWWTKEDLNHYKKSTQAMIDQWDGLKADGGKVDGKLTLAENIADNGGVMASLEALKTEKIQTIKNFLNHGQVFGVKKQPKNKVSPQFSQMFMHHMN

SEQ ID NO: 187 - DNA, 636 bp, Streptococcus agalactiae
atgaccatgattacgccaagcttcattaaggtatctctagatgaaacaaatcgtatgatgcgtatgatatcagatttattaagtttatcgcgcattgataatgaagtaacgcatttagatgttgaaatgacgaattttacagctttcatgacctcaattttgaatcgatttgatcagattagaaatcaaaaaacagtcacaggaaaagtttatgaaattgtcagagattatcctcttaagtcaatttgggtggaaattgatacagataagatgactcaagtgattgataacattttaaataatgcagtcaagtattcaccagatggtggtaagattacagttaatctacgcacaactaaaacgcagatgattttatcaatatcagaccaaggcttaggtattcccaaaaaagatttacctctcatttttgatcgtttttatcgtgttgataaggcgagaagtcgtcaacagggtgggactggacttggtttgtcaattgcaaaagaaattgttaagcagcataagggatttatttgggctaagagtgagtatggtaaagggtctacttttacaatcgtcttgccttatgataaagatgctgtaacttatgaagaatgggaggacgttgaagattaa

SEQ ID NO: 188 - protein, 211 aa, Streptococcus agalactiae
MTMITPSFIKVSLDETNRMMRMISDLLSLSRIDNEVTHLDVEMTNFTAFMTSILNRFDQIRNQKTVTGKVYEIVRDYPLKSIWVEIDTDKMTQVIDNILNNAVKYSPDGGKITVNLRTTKTQMILSISDQGLGIPKKDLPLIFDRFYRVDKARSRQQGGTGLGLSIAKEIVKQHKGFIWAKSEYGKGSTFTIVLPYDKDAVTYEEWEDVED

SEQ ID NO: 189 - DNA, 1236 bp, Streptococcus agalactiae
ttgaaaaaaattattacttctattctattacttagttgcattttttttatgccaaccatctctgctgaatcttttaatgcttccgctaaacatgccttagcagttgatttagattcaggaaaaatcttgtatgaaaaagatgctaacaaacccgctgctattgcttccttgactaaaataatgaccgtttatatggtctataaagaaattgataacggtaacctcaagtggaataccaaagtaaatatatctgactacccttatcaactaacacgcgaatctgatgctagtaatgttcctttagaaaaaaggcgctatactgttaaacaactcgtggacgctgccatgatttctagtgctaacagtgcagccattgctttagctgaacatatttcaggaactgaaagtaaatttgttgataaaatgactgctcaattggaaaagtggggaattcatgatagccacctagtcaatgcttctggcttaaataatagtatgttaggcaatcacatttatccaaaatcgtcacaaaacgacgaaaataaaatgagtgcacgtgatattgctattgctgcctaccatttggtcaacgaatatccttccattcttaagattactagtaagtccgttgctaaatttgataaagatattatgcattcttataactacatgctaccagatatgcctgtctttagaccaggtattacaggtttgaaaactgggacaacggaattagctggccaatcttttattgctacatctactgaaagtggaatgagactactcactgttattatgcatgctgataaggccgataaagacaaatatgctcgctttacagcaactaactctctcttgaactatatcacaaacacctacgaacctaaccttgtattagctaaaggagctgcatataaaggtaaagaagcaagtgtgagagacggaaaagaacaatcggtcatcgctgttgctaaaaacgatttgaaagtagtacagaagaaaaatatcactaaacaaaatcagttaaaaattaactttaaaaaagagcttactgctcctattacaaaaaaagagaacctagggaaagcttattacgttgaccttaataaggttggaaaaggctatctcataaaggaacctagcgttcatttagtggcaaaagatagtattgagcgcagtttcttcctcaaagtgtggtggaatcattttgtgcgctacgttaacgaaaaactttaa

SEQ ID NO: 190 - protein, 411 aa, Streptococcus agalactiae
MKKIITSILLLSCIFFMPTISAESFNASAKHALAVDLDSGKILYEKDANKPAAIASLTKIMTVYMVYKEIDNGNLKWNTKVNISDYPYQLTRESDASNVPLEKRRYTVKQLVDAAMISSANSAAIALAEHISGTESKFVDKMTAQLEKWGIHDSHLVNASGLNNSMLGNHIYPKSSQNDENKMSARDIAIAAYHLVNEYPSILKITSKSVAKFDKDIMHSYNYMLPDMPVFRPGITGLKTGTTELAGQSFIATSTESGMRLLTVIMHADKADKDKYARFTATNSLLNYITNTYEPNLVLAKGAAYKGKEASVRDGKEQSVIAVAKNDLKVVQKKNITKQNQLKINFKKELTAPITKKENLGKAYYVDLNKVGKGYLIKEPSVHLVAKDSIERSFFLKVWWNHFVRYVNEKL

SEQ ID NO: 191 - DNA, 771 bp, Streptococcus agalactiae
atgacgcttcgagaattaacaatagaagaatttaaagaacattcaggaaattatgattcacaatcatttttacaaacacctgagatggctaaacttttagaaaaacgcggctatgatgttaggtatttgggatatcaagtagaaaataaactagagataatcagtttatcttatattatgccagtcactggtggttttcaaatgaaaattgattcaggaccagttcattcaaattctaagtatctaaaacaattttataaagcattgcaaggctatgccaaatccaacggtgttctagaattaatagttgagccttttgatgattaccaattattcactagttcgggagttcctagtaatcagggaaatgataatctgattgaagattttaccagttcaggttatcaccatgatggtttaacaactggttttactggtaaatatttatcttggcactatgttaaaaatttagaaggtgtcacttctgaaacgttactatcttcattctctaagacaggacgagctttggttaagaaagcaatgtcttttggaatcaaggttcgcgttcttaaacgtgatgagctacatttatttaaagagataacaacttctacgtcaaatagacgtgattatatggataagtccttagattattatcaagatttttacgatagctttgaaggcaaggctgaatttgtgattgccactttaaattttagagaatacgaccataacttgcaaataaaagctgaagcattggaaaataagctt

SEQ ID NO: 192 - protein, 257 aa, Streptococcus agalactiae
MTLRELTIEEFKEHSGNYDSQSFLQTPEMAKLLEKRGYDVRYLGYQVENKLEIISLSYIMPVTGGFQMKIDSGPVHSNSKYLKQFYKALQGYAKSNGVLELIVEPFDDYQLFTSSGVPSNQGNDNLIEDFTSSGYHHDGLTTGFTGKYLSWHYVKNLEGVTSETLLSSFSKTGRALVKKAMSFGIKVRVLKRDELHLFKEITTSTSNRRDYMDKSLDYYQDFYDSFEGKAEFVIATLNFREYDHNLQIKAEALENKL

SEQ ID NO: 193 - DNA, 534 bp, Streptococcus agalactiae
ttgtcattaagtttggttgcagtgttaaatcttatccctcctaaaatcatgggatcagttattgatgctattacaactggaaaattaacaagaccacaattactatggaatttattaggtttggttttgtcagctttagctatgtatgggctgcgttatatttggcgtatgtatattttagggacttcttacaaattaggccaagttgtcagataccgtttatttgaacattttacaaaaatgtctccttctttttatcagaaatatcgtacaggtgatttaatggcgcacgcgaccaacgacatcaattctctaacacgtcttgcaggaggaggagttatgtcagcagtggatgcctctatcacagcattagtaacgcttatcaccatgttctttactatttcgtggcaaatgacattaattgcggttatccctttgcccttaatggccttagcactagtaaattggggcgaaaaacccatgaaaccttcaaagaatctcaggcagcccttttcagaattaaataataaagtg

SEQ ID NO: 194 - protein, 178 aa, Streptococcus agalactiae
MSLSLVAVLNLIPPKIMGSVIDAITTGKLTRPQLLWNLLGLVLSALAMYGLRYIWRMYILGTSYKLGQVVRYRLFEHFTKMSPSFYQKYRTGDLMAHATNDINSLTRLAGGGVMSAVDASITALVTLITMFFTISWQMTLIAVIPLPLMALALVNWGEKPMKPSKNLRQPFSELNNKV

SEQ ID NO: 195 - DNA, 440 bp, Streptococcus agalactiae
atgcatattgagactgttattgatttcaaagaattaggaaaaagatatcgttttaaaaatcctacaaaagaattaatagctgatactttagaacaagtcttagaagtgataaaagaagttgattattatcaatctcaaaattattatgttgttggttatttatcttatgaagcatctgctgcttttgattcacattttaaagtttctcaacagaagttggctggagaacatctagcttattttacagtacataaagattgtgagaacgaagcttttcctttaagttatgaaaatgttagattagcagataattggactgctaatgtttctgagcaagaatatcaagaggcaattgctaatattaaaggacaaattagacaaggaaatacttatcaagtaaattatacactagagcttagccaacaattatgctcggatcc

SEQ ID NO: 196 - protein, 146 aa, Streptococcus agalactiae
MHIETVIDFKELGKRYRFKNPTKELIADTLEQVLEVIKEVDYYQSQNYYVVGYLSYEASAAFDSHFKVSQQKLAGEHLAYFTVHKDCENEAFPLSYENVRLADNWTANVSEQEYQEAIANIKGQIRQGNTYQVNYTLELSQQLCSD

SEQ ID NO: 197 - DNA, 1119 bp, Streptococcus agalactiae
gtgaataatatgttttatctcaaaatagcctggcataatttaaaacattctatagaccagtacataccattcctcttagccagtttattactttattcattgacttgttctacgctactaatcttaatgagtgctgttggaagagatatggggacagcggcaacggttctttttcttggagtgattgttttgtcaatctttgcggtagtcatggaacattatagctacaatatcttgatgaaacagcgtagtagtgaatttggactgtataacattttggggatgaataaacgtcaagttgcgcgtgtagctagtctagagctgtttattatttatatatttcttatttctataggaagtctgtttagtgctttttttgctaaatttatttatttaatttttgtcaacattattaactatcatgcactaaatcttagtttaagtttatggccatttattatttgtatcgttatatttacaggtatttttctgactttagaagttccagttattcgacatgttcatttatcatccccattaagtctttttagaaagaaacaacagggagaaaaagaaccaaaaggtaatcttatacttgcaattttagcgttagtagctatcgccatcgcttatacaatggctcttacttcaggtaaagcacctgcattagctgttatctatcgtttcttctttgcagtacttttagtaattgctggtacttatcttttttatattagttttatgacatggtacttaaaaaggttgcgtcaaaacaagcattattattataaatctgagcattttgtatcaacttcgcaaatgatttttcgaatgaagcaaaatgcagtagggttagcaagtatcactttattagctgttatggctctagttactattgctacaacagtctcactctattcaaatacacaaaatgttgttaccggactatttccaaaatcagtaagtttatcaatagataattcaaaaggtgacgcgaaaaatatatttgaagaaaagattttgaagaaactaggtaagtcatctaaggaagctatcacttataatcagacaatgatttcgatgccagttagtcaatcaagtgacttaatatcacatcta

SEQ ID NO: 198 - protein, 373 aa, Streptococcus agalactiae
MNNMFYLKIAWHNLKHSIDQYIPFLLASLLLYSLTCSTLLILMSAVGRDMGTAATVLFLGVIVLSIFAVVMEHYSYNILMKQRSSEFGLYNILGMNKRQVARVASLELFIIYIFLISIGSLFSAFFAKFIYLIFVNIINYHALNLSLSLWPFIICIVIFTGIFLTLEVPVIRHVHLSSPLSLFRKKQQGEKEPKGNLILAILALVAIAIAYTMALTSGKAPALAVIYRFFFAVLLVIAGTYLFYISFMTWYLKRLRQNKHYYYKSEHFVSTSQMIFRMKQNAVGLASITLLAVMALVTIATTVSLYSNTQNVVTGLFPKSVSLSIDNSKGDAKNIFEEKILKKLGKSSKEAITYNQTMISMPVSQSSDLISHL

SEQ ID NO: 199 - DNA, 735 bp, Streptococcus agalactiae
atggttgagccaattatttcaatacaaggacttcataaaagttttgggaaaaatgaggttttaaaaggcattgacttggatattcatcaaggagaagtggtggttattattggcccttctggctctggtaagtcaacatttttaagaacaatgaatctcttggaagtaccaacaaagggaacagtgacttttgaagggattgatataacagacaaaaagaatgatatttttaaaatgcgcgaaaaaatgggcatggtttttcaacagttcaatctatttcccaatatgactgtactagaaaatattactttatcacctattaagacaaagggactttctaagcttgatgctcagacaaaagcatacgagctacttgaaaaagttggactcaaagagaaggctaatgcttatccagcaagcttatctggaggacaacaacaacggattgctattgcaagaggtcttgcaatgaatcctgatgtccttctttttgatgaacctacttcagctcttgatcctgaaatggtaggtgaagtcttgactgttatgcaagatttagctaaatctggtatgacgatggttattgtcactcatgaaatgggttttgcacgtgaagtagcggatcgtgtcatttttatggatgcagggattattgttgagcaagggacccctaagaaagtatttgagcagacaaaagaaatccgcacaagagacttcttaagtaaagtattataa

SEQ ID NO: 200 - protein, 244 aa, Streptococcus agalactiae
MVEPIISIQGLHKSFGKNEVLKGIDLDIHQGEVVVIIGPSGSGKSTFLRTMNLLEVPTKGTVTFEGIDITDKKNDIFKMREKMGMVFQQFNLFPNMTVLENITLSPIKTKGLSKLDAQTKAYELLEKVGLKEKANAYPASLSGGQQQRIAIARGLAMNPDVLLFDEPTSALDPEMVGEVLTVMQDLAKSGMTMVIVTHEMGFAREVADRVIFMDAGIIVEQGTPKKVFEQTKEIRTRDFLSKVL

SEQ ID NO: 201 - DNA, 348 bp, Streptococcus agalactiae
atgtctcastatcaagagtggttagaaaacgactcactcggtaaagatattaagtcagatttagaagctattaaaggagatgaatctgaaattcaggatcgtttttacaaaacattagaatttggaacggcgggattgagaggtaaacttggagcaggaaccaatcgtatgaatacttatatggtggggaaagcagcacaagcattagctaatcgattattgatcatggccctgaagctattgcacgtggaattgcagttagttatgatgtcccgttatcaatctaaggaatttgcagaattaacttggtccattatggcagcaaatggtattaaagccttatattta

SEQ ID NO: 202 - protein, 122 aa, Streptococcus agalactiae
MSHMNYKEIYQEWLENDSLGKDIKSDLEAIKGDESEIQDRFYKTLEFGTAGLRGKLGAGTNRMNTYMVGKAAQALANRLLIMALKLLHVELQLVMMSRYQSKEFAELTWSIMAANGIKALYL

SEQ ID NO: 203 - DNA, 1068 bp, Streptococcus agalactiae
atgcaacctgtaaaagtcgatgaaccttctgttgaagaaaccattactattttgaaaggtatccaaaaaaaatacgaagattatcatcacgtaaaatataataatgatgccatagaagcagctgcagtactatctaatcgttatatccaagaccgctttttacctgataaagcaatagacttattagatgaagctggttctaaaatgaacctaacactaaattttgttgatccaaaagaaattgatcaacgtctcattgaagcagaaaatttaaaagcgcaagcgactcgtgaagaagattacgaacgtgcagcttacttccgtgaccagattgcaaaatataaagaaatgcagcaacaaaaggtcgacgatcaagatacacctattattaccgaaaaaacaattgagcacatcattgaagaaaaaacgaatatccctgttggtgatttaaaagaaaaagaacaatctcaattaattaatctcgcagatgacttgaaacagcatgtgatcggccaggatgacgctgtcattaagattgcaaaagctattcgtcgtaatcgagttggtcttggtagcccaaaccgtcctattggttcctttttatttgtaggaccaaccggtgttggtaaaactgaactttctaaacaactagcaattgagctctttggttcagctgatagtatgattcgttttgatatgtcagagtacatggaaaagcatgctgttgctaaattagtcggagcgcctccaggatacgtgggatacgaggaagctggacaactaactgaaaaggttcgtcgaaatccttactcgctcatccttctagatgaaattgaaaaagctcatcccgatgtcatgcatatgttcttgcaggtccttgatgacggtcgattaacagatggacaaggaagaactgttagttttaaagataccattatcatcatgacctcaaatgctggttctggtaaaactgaagcaagtgttggctttggtgcctcacgagaaggtaggacgaattcgagctcggtacccggggatcctctagagtcgacctgcaggcatgcaagc

SEQ ID NO: 204 - protein, 356 aa, Streptococcus agalactiae
MQPVKVDEPSVEETITILKGIQKKYEDYHHVKYNNDAIEAAAVLSNRYIQDRFLPDKAIDLLDEAGSKMNLTLNFVDPKEIDQRLIEAENLKAQATREEDYERAAYFRDQIAKYKEMQQQKVDDQDTPIITEKTIEHIIEEKTNIPVGDLKEKEQSQLINLADDLKQHVIGQDDAVIKIAKAIRRNRVGLGSPNRPIGSFLFVGPTGVGKTELSKQLAIELFGSADSMIRFDMSEYMEKHAVAKLVGAPPGYVGYEEAGQLTEKVRRNPYSLILLDEIEKAHPDVMHMFLQVLDDGRLTDGQGRTVSFKDTIIIMTSNAGSGKTEASVGFGASREGRTNSSSVPGDPLESTCRHAS

SEQ ID NO: 205 - DNA, 582 bp, Streptococcus agalactiae
atgagagggaaggttatttacggcacaacccttataggtctttttctattcttatttttctatttttggattcctaagcatcacatcgagagaatacatcatcatcgtataaagcaggtagatgcgaagagtgatttaacaggatttaaaacccatttgcccattatcagcattgatacaaagcaacaagttattcctcttgttacaaaagaaggcggaaaatatgtcaaagctagggataatattaatgttgatatcgaattacgggattctccaagtagatcacatcatttatcagaaaagccgagaattaggacaaaagggttaatatcatatagaggaaattcctctcgttactttgataagaagtcattgaaagttaagtttgttactaataagttaaaggaaaagaagcatcgattagcaggaatgcctaaagaatcggagtgggtattgcatggtccctttctagacagaacattattaagaaattatctgagttataatattgctggtgagattatgcctatgccccaaacgttcgctactgtgagttatttgtcaatggtgagtatcagggag

SEQ ID NO: 206 - protein, 194 aa, Streptococcus agalactiae
MRGKVIYGTTLIGLFLFLFFYFWIPKHHIERIHHHRIKQVDAKSDLTGFKTHLPIISIDTKQQVIPLVTKEGGKYVKARDNINVDIELRDSPSRSHHLSEKPRIRTKGLISYRGNSSRYFDKKSLKVKFVTNKLKEKKHRLAGMPKESEWVLHGPFLDRTLLRNYLSYNIAGEIMPMPQTFATVSYLSMVSIRE

SEQ ID NO: 207 - DNA, 498 bp, Streptococcus agalactiae
cttcacattttattgatcactatctgacaaatgttaatcaaacagcagttcttattttagtgggatattattcaatgtatgtcttgcagaccttaattcaatattttgggaatctcttttttgcgcgtgtttcttatagtattgttagagatattcgtagagatgcttttgctaatatggaaaggctaggcatgtcttattttgataggacaccggcaggatctattgtgtcacgtattactaatgatactgaagcaatatctgatatgttttcgggtattttatcaagttttatctcggcgatatttatttttacagttactctgtacactatgttgatgctagacattaaactaacaggactcgtcgctcttttgttacctgttatctttatattagtgaatgtctatcggaaaaaatcagtcactgtcattgctaaaacgagaagtttacttagtgatatcaacagtaaattatcagaaagtattgaaggaattc

SEQ ID NO: 208 - protein, 165 aa, Streptococcus agalactiae
SHFIDHYLTNVNQTAVLILVGYYSMYVLQTLIQYFGNLFFARVSYSIVRDIRRDAFANMERLGMSYFDRTPAGSIVSRITNDTEAISDMFSGILSSFISAIFIFTVTLYTMLMLDIKLTGLVALLLPVIFILVNVYRKKSVTVIAKTRSLLSDINSKLSESIEGI

SEQ ID NO: 209 - DNA, 681 bp, Streptococcus agalactiae
atgtaccatattgaattaaaaaaggaagctttactaccaagagaacgcctagttgatttaggcgcagatagattgagtaatcaggagttattagccattctcttacgtacaggtattaaagaaaaacctgttcttgaaatttcaacgcaaattttagaaaacataagcagtttagcagattttggtcaattatccttacaggagttgcaatccattaaaggaatcggtcaggttaaatccgtcgaaataaaagctatgctagaactagcaaaacggattcacaaagctgaatatgatcgtaaagagcaaattttaagtagtgaacaattagcgaggaaaatgatgctcgaattaggggataaaaaacaagaacatttagtagctatttatatggatacacaaaatcgtattatcgaacagagaactatttttattggtactgtacgtcgttcagtagcagagccaagagaaattctacattatgcttgtaaaaacatggcaacttctttgattattatacataatcatccctcaggttctccaaatcccagtgaaagtgatttaagtttcactaaaaaaataaaacgatcatgtgatcatctgggaattgtctgcctagatcacatcatcgttggaaaaaataaatattatagttttcgagaagaagcagatattttataa

SEQ ID NO: 210 - protein, 226 aa, Streptococcus agalactiae
MYHIELKKEALLPRERLVDLGADRLSNQELLAILLRTGIKEKPVLEISTQILENISSLADFGQLSLQELQSIKGIGQVKSVEIKAMLELAKRIHKAEYDRKEQILSSEQLARKMMLELGDKKQEHLVAIYMDTQNRIIEQRTIFIGTVRRSVAEPREILHYACKNMATSLIIIHNHPSGSPNPSESDLSFTKKIKRSCDHLGIVCLDHIIVGKNKYYSFREEADIL

SEQ ID NO: 211 - DNA, 579 bp, Streptococcus agalactiae
tggttaaaagtagtgatagcttgtattccatctattttaattgctttaccatttgataattggtttgaagctcattttaatttcatgattccgattgcaatagccctaatcttttatggttttgtcttcatatgggttgaaaaacgtaatgcacacctcaaaccacaggtaaccgaattggcaagtatgtcttacaagacagctttcttgattggatgtttccaggttctcagtattgttccgggaaccagtcgttctggagctactattttaggagcaattattattggaactagtcgttcggtcgctgctgactttactttcttccttgccatcccaactatgtttggttatagtggacttaaggcggttaaatattttttagatggtaacgtcttgagtttagaccaatctttaatacttttagtagcaagtctgacagctttcgtagttagtttatatgttattcgtttcttgacagactatgtcaaacgacacgatttcaccatctttggtaagtatcgtatagtcttaggaagtttactcatcctctactggttagttgttcatttattctaa

SEQ ID NO: 212 - protein, 192 aa, Streptococcus agalactiae
WLKVVIACIPSILIALPFDNWFEAHFNFMIPIAIALIFYGFVFIWVEKRNAHLKPQVTELASMSYKTAFLIGCFQVLSIVPGTSRSGATILGAIIIGTSRSVAADFTFFLAIPTMFGYSGLKAVKYFLDGNVLSLDQSLILLVASLTAFVVSLYVIRFLTDYVKRHDFTIFGKYRIVLGSLLILYWLVVHLF

SEQ ID NO: 213 - DNA, 547 bp, Streptococcus agalactiae
atggaaatgaaacaaatcagtgaaacaacactgaaaattacaattagtatggaagatttagaagatcgtggtatggagctgaaagatttcctaatccctcaggagaagactgaggaatttttctattctgtcatggatgaattagacttgccagaaaactttaaaaatagtggtatgttaagttttcgagtaacacctaaaaaagatcgcattgatgtttttgttacaaagtctgaattaagtaaagatttaaatttagaagaattagcagatttgggtgacatttcaaaaatgtctccagaagacttttttaaaaccttggaacaatcgatgttggaaaaaggggatacggatgcccatgccaaattagcagaaattgaaaatatgatggataaagcaactcaagaagtagttgaggaaaatgtttctgaagaacaacctgaaaaggaagtagaaacgattggatatgttcactatgtctttgattttgataatattgaagctgtagttcgattttcacaaacgattgattttccaatagaagctt

SEQ ID NO: 214 - protein, 182 aa, Streptococcus agalactiae
MEMKQISETTLKITISMEDLEDRGMELKDFLIPQEKTEEFFYSVMDELDLPENFKNSGMLSFRVTPKKDRIDVFVTKSELSKDLNLEELADLGDISKMSPEDFFKTLEQSMLEKGDTDAHAKLAEIENMMDKATQEVVEENVSEEQPEKEVETIGYVHYVFDFDNIEAVVRFSQTIDFPIEA

SEQ ID NO: 215 - DNA, 447 bp, Streptococcus agalactiae
ggaaaccaacggccagtacaatcgtcaagggtagattatcctaaacgtagtcgtgccaagattgtagaagtttattttagacaagcttctactactgattattctggtgtttacaaaggttactatattgactttgaagccaaagaaacccggcagaaaactgctatgcctatgaaaaattttcatgctcaccaaatagagcacatggcaaatgtattacagcaaaaagggatttgctttgtcttgcttcatttttccacacttaaggaaacctatctactccctgctaatgagttaatttcattttatcagattgataaaggcaataaatcaatgcctattgattatatcagaaaaaatggatttttcgtaaaggagagtgcctttcctcaagtcccttacttagatattattgaagaaaaattattaggcggtgattacaattaa

SEQ ID NO: 216 - protein, 148 aa, Streptococcus agalactiae
GNQRPVQSSRVDYPKRSRAKIVEVYFRQASTTDYSGVYKGYYIDFEAKETRQKTAMPMKNFHAHQIEHMANVLQQKGICFVLLHFSTLKETYLLPANELISFYQIDKGNKSMPIDYIRKNGFFVKESAFPQVPYLDIIEEKLLGGDYN

SEQ ID NO: 217 - DNA, 433 bp, Streptococcus agalactiae
ggatcctaaaaacgctaaggtttatcaaaaaaatgctgatcaatttagtgacaaggcaatggctattgcagagaagtataagccaaaatttaaagctgcaaagtctaaatactttgtgacttcacatacagcattctcatacttagctaagcgatacggattgactcagttaggtattgcaggtgtctcaaccgagcaagaacctagtgctaaaaaattagccgaaattcaggagtttgtgaaaacatataaggttaagactatttttgttgaagaaggagtctcacctaaattagctcaagcagtagcttcagctactcgagttaaaattgcaagtttaagtcctttagaagcagttcccaaaaacaataaagattacttagaaaatttggaaactaatcttaaggtacttgtcaaatcgttaaatcaatag

SEQ ID NO: 218 - protein, 143 aa, Streptococcus agalactiae
DPKNAKVYQKNADQFSDKAMAIAEKYKPKFKAAKSKYFVTSHTAFSYLAKRYGLTQLGIAGVSTEQEPSAKKLAEIQEFVKTYKVKTIFVEEGVSPKLAQAVASATRVKIASLSPLEAVPKNNKDYLENLETNLKVLVKSLNQ

SEQ ID NO: 219 - DNA, 717 bp, Streptococcus agalactiae
atgaaaaaagtcatcgatttaaaaaaactacaaaaagcatacgcctcagaaactgttttaaataatattaatttggaggtgtttaaaggagaaataattggattaataggaccctctggagcagggaaatctaccttgattaaaactatgcttggcatggaaaaagcagataagggaacagctcttgttcttgatactcaaatgccagatcgtaatattttaaatcaaattggctatatggctcaatctgatgccttacacgagtctttaactggcttagaaaatttattattctttggaaaaatgaaaggtattcaaaaaactgaattaaaacagcagataactcatatttctaaagtagtagatctagaaaaccaacttgataaatttgtctcaggttactcagaaggtatgaaaagacggctttctctagccatcgccctacttggaaaccccacagttttaatcctagatgaacctaccgttggaattgatccatccttgaggagaaaaatctggcaagagctaattaatattaaggatgaaggacgttctatctttattacaacccacgttatggatgaagcagaattaacaagtaaggttgcactactattacgtggaaacattattgcctttgatactccattacatttaaaaaaacaatttaatgtgagtactattgaggaagttttcttaaaagctgaaggagaataa

SEQ ID NO: 220 - protein, 238 aa, Streptococcus agalactiae
MKKVIDLKKLQKAYASETVLNNINLEVFKGEIIGLIGPSGAGKSTLIKTMLGMEKADKGTALVLDTQMPDRNILNQIGYMAQSDALHESLTGLENLLFFGKMKGIQKTELKQQITHISKVVDLENQLDKFVSGYSEGMKRRLSLAIALLGNPTVLILDEPTVGIDPSLRRKIWQELINIKDEGRSIFITTHVMDEAELTSKVALLLRGNIIAFDTPLHLKKQFNVSTIEEVFLKAEGE

SEQ ID NO: 221 - DNA, 591 bp, Streptococcus agalactiae
atggtacaaatgatacatgatatgattaaaacaattgagcattttgctgagacacaagctgattttccagtgtatgatattttaggggaagtccatacttatggacaacttaaagtagactctgactctctagctgctcatattgatagcctaggccttgttgaaaaatcacctgtcttagtattcggtggtcaagaatatgaaatgttggcgacatttgttgctttaacaaagtcagggcatgcttatataccggttgaccaacactctgctttggatagaatacaggctattatgacagttgctcaaccaagccttatcatttcaattggtgaatttcctcttgaagttgataatgtcccaatcctagacgtttctcaagtttcagctatttttgaagaaaagactccttatgaggtaacacattctgttaaaggtgatgataattactatattattttcacttcagggactactggtttaccaaaaggtgtgcaaatttcacatgacaatttattgagctttacaaattggatgatttctgatgatgagttttcagttcctgaaagaccgcaaatgttggctcaaccc

SEQ ID NO: 222 - protein, 197 aa, Streptococcus agalactiae
MVQMIHDMIKTIEHFAETQADFPVYDILGEVHTYGQLKVDSDSLAAHIDSLGLVEKSPVLVFGGQEYEMLATFVALTKSGHAYIPVDQHSALDRIQAIMTVAQPSLIISIGEFPLEVDNVPILDVSQVSAIFEEKTPYEVTHSVKGDDNYYIIFTSGTTGLPKGVQISHDNLLSFTNWMISDDEFSVPERPQMLAQP

SEQ ID NO: 223 - DNA, 1179 bp, Streptococcus agalactiae
atggaaaatcatcgttatgaagatgaaggtaaattccagcgtaagatgaccagtcgtcatctctttatgttatcgctaggtggtgttatcgggactgggcttttcttgagttcaggttataccattgcacaggctggtccgcttggagctgtgctgtcttatttgattggtgccgttgtggtttatttggtcatgctatcacttggggaattggcggttgccatgccggtgacggggtcattccacacttatgccactaagtttatcagtcctggaacaggttttactgttgcttggctatattggatttgttggacggtcgccttggggactgaatttttaggtgctgccatgctgatgcagcgctggttcccaaatgtgccggcttgggcatttgcttccttttttgcccttgtgatttttggtttaaatgctcttagcgtacgcttttttgcagaagcagagtctttcttctcaagtattaaggttattgctatcattatctttattatcttgggcttaggtgctatgtttggtctagtttcctttgaaggtcagcacaaggctattctcttcactcatctgactgccaatggtgcctttccaaatggtatcgttgcagttgtctcagtcatgttggctgttaactatgccttctctggtactgagttaattggtattgcggctggtgaaacggataatcccaaagaagctgtaccaagggctattaaaacgacaatcggtcgcttggttgttttctttgtactgacaattgttgtcctagcttcgctattgccaatgaaagaggcaggcgtatccacagcaccattcgttgatgtctttgacaagatgggaatcccttttacggcggatatcatgaacttcgttatcttgacagccatcctgtctgctggtaactcaggtctctacgcatcaagccgtatgctctggtcccttgccaatgaaggtatgttgtcaaaatctgttgtgaaaatcaataaacacggtgtcccaatgcgtgctcttctcttgtcaatggcaggagcagtgctgtcgctcttttcaagtatttacgctgcagacacagtttatctagccttggtttcaatcgcgggctttgctgttgttgtcgtatggctagccattccagtcgcacaaatcaatttccgcaaggaattc

SEQ ID NO: 224 - protein, 393 aa, Streptococcus agalactiae
MENHRYEDEGKFQRKMTSRHLFMLSLGGVIGTGLFLSSGYTIAQAGPLGAVLSYLIGAVVVYLVMLSLGELAVAMPVTGSFHTYATKFISPGTGFTVAWLYWICWTVALGTEFLGAAMLMQRWFPNVPAWAFASFFALVIFGLNALSVRFFAEAESFFSSIKVIAIIIFIILGLGAMFGLVSFEGQHKAILFTHLTANGAFPNGIVAVVSVMLAVNYAFSGTELIGIAAGETDNPKEAVPRAIKTTIGRLVVFFVLTIVVLASLLPMKEAGVSTAPFVDVFDKMGIPFTADIMNFVILTAILSAGNSGLYASSRMLWSLANEGMLSKSVVKINKHGVPMRALLLSMAGAVLSLFSSIYAADTVYLALVSIAGFAVVVVWLAIPVAQINFRKEF

SEQ ID NO: 225 - DNA, 636 bp, Streptococcus agalactiae
tcagaaaatgcagaggcagcaacggttgccacaaacttggttaccaaaggagctaatgtcattatcggaccagcaacatcgggtgcagctgcatcttcaactccaaaagtaaatgcagcagcagttccaatgattgcacctgctgcgacacaagacaatttagtctatggttctgatggaaaaaccttaaatcagtatttcttccgagctacttttgtcgataattatcaaggaaagctattgtctcagtatgctacagacaaccttaaagctaaaaaagttgttctattttatgataattcatcagattactcaaagggggtagcaaaatcatttaaggaaagttatagtggaaaaattgttgatagtatgacattctccgctggtgatactgatttccaagcgtcattgactaagttgaaagggaaagaatatgatgctattgtgatgccaggttactataccgagacaggattaatagttaagcaagcgcgtgatttaggtatctctaaaccggttcttgggcctgatggttttgatagtccgaaatttgtgcaatcggcaacacctgtaggagcttcaaacgtttattatttgacaggtttcactacacaaggatcaaccaaagctaaagct

SEQ ID NO: 226 - protein, 212 aa, Streptococcus agalactiae
SENAEAATVATNLVTKGANVIIGPATSGAAASSTPKVNAAAVPMIAPAATQDNLVYGSDGKTLNQYFFRATFVDNYQGKLLSQYATDNLKAKKVVLFYDNSSDYSKGVAKSFKESYSGKIVDSMTFSAGDTDFQASLTKLKGKEYDAIVMPGYYTETGLIVKQARDLGISKPVLGPDGFDSPKFVQSATPVGASNVYYLTGFTTQGSTKAKA

SEQ ID NO: 227 - DNA, 270 bp, Streptococcus agalactiae
ttgggacttaaagaccatgctttagtctatccattttcattatctggggggcaaaagcaacgtgtcgcactagctcgtgcgatgatgattgatccacagattattggttatgatgagccaactagcgctcttgatccagagttgcgtcaagaagtagaaaaactaattttacaaaatagagaaacaggtatgacacaaattgtagtaacacatgatcttcaatttgctgaaagtatatctgatacgattctcaaaattaatcctaagtag

SEQ ID NO: 228 - protein, 89 aa, Streptococcus agalactiae
MGLKDHALVYPFSLSGGQKQRVALARAMMIDPQIIGYDEPTSALDPELRQEVEKLILQNRETGMTQIVVTHDLQFAESISDTILKINPK

SEQ ID NO: 229 - DNA, 204 bp, Streptococcus agalactiae
atgactaatatctcagatgttccaaaagctattagaacacaggcacagtatgttctcttgggaatgagagttatggatcagtcggtattaccgaaaacatataattcaaaagaaccttatttgaaaccagatatgatttatattcatgatagaagacaagagacaatgcttaaaatcactcaagaaatagaaatggagcattga

SEQ ID NO: 230 - protein, 67 aa, Streptococcus agalactiae
MTNISDVPKAIRTQAQYVLLGMRVMDQSVLPKTYNSKEPYLKPDMIYIHDRRQETMLKITQEIEMEH

SEQ ID NO: 231 - DNA, 1411 bp, Streptococcus agalactiae
aagcttgcatgcctgcaggtcgactctagaggatcttggggaatataaatttggatttcatgacgatgtaaagccaatttattctacgggaaaaggtctaaatgaggctgttattcgtgagttatctgcagctaagggtgaacctgagtggatgttggactttcgtctaaaatccttggaaacgtttaataaaatgccgatgcagacctggggagcagatttatcagatattgattttgatgatattatttattatcaaaaagcatctgataaacctgcgcgtgattgggatgatgttccagaaaaaatcaaagaaacttttgaaagaattgggattccagaagctgaaagagcctatcttgcaggagcatcagcacaatatgaatcagaagtagtttatcacaatatgaaagaagaatatgataagctgggtattgtttttacggatactgactctgcacttaaagagtacccagagctattcaaaaaatattttgctaaacttgtccctccaacagataataaattagctgctctgaactctgctgtatggtcaggtggaacatttatttatgttcctaaaggtgttaaggtggatattccacttcaaacttacttccgtattaataatgaaaatactggacaatttgaacgtactctcattattgttgatgagggagcaagtgttcactatgttgaaggttgtaccgccccaacttattcttcaaatagtttacatgcagctatagttgaaatttttgcacttgatggagcttatatgcgctatacgactattcaaaattggtccgataatgtctataatttagtgacaaaacgtgctaccgctaaaaaagatgcaacagttgagtggatagatggaaatctaggagctaaaacaacaatgaaatacccatcggtttaccttgatggtgaaggagcacgtggcacgatgttgtctattgcttttgcaaacaaaggacaacaccaagatacgggtgcaaagatgattcataatgccccccatactagttcatccattgtctctaaatcaattgctaagggtgggggaaaagttgattatcgaggtcaagtgacatttaataaagattccaaaaaatcagtgtcacatatagaatgtgacaccatattgatggatgatatttcaaaatcagataccataccgtttaatgagattcataattcacaggttgctttagagcatgaagcaaaggtgtctaagatttctgaagagcaactgtactacttgatgagtcgaggtttatctgaagctgaagcaacagaaatgattgttatggggtttgttgagccctttacgaaagaattaccaatggaatatgcggtagagttaaatcgtttaatttcctatgaaatggaaggttcagttggttaa

SEQ ID NO: 232 - protein, 468 aa, Streptococcus agalactiae
MHACRSTLEDLGEYKFGFHDDVKPIYSTGKGLNEAVIRELSAAKGEPEWMLDFRLKSLETFNKMPMQTWGADLSDIDFDDIIYYQKASDKPARDWDDVPEKIKETFERIGIPEAERAYLAGASAQYESEVVYHNMKEEYDKLGIVFTDTDSALKEYPELFKKYFAKLVPPTDNKLAALNSAVWSGGTFIYVPKGVKVDIPLQTYFRINNENTGQFERTLIIVDEGASVHYVEGCTAPTYSSNSLHAAIVEIFALDGAYMRYTTIQNWSDNVYNLVTKRATAKKDATVEWIDGNLGAKTTMKYPSVYLDGEGARGTMLSIAFANKGQHQDTGAKMIHNAPHTSSSIVSKSIAKGGGKVDYRGQVTFNKDSKKSVSHIECDTILMDDISKSDTIPFNEIHNSQVALEHEAKVSKISEEQLYYLMSRGLSEAEATEMIVMGFVEPFTKELPMEYAVELNRLISYEMEGSVG

SEQ ID NO: 233 - DNA, 261 bp, Streptococcus agalactiae
atgatagaattcttttctaatatcagaacagagattccgcagatgcctttacttatccatagtttgattttatctgtcttaccttttctgatgtggctgactttggttaatagagataagcctttgtataaaactatttggagtatccttttaggacttcagttaattacgatttatacttggtttttctgggcaaaattgcctttatctgaaagtcttcccctttaccattgtcgaataggcatgtttgtcggtctctta

SEQ ID NO: 234 - protein, 87 aa, Streptococcus agalactiae
MIEFFSNIRTEIPQMPLLIHSLILSVLPFLMWLTLVNRDKPLYKTIWSILLGLQLITIYTWFFWAKLPLSESLPLYHCRIGMFVGLL

SEQ ID NO: 235 - DNA, 486 bp, Streptococcus agalactiae
aagcttgtgcaaagtattaaagagataggattagctaatgcgcatttattagctgttgctccgacagggtcaatcagttatctttcttcttgtactccgagccttcaaccggttgtatcacctgtcgaagtacgcaaggaaggagcactggggagggtttatgtagctgcttataagattgatgcagataattatgtctactacaaaaaaggagcttatgaagtgggatctgaggcgattatcaatattgcagctgccgctcaaaaacacattgatcaagctatttcgttaacgcttttcatgacagatcaagcaactacgcgagatttaaataaagcctatattcaagcatttaaacaaaaatgtgcctctatttattatgtacgagtgagacaggacatcctagaaggtagcgagagttatgatgatatgctggatgatttcacttcatcggacttagaagactgtcaatcctgcatgatttaa

SEQ ID NO: 236 - protein, 161 aa, Streptococcus agalactiae
KLVQSIKEIGLANAHLLAVAPTGSISYLSSCTPSLQPVVSPVEVRKEGALGRVYVAAYKIDADNYVYYKKGAYEVGSEAIINIAAAAQKHIDQAISLTLFMTDQATTRDLNKAYIQAFKQKCASIYYVRVRQDILEGSESYDDMLDDFTSSDLEDCQSCMI

SEQ ID NO: 237 - DNA, 413 bp, Streptococcus agalactiae
gtgaggacatatattacaaacttgaatggacattcaatcactagtacagcacaaatagctcaaaacatggtaacagatatagcagtaagcttaggttttcgtgagctgggaatacattcttatccgattgatactgattctcctgaggaaatgagtaagcgtttagatggaatctgttccggacttagaaaaaatgatattgtcatatttcagacacctacatggaacactacaacttttgatgaaaaattatttcacaaattaaaaatatttggtgtaaagattgttatttttatacatgatgttgtaccgctaatgtttgatggaaatttttatttgatggatagaactatagcttattataatgaagcagatgtttaatagcccctagtcaagcaatggtcgataagctt

SEQ ID NO: 238 - protein, 138 aa, Streptococcus agalactiae
MRTYITNLNGHSITSTAQIAQNMVTDIAVSLGFRELGIHSYPIDTDSPEEMSKRLDGICSGLRKNDIVIFQTPTWNTTTFDEKLFHKLKIFGVKIVIFIHDVVPLMFDGNFYLMDRTIAYYNEADVLIAPSQAMVDKL

SEQ ID NO: 239 - DNA, 261 bp, Streptococcus agalactiae
catggaaatgaagttgatgatgttattagaagggcatttgaatataatcaccttatctttgcttttgataatacctgtcataacagagagttagtattagatagcaatatcatttctcacacaacctgtgaacaattgataaatttaatgaaaaatttatcaggctccattatgtatttgctagagcaacaaagagaacaaacaagtaatgaaacaaaagagcgttataaagaaatattaggagggtatggaaatgcctaa

SEQ ID NO: 240 - protein, 86 aa, Streptococcus agalactiae
HGNEVDDVIRRAFEYNHLIFAFDNTCHNRELVLDSNIISHTTCEQLINLMKNLSGSIMYLLEQQREQTSNETKERYKEILGGYGNA

SEQ ID NO: 241 - DNA, 312 bp, Streptococcus agalactiae
acatttttatattatgtatttgaagacgtagccacccagtcaaatatgactgggaagatttttagtatgtctaaagaagagttgtcatatttacccgttattaaactttttaagaatcaaggtgtatacaacggcttgattggtctattcctcctttatgggttatatatttcacagaatcaagaaattgtagctatttttttaatcaatgtgttgctagttgctgtttatggtgctttgacagttgataaaaaaatcttattaaaacagggtggtttacctatattagctcttttaacattcttattttaa

SEQ ID NO: 242 - protein, 103 aa, Streptococcus agalactiae
TFLYYVFEDVATQSNMTGKIFSMSKEELSYLPVIKLFKNQGVYNGLIGLFLLYGLYISQNQEIVAIFLINVLLVAVYGALTVDKKILLKQGGLPILALLTFLF

SEQ ID NO: 243 - DNA, 588 bp, Streptococcus agalactiae
atgaaattaagtgtccttgattatgggcttattgattatggaaaaactgcaagtgatgcaatacaagaaacgattcttttatcacaagaggcggagcaactaggctatcatcaattttgggtggctgaacatcacggtgttaaggcattcagtattagcaatccagaattaatgataatgcatttggctaaccagactaaatctatcaaaattggctctggaggtataatgcctctgcactatagtagttttaaactcgcggagactctcaagacattagagacatgtcatccgaatcgagtaagtattggtttaggaaattcactagggacagttaaagtttcaaatgcacttcgtagcttacataaagcacatgattacgaagaggtactggaggaattgaagtcatggcttattgatgaatcatccagtaaggaaccattagttcaaccgactctttctagcttcccagacttatatgtgttggggagtggtcaaaaatcagcttatttagcggctaaacttggcttaggctttaccttcggtgtttttccttttatggacaaagacccattgacagaagctaaa

SEQ ID NO: 244 - protein, 196 aa, Streptococcus agalactiae
MKLSVLDYGLIDYGKTASDAIQETILLSQEAEQLGYHQFWVAEHHGVKAFSISNPELMIMHLANQTKSIKIGSGGIMPLHYSSFKLAETLKTLETCHPNRVSIGLGNSLGTVKVSNALRSLHKAHDYEEVLEELKSWLIDESSSKEPLVQPTLSSFPDLYVLGSGQKSAYLAAKLGLGFTFGVFPFMDKDPLTEAK

SEQ ID NO: 245 - DNA, 40 bp, Artificial Sequence
  Primer
cgagatctgatatctcacaaacagataacggcgtaaatag

SEQ ID NO: 246 - DNA, 43 bp, Artificial Sequence
  Primer
gaagatcttccccgggatcacaaacagataacggcgtaaatag

SEQ ID NO: 247 - DNA, 42 bp, Artificial Sequence
  Primer
cgagatctgatatccatcacaaacagataacggcgtaaatag

SEQ ID NO: 248 - DNA, 32 bp, Artificial Sequence
  Primer
cgggatccttatggacctgaatcagcgttgtc

SEQ ID NO: 249 - DNA, 23 bp, Artificial Sequence
  Primer
ggatgctttgtttcaggtgtatc

SEQ ID NO: 250 - DNA, 82 bp, Artificial Sequence
  Primer
catgatatcggtacctcaagctcatatcattgtccggcaatggtgtgggctttttttgttttagcggataacaatttcacac

SEQ ID NO: 251 - DNA, 81 bp, Artificial Sequence
  Primer
gcggatcccccgggcttaattaatgtttaaacactagtcgaagatctcgcgaattctcctgtgtgaaattgttatccgcta

SEQ ID NO: 252 - DNA, 24 bp, Artificial Sequence
  Primer
cgccagggttttcccagtcacgac

SEQ ID NO: 253 - DNA, 20 bp, Artificial Sequence
  Primer
tcaggggggcggagcctatg

SEQ ID NO: 254 - DNA, 22 bp, Artificial Sequence
  Primer
tcgtatgttgtgtggaattgtg

SEQ ID NO: 255 - DNA, 26 bp, Artificial Sequence
  Primer
tccggctcgtatgttgtgtggaattg

SEQ ID NO: 256 - DNA, 43 bp, Artificial Sequence
  pTREP1-nuc1 vector
aagtatcagatctgatatctcacaaacagataacggcgtaaat

SEQ ID NO: 257 - DNA, 46 bp, Artificial Sequence
  pTREP1-nuc2 vector
aagtatcagatcttccccgggatcacaaacagataacggcgtaaat

SEQ ID NO: 258 - DNA, 45 bp, Artificial Sequence
  pTREP1-nuc3 vector
aagtatcagatctgatatccatcacaaacagataacggcgtaaat

SEQ ID NO: 259 - DNA, 24 bp, Staphylococcus aureus
tcacaaacagataacggcgtaaat

SEQ ID NO: 260 - DNA, 17 bp, Artificial Sequence
  Primer
cgggatccgccaccatg

SEQ ID NO: 261 - DNA, 10 bp, Artificial Sequence
  Primer
ttgcggccgc

SEQ ID NO: 262 - DNA, 38 bp, Artificial Sequence
  Primer
cggatccgccaccatggcggatcaaactacatcggttc

SEQ ID NO: 263 - DNA, 36 bp, Artificial Sequence
  Primer
ttgcggccgcgttgggataactagtcggtttagtcg

SEQ ID NO: 264 - DNA, 44 bp, Artificial Sequence
  Primer
cggatccgccaccatgaatctttatttccatagtactcccttgc

SEQ ID NO: 265 - DNA, 37 bp, Artificial Sequence
  Primer
ttgcggccgcaaaatgatcagtttgagggtaaaagag

SEQ ID NO: 266 - DNA, 31 bp, Artificial Sequence
  Primer
catgccatggcggatcaaactacatcggttc

SEQ ID NO: 267 - DNA, 37 bp, Artificial Sequence
  Primer
catgccatggcaaaaatagtagtaccagtaatgcctc

SEQ ID NO: 268 - DNA, 32 bp, Artificial Sequence
  Primer
ttgcggccgcctctgaaatagtaatttgtccg

SEQ ID NO: 269 - DNA, 35 bp, Artificial Sequence
  Primer
catgccatgggaaagaaagcaaataatgtcagtcc

SEQ ID NO: 270 - DNA, 31 bp, Artificial Sequence
  Primer
ttgcggccgcattgggtgtaagcattttttc

SEQ ID NO: 271 - DNA, 37 bp, Artificial Sequence
  Primer
catgccatgggaactgagaactggttacatactaaag

SEQ ID NO: 272 - DNA, 33 bp, Artificial Sequence
  Primer
ttgcggccgcattagctttttcaacaatttctc

SEQ ID NO: 273 - DNA, 28 bp, Artificial Sequence
  Primer
ctagctagccgatgtttgcgtgggaaag

SEQ ID NO: 274 - DNA, 40 bp, Artificial Sequence
  Primer
ttgcggccgcataagatttaacaataccaagtaatatagc

SEQ ID NO: 275 - DNA, 39 bp, Artificial Sequence
  Primer
ggggtaccggccaccatggctgaagtaatttcaggaagt

SEQ ID NO: 276 - DNA, 39 bp, Artificial Sequence
  Primer
cggaattccgttaatcctctttttttcttagaaacagat

(19) United States
(12) Patent Application Publication
(10) Pub. No.: US 2003/0170782 A1
(43) Pub. Date: Sep. 11, 2003

(54) Proteins

(76) Inventors: Richard William Falla Le Page, Cambridge (GB); Jeremy Mark Wells, Norwich (GB); Sean Bosco Hanniffy, Norwich (GB)

Correspondence Address: HUNTON & WILLIAMS, INTELLECTUAL PROPERTY DEPARTMENT, 1900 K STREET, N.W., SUITE 1200, WASHINGTON, DC, 20006-1109, US

(21) Appl. No.: 10/091,007

(22) Filed: Mar. 6, 2002

Related U.S. Application Data
(63) Continuation of application No. PCT/GB00/03437, filed on Sep. 7, 2000.

(30) Foreign Application Priority Data
Sep. 7, 1999 (GB) 9921125.2

(51) Int. Cl. C12P 21/02; C12N 1/21; C07H 21/04; C07K 14/315; C12N 15/74

(52) U.S. Cl. 435/69.1; 435/252.3; 435/320.1; 530/350; 536/23.2

(57) ABSTRACT

Novel protein antigens from Group B Streptococcus are described, together with the nucleic acid sequences encoding them. The use of vaccines and screening methods is also described.

The present invention relates to proteins derived from Streptococcus agalactiae, nucleic acid molecules encoding such proteins, and the use of the proteins as antigens and/or immunogens and in detection/diagnosis. It also relates to a method for the rapid screening of bacterial genomes to isolate and characterise bacterial cell envelope associated or secreted proteins.

The Group B Streptococcus (GBS) (Streptococcus agalactiae) is an encapsulated bacterium which emerged in the 1970s as a major pathogen of humans causing sepsis and meningitis in neonates as well as adults. The incidence of early onset neonatal infection during the first 5 days of life varies from 0.7 to 3.7 per 1000 live births and causes mortality in about 20% of cases. Between 25-50% of neonates surviving early onset infections frequently suffer neurological sequalae. Late onset neonatal infections occur from 6 days to three months of age at a rate of about 0.5 - 1.0 per 1000 live births.

There is an established association between the colonisation of the maternal genital tract by GBS at the time of birth and the risk of neonatal sepsis. In humans it has been established that the rectum may act as a reservoir for GBS. Susceptibility in the neonate is correlated with the a low concentration or absence of IgG antibodies to the capsular polysaccharides found on GBS causing human disease. In the USA strains isolated from clinical cases usually belong to capsular serotypes Ia, Ib, II, III although serotype V may be of increasing significance. Type VIII GBS is the major cause of neonatal sepsis in Japan.

A possible means of prevention involves intra or postpartum administration of antibiotics to the mother but there are concerns that this might lead to the emergence of resistant organisms and in some cases allergic reactions. Vaccination of the adolescent females to induce long lasting maternally derived immunity is one of the most promising approaches to prevent GBS infections in neonates. The capsular polysaccharide antigens of these organisms have attracted most attention as with regard to vaccine development. Studies in healthy adult volunteers have shown that serotype Ia, II and III polysaccharides are non-toxic and immunogenic in approximately 65%, 95% and 70% of non-immune adults respectively. One of the problems with using capsule antigens as vaccines is that the response rates vary according to pre-immunisation status and the polysaccharide antigen and not all vaccinees produce adequate levels of IgG antibody as indicated in vaccination studies with GBS polysaccharides in human volunteers.

Some people do not respond despite repeated stimuli. These properties are due to the T-independent nature of polysaccharide antigens. One strategy to enhance the immunogenicity of these vaccines is to enhance the T cell dependent properties of polysaccharides by conjugating them to a protein. The use of polysaccharide conjugates looks promising but there are still unresolved questions concerning the nature of the carrier protein. A conjugate vaccine against GBS would require at least 4 different conjugates to be prepared adding to the cost of a vaccine.

Approaches to vaccination against GBS infections which rely on the use of capsular polysaccharides have the disadvantage that response rates are likely to vary considerably according to pre-immunisation status and the particular type of polysaccharide antigen used. Results of trials with conjugate vaccines in human volunteers have indicated that response rates may only be around 65% for some of the key capsule antigens (Larsson et al., Infection and Immunity 64:3518-3523 (1996)). It is also not clear whether all individuals responding to the vaccine would have adequate levels of polysaccharide specific IgG which can cross the placenta and afford immunity to neonates. By conjugating a protein carrier to the polysaccharide antigen it may be possible to convert them to T-cell dependent antigens and enhance their immunogenicity.

Preliminary studies with GBS type III polysaccharide-tetanus toxoid conjugate have been encouraging (Baker et al., Reviews of Infectious Diseases 7:458-467 (1985), Baker et al., The New England Journal of Medicine 319:1180-1185 (1988), Paoletti et al., Infection and Immunity 64:677-679 (1996), Paoletti et al., Infection and Immunity 62:3236-3243 (1994)) but in developed countries the use of tetanus may be disadvantageous since most adults will have been immunised against tetanus within the past five years. Additional boosters with tetanus toxoid may cause adverse reactions (Boyer., Current Opinions in Pediatrics 7:13-18 (1995)). The polysaccharide conjugate vaccines have the disadvantage of being costly to produce and manufacture in comparison with many other kinds of vaccines. There is also the possible risk of problems caused by the cross reactivity between GBS polysaccharides and sialic acid-containing human glycoproteins.

Recent evidence suggests that bacterial surface proteins also may be useful to confer immunity. A protein called Rib which is found on most serotype III strains but rarely on serotypes Ia, Ib or II confers immunity to challenge with Rib expressing GBS in animal models (Stalhammar-Carlemalm et al., Journal of Experimental Medicine 177:1593-1603 (1993)). Another surface protein of interest as a component of a vaccine is the alpha antigen of the C proteins which protected vaccinated mice against lethal infection with strains expressing alpha protein. The amount of this antigen expressed by GBS strains varies markedly, however an alternative to polysaccharides as antigens is the use of protein antigens derived from GBS. Recent evidence suggest that the GBS surface associated proteins Rib and alpha C protein may be used to confer immunity to GBS infections in experimental model systems (Stalhammar-Carlemalm et al., (1993) [supra], Larsson et al., (1996) [supra]). However these two proteins are not conserved in all serotypes of GBS which cause disease in humans. Assuming that these antigens would be immunogenic and elicit protective level responses in humans they would not confer protection against all infections caused by GBS as 10% of infectious Group B streptococci do not express Rib or C protein alpha.

This invention seeks to overcome the problem of vaccination against GBS by using a novel screening method specifically designed to identify those Group B Streptococcus genes encoding bacterial cell surface associated or secreted proteins. The proteins expressed by these genes may be immunogenic, and therefore may be useful in the prevention and treatment of Group B Streptococcus infection. For the purposes of this application, the term immunogenic means that these proteins will elicit a protective immune response within a subject. Using this novel screening method a number of genes encoding novel Group B Streptococcus proteins have been identified.

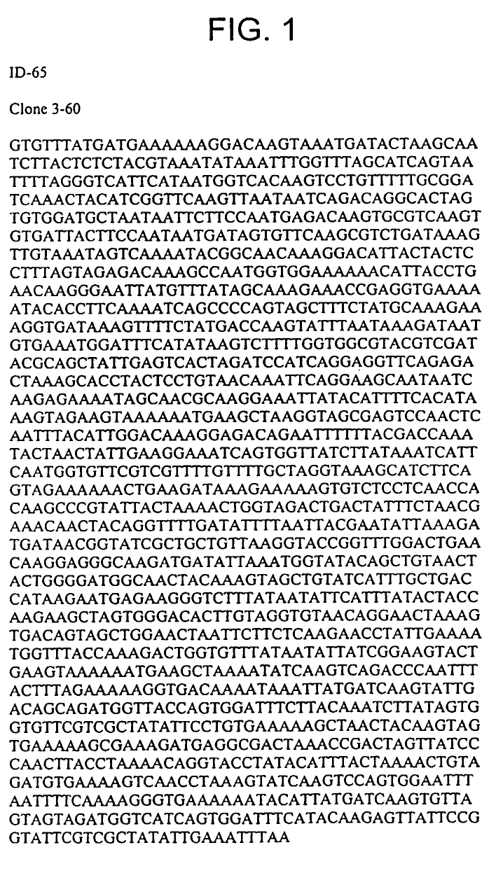

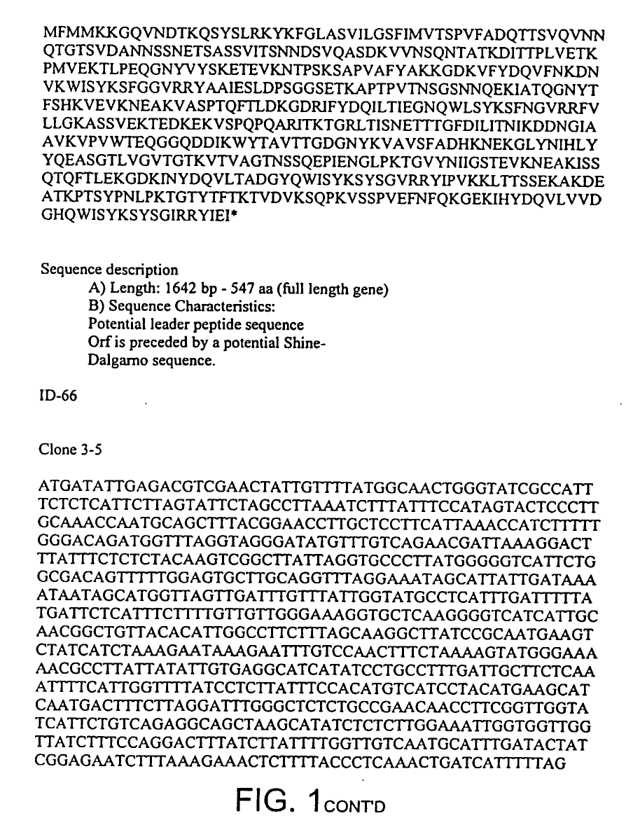

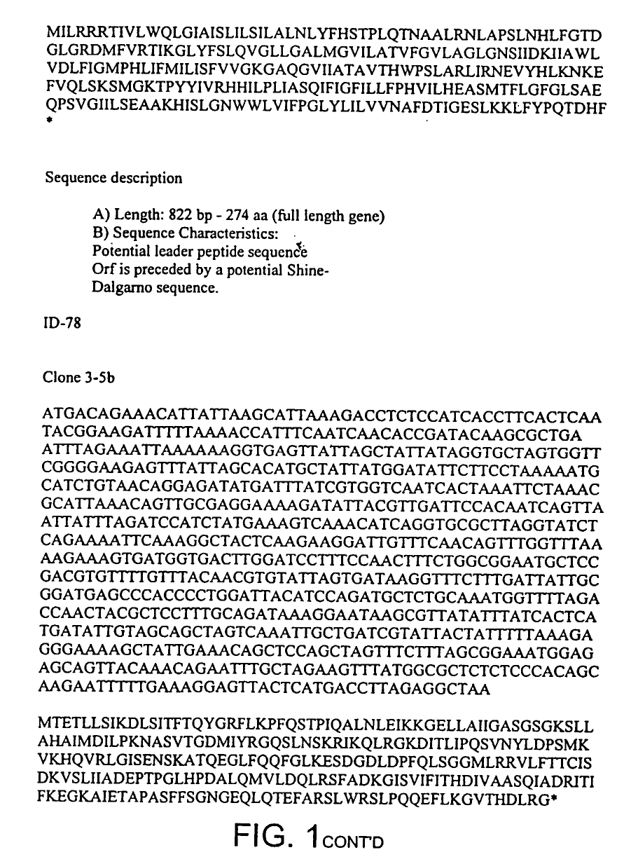

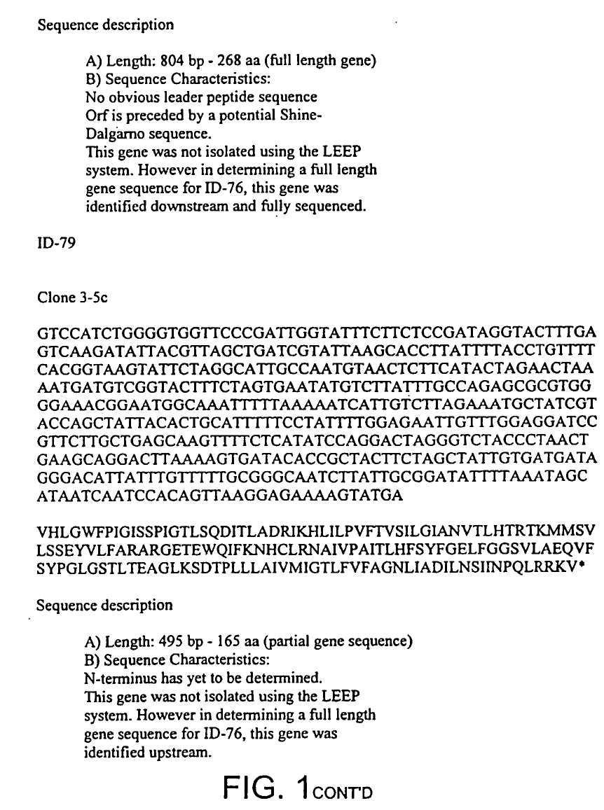

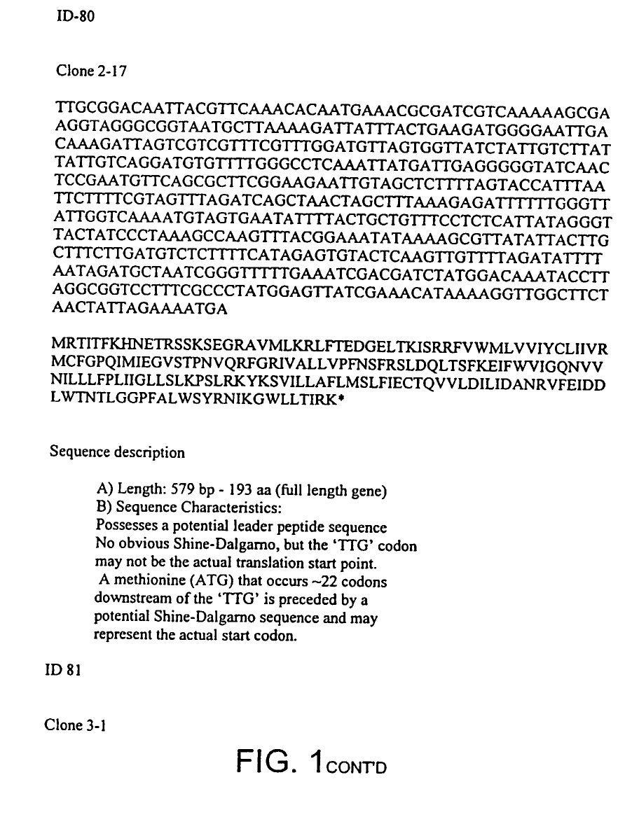

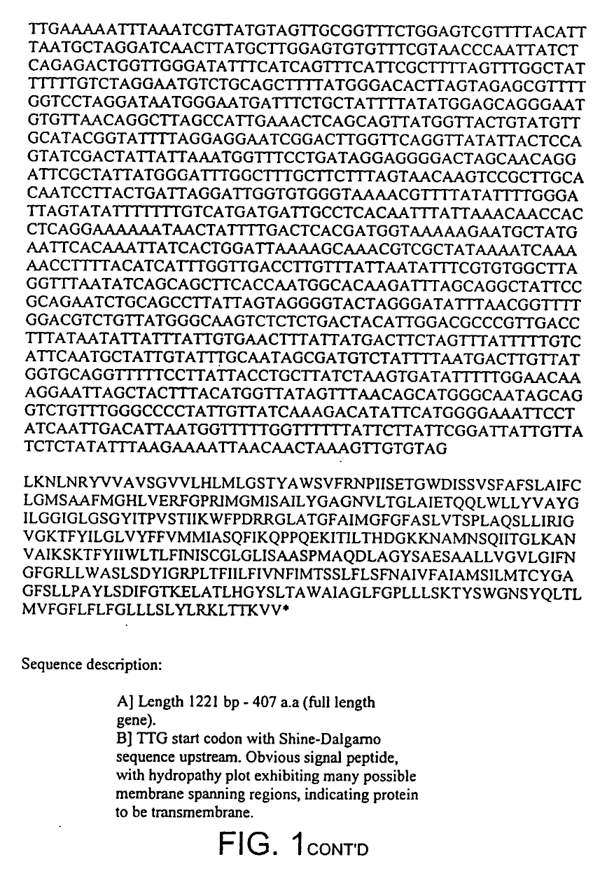

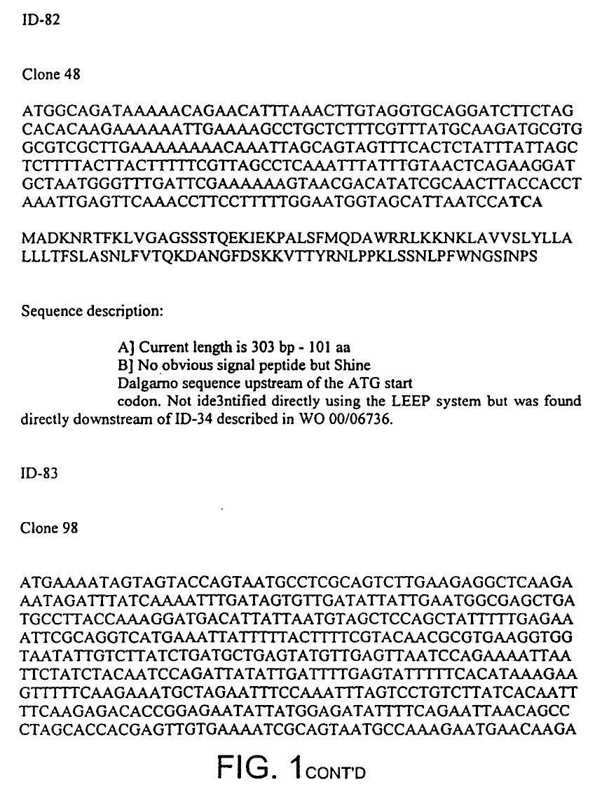

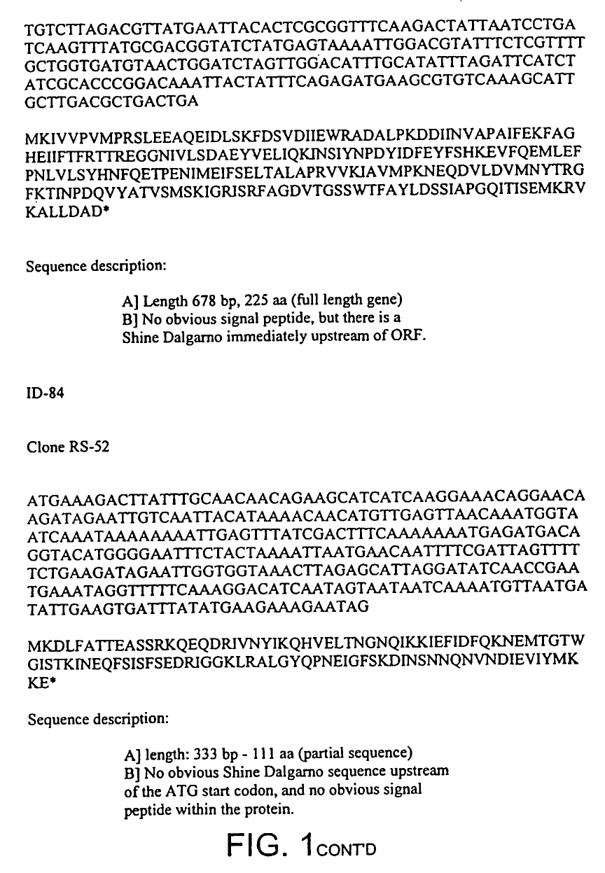

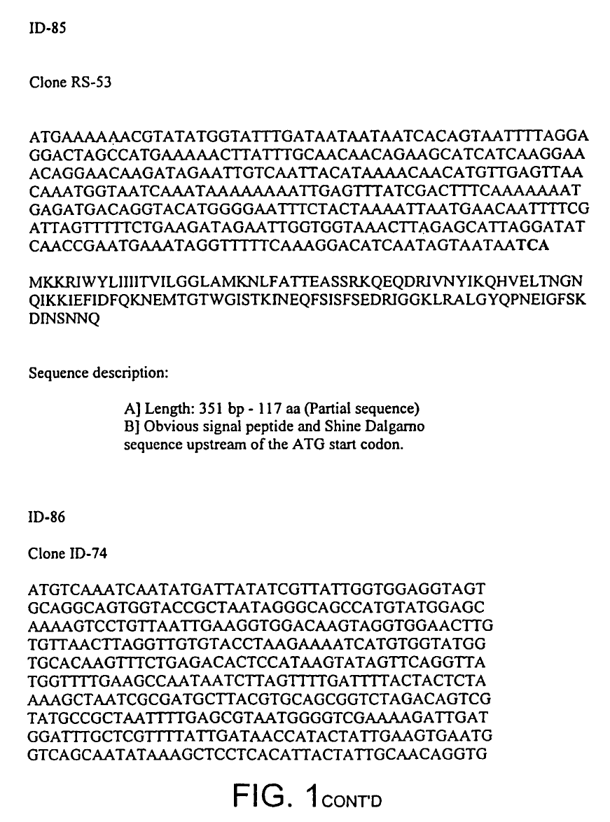

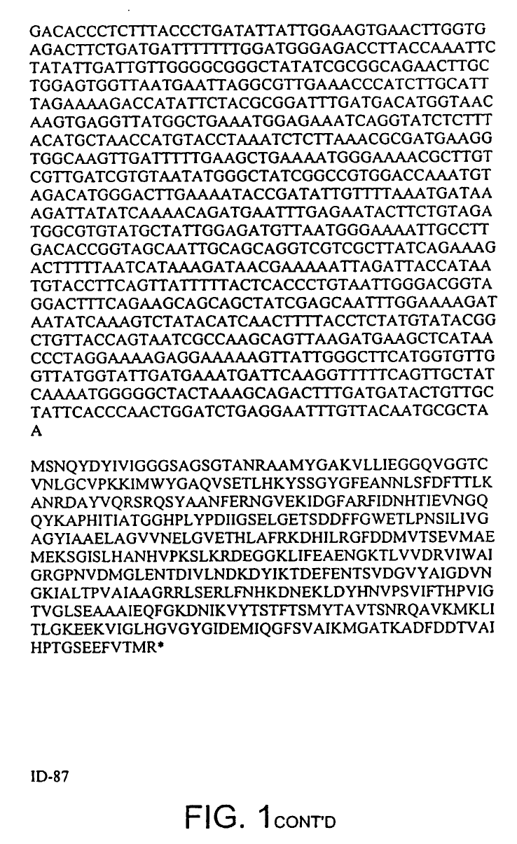

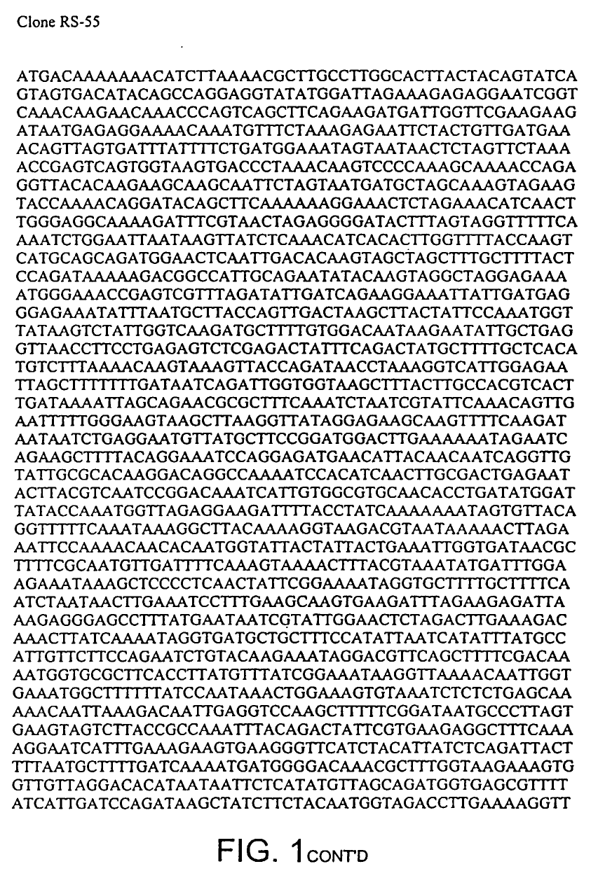

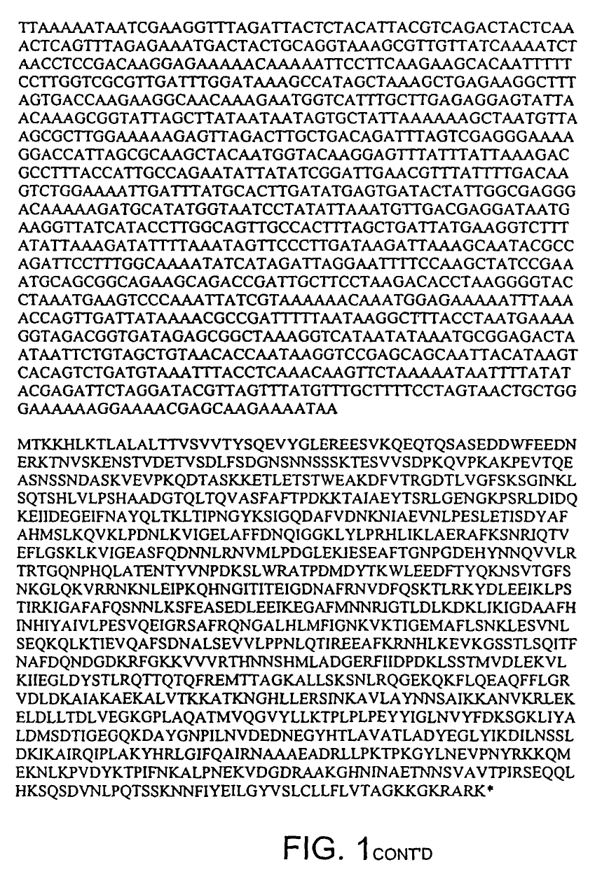

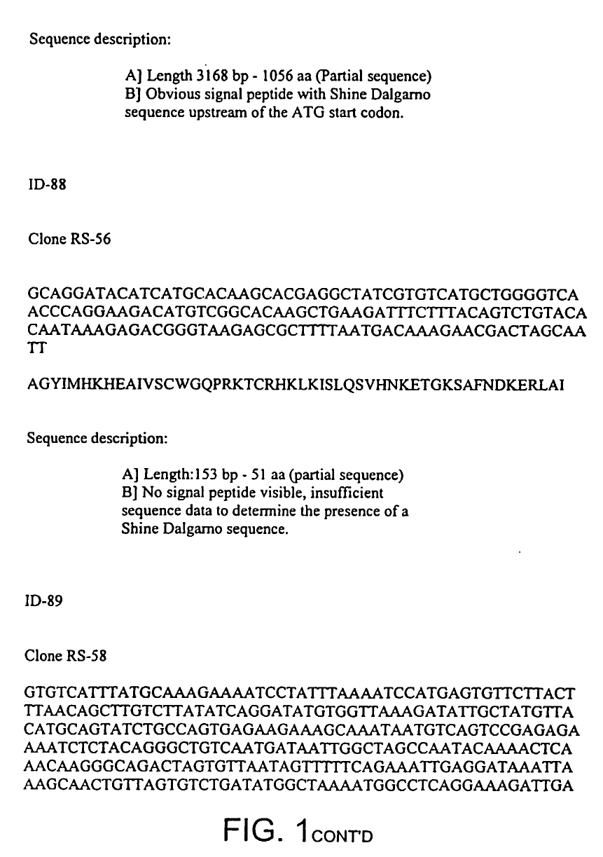

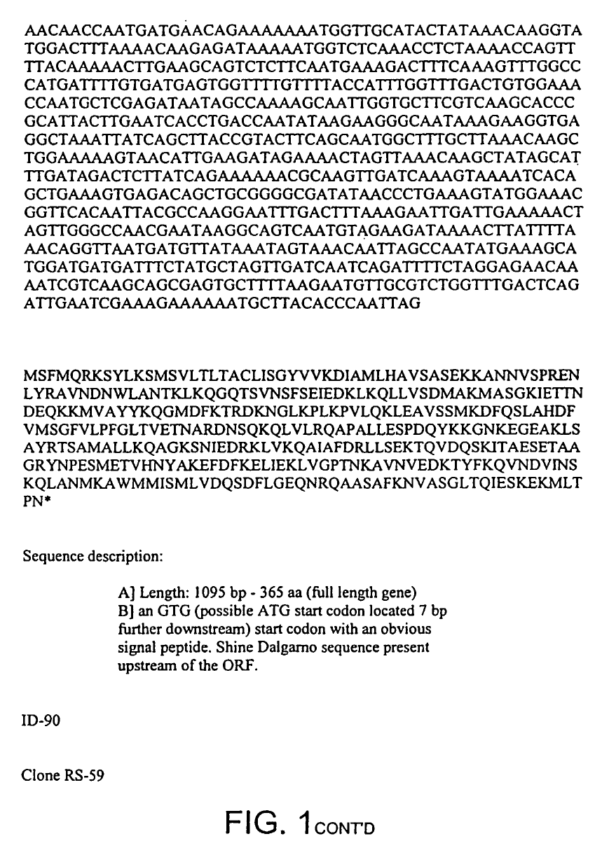

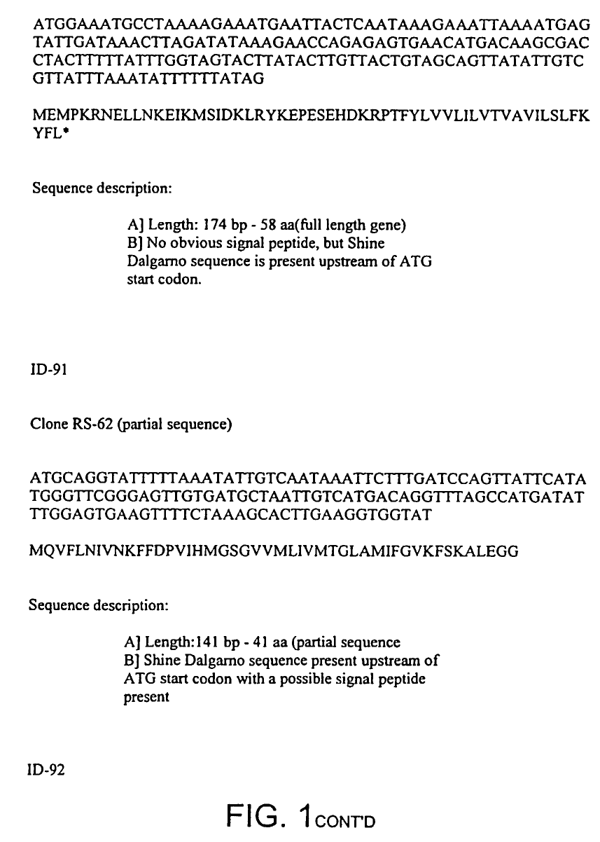

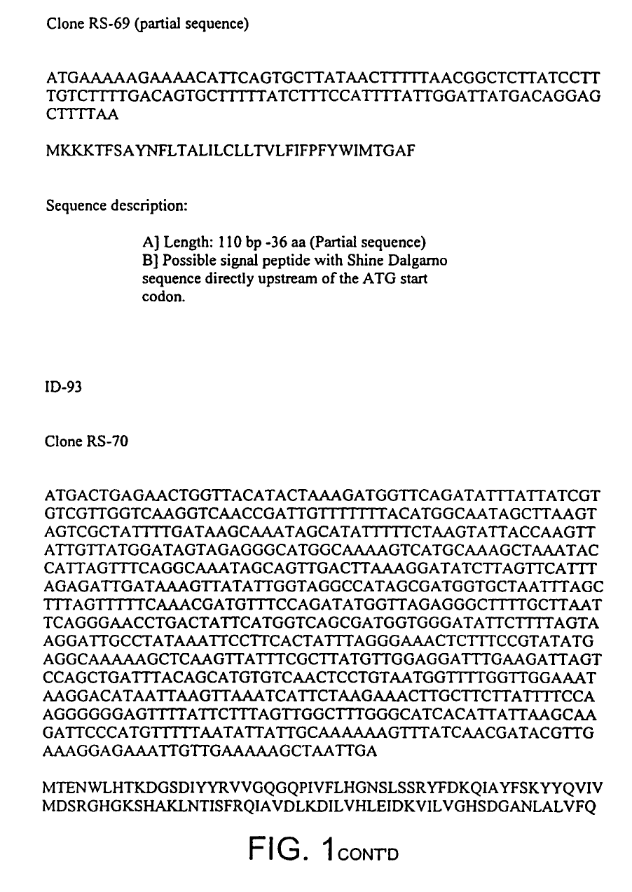

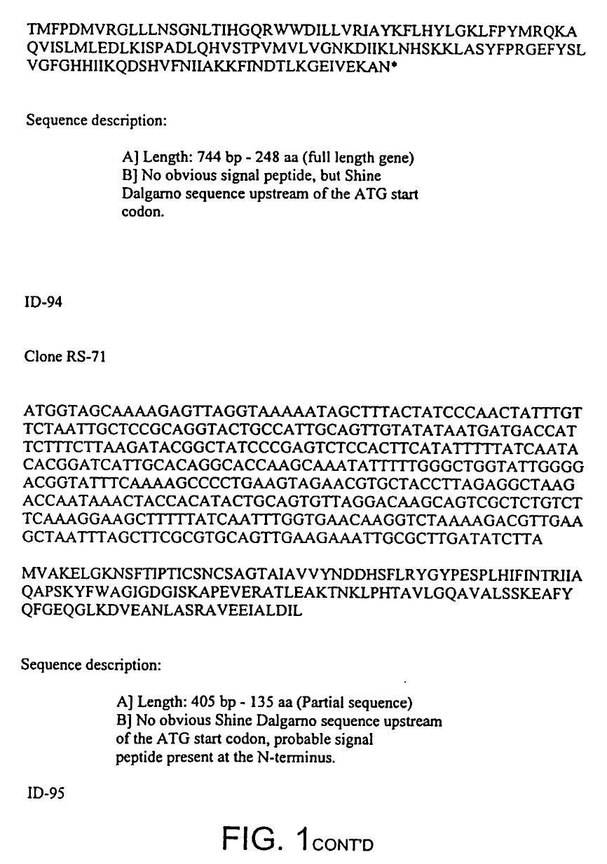

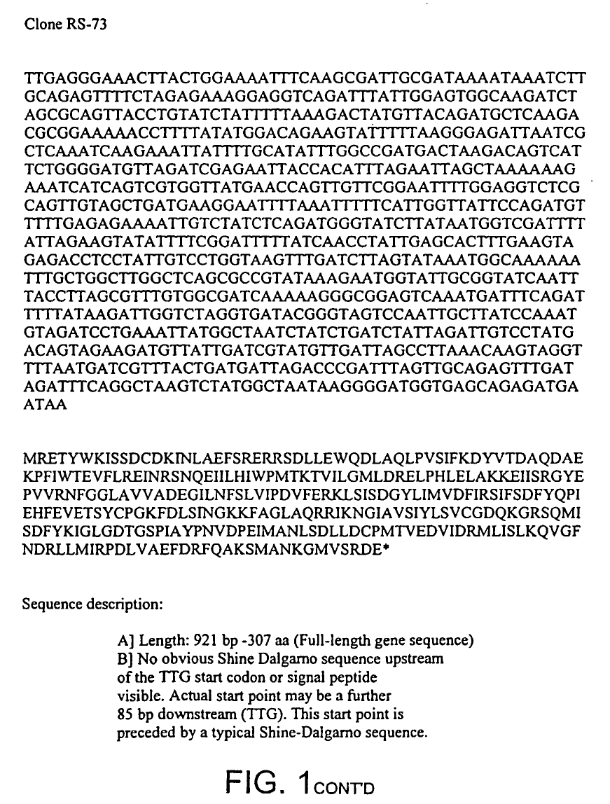

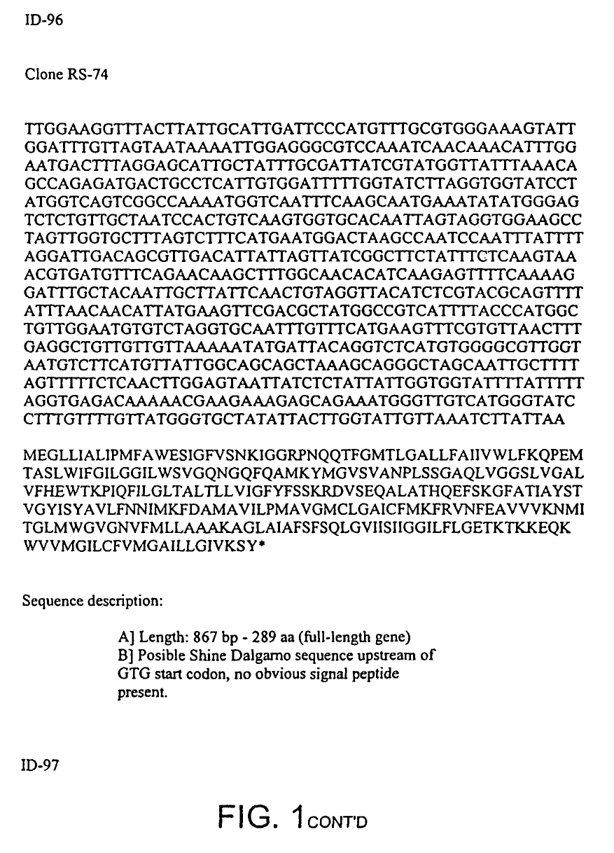

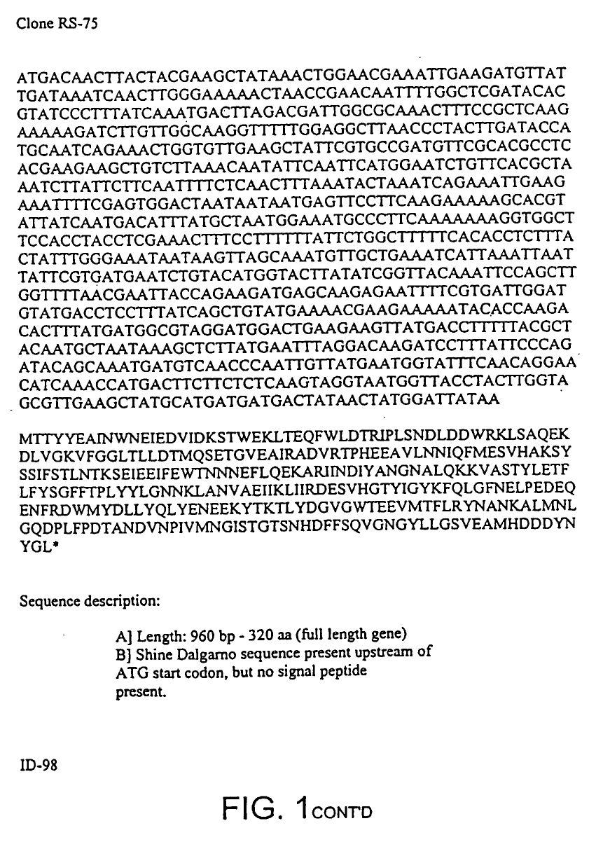

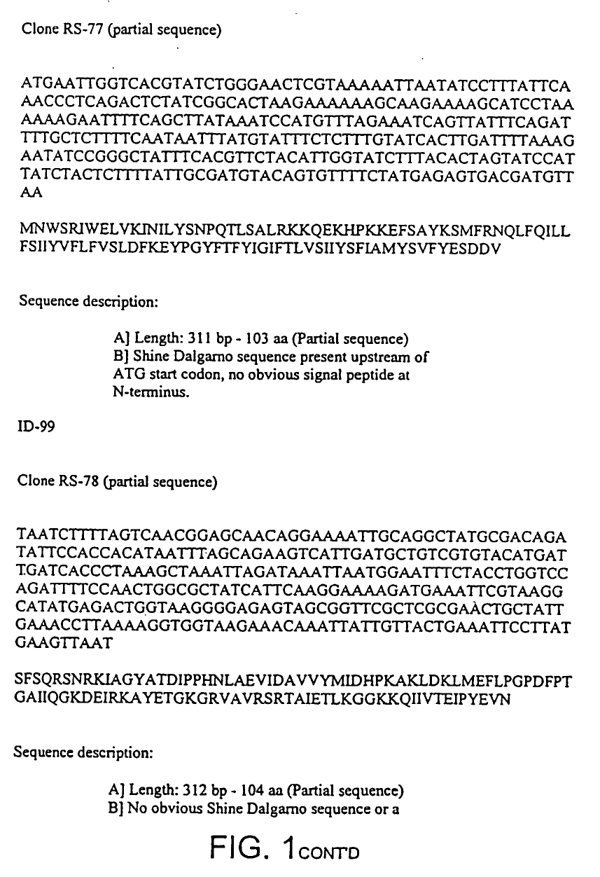

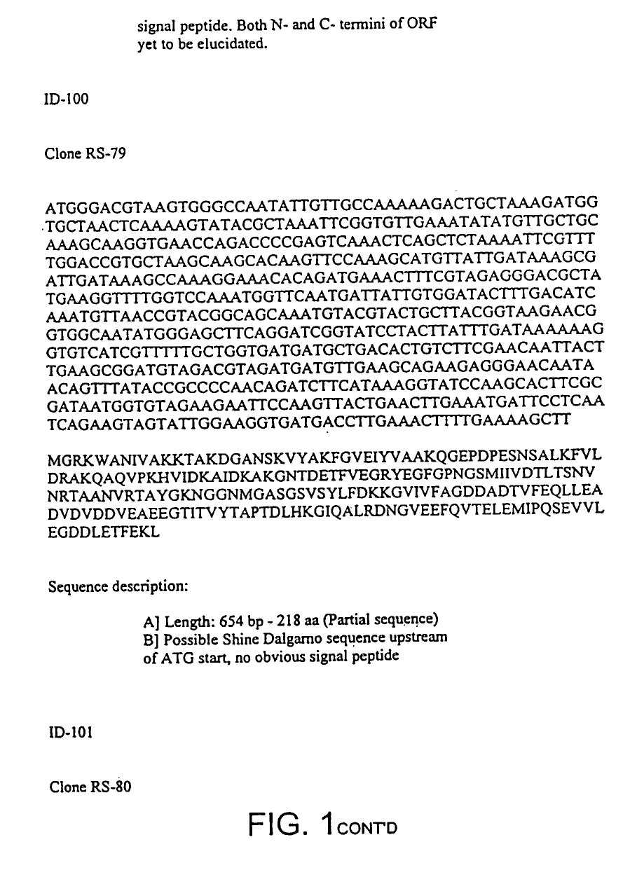

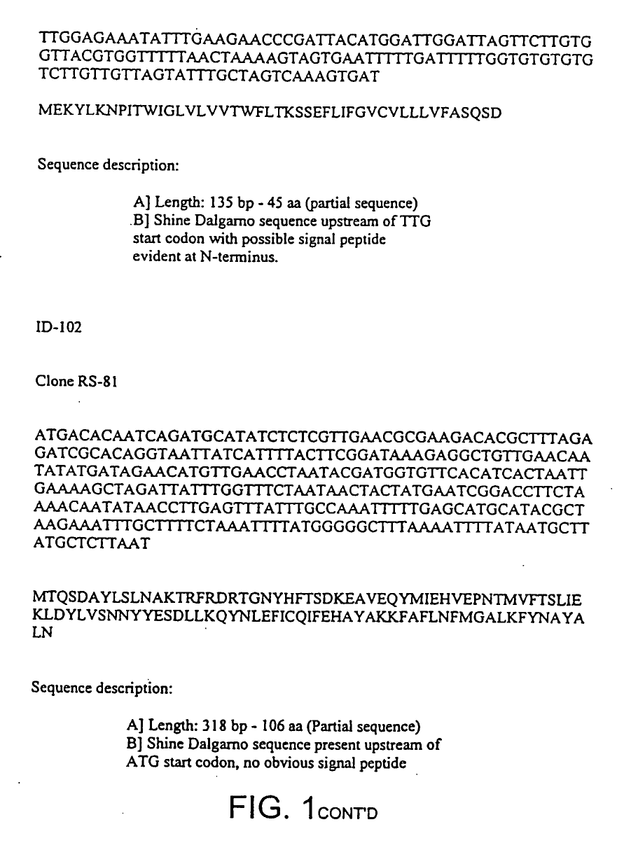

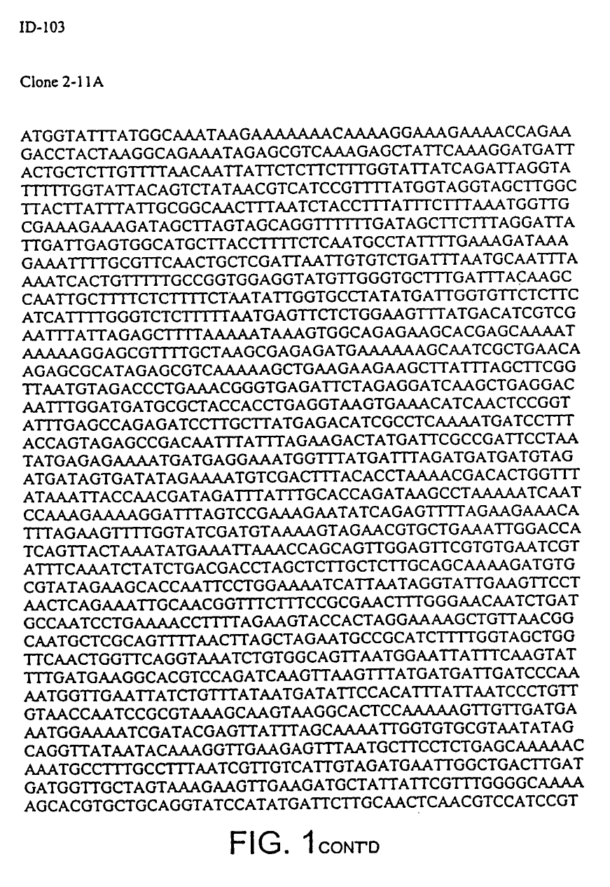

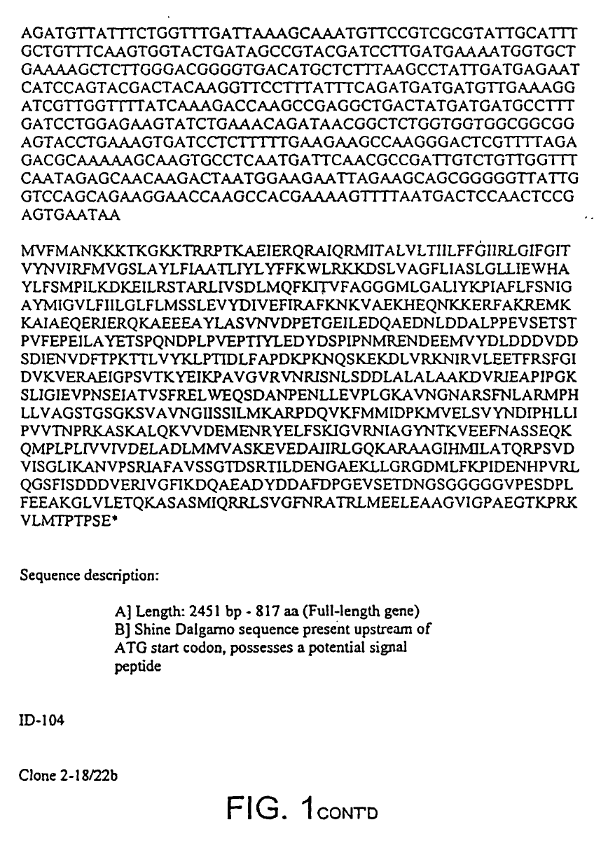

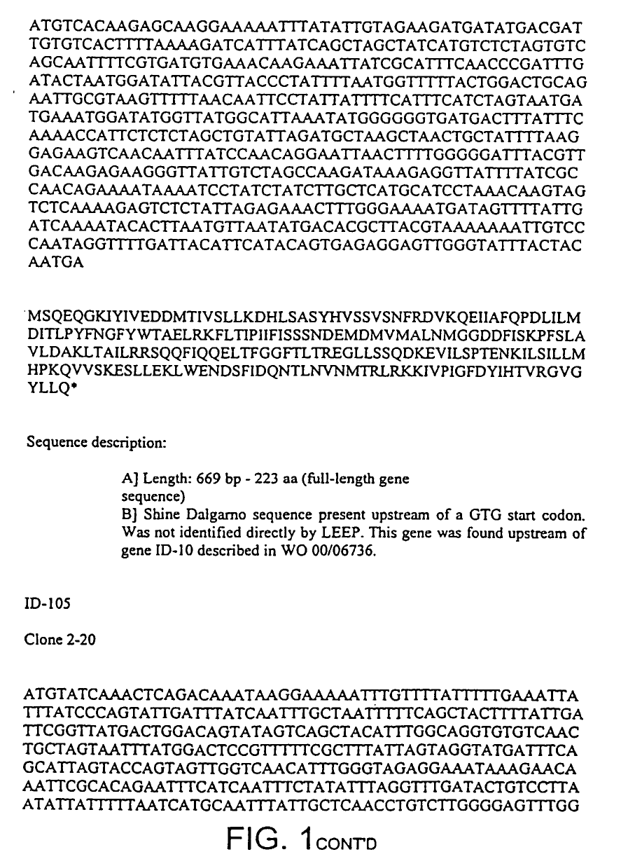

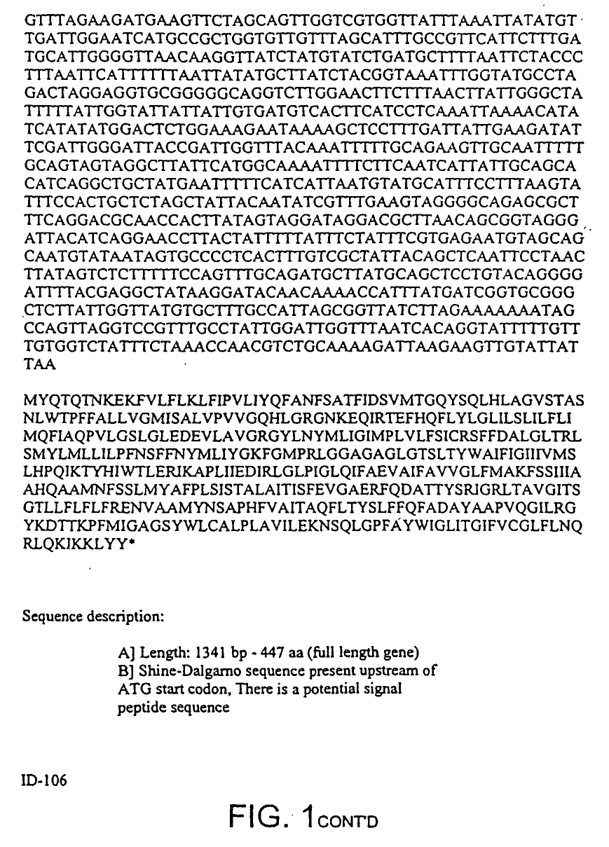

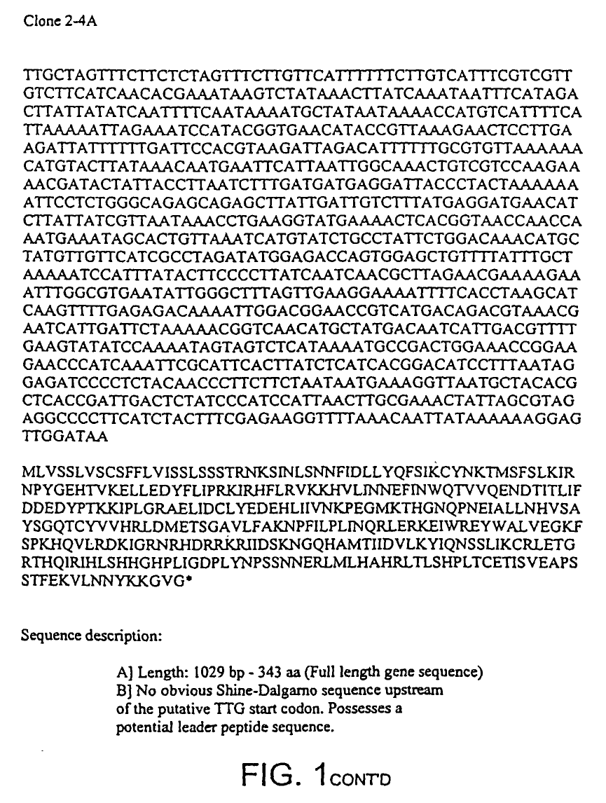

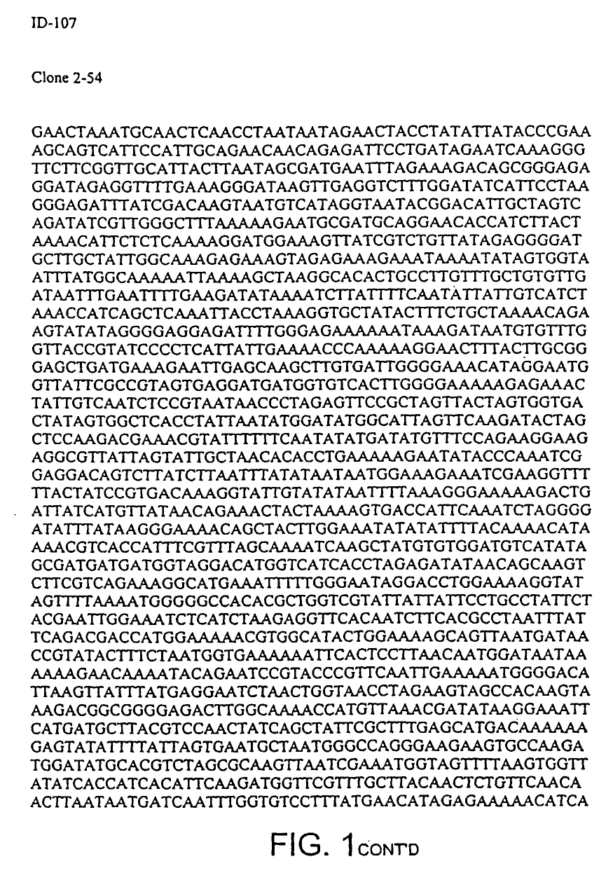

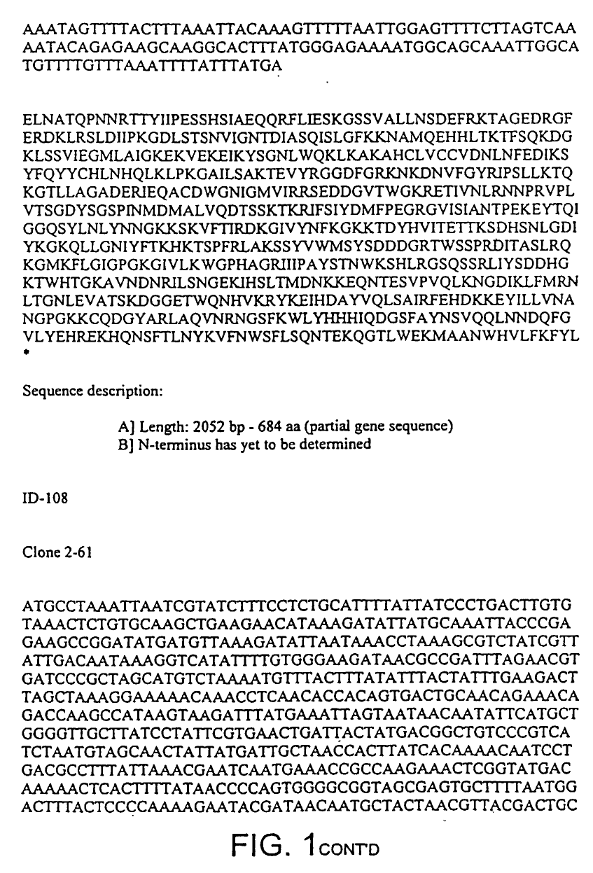

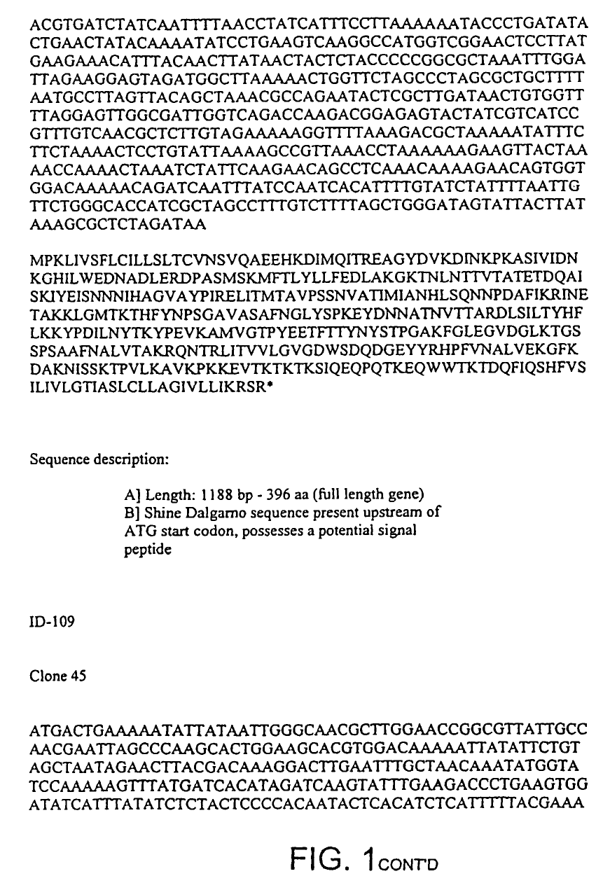

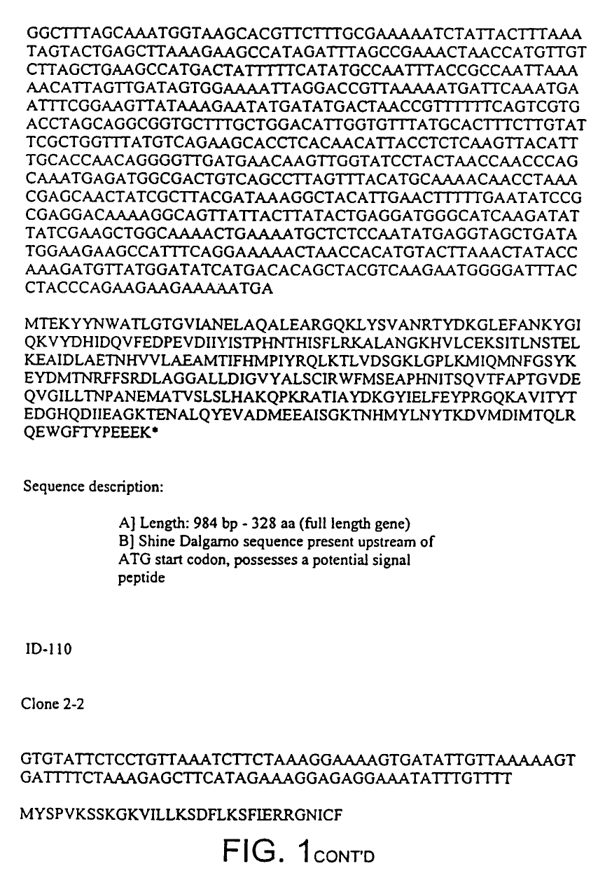

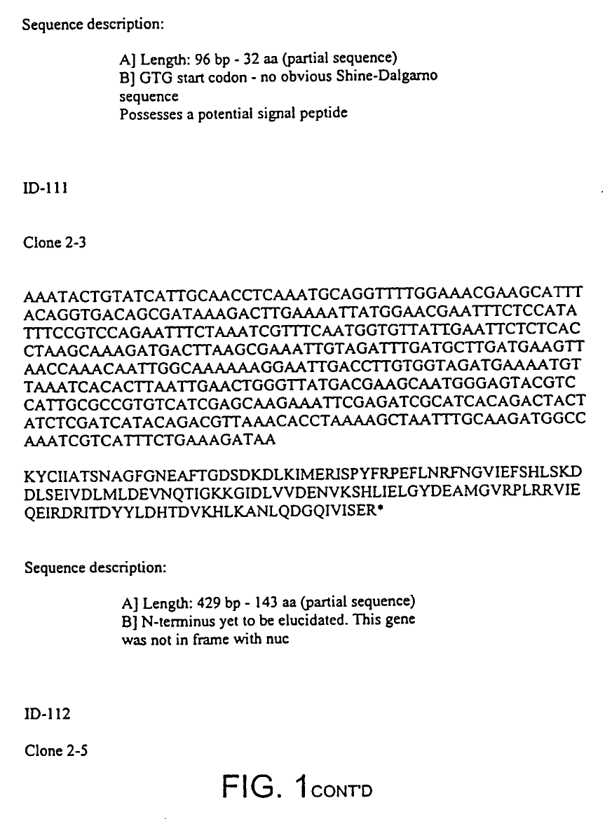

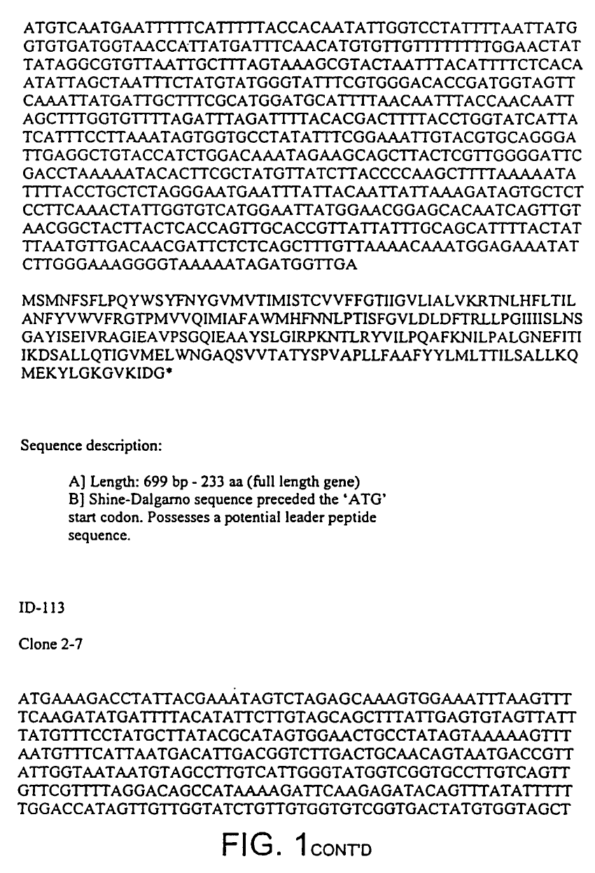

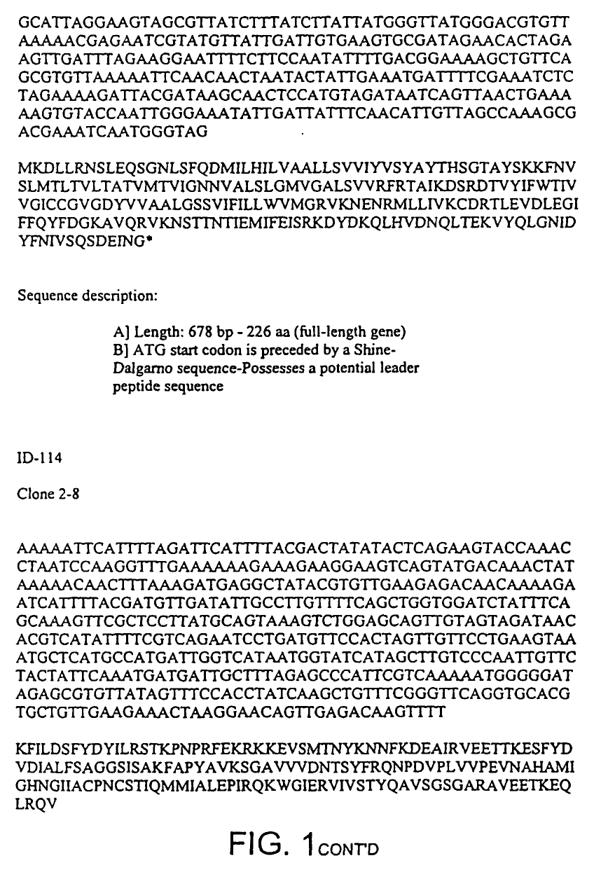

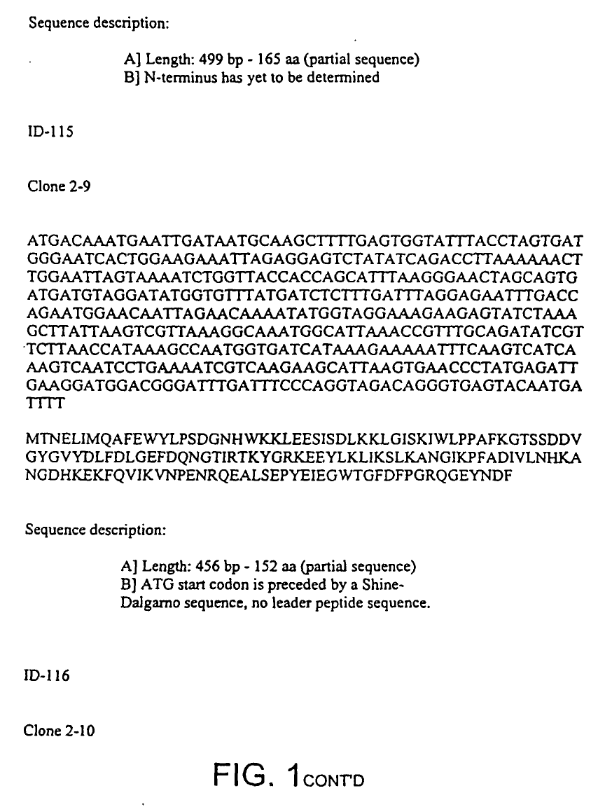

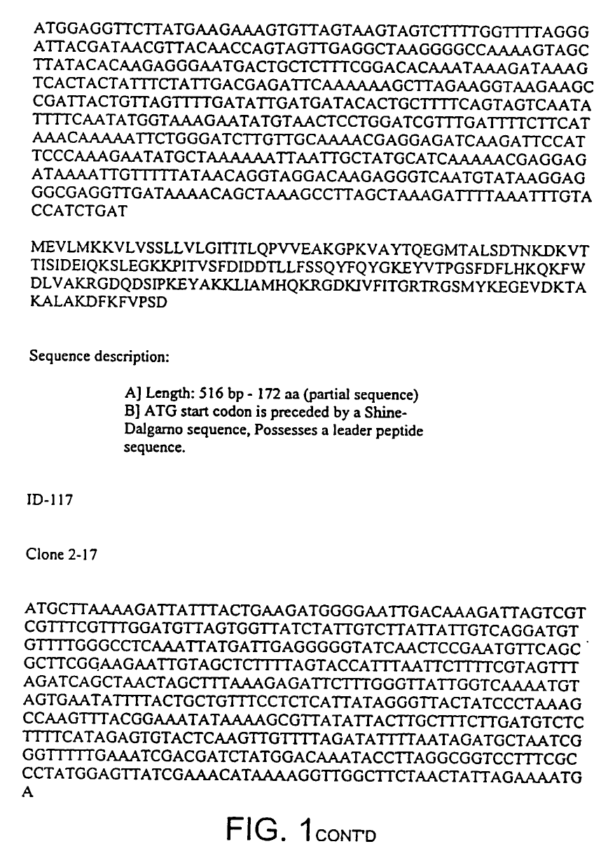

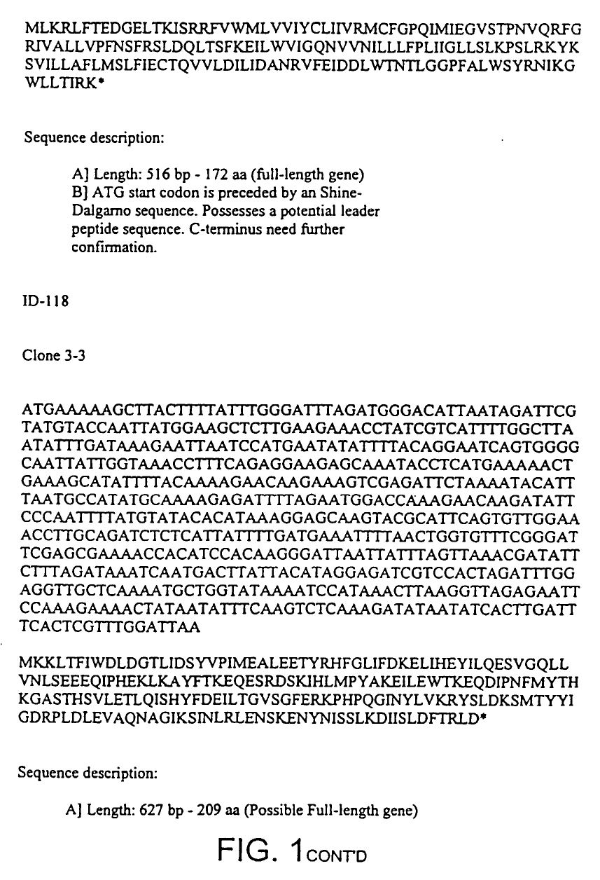

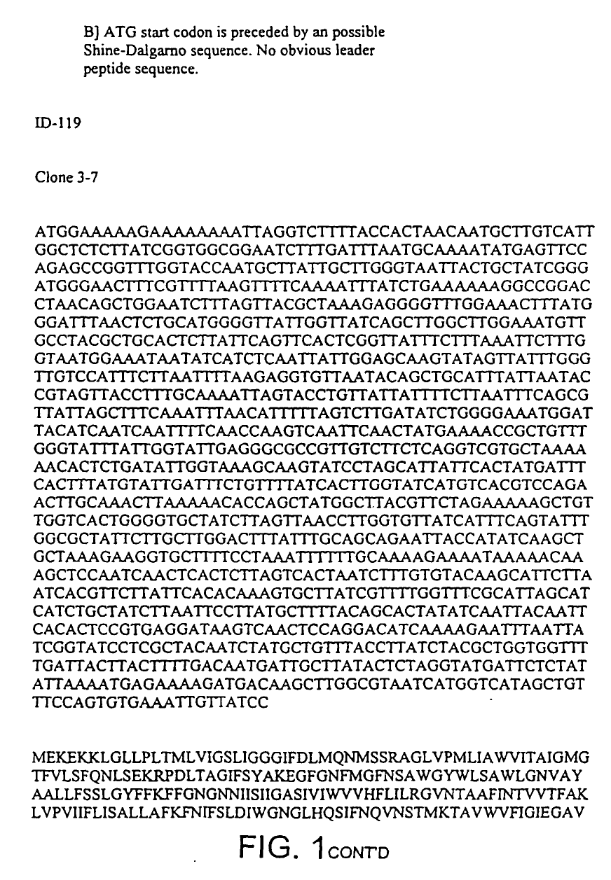

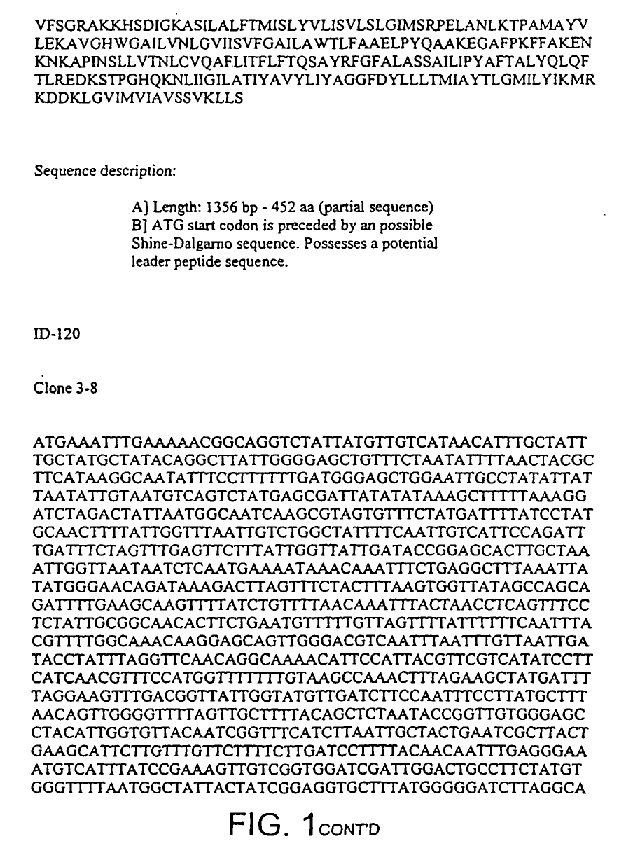

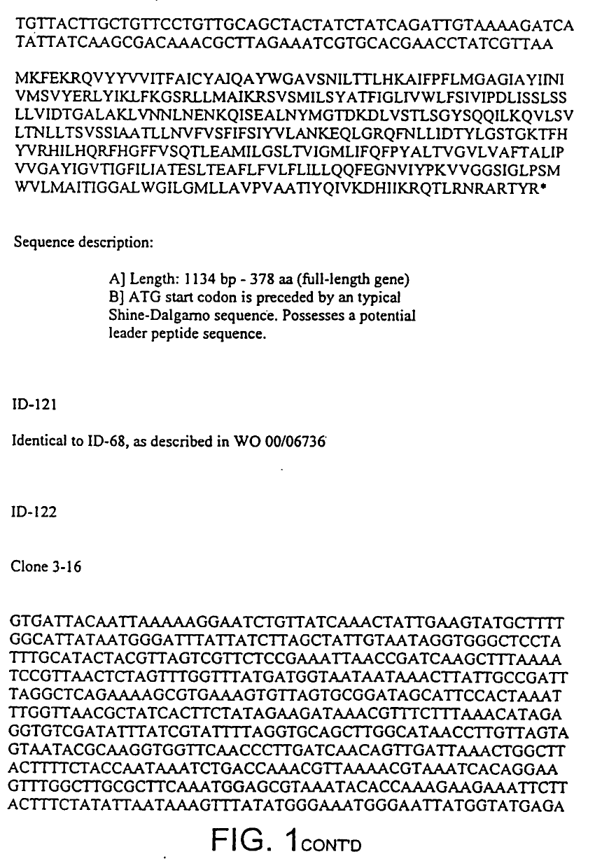

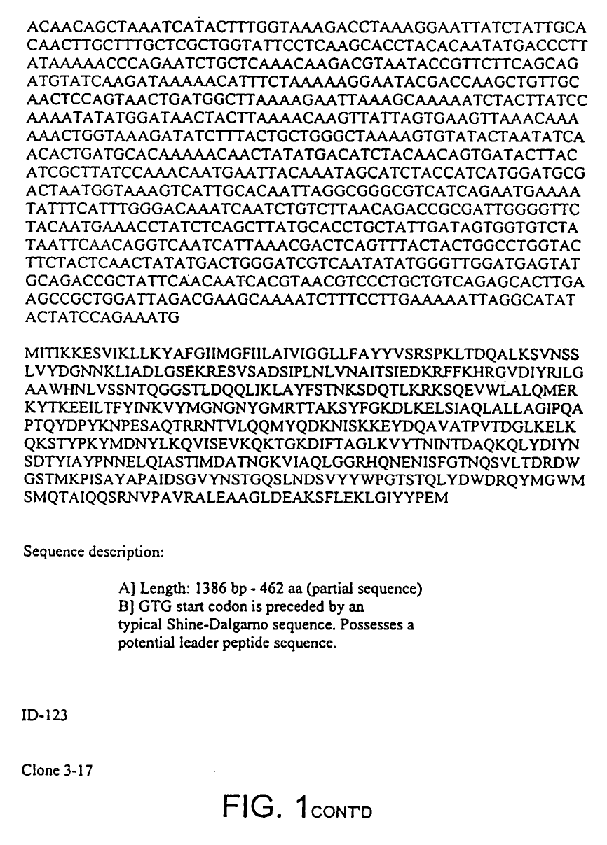

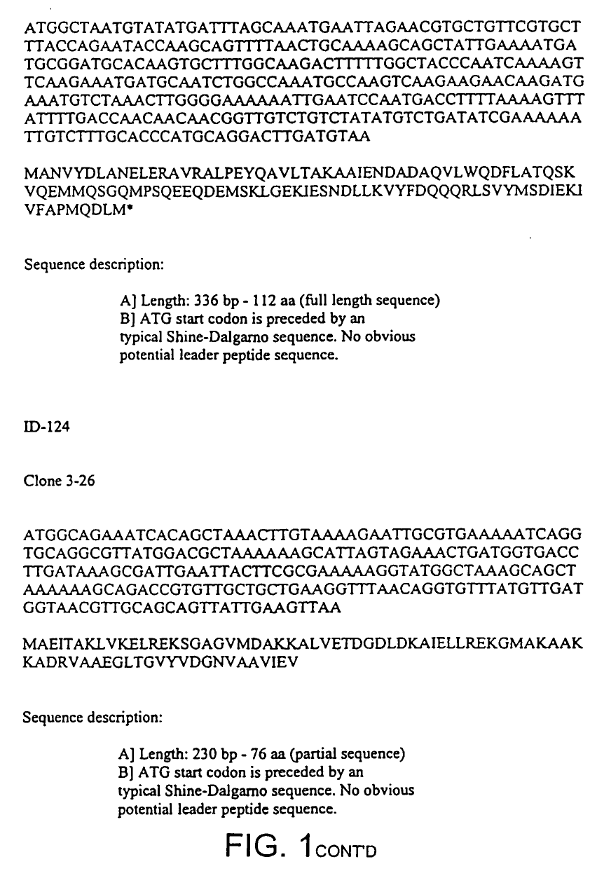

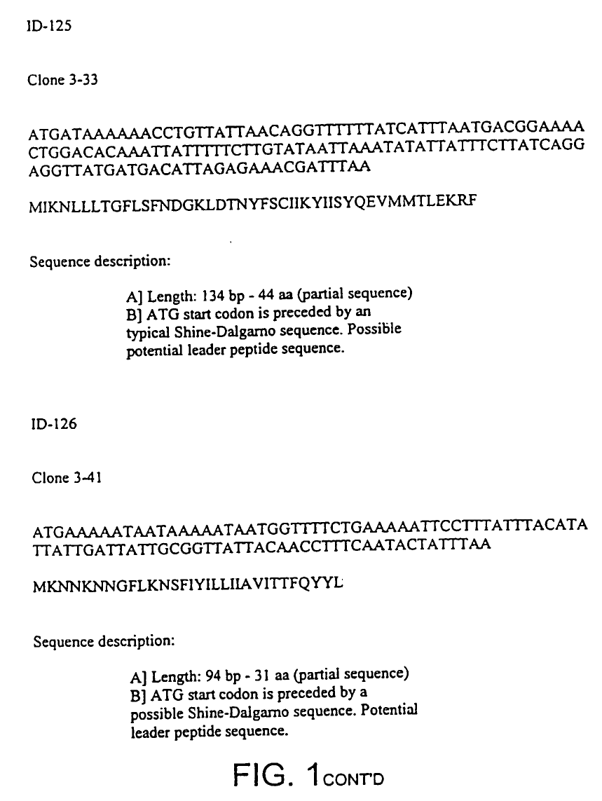

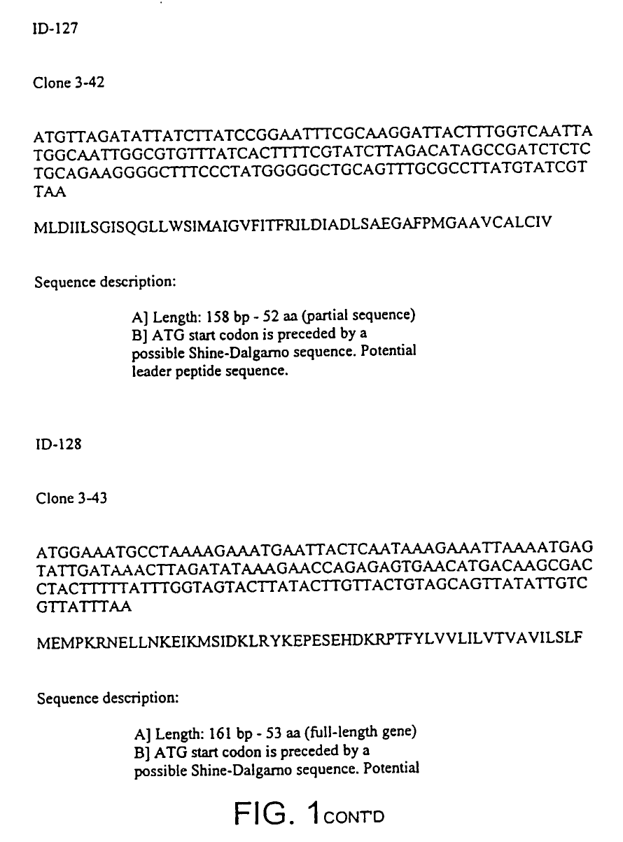

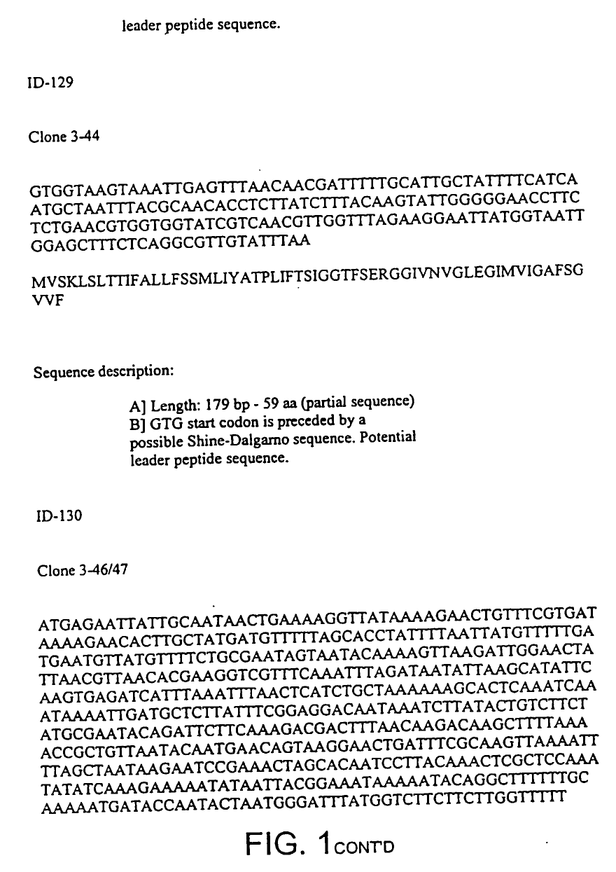

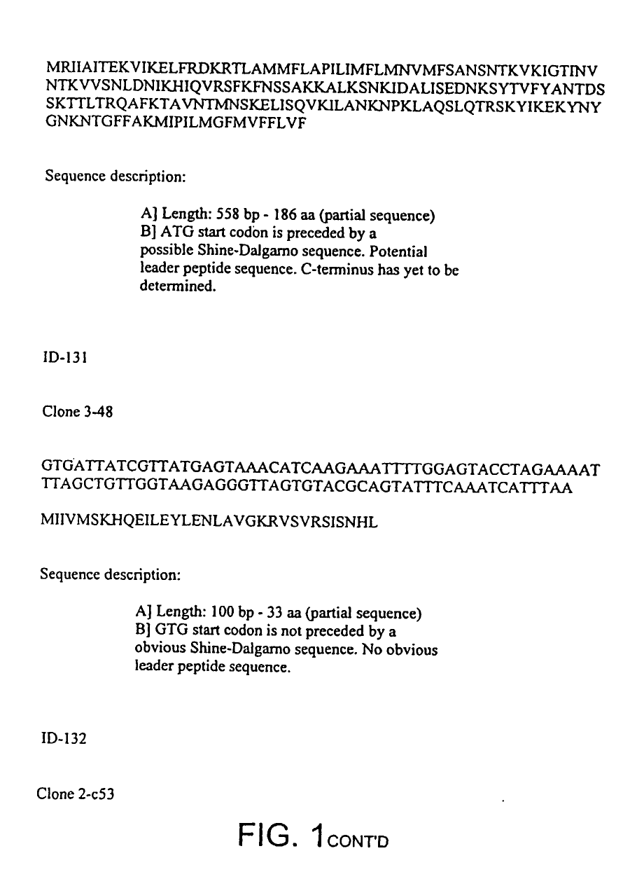

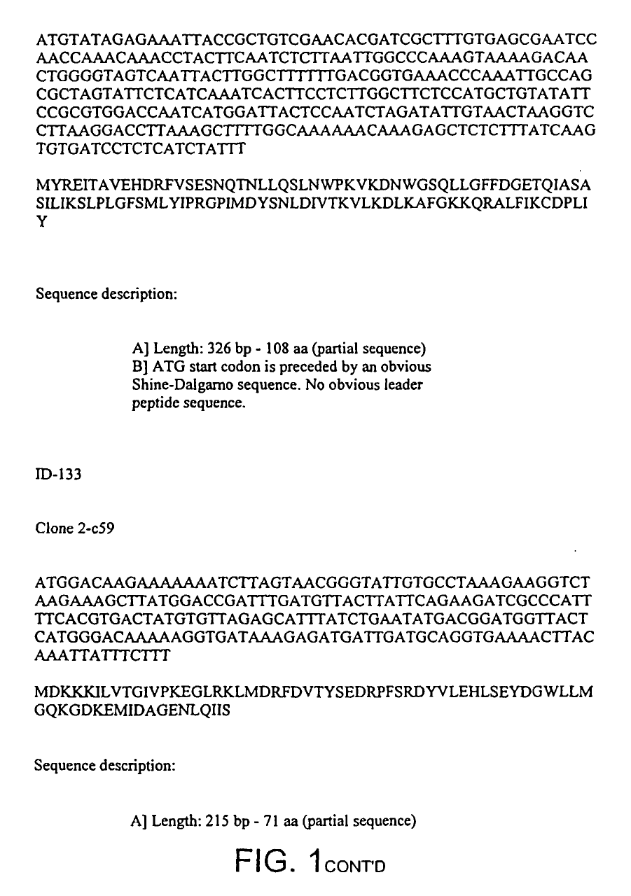

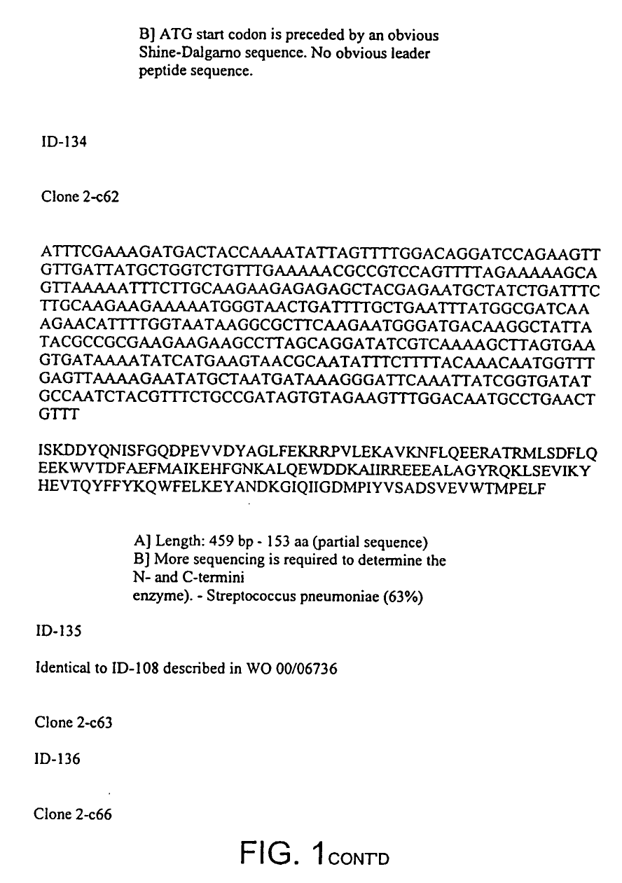

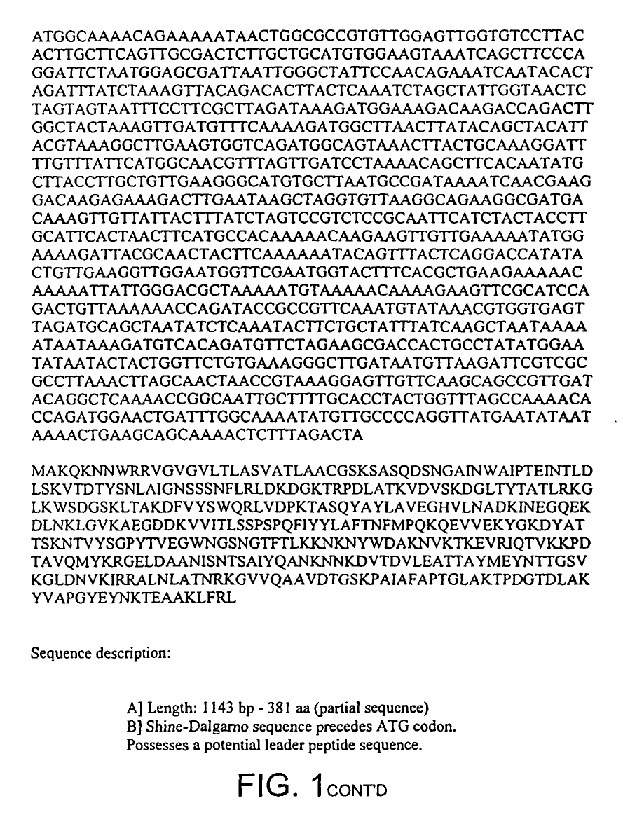

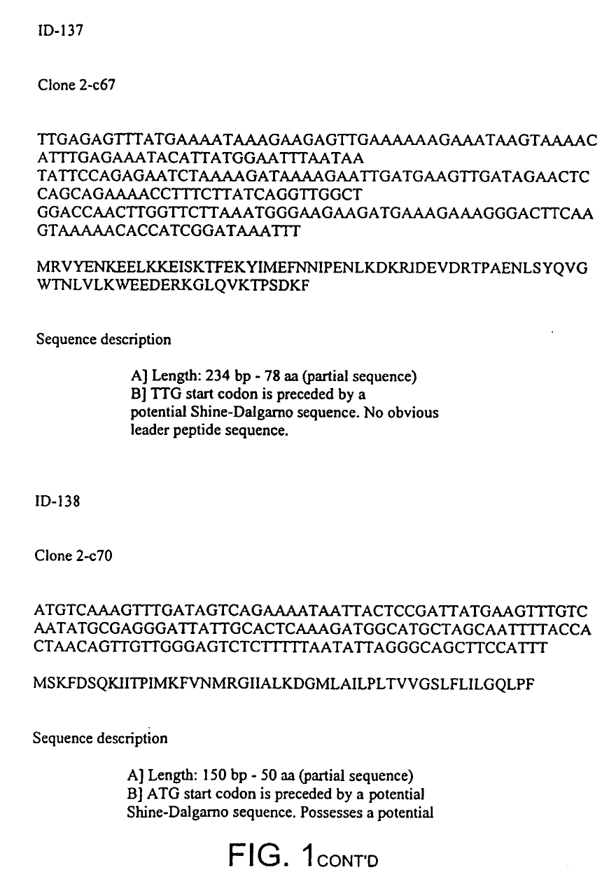

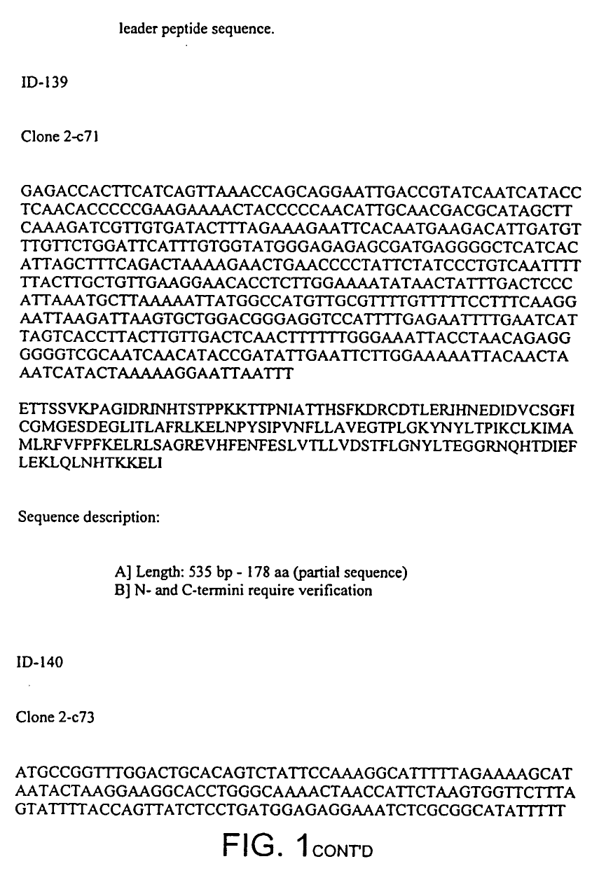

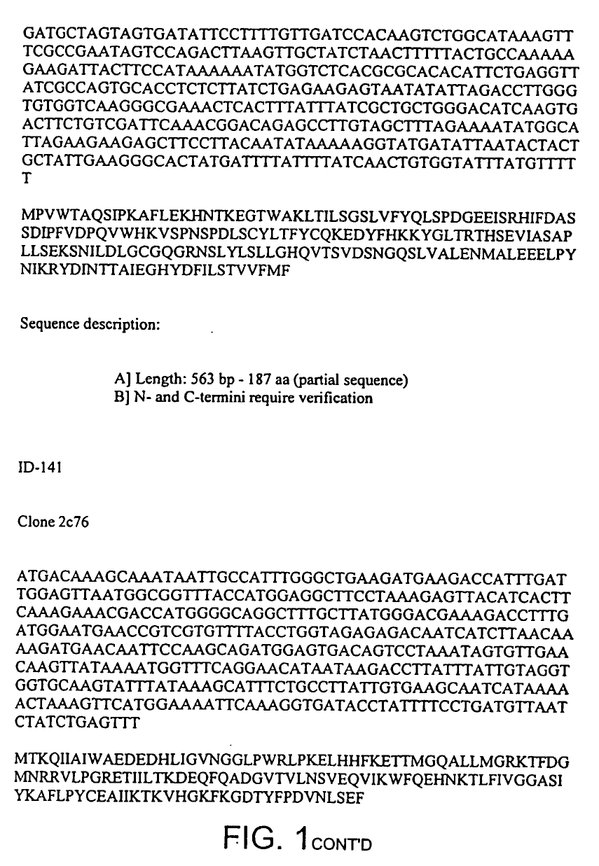

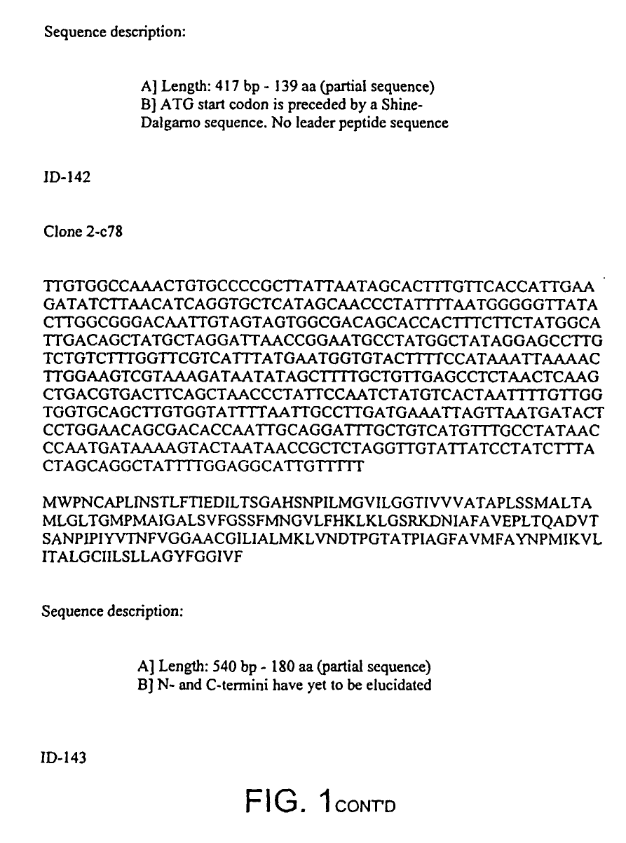

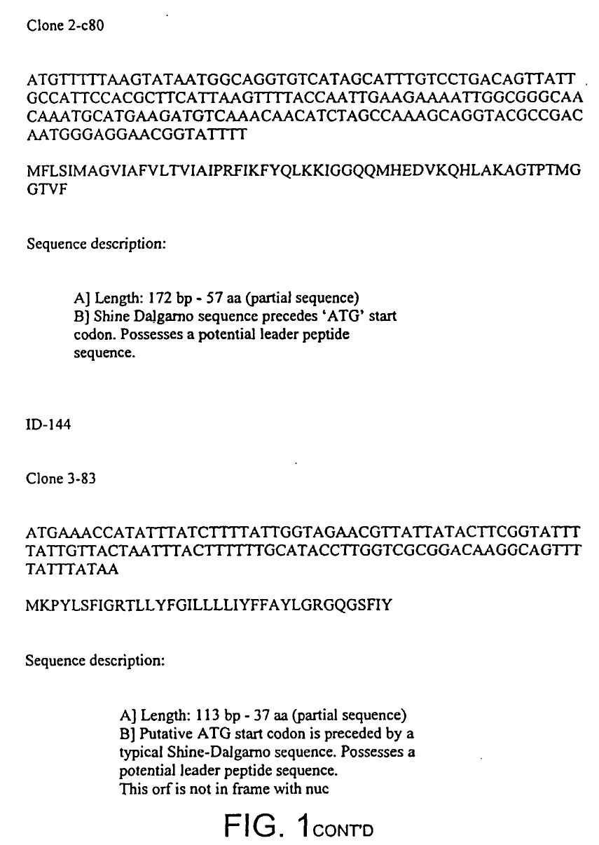

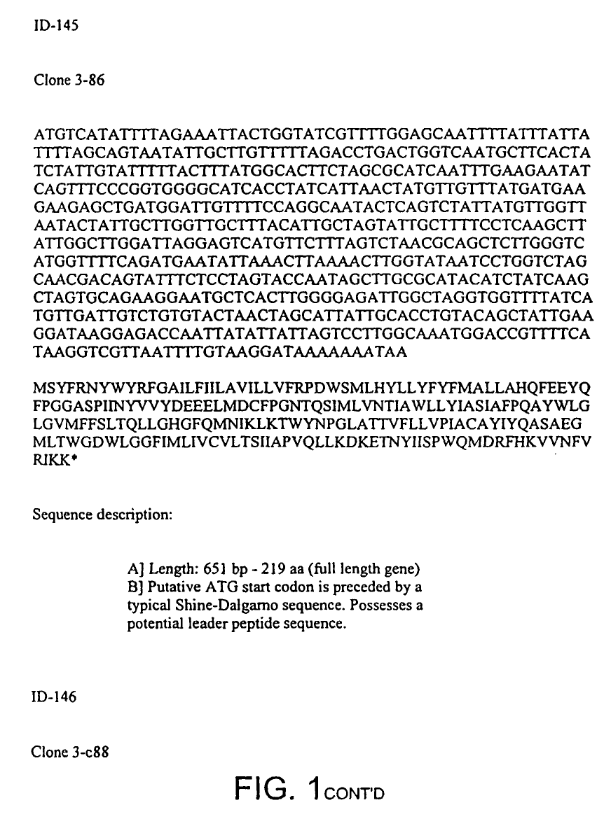

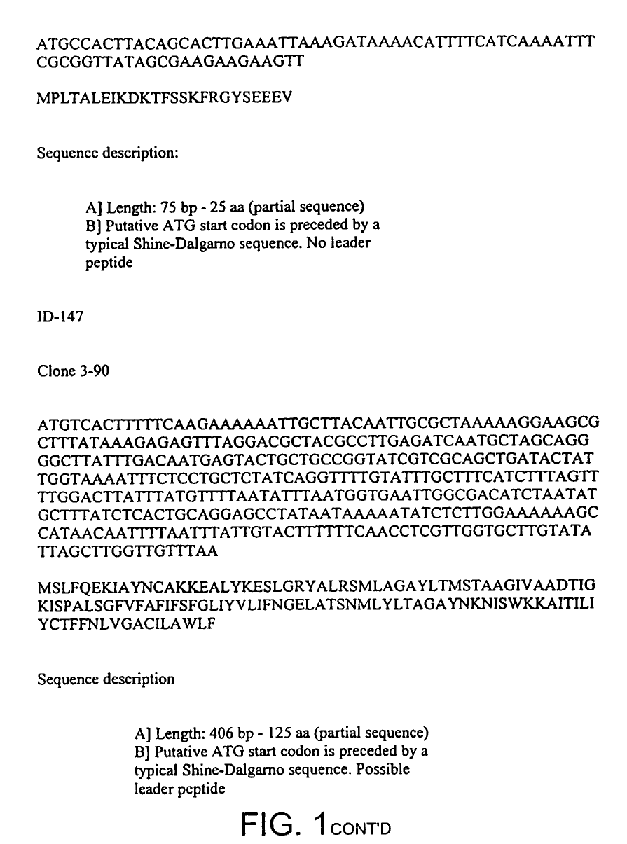

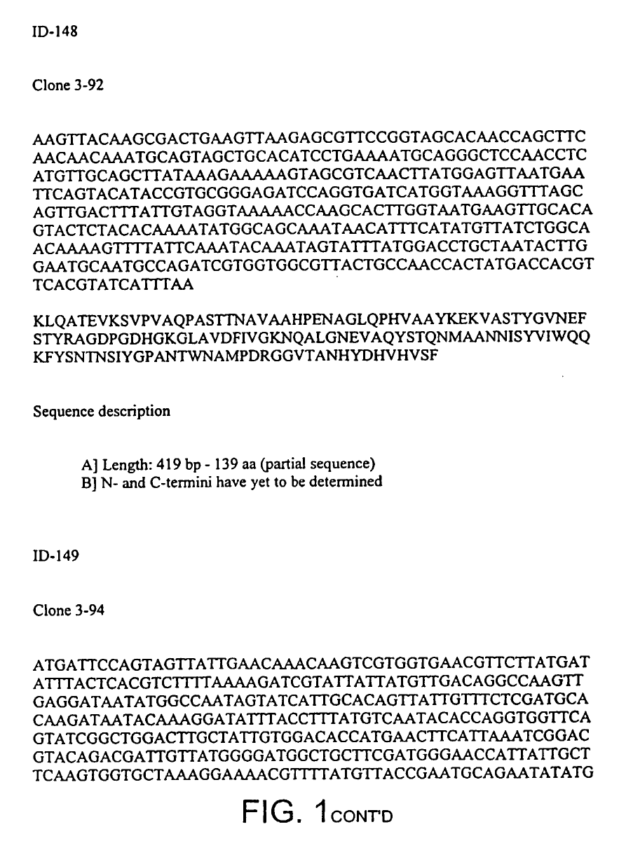

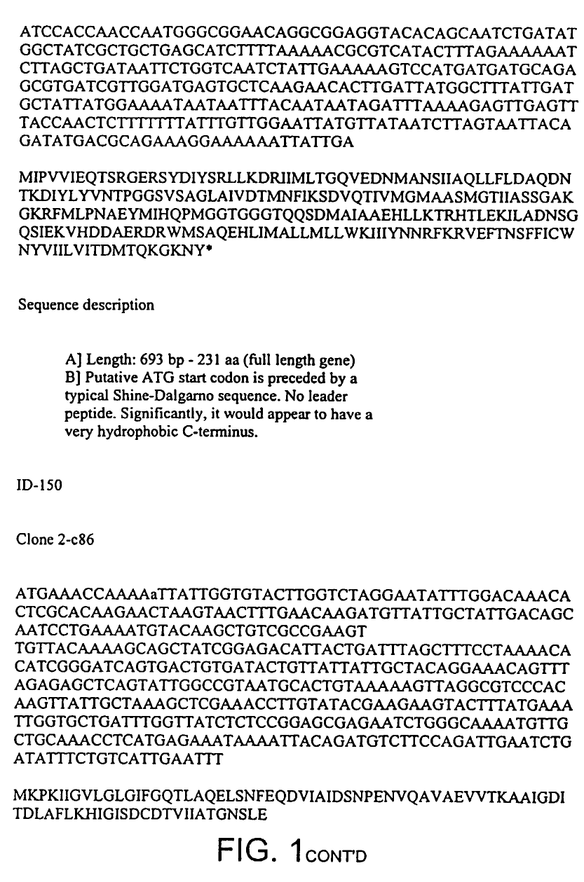

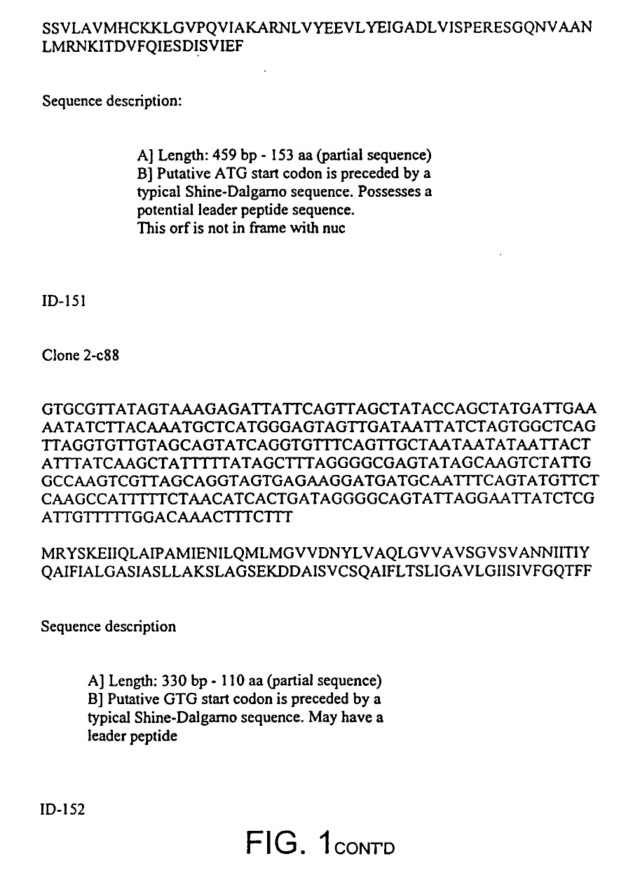

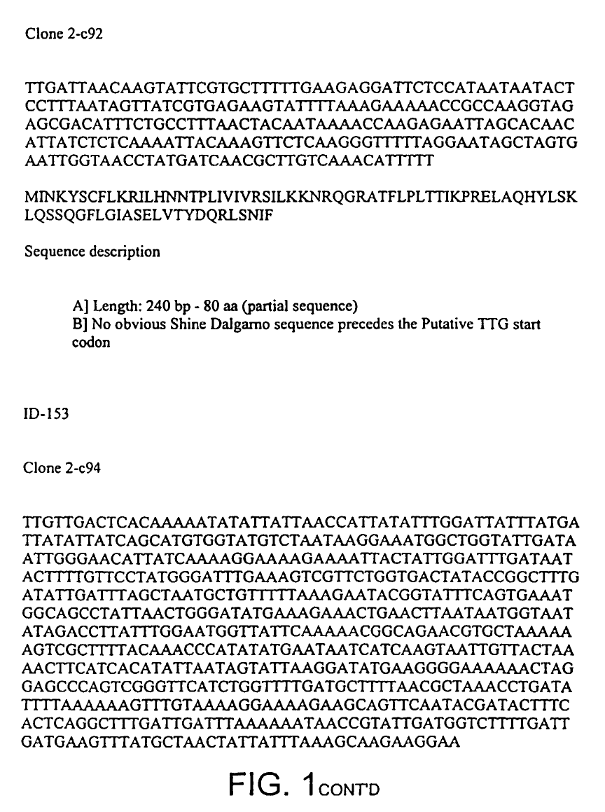

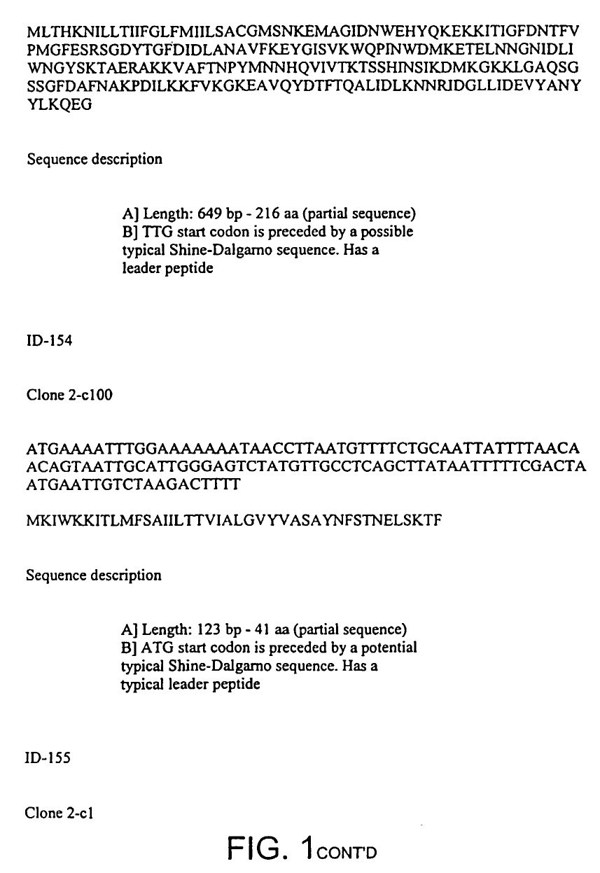

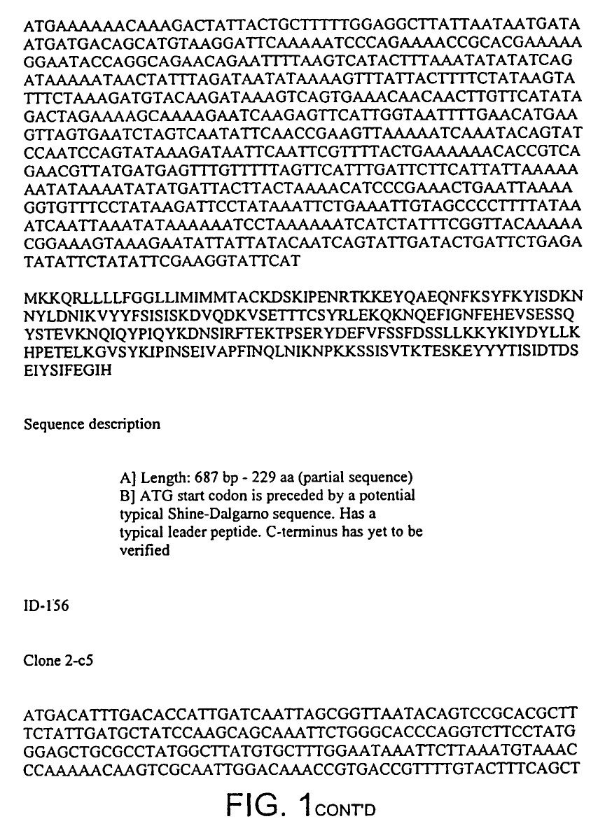

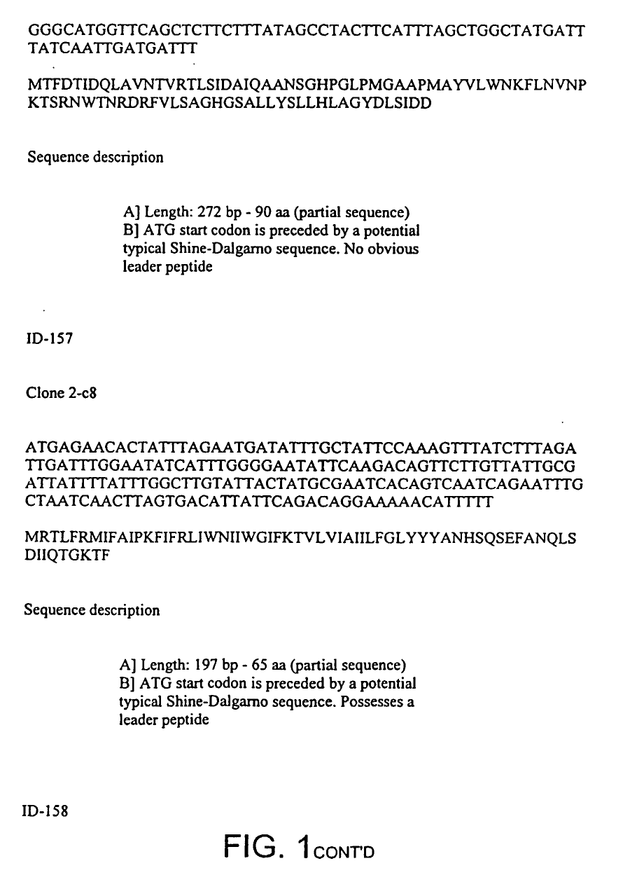

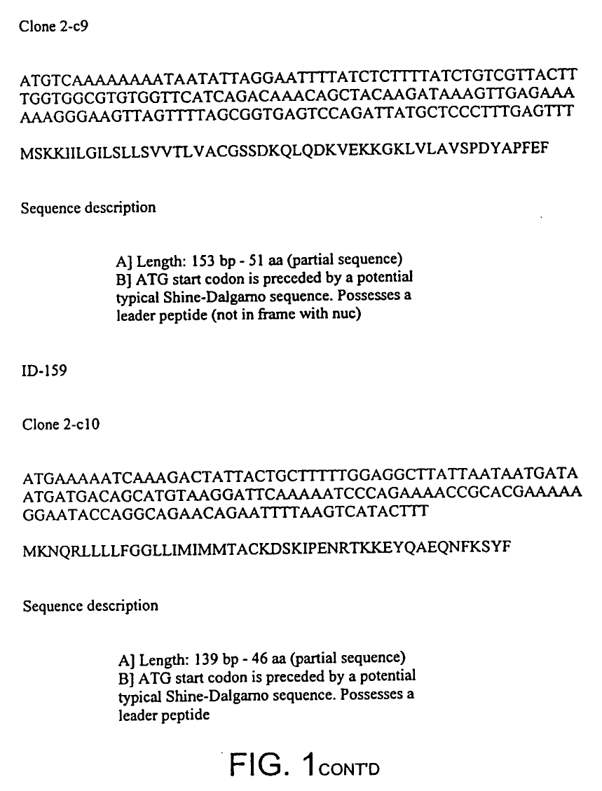

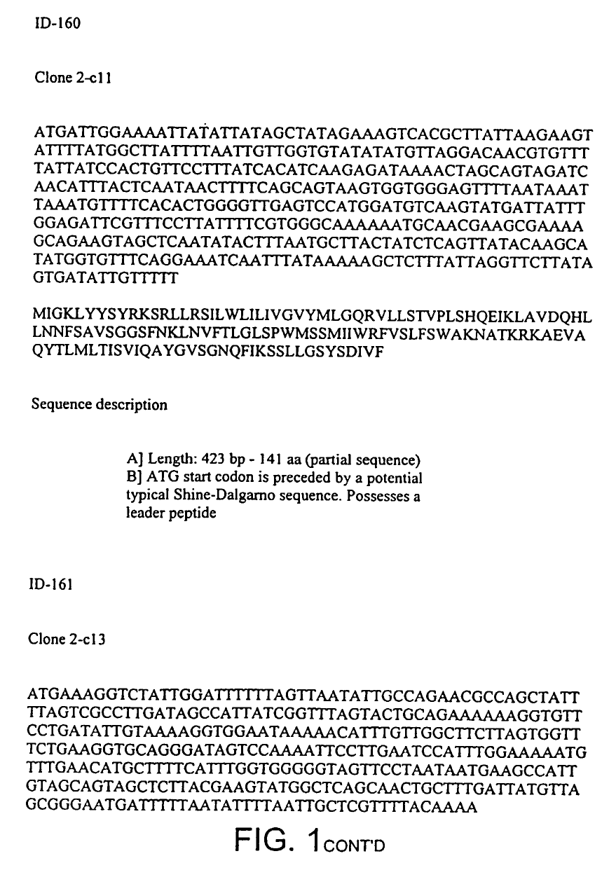

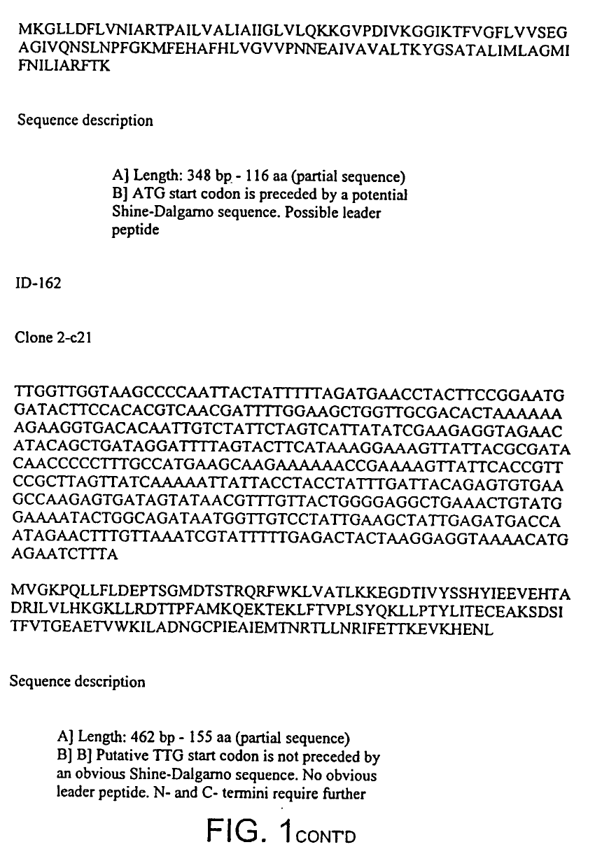

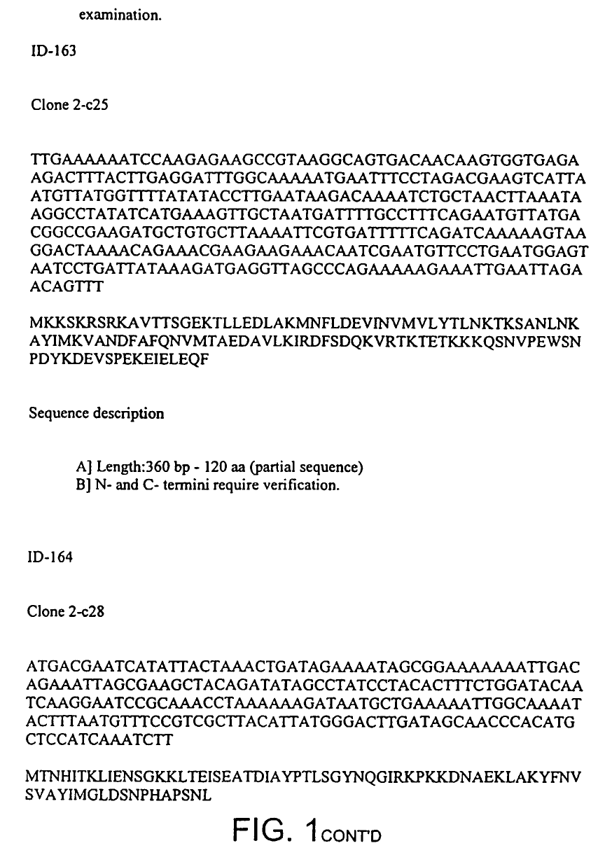

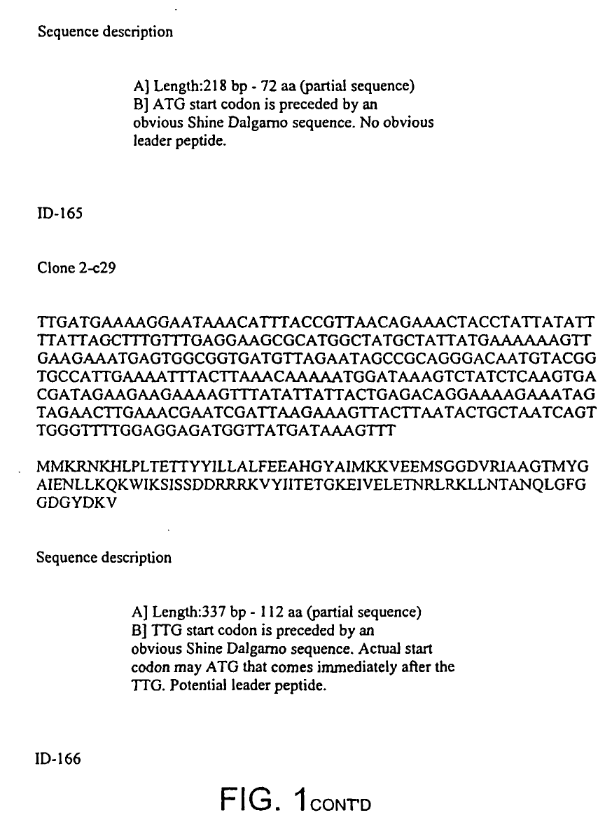

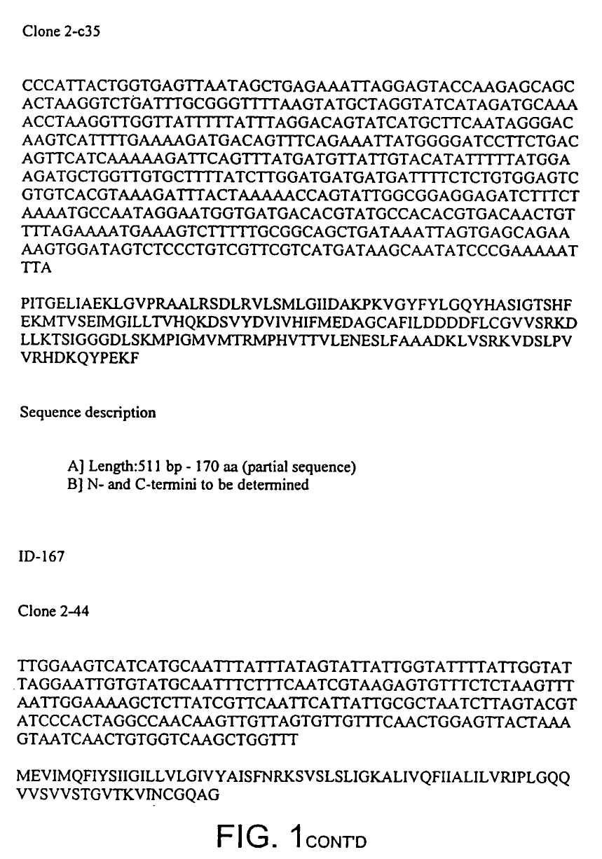

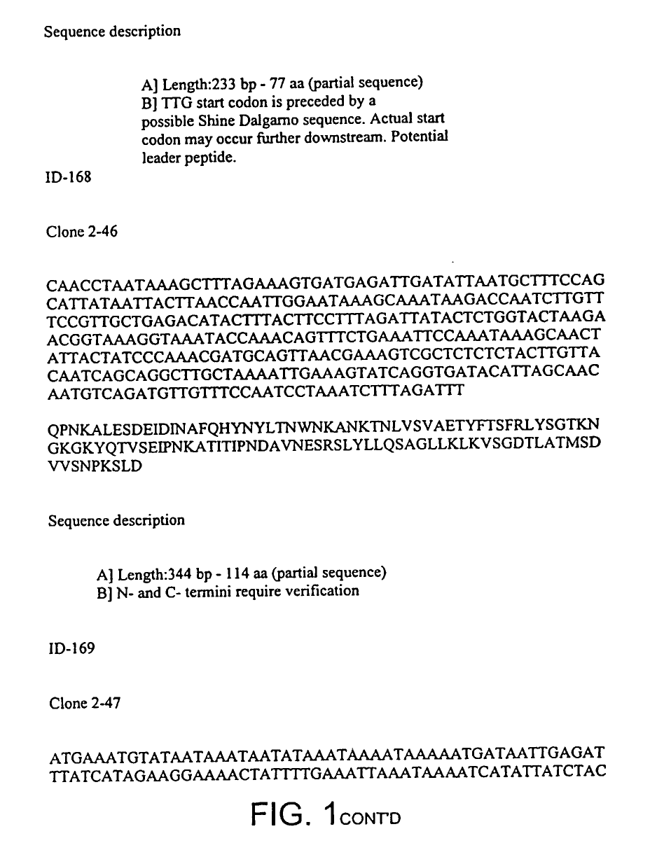

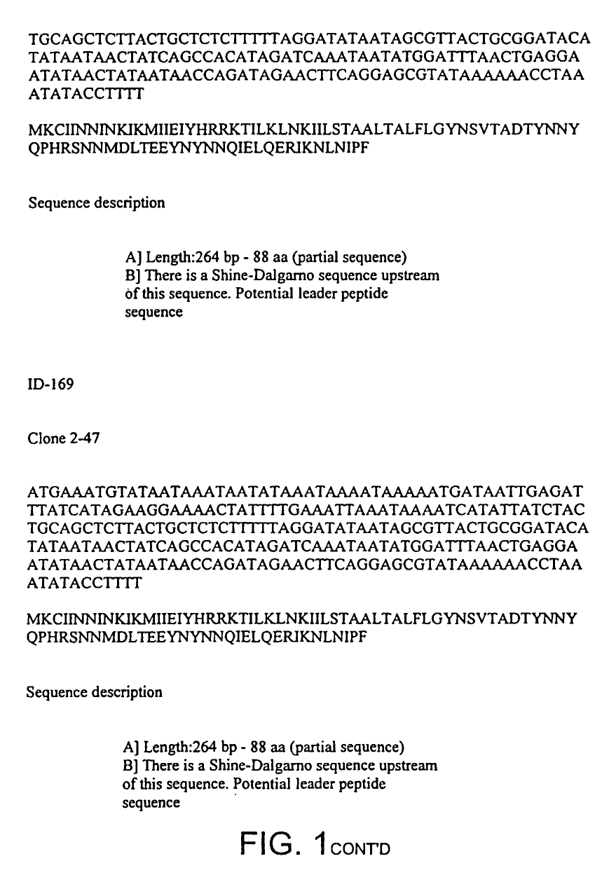

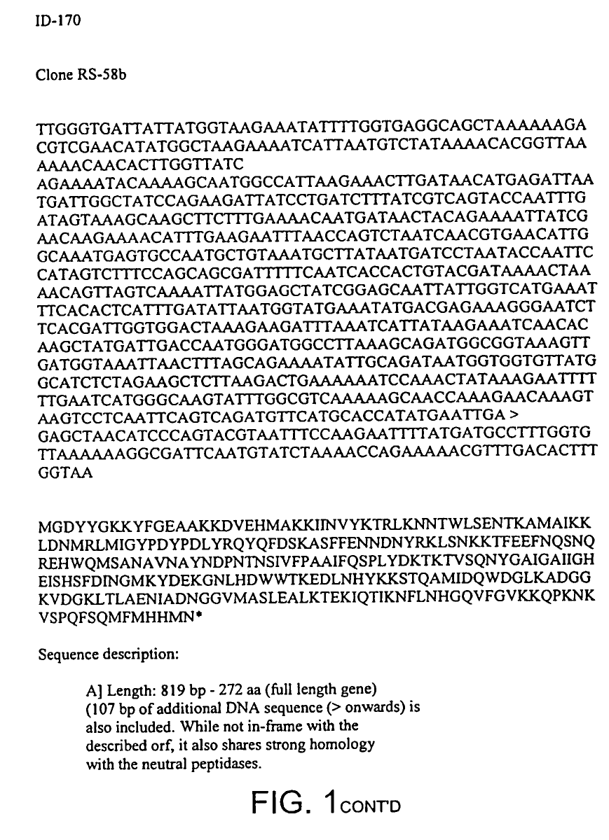

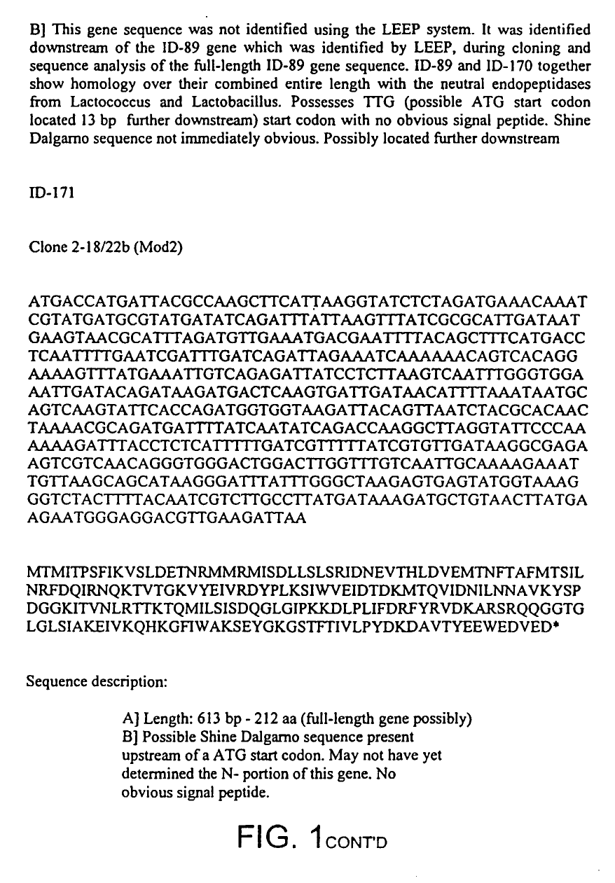

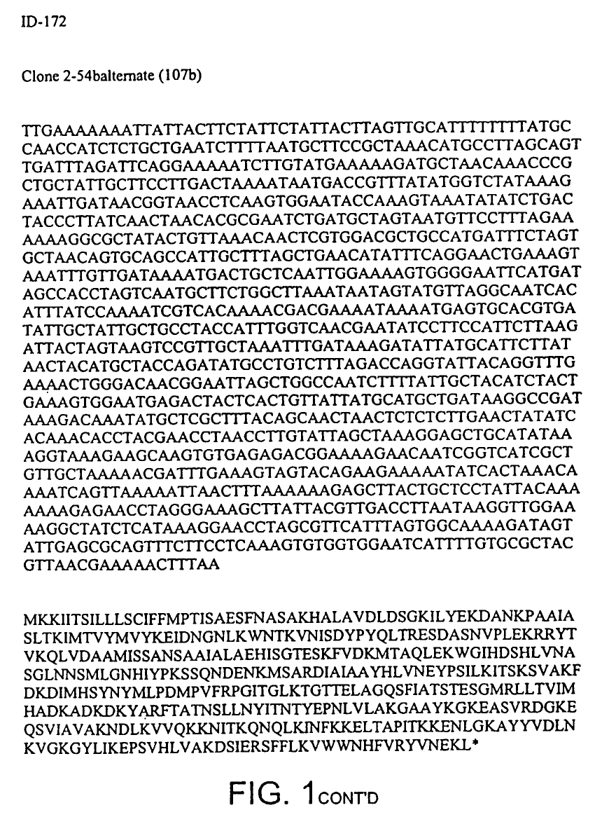

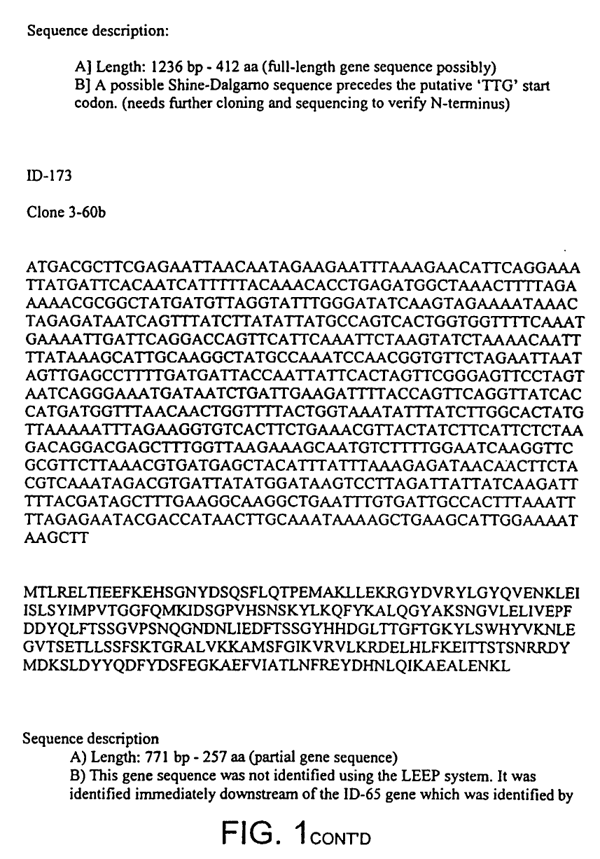

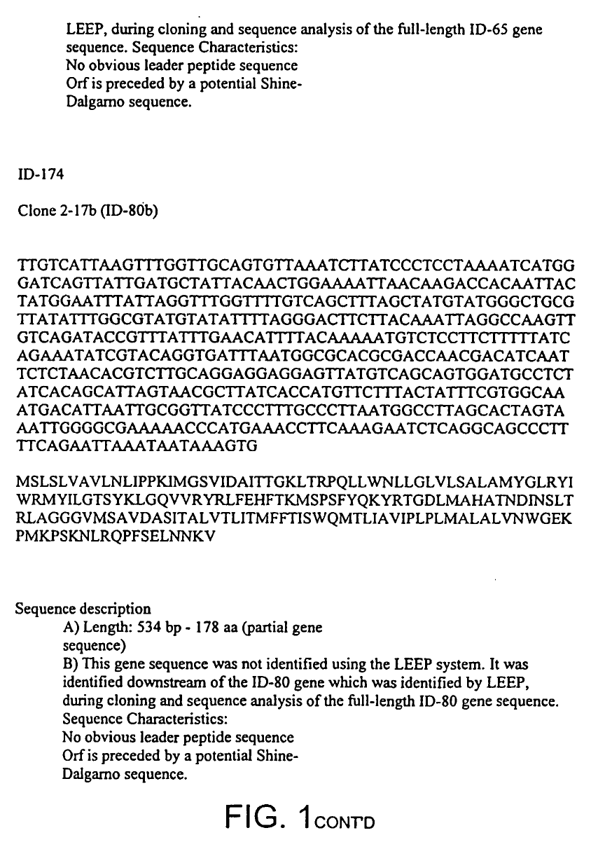

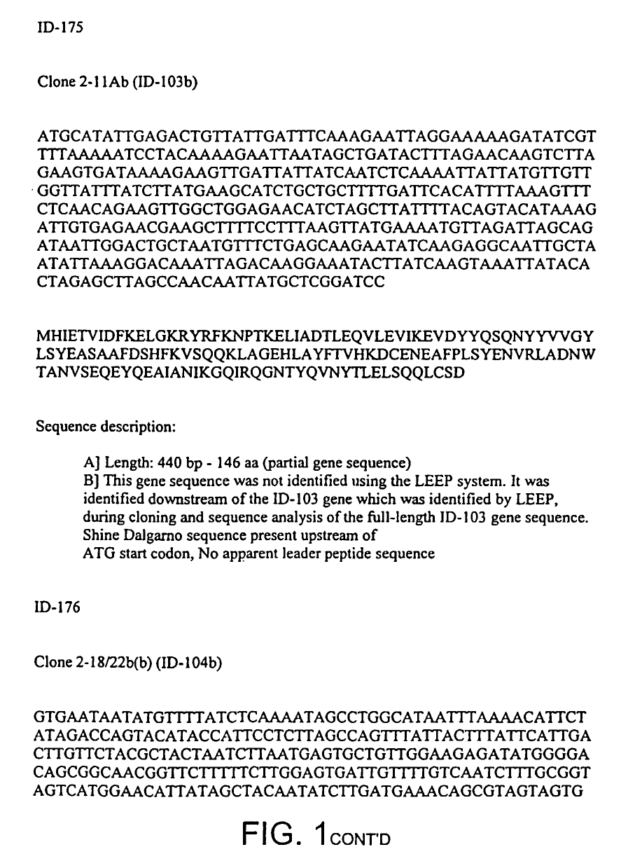

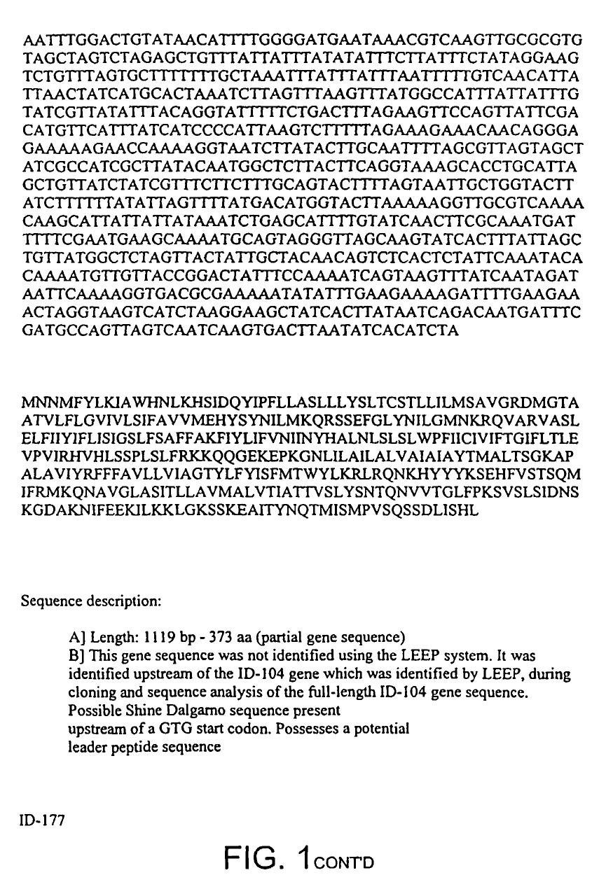

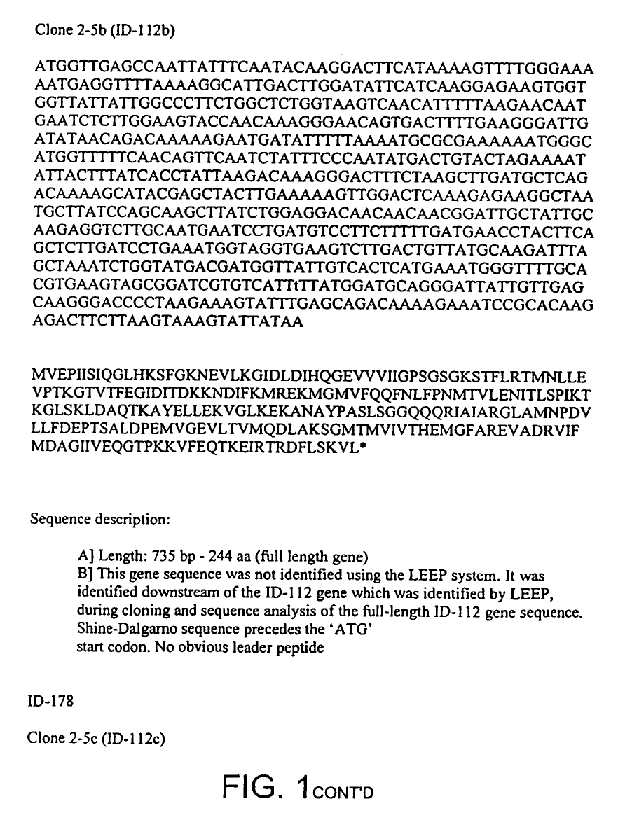

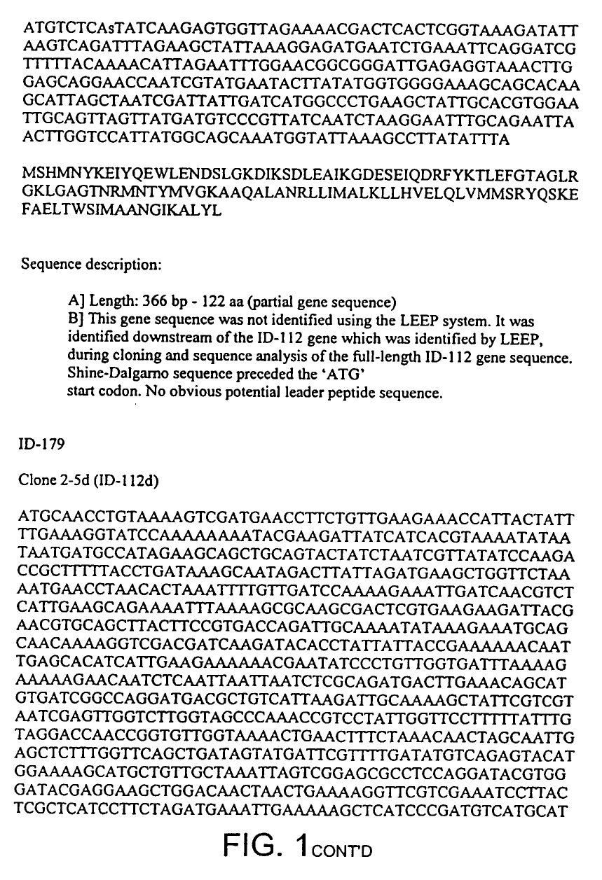

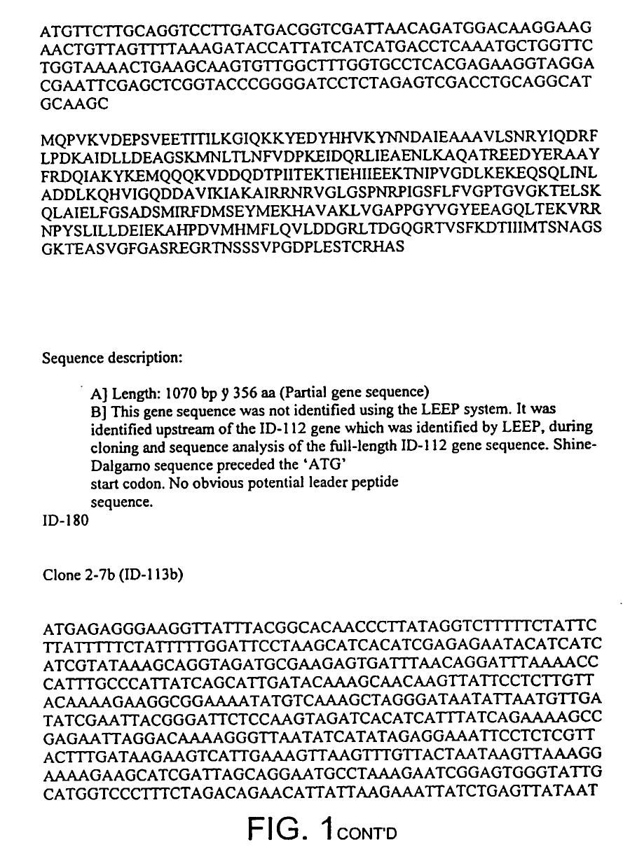

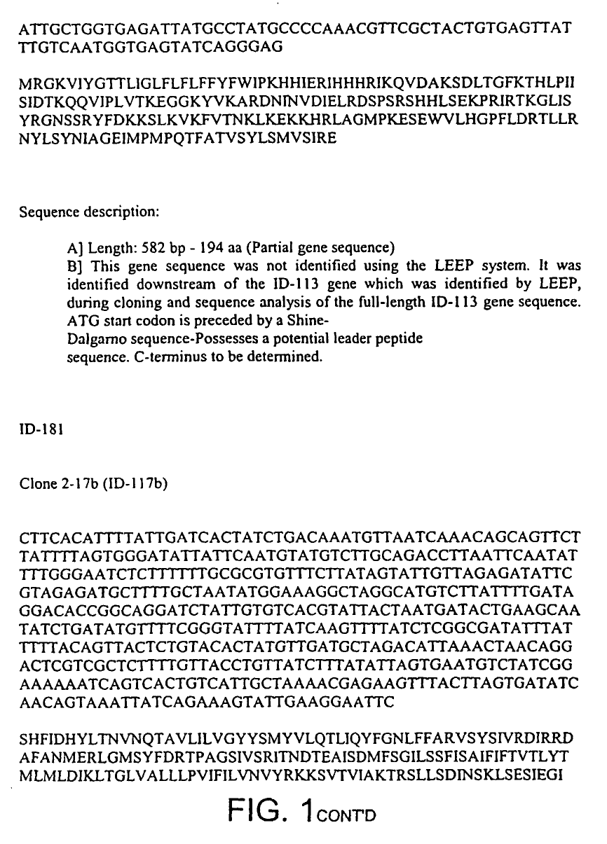

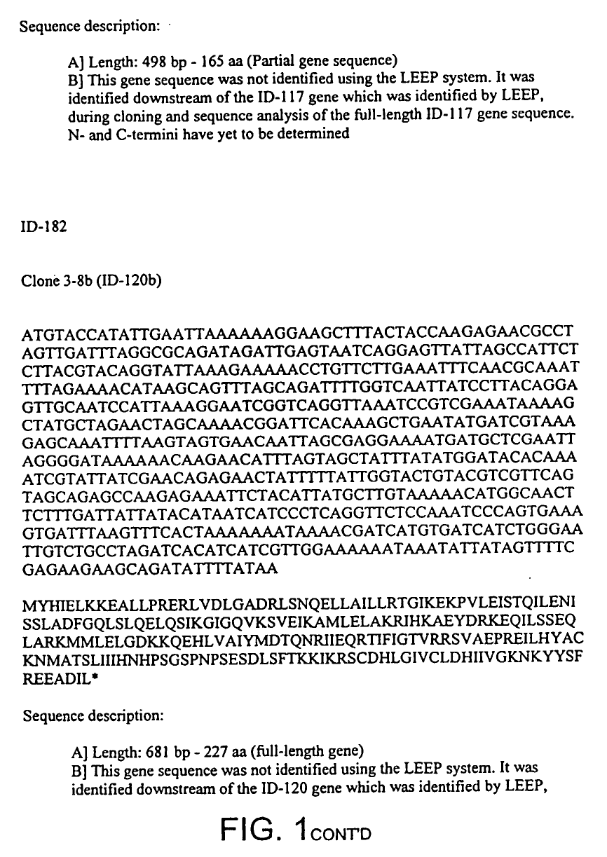

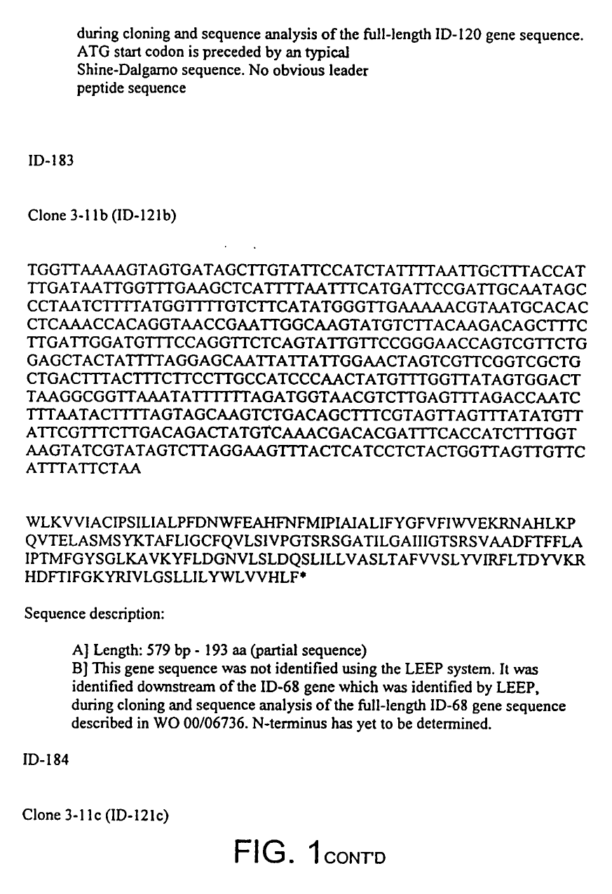

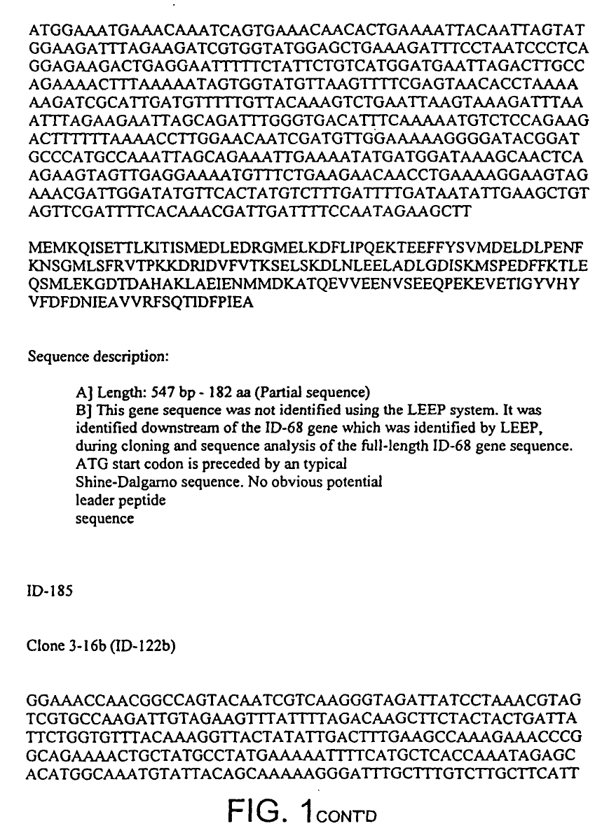

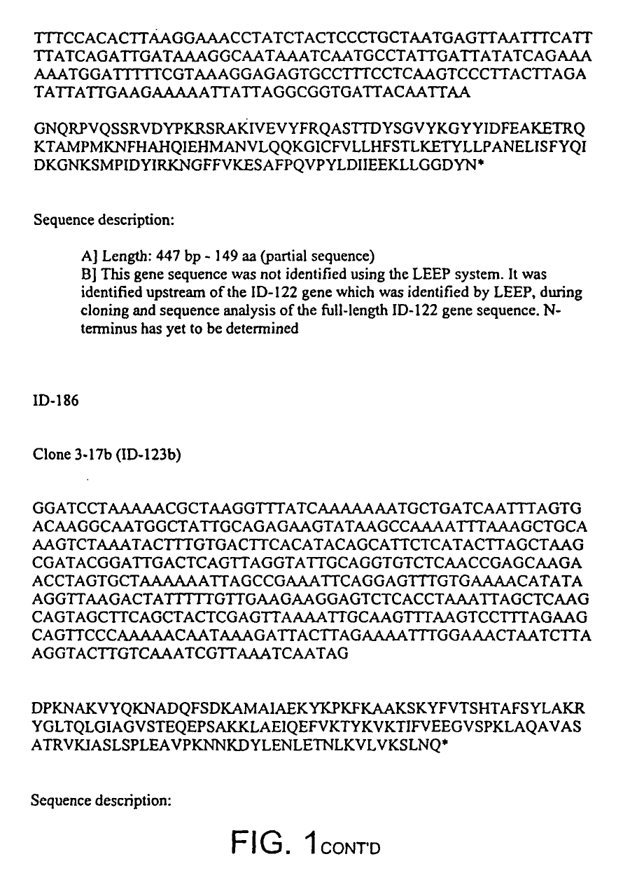

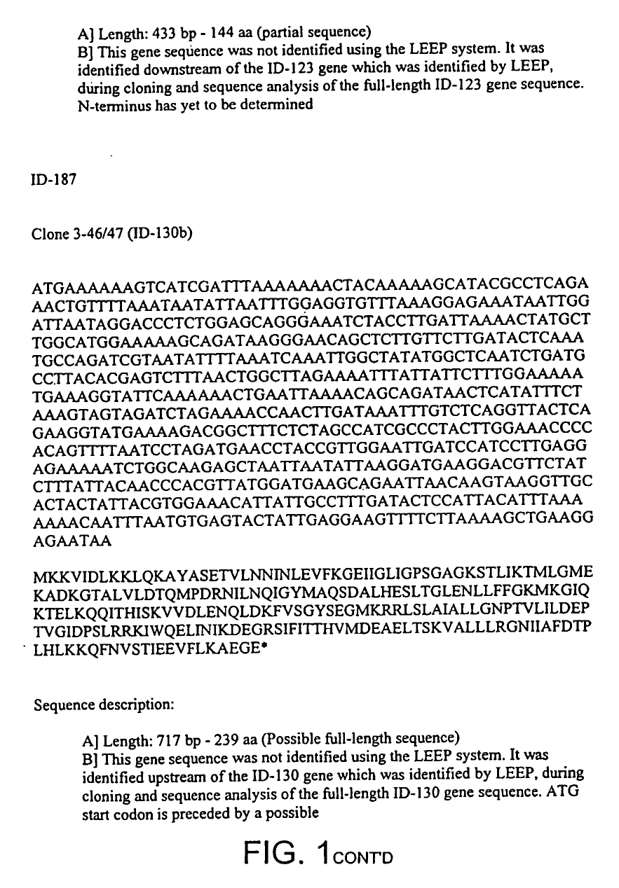

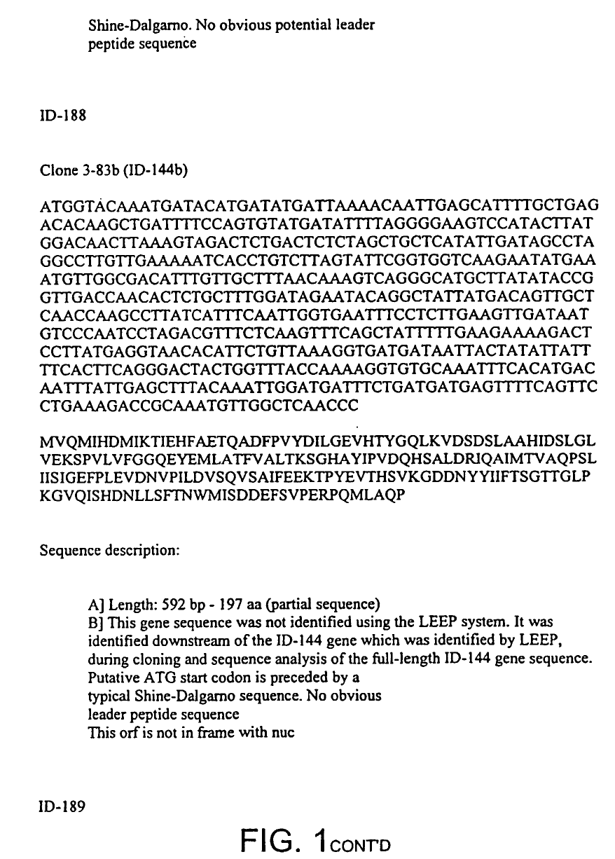

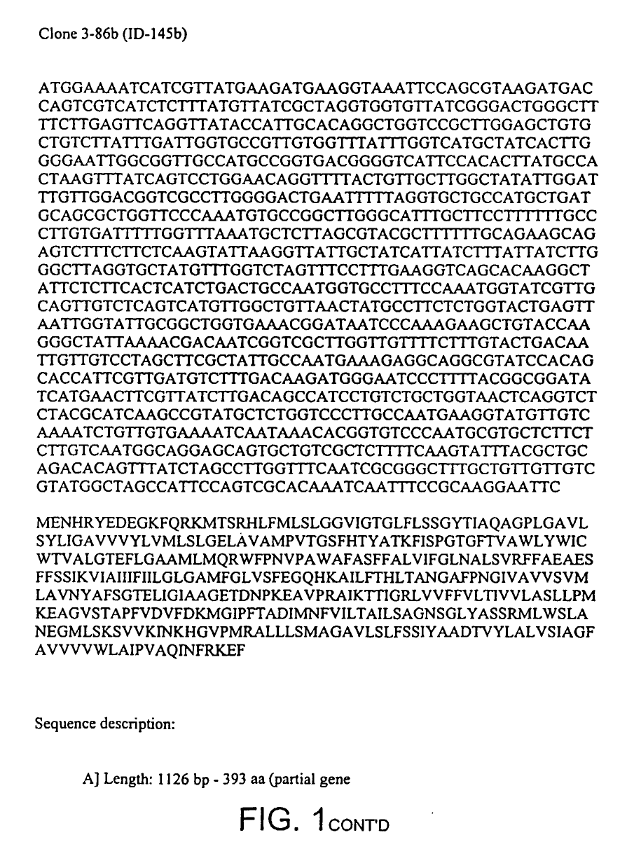

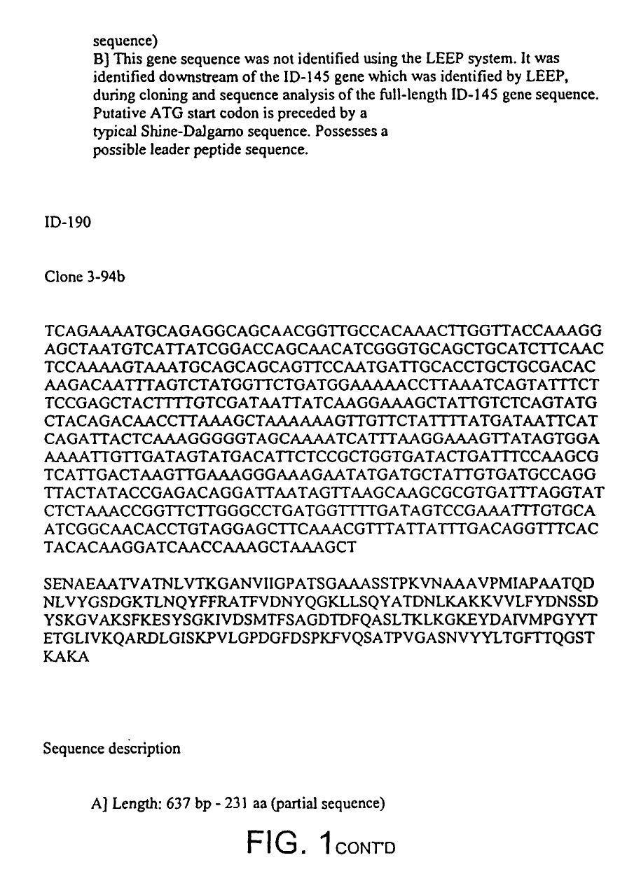

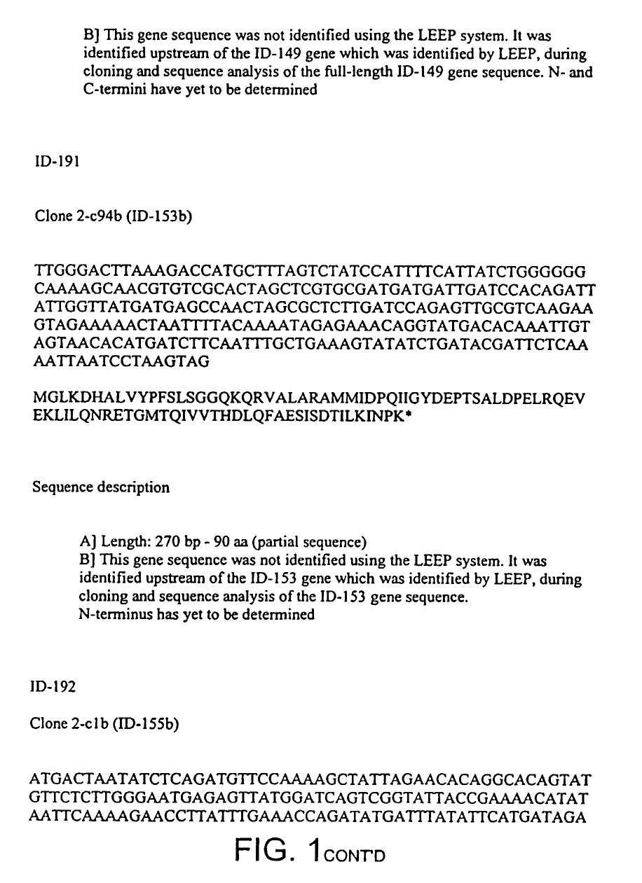

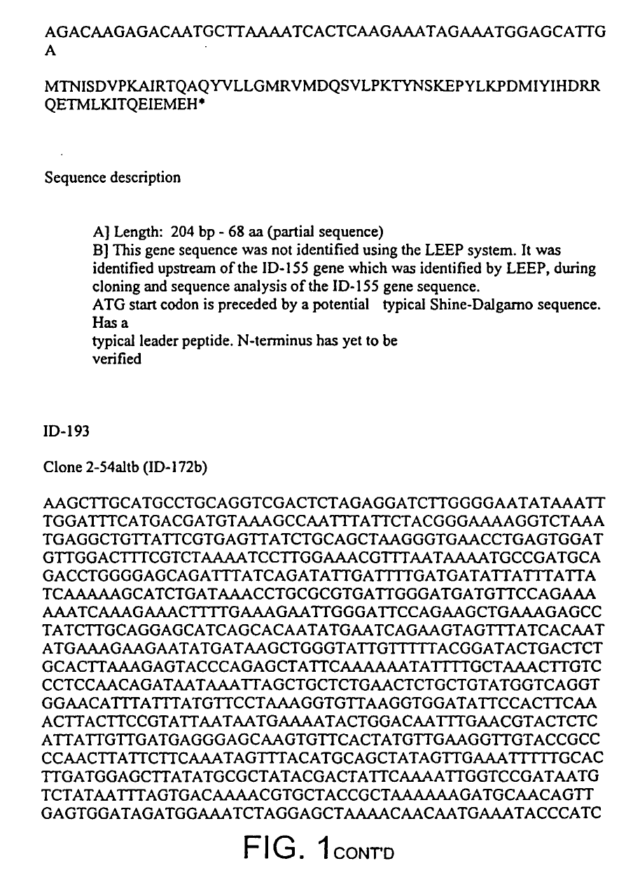

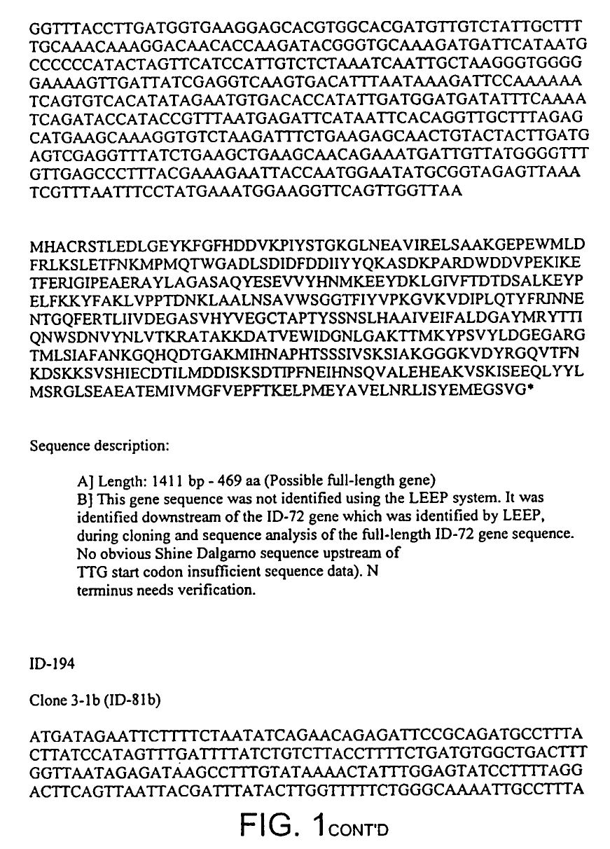

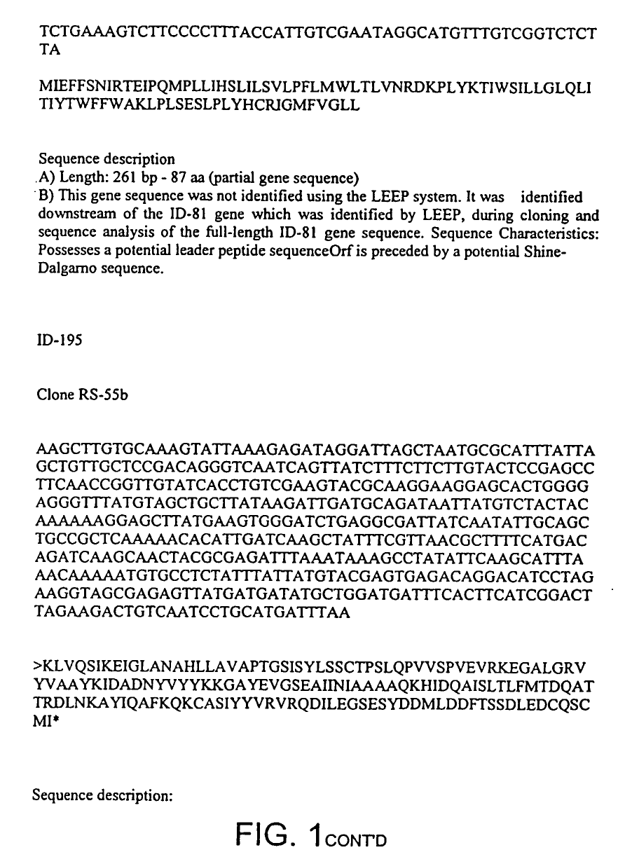

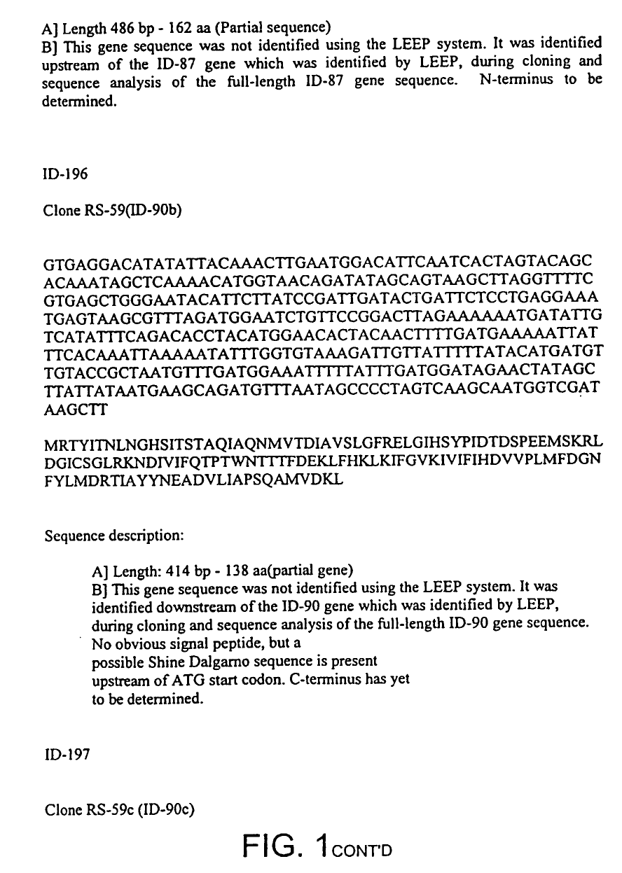

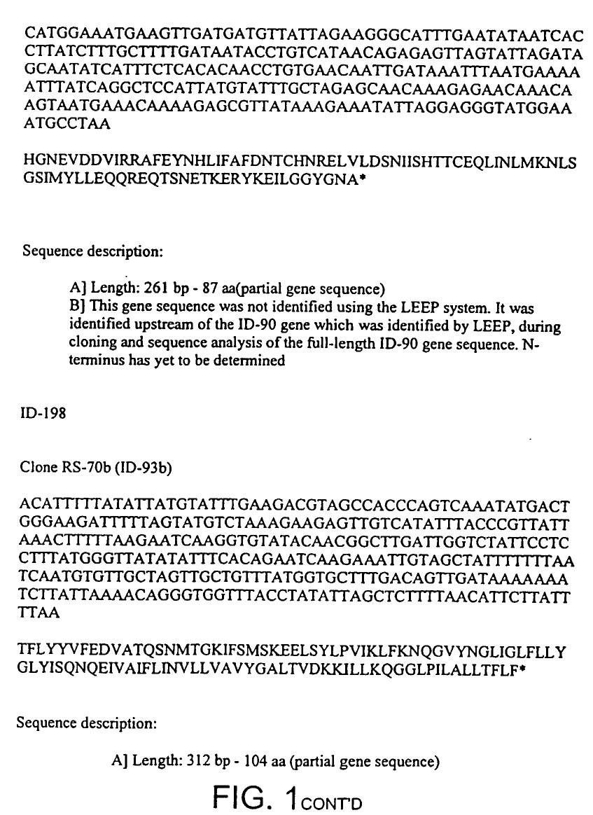

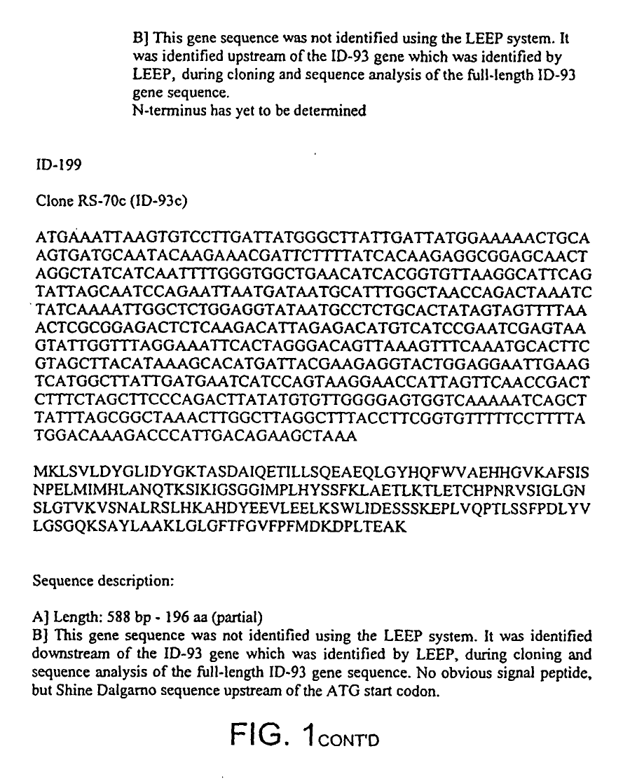

Thus in a first aspect, the present invention provides a Group B Streptococcus protein, polypeptide or peptide having a sequence selected from those shown in FIG. 1, or fragments or derivatives thereof.

It will be apparent to the skilled person that proteins and polypeptides included within this group may be cell surface receptors, adhesion molecules, transport proteins, membrane structural proteins, and/or signalling molecules.

Alterations in the amino acid sequence of a protein can occur which do not affect the function of a protein. These include amino acid deletions, insertions and substitutions and can result from alternative splicing and/or the presence of multiple translation start sites and stop sites. Polymorphisms may arise as a result of the infidelity of the translation process. Thus changes in amino acid sequence may be tolerated which do not affect the protein's function.

Thus, the present invention includes derivatives or variants of the proteins, polypeptides, and peptides of the present invention which show at least 50% identity to the proteins, polypeptides and peptides described herein. Preferably the degree of sequence identity is at least 60% and preferably it is above 75%. More preferably still it is above 80%, 90% or even 95%.

The term identity can be used to describe the similarity between two polypeptide sequences. A software package well known in the art for carrying out this procedure is the CLUSTAL program. It compares the amino acid sequences of two polypeptides and finds the optimal alignment by inserting spaces in either sequence as appropriate. The amino acid identity or similarity (identity plus conservation of amino acid type) for an optimal alignment can also be calculated using a software package such as BLASTx. This program aligns the largest stretch of similar sequence and assigns a value to the fit. For any one pattern comparison several Legions of similarity may be found, each having a different score. One skilled in the art will appreciate that two polypeptides of different lengths may be compared over the entire length of the longer fragment. Alternatively small regions may be compared. Normally sequences of the same length are compared for a useful comparison to be made.

Manipulation of the DNA encoding the protein is a particularly powerful technique for both modifying proteins and for generating large quantities of protein for purification purposes. This may involve the use of PCR techniques to amplify a desired nucleic acid sequence. Thus the sequence data provided herein can be used to design primers for use in PCR so that a desired sequence can be targeted and then amplified to a high degree.

Typically primers will be at least five nucleotides long and will generally be at least ten nucleotides long (e.g. fifteen to twenty-five nucleotides long). In some cases primers of at least thirty or at least thirty-five nucleotides in length may be used.

As a further alternative chemical synthesis may be used. This may be automated. Relatively short sequences may be chemically synthesised and ligated together to provide a longer sequence.

Thus in a further aspect, the present invention provides, a nucleic acid molecule comprising or consisting of a sequence which is:

(i) any of the DNA sequences set out in FIG. 1 herein or their RNA equivalents;

(ii) a sequence which is complementary to any of the sequences of (i);

(iii) a sequence which codes for the same protein or polypeptide, as those sequences of (i) or (ii);

(iv) a sequence which is shows substantial identity with any of those of (i), (ii) and (iii); or

(v) a sequence which codes for a derivative or fragment of a nucleic acid molecule shown in FIG. 1.

The term identity can also be used to describe the similarity between two individual DNA sequences. The ‘bestfit’ program (Smith and Waterman, Advances in applied Mathematics, 482-489 (1981)) is one example of a type of computer software used to find the best segment of similarity between two nucleic acid sequences, whilst the GAP program enables sequences to be aligned along their whole length and finds the optimal alignment by inserting spaces in either sequence as appropriate.

The present invention includes nucleic acid sequences which show at least 50% identity to the nucleic acid sequences described herein. Preferably the degree of sequence identity is at least 60% and preferably it is above 75%. More preferably still it is above 80%, 90% or even 95%.

The term ‘RNA equivalent’ when used above indicates that a given RNA molecule has a sequence which is complementary to that of a given DNA molecule, allowing for the fact that in RNA ‘U’ replaces ‘T’ in the genetic code. The nucleic acid molecule may be in isolated, recombinant or chemically synthetic form.

DNA constructs can readily be generated using methods well known in the art. These techniques are disclosed, for example in J. Sambrook et al, Molecular Cloning 2^{nd} Edition, Cold Spring Harbour Laboratory Press (1989). Modifications of DNA constructs and the proteins expressed such as the addition of promoters, enhancers, signal sequences, leader sequences, translation start and stop signals and DNA stability controlling regions, or the addition of fusion partners may then be facilitated.

Normally the DNA construct will be inserted into a vector which may be any suitable vector, including plasmid, virus, bacteriophage, transposon, minichromosome, liposome or mechanical carrier. The expression vectors of the invention are DNA constructs suitable for expressing DNA which encodes the desired protein product which may include: (a) a regulatory element (e.g. a promoter, operator, activator, repressor and/or enhancer), (b) a structural or coding sequence which is transcribed into mRNA and (c) appropriate transcription, translation, initiation and termination sequences. The vector may further comprise a selectable marker, for example antibiotic resistance, which facilitates the selection and/or identification of cells containing the vector.

Expression of the protein is achieved by the transformation or transfection of the vector into a host cell which may be of eukaryotic or prokaryotic origin. For the production of recombinant protein, expression may be inducible expression or expression only in certain types of cells or both inducible and cell-specific. Particularly preferred among inducible vectors are vectors that can be induced for expression by environmental factors that are easy to manipulate, such as temperature and nutrient additives. A variety of suitable vectors, including constitutive and inducible expression vectors for use in prokaryotic and eukaryotic hosts, are well known and employed routinely by those skilled in the art.

A great variety of expression vectors can be used to express the Group B Streptococcus protein(s) of the invention. Such vectors include, among others, chromosomal, episomal and virus-derived vectors, for example, vectors derived from bacterial plasmids, from bacteriophage, from transposons, from yeast elements, from viruses such as baculoviruses, papova viruses, such as SV40, vaccinia viruses, adenoviruses and retroviruses, and vectors derived from combinations thereof, such as those derived from plasmid and bacteriophage genetic elements, such as cosmids and phagemids, all may be used in accordance with the invention. Generally, any vector suitable to maintain, propagate or express. nucleic acid to express a polypeptide in a host may be used for expression in this regard. Such vectors thus form yet a further aspect of the invention.

The appropriate DNA sequence may be inserted into the vector by any of a variety of well-known and routine techniques.

The nucleic acid sequence in the expression vector is operatively linked to appropriate expression control sequence(s) including, for instance, a promoter to direct mRNA transcription. Representatives of such promoters include, but are not limited to, the phage lambda PL promoter, the T3 and T7 promoters, the E. coli lac, trp, tac, and λP_{L} promoters, the microbial eukaryote GAL, glucoamylase and cellobiohydrolase promoters and the mammalian metallothionein (mouse) and heat-shock (human) promoters.

In general, expression vectors will contain sites for transcription initiation and termination, and, in the transcribed region, a ribosome binding site for translation. The coding portion of mature transcripts expressed by the constructs will generally include a translation initiating AUG at the beginning and a termination codon appropriately positioned at the end of the polypeptide to be translated.

Representative examples of appropriate hosts for recombinant expression of the Group B Streptococcus protein(s) of the invention include bacterial cells, such as streptococci, staphylococci, E. coli, streptomyces and Bacillus subtilis cells; fungal cells, such as yeast cells and Aspergillus cells; insect cells such as Drosophila S2 and Spodoptera Sf9 cells; animal cells such as CHO, COS, HeLa and Bowes melanoma cells; and plant cells. Such host cells form yet a further aspect of the present invention.

Microbial cells employed in the expression of proteins can be disrupted by any convenient method, including freeze-thaw cycling, sonication, mechanical disruption, or use of cell lysing agent, such methods which are known to those skilled in the art.

The polypeptide can be recovered and purified from recombinant cell cultures by well-known methods including ammonium sulphate or ethanol precipitation, acid extraction, anion or cation exchange chromatography, phosphocellulose, chromatography, hydrophobic interaction chromatography, affinity chromatography, hydroxylapatite chromatography and lectin chromatography. Well known techniques for refolding protein may be employed to regenerate active conformation when the polypeptide is denatured during isolation and or purification.

The Group B Streptococcus proteins described herein can additionally be used as target antigens to raise antibodies, or to generate affibodies. These can be used to detect Group B Streptococcus.

Thus in a further aspect the present invention provides, an antibody, affibody, or a derivative thereof which binds to any one or more of the proteins, polypeptides, peptides, fragments or derivatives thereof, as described herein.

Antibodies within the scope of the present invention may be monoclonal or polyclonal. Polyclonal antibodies can be raised by stimulating their production in a suitable animal host (e.g. a mouse, rat, guinea pig, rabbit, sheep, goat or monkey) when a protein as described herein, or a homologue, derivative or fragment thereof, is injected into the animal. If desired, an adjuvant may be administered together with the protein. Well-known adjuvants include Freund's adjuvant (complete and incomplete) and aluminium hydroxide. The antibodies can then be purified by virtue of their binding to a protein as described herein and by many other means well-known to those skilled in the art.

Monoclonal antibodies can be produced from hybridomas. These can be formed by fusing myeloma cells and spleen cells which produce the desired antibody in order to form an immortal cell line. Thus the well-known Kohler & Milstein technique (Nature 256 (1975)) or subsequent variations upon this technique can be used.

Techniques for producing monoclonal and polyclonal antibodies that bind to a particular polypeptide/protein are now well developed in the art. They are discussed in standard immunology textbooks, for example in Roitt et al, Immunology second edition (1989), Churchill Livingstone, London.

In addition to whole antibodies, the present invention includes derivatives thereof which are capable of binding to proteins etc as described herein. Thus the present invention includes antibody fragments and synthetic constructs. Examples of antibody fragments and synthetic constructs are given by Dougall et al., Tibtech 12 372-379 (September 1994).

Antibody fragments include, for example, Fab, F(ab′)₂ and Fv fragments. Fv fragments can be modified to produce a synthetic construct known as a single chain Fv (scFv) molecule. This includes a peptide linker covalently joining Vₕ and Vₗ regions, which contributes to the stability of the molecule. Other synthetic constructs that can be used include CDR peptides. These are synthetic peptides comprising antigen-binding determinants. Peptide mimetics may also be used. These molecules are usually conformationally restricted organic rings that mimic the structure of a CDR loop and that include antigen-interactive side chains.

Synthetic constructs include chimaeric molecules. Thus, for example, humanised (or primatised) antibodies or derivatives thereof are within the scope of the present invention. An example of a humanised antibody is an antibody having human framework regions, but rodent hypervariable regions. Ways of producing chimaeric antibodies are discussed for example by Morrison et al in PNAS, 81, 6851-6855 (1984) and by Takeda et al in Nature. 314, 452-454 (1985).

Synthetic constructs also include molecules comprising an additional moiety that provides the molecule with some desirable property in addition to antigen binding. For example the moiety may be a label (e.g. a fluorescent or radioactive label). Alternatively, it may be a pharmaceutically active agent.

Affibodies are proteins which are found to bind to target proteins with a low dissociation constant. They are selected from phage display libraries expressing a segment of the target protein of interest (Nord K, Gunneriusson E, Ringdahl J, Stahl S, Uhlen M, Nygren Pa., Department of Biochemistry and Biotechology, Royal Institute of Technology (KTH), Stockholm, Sweden).

In a further aspect the invention provides an immunogenic composition comprising one or more proteins, polypeptides, peptides, fragments or derivatives thereof, or nucleotide sequences described herein. The immunogenic composition may include nucleic acid sequences ID-65 and/or ID-66 as described herein. Alternatively, the immunogenic composition may comprise proteins/polypeptides including ID-65, ID-83, ID-89, ID-93 and/or ID-96 as described herein, or fragments or derivatives thereof. A composition of this sort may be useful in the treatment or prevention of Group B Streptococcus infection in subject. In a preferred aspect of the invention the immunogenic composition is a vaccine.

In other aspects the invention provides:

i) Use of an immunogenic composition as described herein in the preparation of a medicament for the treatment or prophylaxis of Group B Streptococcus infection. Preferably the medicament is a vaccine.

ii) A method of detection of Group B Streptococcus which comprises the step of bringing into contact a sample to be tested with at least one antibody, affibody, or a derivative thereof, as described herein.

iii) A method of detection of Group B Streptococcus which comprises the step of bringing into contact a sample to be tested with at least one protein, polypeptide, peptide, fragments or derivatives as described herein.

iv) A method of detection of Group B Streptococcus which comprises the step of bringing into contact a sample to be tested with at least one nucleic acid molecule as described herein.

v) A kit for the detection of Group B Streptococcus comprising at least one antibody, affibody, or derivatives thereof, described herein.

vi) A kit for the detection of Group B Streptococcus comprising at least one Group B Streptococcus protein, polypeptide, peptide, fragment or derivative thereof, as described herein.

vii) A kit for the detection of Group B Streptococcus comprising at least one nucleic acid of the invention.

As described previously, the novel proteins described herein are identified and isolated using a screening method which specifically identifies those Group B Streptococcus genes encoding bacterial cell envelope associated or secreted proteins.

Given that the inventors have identified a group of important proteins, such proteins are potential targets for anti-microbial therapy. It is necessary, however, to determine whether each individual protein is essential for the organism's viability. Thus, the present invention also provides a method of determining whether a protein or polypeptide as described herein represents a potential anti-microbial target which comprises inactivating said protein and determining whether Group B Streptococcus is still viable.

A suitable method for inactivating the protein is to effect selected gene knockouts, ie prevent expression of the protein and determine whether this results in a lethal change. Suitable methods for carrying out such gene knockouts are described in Li et al, P.N.A.S., 94:13251-13256 (1997) and Kolkman et al., Journal of Biological Chemistry 272: 19502-19508 (1997); Kolkman et al., Journal of Bacteriology 178: 3736-3741 (1996).

In a final aspect the present invention provides the use of an agent capable of antagonising, inhibiting or otherwise interfering with the function or expression of a protein or polypeptide of the invention in the manufacture of a medicament for use in the treatment or prophylaxis of Group B Streptococcus infection.

The invention will now be described by means of the following examples which should not in any way be construed as limiting. The examples refer to the figures in which:

FIG. 1: (A) Shows a number of full length nucleotide sequences encoding antigenic Group B Streptococcus proteins and the corresponding amino acid sequences.

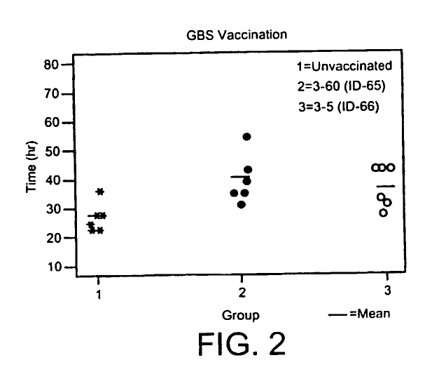

FIG. 2: Shows the results of vaccine trials using the proteins ID-65 and ID-66;

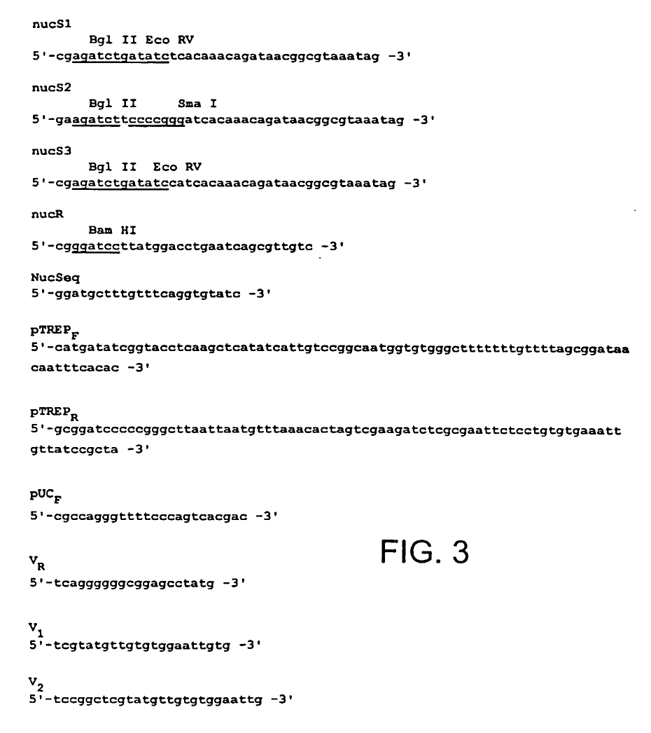

FIG. 3: Shows a number of oligonucleotide primers used in the screening process

nucS1 primer designed to amplify a mature form of the nuc A gene

nucS2—primer designed to amplify a mature form of the nuc A gene.

nucS3 primer designed to amplify a mature form of the nuc A gene

nucR primer designed to amplify a mature form of the nuc A gene

nucseq primer designed to sequence DNA cloned into the pTREP-Nuc vector

pTREPF nucleic acid sequence containing recognition site for ECORV. Used for cloning fragments into pTREX7.

pTREPR nucleic acid sequence containing recognition site for BAMH 1. Used for cloning fragments into pTREX7.

PUCF forward sequencing primer, enables direct sequencing of cloned DNA fragments.

VR example of gene specific primer used to obtain further antigen DNA sequence by the method of DNA walking.

V1 example of gene specific primer used to obtain further antigen DNA sequence by the method of DNA walking.

V2 example of gene specific primer used to obtain further antigen DNA sequence by the method of DNA walking.

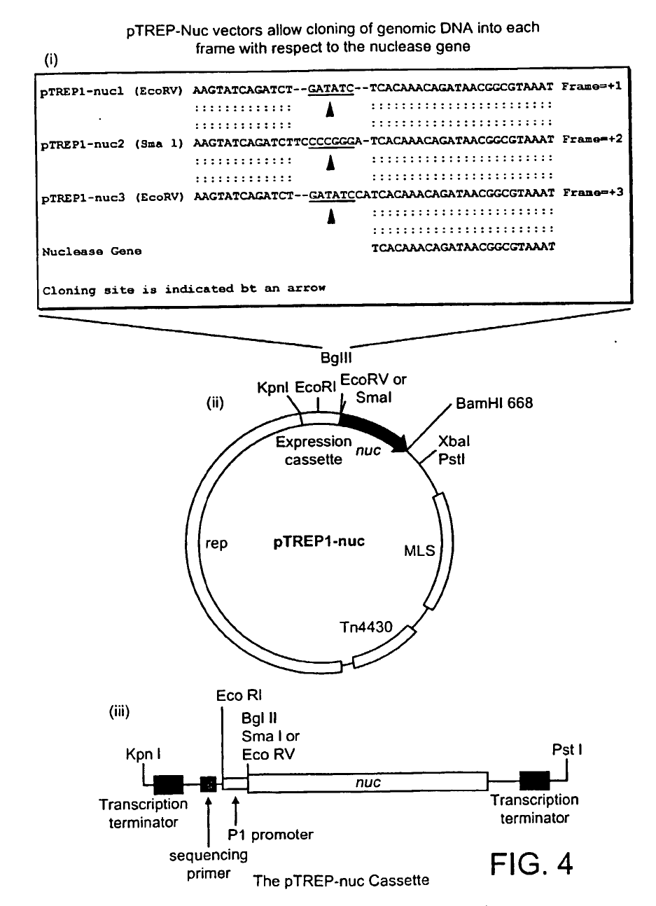

FIG. 4: (i) Schematic presentation of the nucleotide sequence of the unique gene cloning site immediately upstream of the mature nuc gene in pTREP1-nuc1, pTREP1-nuc2 and pTREP1-nuc3. Each of the pTREP-nuc vectors contain an EcoRV (a Smal site in pTREP1-nuc2) cleavage site which allows cloning of genomic DNA fragments in 3 different frames with respect to the mature nuc gene.

(ii) A physical and genetic summary map of the pTREP1-nuc vectors. The expression cassette incorporating nuc, the macrolides, lincosamides and streptogramin B (MLS) resistance determinant, and the replicon (rep) Ori-pAMβ1 are depicted (not drawn to scale).

(iii) Schematic presentation of the expression cassette showing the various sequence elements involved in gene expression and location of unique restriction endonuclease sites (not drawn to scale).

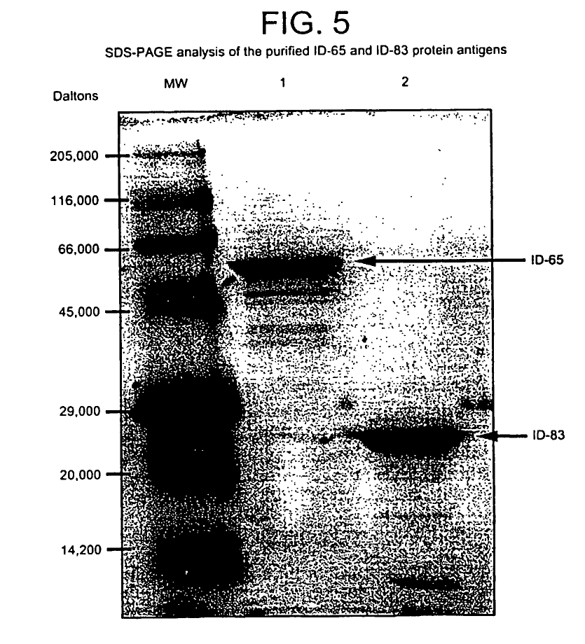

FIG. 5: SDS-PAGE analysis of a purified preparation of the His-tagged ID-65 and ID-83 protein antigens (predicted molecular weights of 57,144 and 25,000 daltons respectively) on a 12% polyacrylamide gel. Lanes: MW, molecular weight standards; 1, His-tagged ID-65 protein; 2, His-tagged ID-83 protein

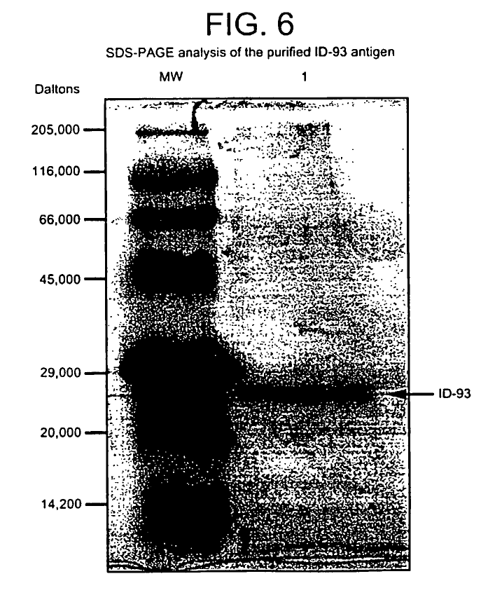

FIG. 6: SDS PAGE analysis of a purified preparation of the His-tagged ID-93 protein antigen (predicted molecular weight=28,000 daltons) on a 12% polyacrylamide gel. Lanes: MW, molecular weight standards; 1, His-tagged ID-93 protein.

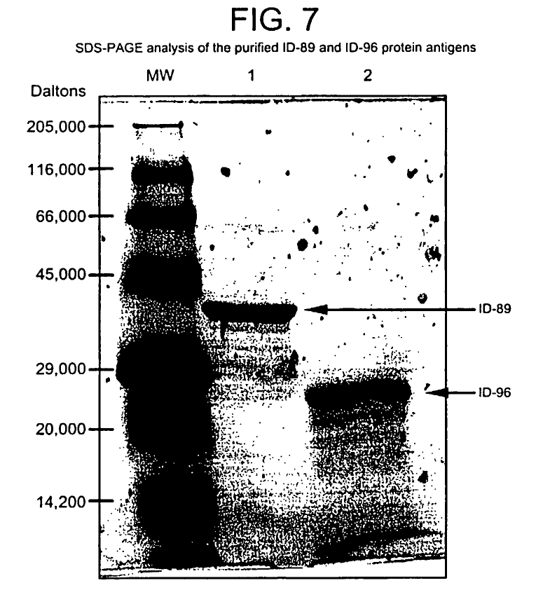

FIG. 7: SDS PAGE analysis of a purified preparation of the His-tagged ID-89 and ID-96 protein antigens (predicted molecular weights of 35,000 and 31,000 daltons respectively) on a 12% polyacrylamide gel. Lanes: MW, molecular weight standards; 1, His-tagged ID-89 protein; 2, His-tagged ID-96 protein.

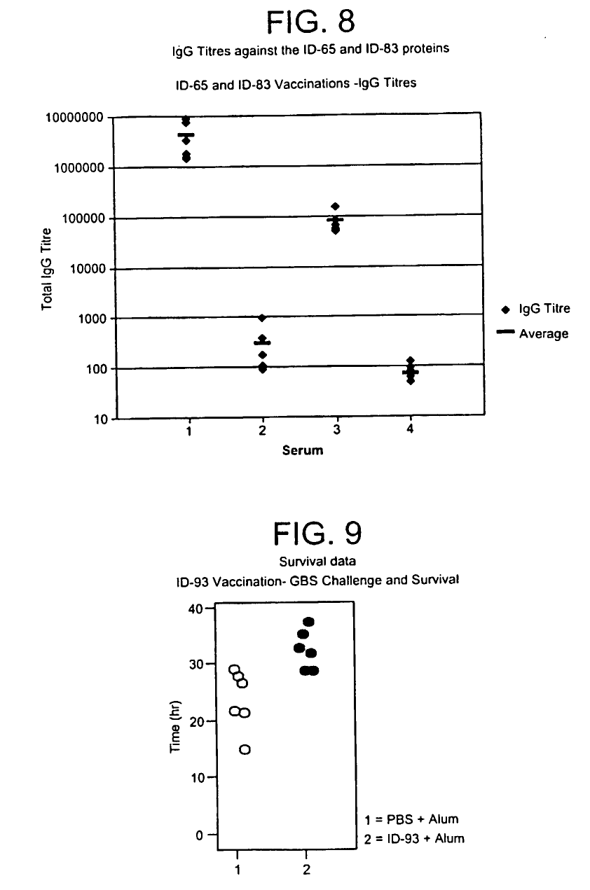

FIG. 8: IgG Titres against the ID-65 and ID-83 proteins

1=ID-65+Alum Group−Bleed at 5 weeks

2=PBS+Alum Control Group−Bleed at 5 weeks

(For groups 1 and 2, ELISAs were performed on purified ID-65 protein)

3=ID-83+Alum Group−Bleed at 5 weeks

4=PBS+Alum Control Group−Bleed at 5 weeks

(For groups 3 and 4, ELISAs were performed on purified ID-83 protein)

FIG. 9: Shows the results of vaccine trials using the protein ID-93.

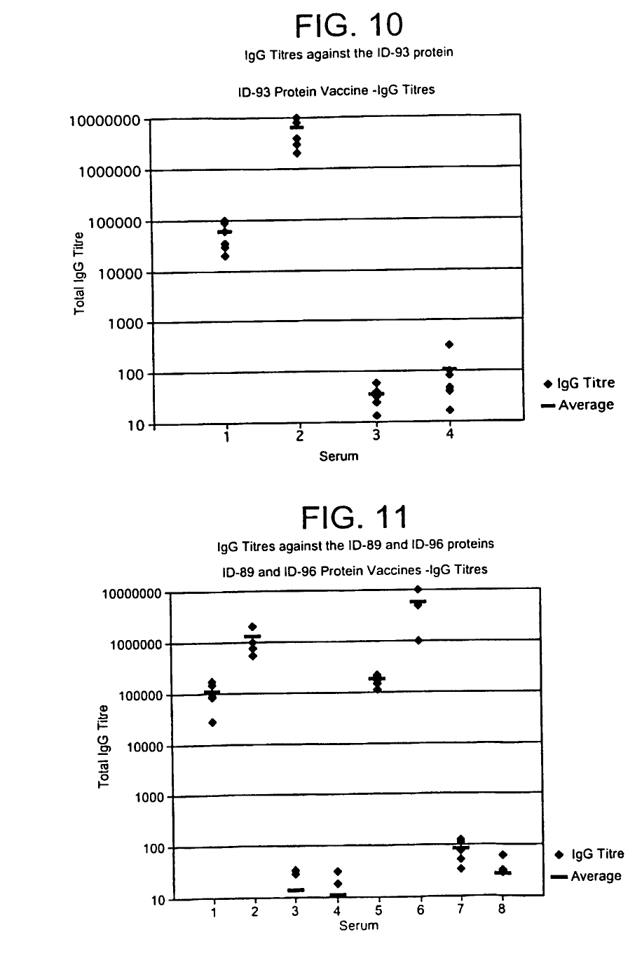

FIG. 10: IgG titres against the ID-93 protein.

1=ID-93+Alum Group−Bleed at 3 weeks

2=ID-93+Alum Group−Bleed at 6 weeks

3=PBS+Alum Control Group−Bleed at 3 weeks

4=PBS+Alum Control Group−Bleed at 6 weeks

FIG. 11: IgG titres against the ID-89 and ID-96 proteins

1=ID-89+TitreMax Gold Group−Bleed at 3 weeks

2=ID-89+TitreMax Gold−Bleed at 6 weeks

3=PBS+TitreMax Gold Control Group−Bleed at 3 weeks

4=PBS+TitreMax Gold Control Group−Bleed at 6 weeks

5=ID-96+TitreMax Gold Group−Bleed at 3 weeks

6=ID-96+TitreMax Gold Group−Bleed at 6 weeks

7=PBS+TitreMax Gold Control Group−Bleed at 3 weeks

8=PBS+TitreMax Gold Control Group−Bleed at 6 weeks

For Groups 1-4, ELISAs were performed on purified ID-89 protein.

For Groups 5-6, ELISAs were performed on purified ID-96 protein.

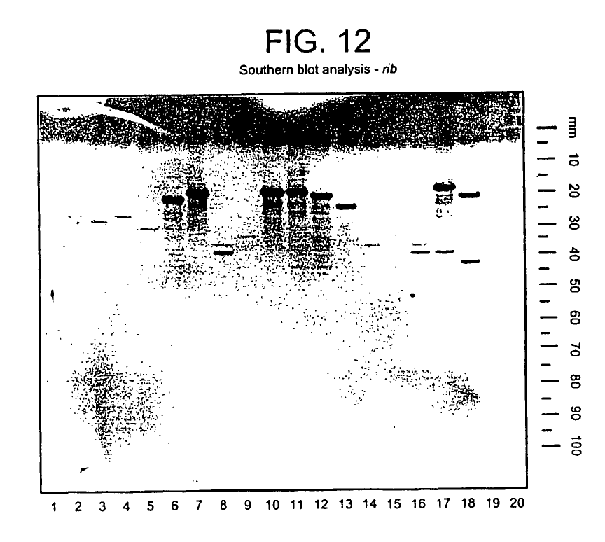

FIG. 12: Southern blot analysis of genomic DNA. Genomic DNA from each of the strains listed in Table 7 was digested completely with Hin DIII (NEB) and electrophoresed at 40 Volts for 6 hours in 0.8% agarose, transferred onto Hybond N⁺ (Amersham) membrane by Southern blot and hybridised with the digoxigenin-labelled rib gene probe. Specifically bound DNA probe was identified using the DIG Nucleic Acid Detection Kit (Boehringer Mannheim).

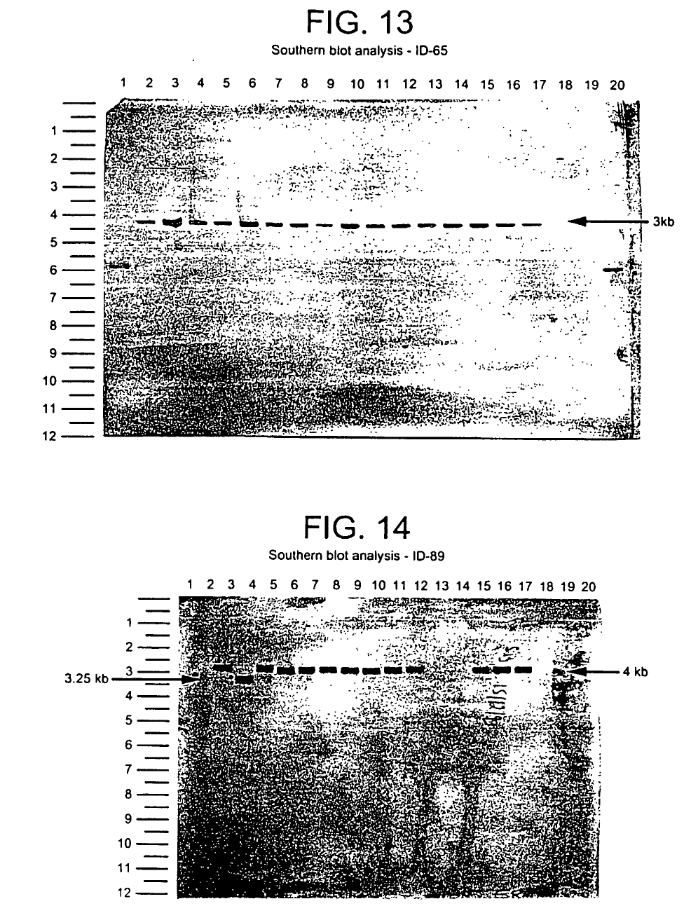

FIG. 13: Southern blot analysis of genomic DNA. Genomic DNA from each of the strains listed in Table 6 was digested completely with Hin DIII (NEB) and electrophoresed at 40 Volts for 6 hours in 0.8% agarose, transferred onto Hybond N⁺ (Amersham) membrane by Southern blot and hybridised with the digoxigenin-labelled ID-65 gene probe. Specifically bound DNA probe was identified using the DIG Nucleic Acid Detection Kit (Boehringer Mannheim).

FIG. 14: Southern blot analysis of genomic DNA. Genomic DNA from each of the strains listed in Table 6 was digested completely with Hin DIII (NEB) and electrophoresed at 40 Volts for 6 hours in 0.8% agarose, transferred onto Hybond N⁺ (Amersham) membrane by Southern blot and hybridised with the digoxigenin-labelled ID-89 gene probe. Specifically bound DNA probe was identified using the DIG Nucleic Acid Detection Kit (Boehringer Mannheim).

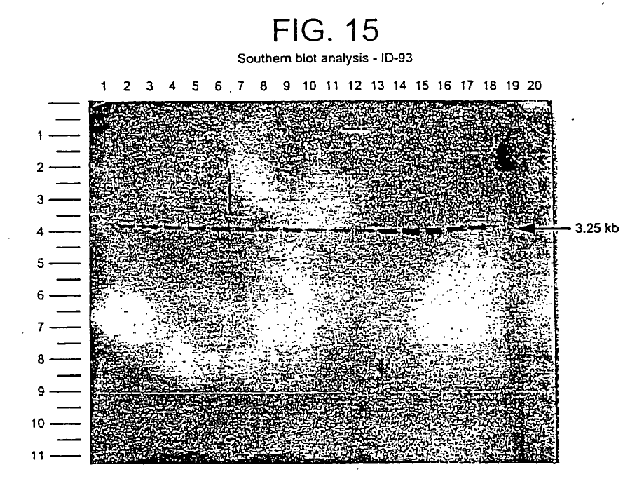

FIG. 15: Southern blot analysis of genomic DNA. Genomic DNA from each of the strains listed in Table 6 was digested completely with Hin DIII (NEB) and electrophoresed at 40 Volts for 6 hours in 0.8% agarose, transferred onto Hybond N⁺ (Amersham) membrane by Southern blot and hybridised with the digoxigenin-labelled ID-93 gene probe. Specifically bound DNA probe was identified using the DIG Nucleic Acid Detection Kit (Boehringer Mannheim).

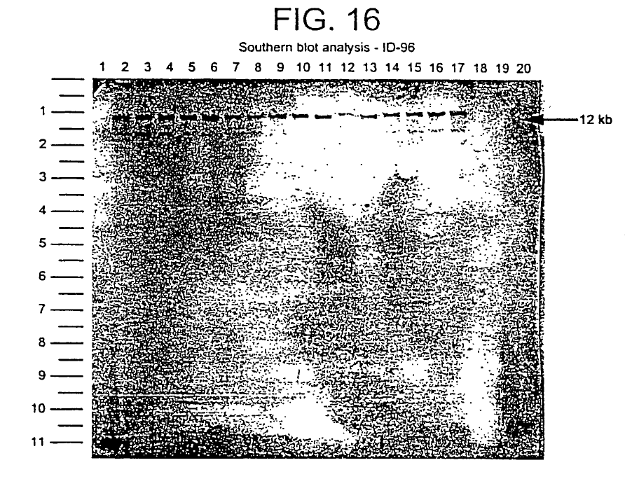

FIG. 16: Southern blot analysis of genomic DNA. Genomic DNA from each of the strains listed in Table 6 was digested completely with Eco RI (NEB) and electrophoresed at 40 Volts for 6 hours in 0.8% agarose, transferred onto Hybond N⁺ (Amersham) membrane by Southern blot and hybridised with the digoxigenin-labelled ID-96 gene probe. Specifically bound DNA probe was identified using the DIG Nucleic Acid Detection Kit (Boehringer Mannheim).

### EXAMPLE 1

Gene/partial gene sequences putatively encoding exported proteins in S. agalactiae have been identified, unless stated otherwise, using the nuclease screening system described herein vis, the LEEP (Lactococcus Expression of Exported Proteins) system. These have been further analysed to remove artefacts. The nucleotide sequences of genes identified using the screening system have been characterised using a number of parameters described below.

1. All putative surface proteins are analysed for leader/signal peptide sequences. Bacterial signal peptide sequences share a common design. They are characterised by a short positively charged N-terminus (N region) immediately preceding a stretch of hydrophobic residues (central portion-h region) followed by a more polar C-terminal portion which contains the cleavage site (c-region). Computer software is used to perform hydropathy profiling of putative proteins (Marcks, Nuc. Acid. Res., 16:1829-1836 (1988)) which is used to identify the distinctive hydrophobic portion (h-region) typical of leader peptide sequences. In addition, the presence/absence of a potential ribosomal binding site (Shine-Dalgarno sequence required for translation) is also noted.

2. All putative surface protein sequences are used to search the OWL sequence database which includes a translation of the GENBANK and SWISSPROT database.. This allows identification of similar sequences which may have been previously characterised not only at the sequence level but at a functional level. It may also provide information indicating that these proteins are indeed surface related and not artefacts.

3. Putative S. agalactiae surface proteins are also assessed for their novelty. Some of the identified proteins may or may not possess a typical leader peptide sequence and may not show homology with any DNA/protein sequences in the database. Indeed these proteins may indicate the primary advantage of our screening method, i.e. isolating atypical surface-related proteins, which would have been missed in all previously described screening protocols.

The construction of three reporter vectors and their use in L. lactis to identify and isolate genomic DNA fragments from pathogenic bacteria encoding secreted or surface associated proteins is now described.

Construction of the pTREP1-nuc series of reporter vectors

(a) Construction of expression plasmid PTREP1

The pTREP1 plasmid is a high-copy number (40-80 per cell) theta-replicating gram positive plasmid, which is a derivative of the pTREX plasmid which is itself a derivative of the previously published pIL253 plasmid. pIL253 incorporates the broad Gram-positive host range replicon of pAMβ1 (Simon and Chopin, Biochemie 70: 559-566 (1988))L lactis sex-factor. pIL253 also lacks the tra function which is necessary for transfer or efficient mobilisation by conjugative parent plasmids exemplified by pIL501. The Enterococcal pAMβ1 replicon has previously been transferred to various species including Streptococcus, Lactobacillus and Bacillus species as well as Clostridium acetobutylicum, (LeBlanc et al., Proceedings of the National Academy of Science USA 75:3484-3487 (1978)) indicating the potential broad host range utility. The pTREP1 plasmid represents a constitutive transcription vector.

The pTREX vector was constructed as follows. An artificial DNA fragment containing a putative RNA stabilising sequence, a translation initiation region (TIR), a multiple cloning site for insertion of the target genes and a transcription terminator was created by annealing 2 complementary oligonucleotides and extending with Tfl DNA polymerase. The sense and anti-sense oligonucleotides contained the recognition sites for NheI and BamHI at their 5′ ends respectively to facilitate cloning. This fragment was cloned between the XbaI and BamHI sites in pUC19NT7, a derivative of pUC19 which contains the T7 expression cassette from pLET1 (Wells et al., J. Appl. Bacteriol. 74:629-636 (1993)) cloned between the EcoRI and HindIII sites. The resulting construct was designated pUCLEX. The complete expression cassette of pUCLEX was then removed by cutting with HindIII and blunting followed by cutting with EcoRI before cloning into EcoRI and SacI (blunted) sites of pIL253 to generate the vector pTREX (Wells and Schofield, In Current advances in metabolism, genetics and applications-NATO ASI Series. H 98:37-62. (1996)). The putative RNA stabilising sequence and TIR are derived from the Escherichia coli T7 bacteriophage sequence and modified at one nucleotide position to enhance the complementarity of the Shine Dalgarno (SD) motif to the ribosomal 16s RNA of Lactococcus lactis (Schofield et al. pers. coms. University of Cambridge Dept. Pathology.).

A Lactococcus lactis MG1363 chromosomal DNA fragment exhibiting promoter activity which was subsequently designated P7 was cloned between the EcoRI and BglII sites present in the expression cassette, creating pTREX7. This active promoter region had been previously isolated using the promoter probe vector pSB292 (Waterfield et al., Gene 165:9-15 (1995)). The promoter fragment was amplified by PCR using the Vent DNA polymerase according to the manufacturer.

The pTREP1 vector was then constructed as follows. An artificial DNA fragment which included a transcription terminator, the forward pUC sequencing primer, a promoter multiple cloning site region and a universal translation stop sequence was created by annealing two overlapping partially complementary synthetic oligonucleotides together and extending with sequenase according to manufacturers instructions. The sense and anti-sense (pTREP_{F} and pTREP_{R}) oligonucleotides contained the recognition sites for EcoRV and BamHI at their 5′ ends respectively to facilitate cloning into pTREX7. The transcription terminator was that of the Bacillus penicillinase gene, which has been shown to be effective in Lactococcus (Jos et al., Applied and Environmental Microbiology 50:540-542 (1985)). This was considered necessary as expression of target genes in the pTREX vectors was observed to be leaky and is thought to be the result of cryptic promoter activity in the origin region (Schofield et al. pers. coms. University of Cambridge Dept. Pathology.). The forward pUC primer sequencing was included to enable direct sequencing of cloned DNA fragments. The translation stop sequence which encodes a stop codon in 3 different frames was included to prevent translational fusions between vector genes and cloned DNA fragments. The pTREX7 vector was first digested with EcoRI and blunted using the 5′-3′ polymerase activity of T4 DNA polymerase (NEB) according to manufacturer's instructions. The EcoRI digested and blunt ended pTREX7 vector was then digested with Bgl II thus removing the P7 promoter. The artificial DNA fragment derived from the annealed synthetic oligonucleotides was then digested with EcoRV and Bam HI and cloned into the EcoRI(blunted)-Bgl II digested pTREX7 vector to generate pTREP. A Lactococcus lactis MG1363 chromosomal promoter designated P1 was then cloned between the EcoRI and BglII sites present in the pTREP expression cassette forming pTREP1. This promoter was also isolated using the promoter probe vector pSB292 and characterised by Waterfield et al., (1995) [supra]. The P1 promoter fragment was originally amplified by PCR using vent DNA polymerase according to manufacturers instructions and cloned into the pTREX as an EcoRI-BglII DNA fragment. The EcoRI-BglII P1 promoter containing fragment was removed from pTREX1 by restriction enzyme digestion and used for cloning into pTREP (Schofield et al. pers. coms. University of Cambridge, Dept. Pathology.).

(b) PCR amplification of the S. aureus nuc gene.

The nucleotide sequence of the S. aureus nuc gene (EMBL database accession number V01281) was used to design synthetic oligonucleotide primers for PCR amplification. The primers were designed to amplify the mature form of the nuc gene designated nucA which is generated by proteolytic cleavage of the N-terminal 19 to 21 amino acids of the secreted propeptide designated Snase B (Shortle, 1983 [supra]). Three sense primers (nucS1, nucS2 and nucS3, shown in FIG. 3) were designed, each one having a blunt-ended restriction endonuclease cleavage site for EcoRV or SmaI in a different reading frame with respect to the nuc. gene. Additionally BglII and BamHI were incorporated at the 5′ ends of the sense and anti-sense primers respectively to facilitate cloning into BamHI and BglII cut pTREP1. The sequences of all the primers are given in FIG. 3. Three nuc gene DNA fragments encoding the mature form of the nuclease gene (NucA) were amplified by PCR using each of the sense primers combined with the anti-sense primer. The nuc gene fragments were amplified by PCR using S. aureus genomic DNA template, Vent DNA Polymerase (NEB) and the conditions recommended by the manufacturer. An initial denaturation step at 93° C. for 2 min was followed by 30 cycles of denaturation at 93° C. for 45 sec, annealing at 50° C. for 45 seconds, and extension at 73° C. for 1 minute and then a final 5 min extension step at 73° C. The PCR amplified products were purified using a Wizard clean up column (Promega) to remove unincorporated nucleotides and primers.

(c) Construction of the pTREP1-nuc vectors

The purified nuc gene fragments described in section b were digested with Bgl II and BamHI using standard conditions and ligated to BamHI and BglII cut and dephosphorylated pTREP1 to generate the pTREP1-nuc1, pTREP1-nuc2 and pTREP1-nuc3 series of reporter vectors. These vectors are described in FIG. 4. General molecular biology techniques were carried out using the reagents and buffers supplied by the manufacturer or using standard techniques (Sambrook and Maniatis, Molecular cloning: A laboratory manual. Cold Spring Harbor Laboratory Press: Cold Spring Harbour (1989)). In each of the pTREP1-nuc vectors the expression cassette comprises a transcription terminator, lactococcal promoter P1, unique cloning sites (Bgl II, EcoRV or SmaI) followed by the mature form of the nuc gene and a second transcription terminator. Note that the sequences required for translation and secretion of the nuc gene were deliberately excluded in this construction. Such elements can only be provided by appropriately digested foreign DNA fragments (representing the target bacterium) which can be cloned into the unique restriction sites present immediately upstream of the nuc gene.

(d) Screening for secreted proteins in Group B Streptococcus.

Genomic DNA isolated from Group B Streptococcus (S. agalactiae) was digested with the restriction enzyme Tru9I. This enzyme which recognises the sequence 5′-TTAA -3′ was used because it cuts A/T rich genomes efficiently and can generate random genomic DNA fragments within the preferred size range (usually averaging 0.5-1.0 kb). This size range was preferred because there is an increased probability that the P1 promoter can be utilised to transcribe a novel gene sequence. However, the P1 promoter may not be necessary in all cases as it is possible that many Streptococcal promoters are recognised in L. lactis. DNA fragments of different size ranges were purified from partial Tru9I digests of S. agalactiae genomic DNA. As the Tru 9I restriction enzyme generates staggered ends the DNA fragments had to be made blunt ended before ligation to the EcoRV or SmaI cut pTREP1-nuc vectors. This was achieved by the partial fill-in enzyme reaction using the 5′-3′ polymerase activity of Klenow enzyme. Briefly Tru9I digested DNA was dissolved in a solution (usually between 10-20 μl in total) supplemented with T4 DNA ligase buffer (New England Biolabs; NEB) (1×) and 33 μM of each of the required dNTPs, in this case dATP and dTTP. Klenow enzyme was added (1 unit Klenow enzyme (NEB) per μg of DNA) and the reaction incubated at 25° C. for 15 minutes. The reaction was stopped by incubating the mix at 75° C. for 20 minutes. EcoRV or SmaI digested pTREP-nuc plasmid DNA was then added (usually between 200-400 ng). The mix was then supplemented with 400 units of T4 DNA ligase (NEB) and T4 DNA ligase buffer (1×) and incubated overnight at 16° C. The ligation mix was precipitated directly in 100% Ethanol and 1/10 volume of 3M sodium acetate (pH 5.2) and used to transform L. lactis MG1363 (Gasson, J. Bacteriol. 154:1-9 (1983)). Alternatively, the gene cloning site of the pTREP-nuc vectors also contains a BglII site which can be used to clone for example Sau3AI digested genomic DNA fragments.

L. lactis transformant colonies were grown on brain heart infusion agar and nuclease secreting (Nuc⁺) clones were detected by a toluidine blue-DNA-agar overlay (0.05 M Tris pH 9.0, 10 g of agar per litre, 10 g of NaCl per liter, 0.1 mM CaCl2, 0.03% wt/vol. salmon sperm DNA and 90 mg of Toluidine blue O dye) essentially as described by Shortle, 1983 [supra], and Le Loir et al., 1994 [supra]). The plates were then incubated at 37° C. for up to 2 hours. Nuclease secreting clones develop an easily identifiable pink halo. Plasmid DNA was isolated from Nuc⁺ recombinant L. lactis clones and DNA inserts were sequenced on one strand using the NucSeq sequencing primer described in FIG. 3, which sequences directly through the DNA insert.

### EXAMPLE 2

Preparation of a S. agalactiae Standard Inoculum

Strain validation

S. agalactiae serotype III (strain 97/0099) is a recent clinical isolate derived from the cerebral spinal fluid of a new born baby suffering from meningitis. This haemolytic strain of Group B Streptococcus was epidemiologically tested and validated at the Respiratory and Systemic Infection Laboratory, PHLS Central Public Health Laboratory, 61 Colindale Avenue, London NW9 5HT. The strain was subcultured only twice prior to its arrival in the laboratory. Upon its arrival on an agar slope, a sweep of 4-5 colonies was immediately used to inoculate a Todd Hewitt/5% horse blood broth which was incubated overnight statically at 37° C. 0.5 ml aliquots of this overnight culture were then used to make 20% glycerol stocks of the bacterium for long-term storage at −70° C. Glycerol stocks were streaked on Todd Hewitt/5% horse blood agar plates to confirm viability.

In Vivo passaging of Group B Streptoccocus

A frozen culture (described under strain validation) of S. agalactiae serotype III (strain 97/0099) was streaked to single colonies on Todd-Hewitt/5% blood agar plates, which were incubated overnight at 37° C. A sweep of 4-5 colonies was used to inoculate a Todd Hewitt/5% horse blood broth, which was again incubated overnight. A 0.5 ml aliquot from this overnight culture was used to inoculate a 50 ml Todd Hewitt broth (1:100 dilution) which was incubated at 37° C. 10-fold serial dilutions of the overnight culture were made (since virulence of this strain was unknown) and each was passaged intra-peritoneally (IP) in CBA/ca mice in duplicate. Viable counts were performed on the various inocula used in the passage. Groups of mice were challenged with various concentrations of the pathogen ranging from 10⁸ to 10⁴ colony forming units (cfu). Mice that developed symptoms were terminally anaesthetized and cardiac punctures were performed (Only mice that had been challenged with the highest doses, i.e. 1×10⁸ cfu, developed symptoms). The retrieved unclotted blood was used to inoculate directly a 50 ml serum broth (Todd Hewitt/20% inactivated foetal calf serum). The culture was constantly monitored and allowed to grow to late logarithmic phase. The presence of blood in the medium interfered with OD_{600 nm} readings as it was being increasingly lysed with increasing growth of the bacterium, hence the requirement to constantly monitor the culture. Upon reaching late logarithmic phase/early stationary phase, the culture was transferred to a fresh 50 ml tube in order to exclude dead bacterial cells and remaining blood cells which would have sedimented at the bottom of the tube. 0.5 ml aliquots were then transferred to sterile cryovials, frozen in liquid nitrogen and stored at −70° C. A viable count was carried out on a single standard inoculum aliquot in order to determine bacterial numbers. This was determined to be approximately 5×10⁸ cfu per ml.

Intra-peritoneal challenge and virulence testing of Group B Streptococcus standard inoculum

To determine if the standard inoculum was suitably virulent for use in a vaccine trial, challenges were carried out using a dose range. Frozen standard inoculum strain aliquots were allowed to thaw at room temperature. From viable count data the number of cfu per ml was already known for the standard inoculum. Initially, serial dilutions of the standard inoculum were made in Todd Hewitt broth and mice were challenged intra-peritoneally with doses ranging from 1×10⁸ to 1×10⁴ cfu in a 500 μl volume of Todd Hewitt broth. The survival times of mouse groups injected with different doses of the bacterium were compared. The standard inoculum was determined to be suitably virulent and a dose of 1×10⁶ cfu was considered close to optimal for further use in vaccine trials. Further optimisation was carried out by comparing mice challenged with doses ranging between 5×10⁵ and 5×10⁶ cfu. The optimal dose was estimated to be approximately 2.5×10⁶ cfu. This represented a 100% lethal dose and was repeatedly consistent with end-points as determined by survival times being clustered within a narrow time-range. Throughout all these experiments, challenged mice were constantly monitored to clarify symptoms, stages of symptom development as well as calculating survival times.

Screening Group B Streptococcal LEEP Derived Genes in DNA Vaccination Experiments.

pcDNA3.1+ as a DNA vaccine vector

The commercially available pcDNA3.1+plasmid (Invitrogen), referred to as

pcDNA3.1 henceforth, was used as a vector in all DNA immunisation experiments involving gene targets derived using the LEEP system unless stated otherwise.

pcDNA 3.1 is designed for high-level stable and transient expression in mammalian cells and has been used widely and successfully as a host vector to test candidate genes from a variety of pathogens in DNA vaccination experiments (Zhang et al., Infection and Immunity 176: 1035-40 (1997); Kurar and Splitter, Vaccine 15: 1851-57 (1997); Anderson et al., Infection and Immunity 64: 3168-3173 (1996)).

The vector possesses a multiple cloning site which facilitates the cloning of multiple gene targets downstream of the human cytomegalovirus (CMV) immediate-early promoter/enhancer which permits efficient, high-level expression of the target gene in a wide variety of mammalian cells and cell types including both muscle and immune cells. This is important for optimal immune response as it remains unknown as to which cells types are most important in generating a protective response in vivo. The plasmid also contains the ColE1 origin of replication which allows convenient high-copy number replication and growth in E. coli and the ampicillin resistance gene (B-lactamase) for selection in E. coli. In addition pcDNA 3.1 possesses a T7 promoter/priming site upstream of the MCS which allows for in vitro transcription of a cloned gene in the sense orientation.

Preparation of DNA vaccines

Oligonucleotide primers were designed for each individual gene of interest derived using the LEEP system unless stated otherwise. Each gene was examined thoroughly, and where possible, primers were designed such that they targeted that portion of the gene believed to encode only the mature portion of the protein (APPENDIX I); the intention being to express those sequences that encode only the mature portion of a target gene protein to would facilitate its correct folding when expressed in mammalian cells. For example, in the majority of cases primers were designed such that putative N-terminal signal peptide sequences would not be included in the final amplification product to be cloned into the pcDNA3. 1 expression vector. The signal peptide directs the polypeptide precursor to the cell membrane via the protein export pathway where it is normally cleaved off by signal peptidase I (or signal peptidase II if a lipoprotein). Hence the signal peptide does not make up any part of the mature protein whether it be displayed on the bacterium's surface or secreted. Where an N-terminal leader peptide sequence was not immediately obvious, primers were designed to target the whole of the gene sequence for cloning and ultimately, expression in pcDNA3.1.

All forward and reverse oligonucleotide primers incorporated appropriate restriction enzyme sites to facilitate cloning into the pcDNA3.1 MCS region. All forward primers were also designed to include the conserved Kozak nucleotide sequence 5′-gccacc-3′ immediately upstream of an ‘atg’ translation initiation codon in frame with the target gene insert. The Kozak sequence facilitates the recognition of initiator sequences by eukaryotic ribosomes. Typically, a forward primer incorporating a BamH1 restriction enzyme site the primer would begin with the sequence 5′-cggatccgccaccatg-3′, followed by a sequence homologous to the 5′ end of that part of a gene being amplified. All reverse primers incorporated a Not I restriction enzyme site sequence 5′-ttgcggccgc-3′. All gene-specific forward and reverse primers were designed with compatible melting temperatures to facilitate their amplification.

All gene targets were amplified by PCR from S. agalactiae genomic DNA template using Vent DNA polymerase (NEB) or rTth DNA polymerase (PE Applied Biosystems) using conditions recommended by the manufacturer. A typical amplification reaction involved an initial denaturation step at 95° C. for 2 minutes followed by 35 cycles of denaturation at 95° C. for 30 seconds, annealing at the appropriate melting temperature for 30 seconds, and extension at 72° C. for 1 minute (1 minute per kilobase of DNA being amplified). This was followed by a final extension period at 72° C. for 10 minutes. All PCR amplified products were extracted once with phenol chloroform (2:1:1) and once with chloroform (1:1) and ethanol precipitated. Specific DNA fragments were isolated from agarose gels using the QIAquick Gel Extraction Kit (Qiagen). The purified amplification gene DNA fragments were digested with the appropriate restriction enzymes and cloned into the pcDNA3.1 plasmid vector using E. coli as a host. Successful cloning and maintenance of genes was confirmed by restriction mapping and by DNA sequencing. Recombinant plasmid DNA was isolated on a large scale (>1.5 mg) using Plasmid Mega Kits (Qiagen).

DNA vaccination trials

DNA vaccine trials in mice were accomplished by the administration of DNA to 6 week old CBA/ca mice (Harlan, UK). Mice to be vaccinated were divided into groups of six and each group was immunised with recombinant pcDNA3.1 plasmid DNA containing a specific target-gene sequence derived using the LEEP system unless stated otherwise. A total of 100 μg of DNA in Dulbecco's PBS (Sigma) was injected intramuscularly into the tibialis anterior muscle of both hind legs. Four weeks later this procedure was repeated using the same amount of DNA. For comparison, control mice groups were included in all vaccine trials. These control groups were either not DNA-vaccinated or were immunised with non-recombinant pcDNA3.1 plasmid DNA only, using the same time course described above. Four weeks after the second immunisation, all mice groups were challenged intra-peritoneally with a lethal dose of S. agalactiae serotype III (strain 97/0099). The actual number of bacteria administered was determined by plating serial dilutions of the inoculum on Todd-Hewitt/5% blood agar plates. All mice were killed 3 or 4 days after infection. During the infection process, challenged mice were monitored for the development of symptoms associated with the onset of S. agalactiae induced-disease. Typical symptoms in an appropriate order included piloerection, an increasingly hunched posture, discharge from eyes, increased lethargy and reluctance to move which was often the result of apparent paralysis in the lower body/hind leg region. The latter symptoms usually coincided with the development of a moribund state at which stage the mice were culled to prevent further suffering. These mice were deemed to be very close to death, and the time of culling was used to determine a survival time for statistical analysis. Where mice were found dead, a survival time was calculated by averaging the time when a particular mouse was last observed alive and the time when found dead, in order to determine a more accurate time of death. The results of this trial are shown in Table land presented graphically in FIG. 2.

Interpretation of Results

A positive result was taken as any DNA sequence that was cloned and used in challenge experiments as described above and gave protection against that challenge. DNA sequences were determined to be protective;

if that DNA sequence gave statistically significant protection to mice as compared to control mice (to a 95% confidence level (p>0.05) as determined using the Mann-Whitney U test.

if that DNA sequence was marginal or non-signficant using Mann-Whitney but showed some protective features. For example, one or more outlying mice may survive for significantly longer time periods when compared with control mice. Alternatively, the time to first death may also be prolonged when compared to counterpart mice in control groups. It is acceptable to allow marginal or non-significant results to be considered as potential positives when it is possible that the clarity of some results may be affected by problems associated with the administration of the DNA vaccine. Indeed, much varied survival times may reflect different levels of immune response between different members of a given group.

TABLE 1
LEEP DNA immunisation and GBS challenge Experiment
Statistical analysis of survival times

| | Mean Survival Times (hours) | | |
|---|---|---|---|
| | UnVacc | 3-60(ID-65) | 3-5(ID-66) |
| 1 | 27.583 | 54.416 | 42.916 |
| 2 | 27.583 | 31.000 | 42.916 |
| 3 | 24.583 | 43.000 | 32.874 |
| 4 | 22.250 | 34.916 | 42.916 |
| 5 | 35.916 | 38.958 | 27.333 |
| 6 | 22.250 | 34.916 | 30.916 |
| Mean | 27.583 | 40.458 | 37.791 |
| sd | 5.1691 | 8.9959 | 7.2860 |
| p value | | 0.0098 | 0.0215 |

p value refers to statistical significance when compared to unvaccinated controls.

Comment

ID-65 (3-60)

Mice immunised with the ‘3-60 (ID-65)’ DNA vaccine exhibited significantly longer survival times when compared with the unvaccinated control group.

ID-66 (3-5)

Mice immunised with the ‘3-5 (ID-66)’ DNA vaccine exhibited significantly longer survival times when compared with the unvaccinated control group.

### EXAMPLE 3

Expression and Screening Group B Streptococcal LEEP Derived Proteins in Protein Vaccination Experiments.

Expression of proteins

Prioritised genes ie, those selected on the basis of predicted expression features as deduced from sequence characteristics (as described in FIG. 1), were cloned and expressed as recombinant proteins using the pET system (Novagen, Inc., Madison, Wis.) utilising Escherichia coli as a host. Target genes were cloned into the pET28b(+) plasmid expression vector. The pET28b(+) vector is designed for high level expression and purification of target proteins. This vector carries a T7 promoter for transcription of a target gene, followed by an N-terminal His●Tag®/thrombin/T7●Tag® configuration, a multi-cloning site containing unique restriction enzyme sites for cloning purposes, and an optional C-terminal His●Tag sequence. The vector also carries a kanamycin resistance gene for selection purposes and for maintaining target gene expression (pET System Manual, 8^{th} edition, Novagen).

Preparation of protein vaccines

Oligonucleotide primers were designed for each individual target gene derived using the LEEP system unless stated otherwise. Each gene was examined thoroughly. Where possible primers were designed so that they would target that part of the gene predicted to encode only the mature portion of the protein (APPENDIX II). It is hoped that expressing those corresponding to the predicted mature protein only, might facilitate its correct folding when finally expressed in vitro. Oligonucleotide primers were designed so that sequences, encoding the putative N-terminal signal peptide of the target protein, would not be included in the final amplification product to be cloned pET28b(+). The signal peptide directs the polypeptide precursor to the cell membrane via the protein export pathway where it is normally cleaved off by signal peptidase I (or signal peptidase II if a lipoprotein). Hence the signal peptide would not be expected to form any part of the mature target protein, whether it be displayed on the bacterium's surface or secreted. For this purpose, classical signal peptides and their cleavage sites were predicted using the DNA Strider™ Program (CEA, France) and the SignalP V1.1 program, which predicts the presence and location of signal peptide cleavage sites in amino acid sequences from different organisms (Nielsen et al., Protein Engineering 10: 1-6 (1997)). Where a N-terminal leader peptide sequence was not obvious, primers were designed to include the whole of the gene sequence for cloning and expression.

All oligonucleotide primers were designed to incorporate appropriate restriction enzyme sites to facilitate cloning into the pcDNA3.1 MCS region (APPENDIX II). Forward primers included an Nco I (5′-ccatgg-3′) or Nhe I (5′-gctagc-3′) restriction enzyme site and an ‘ATG’ start codon in-frame with the target gene open reading frame (orf). All reverse primers included a Not I restriction enzyme site 5′-gcggccgc-3′ and were designed so that the target gene could be expressed in frame with the C-terminal His●Tag (i.e. the stop codon of the target gene was not included). Using the Nco I and Not I, allowed the removal of the N-terminal His●Tag®, thrombin and T7●Tag® DNA sequences. At the same time target genes were cloned immediately downstream of a highly efficient ribosome binding site (from the phage T7 major capsid protein), to facilitate high level expression/translation of the target gene by T7 RNA polymerase, and subsequent purification by means of the C-terminal His●Tag. All target gene-specific forward and reverse primers were designed with compatible melting temperatures to facilitate their amplification.

All gene targets were amplified by PCR from S. agalactiae genomic DNA template using Vent DNA polymerase (NEB) using conditions recommended by the manufacturer. A typical amplification reaction involved an initial denaturation step at 95° C. for 2 minutes followed by 35 cycles of denaturation at 95° C. for 30 seconds, annealing at the appropriate melting temperature for 30 seconds, and extension at 72° C. for 1 minute (1 minute per kilobase of DNA being amplified). This was followed by a final extension period at 72° C. for 10 minutes. All PCR amplified products were extracted .once with phenol:chloroform (2:1:1) and once with chloroform (1:1) and ethanol precipitated. Specific DNA fragments were isolated from agarose gels using the QIAquick Gel Extraction Kit (Qiagen). Purified target gene DNA amplicons were then digested Nco I (or Nhe I) and Not I restriction enzymes, and cloned into Nco I and Not I digested pET28b(+) plasmid vector using E. coli DH5α or E. coli BL21 (DE3) as a host. Successful cloning and maintenance of genes was confirmed by restriction mapping.

Deternination of target protein expression and solubility

Glycerol stocks of E. coli BL21 DE3 pET28b(+) strains expressing recombinant proteins were used to inoculate 10 ml Luria broth containing Kanamycin (30 μg/ml) which were grown overnight at 37° C. with vigorous shaking (300 rpm).

A 20-40 ml Luria broth containing Kanarnycin (30 μg/ml) was inoculated with 1:100 dilution of the overnight culture from step 1 and grown at 37° C. with vigorous shaking (300 rpm). When the culture reached an OD₆₀₀ of between 0.6 and 1.0, IPTG was added to a final concentration of 1 mM. Typically cultures were induced for 3 hours. Cells were then harvested by centrifugation at 7000 g for 10 min. The cell pellet was then resuspended in 1/10 volume of lysis buffer (50 mM NaH₂PO₄, pH.8.0; 300 mM NaCl;10 mM imidazole; 10% glycerol). Lysozyme was then added to a final concentration of 1 mg/ml, and the suspension was incubated on ice for 30 min. The suspension was then sonicated on ice (six 10-sec bursts at 200-300 W with a 10-sec cooling period. The lysate was then centrifuged at 10,000g for 20 min. The supernatant (containing soluble protein) was transferred to a sterile 2 ml eppendorf. The pellet was resuspended in 2 ml of solubilisation buffer (8 M Urea; 50 mM NaH₂PO₄, pH.8.0; 300 mM NaCl; 10% glycerol). This suspension contained the insoluble protein fraction. Aliquots from both the soluble and insoluble fractions were transferred to new eppendorfs. The protein samples were denatured by adding an equal volume of 2×SDS-PAGE buffer and heating at 95° C. for 5 min. Denatured extract samples were then analysed by SDS-PAGE to determine target gene expression and solubility.

Large scale expression of recombinant target proteins

Glycerol stocks of E. coli BL21 DE3 pet28b(+) strains expressing recombinant proteins were used to inoculate 10 ml Luria broth containing Kanamycin ( 30 μg/ml) which were grown overnight at 37° C. with vigorous shaking (300 rpm). 5 ml of an overnight culture of a recombinant strain was used to inoculate a 250 ml Luria broth containing kanamycin (30 μg/ml) which was grown at 37° C. with vigorous shaking (300 rpm). When the culture reached an OD₆₀₀ of between 0.6 and 1.0, IPTG was added to a final concentration of 1 mM. Typically, cultures were induced for 3 hours. Cultures were then centrifuged to a pellet and stored frozen at −20° C.

Purification of target antigens.

Ni-NTA agarose (Qiagen LTD, West Sussex, UK; Cat. No. 30210) was used to purify the His-Tagged recombinant proteins. The 6×His affinity tag which was expressed in frame with the target proteins in pET28b(+), facilitates binding to Ni-NTA. Ni-NTA offers high binding capacity (with minimal non-specific binding) and can bind 5-10 mg of 6×His-tagged protein per ml of resin. The 6×His-tag is poorly immunogenic, and at pH 8.0, the tag is small, uncharged and therefore does not generally interfere with the structure and function of the protein (The QIAexpressionist, Qiagen Handbook, March 1999).

NOTE: All the proteins (LEEP-derived, unless stated otherwise) described here were purified under denaturing conditions except ID-65. ID-65 was prepared and purified under native conditions.

Purification under native conditions

The frozen pellet was allowed to thaw on ice for 15 minutes and then resuspended in 10 ml of lysis buffer (50 mM NaH₂PO₄, pH.8.0; 300 mM NaCl;10 mM imidazole; 10% glycerol). Lysozyme was then added to a final concentration of 1 mg/ml, and the suspension was incubated on ice for 30 min. The suspension was then sonicated on ice (six 10-sec bursts at 200-300 W with a 10-sec cooling period0. Dnase I (5 μg/ml) was then added to the lysate, which was then incubated on ice for 10-15 min. The lysate was then centrifuged at 10,000 rpm for 20 min at 4° C. to pellet cell debris. The clear lysate supernatant was then loaded into a polypropylene column (Qiagen; Cat. No. 34964), bottom cap attached. 1.5 ml of 50% Ni-NTA was then added, the column sealed and the suspension was allowed to mix gently using a rotating wheel for 1-2 hours at 4° C. The column containing the lysate/Ni-NTA mix was then placed upright using a retort stand, and the Ni-NTA was allowed to settle. The bottom cap was removed and the lysate was allowed to flow through. The column was then washed with three to six 4 ml volumes of wash buffer (50 mM NaH₂PO₄, pH.8.0; 300 mM NaCl;20 mM imidazole; 10% glycerol). The protein was then eluted in 0.5 ml aliquots of elution buffer (500 mM NaH₂PO₄, pH.8.0; 300 mM NaCl;500 mM imidazole; 10% glycerol). Eluate fractions were then analysed by SDS-PAGE and those containing the protein were pooled and dialysed against a PBS (pH 7.0)-glycerol (10%) solution.

Purification and refolding under denaturing conditions

The frozen pellet was allowed to thaw on ice for 15 minutes and then resuspended in 10 ml of buffer containing 8 M Urea, 300 mM NaCl, 10% glycerol, 0.1 M NaH₂PO₄, pH.8.0, and 10 mM imidazole. The cells were then lysed by gentle vortexing for 1 hour at room temperature. The lysate was then centrifuged at 10,000 g for 20 minutes to pellet cellular debris. The clear lysate supernatant was then loaded into a polypropylene column (Qiagen; Cat. No. 34964), bottom cap attached. 1.5 ml of 50% Ni-NTA slurry was then added, the column sealed and the suspension was allowed to mix gently using a rotating wheel for 1-2 hours at room temperature. The column containing the lysate/Ni-NTA mix was then placed upright using a retort stand, and the Ni-NTA was allowed to settle. The bottom cap was removed and the lysate was allowed to flow through. The column was then washed with 4-8 ml of buffer containing 8 M Urea, 300 mM NaCl, 10% glycerol, 0.1 M NaH₂PO₄, pH 8.0, and 10 mM imidazole. The resin was then washed with a gradient of 6 to 0 M in a buffer containing 0.1 M NaH₂PO₄, pH.8.0, 300 mM NaCl and 10% glycerol to facilitate the slow removal of urea and gradual refolding of target protein. The resin was then washed with a buffer containing 0.1 M NaH₂PO₄, pH 7.0, 500 mM NaCl and 10% glycerol. The recombinant protein was then eluted in 0.5 ml aliquots with 500 mM Imidazole in 0.1 mM NaH₂PO₄, pH 7.0, 500 mM NaCl and 10% glycerol. The fractions were analysed on SDS-PAGE and those containing the protein were pooled and dialysed against a PBS (pH 7.0)-glycerol (10%) solution.

All purified proteins were analysed by SDS-PAGE, as shown in FIGS. 5, 6 and 7, prior to their use as antigens in immunisation and vaccination experiments.

Protein Vaccinations

Vaccines were composed of the target protein in phosphate buffered saline/10% glycerol and mixed with aluminium hydroxide (alum) (Imject® Alum, Pierce, Rockford, Ill.). Each dose (unless otherwise stated) of vaccine contained 25 μg of purified protein in 50 μl of PBS/10% glycerol, mixed with 50 μl of alum. Groups of 6-8 CBA/ca mice (Harlan, UK) were immunised subcutaneously with the vaccines and again 4 weeks later. A control group received 100 μl dose of PBS/10% glycerol with alum. All vaccinated groups consisted of 6 mice. Mice were challenged at 7 weeks (unless otherwise stated). Mice were injected intraperitoneally (i.p.) with between 2.5-5×10⁶ bacteria diluted in 0.5 ml Todd-Hewitt broth. Deaths were recorded daily for 7 days. The challenged mice were observed daily for signs of illness. Typical symptoms in an appropriate order included piloerection, an increasingly hunched posture, discharge from eyes, increased lethargy and reluctance to move which was often the result of apparent paralysis in the lower body/hind leg region. The latter symptoms usually coincided with the development of a moribund state at which stage the mice were culled to prevent further suffering. These mice were deemed to be very close to death, and the time of culling was used to determine a survival time for statistical analysis. Where mice were found dead, a survival time was calculated by averaging the time when a particular mouse was last observed alive and the time when found dead, in order to determine a more accurate time of death.

Analysis of antibody responses

Mice (6 per group) were immunised with two doses of vaccine with a four week interval. Mice were tail bled at 3 weeks and 6 weeks post primary vaccination to obtain sera. Total Immunoglobulin G (IgG) titres to the vaccine protein component in sera were determined by enzyme-linked immunosorbent assay (ELISA), using the original purified protein as the coating antigen.

Standard ELISA protocol

Solutions

Carbonate/bicarbonate buffer, pH 9.8

0.80 g Na₂CO₃

1.46 g NaHCO₃

pH to 9.6 using HCl

Add distilled water (dH₂O) to a final volume of 500 ml.

n-NITROPHENYL PHOSPHATE SUBSTRATE

Diethanolamine Buffer, pH 9.8

48.5 ml diethanolamine

pH to 9.8 using 1M HCl

Add dH₂O to a final volume of 500 ml

NOTE: ELISAs were optimised for each protein submitted for immunisation.

Protocol

1. ELISA plates (Greiner labortechnik 96 well plates: Cat. No. 655061) with an appropriate concentration of recombinant protein diluted in carbonate/bicarbonate buffer (50 μl/well). Cover plates with plastic or foil and leave overnight at 4° C.

2. Quickly wash plates twice in a tub/container containing PBS/0.05%Tween-20 and then pat dry.

3. Block plates with 3% BSA in PBS/Tween (100 μl/well) for 1 hour at room temperature.

4. Wash the plates 3 times PBS/Tween as before and pat dry as before.

5. Apply (primary antibody) protein-specific antiserum (50μl/well) diluted from 1/50 in a doubling dilution series in PBS/Tween and incubate at room temperature for 90 minutes.

6. Wash plates as before (3 times quickly), followed up by 2×3 minute soaks (in PBS/Tween)

7. Apply diluted secondary antibody alkaline phosphatase conjugate. For anti-mouse Total IgG alkaline phospatase conjugate (Goat Anti-Mouse IgG-AP, Southern Biotechnology Associates, Birmingham, Ala. Cat. No. 1030-04) dilute 1/3000 in PBS/Tween and apply 50 μl per well and incubate at room temperature for 90 minutes.

8. Wash plates as in step 6.

9. Apply substrate. Dissolve one 5 mg tablet of nitrophenyl phosphate (Sigma: kept in freezer) in 5 ml of diethanolamine buffer. Apply 100 μl per well. Cover with foil (a light-sensitive reaction) and leave at room temperature for 30 minutes. Read Optical densities (OD) at a wavelength of 405 nm.

10. Plot curves of OD Vs dilution (log scale). Calculate end-point titres as the dilution giving the same OD as the mean of the OD obtained from the wells containing the 1/50 dilution of pre-immune serum.

ELISA Plate format

| | | | | | | | | | | | |
|---|---|---|---|---|---|---|---|---|---|---|---|
| 2° | 1/50 | 1/100 | 1/200 | 1/400 | 1/800 | 1/1600 | 1/3200 | 1/6400 | 1/12800 | 1/25600 | 1/51200 |
| 1° | Duplicate | | | | | | | | | | |
| Pre | | | | | | | | | | | |
| Pre | | | | | | | | | | | |
| Pre | | | | | | | | | | | |

Table Summary
Pre Replicate wells of pooled pre-inoculation serum (50 μl per well) diluted to 1/50 are included on every plate in order for end point titres to be calculated.
2° Is a blank control well to which no secondary antibody conjugate is applied.
PBS/Tween by itself is applied instead
1° Is a blank control well to which no primary antibody is applied. PBS/Tween by itself is applied instead
Duplicate Each serum is analysed in duplicate

The dilution series used is indicated (see first row of table). Beginning with a 1/50 dilution, sera are diluted two-fold in PBS/Tween in doubling dilution series as indicated.

Protein Immunisation data

ID-65 and ID-83

The ID-65 and ID-83 vaccines were composed of the target proteins in phosphate buffered saline/10% glycerol mixed with aluminium hydroxide (alum) (Imject®Alum, Pierce, Rockford, Ill.). Each dose of vaccine contained 20 μg of purified protein in 100 μl of PBS/10% glycerol, mixed with 50 μl of alum. A group of 6-8 week old CBA/ca mice (Harlan, UK) were immunised subcutaneously with the ID65 and ID-83 vaccine and again 4 weeks later. A control group received a 150 μl dose of PBS/10% glycerol (2:1) with alum. All groups consisted of 6 mice. Mice were tail bled at 5 weeks post primary vaccination to obtain sera. The presence of total Immunoglobulin G (IgG) antibodies to the ID-65 and ID-83 protein in sera was determined by enzyme-linked immunosorbent assay (ELISA), using the purified protein as the coating antigen. ELISA was also performed using sera obtained at 6 weeks post-primary vaccination from the PBS/10% glycerol immunised control group.

NOTE: ELISA plates were coated with the ID-65 or ID-83 proteins at a concentration of 1 μg/ml.

Protein Vaccination—ELISA Results for ID-65 and ID-83

Mice (6 per group) were immunised with two doses of the ID-65 and ID-83 vaccines with a four week interval. Mice were tail bled at 5 weeks post primary vaccination to obtain sera. The Immunoglobulin G (IgG) titres to the vaccine protein component in sera were determined by enzyme-linked immunosorbent assay (ELISA), using the purified ID-65 and ID-83 proteins as the coating antigen. Subsequent to optimisation, ELISA plates were coated at a concentration 1 ug/ml for both the purified ID65 and ID-93 proteins. Total IgG titres were measured against pre-immune serum (1/50 dilution). The results are shown in Table 2 and graphically in FIG. 8.

TABLE 2

| | Serum (Group) | | | |
|---|---|---|---|---|
| | ID-65 + Alum | PBS + Alum | ID-83 + Alum | PBS + Alum |
| | (n = 6) | (n = 6) | (n = 6) | (n = 6) |
| | Coating antigen | | | |
| | ID-65 | | ID-83 | |
| Bleed | 5 weeks | 5 weeks | 5 weeks | 5 weeks |
| Total IgG | 7535763 | 965 | 82081 | 61 |
| Titres | 1557649 | 90 | 50027 | 50 |
| (mouse | 3319737 | 108 | 154670 | 80 |
| 1-6) | 1832259 | 176 | 57901 | 96 |
| | 8794360 | 371 | 66497 | 125 |
| | 1445728 | 0 | 49928 | 0 |
| Average | 4080916 | 285 | 76851 | 69 |
| Standard | 3258818 | 355 | 39985 | 43 |
| Deviation | | | | |

Protein Immunisation and Challenge data (ID-93)

ID-93

The ID-93 vaccine was composed of the target ID-93 protein in phosphate buffered saline/10% glycerol mixed with aluminium hydroxide (alum) (Imject®Alum, Pierce, Rockford, Ill.). Each dose of vaccine contained 25 μg of purified protein in 100 μl of PBS/10% glycerol, mixed with 100 μl of alum. A group of 6-8 week old CBA/ca mice (Harlan, UK) were immunised subcutaneously with the ID-93 vaccine and again 4 weeks later. A control group received PBS/10% glycerol with alum. Both groups consisted of 6 mice. Mice were challenged at 7 weeks (unless otherwise stated). Mice were injected intraperitoneally (i.p.) with 5×10⁶ bacteria diluted in 0.5 ml Todd-Hewitt broth. The challenged mice were observed daily for signs of illness. Deaths were recorded daily for 7 days. Survival data are shown in Table 3 and graphically in FIG. 9.

Mice were tail bled at 3 weeks and 6 weeks post primary vaccination to obtain sera. The presence of total Immunoglobulin G (IgG) antibodies to the ID-93 protein in sera was determined by enzyme-linked immunosorbent assay (ELISA), using the pure ID-93 protein as the coating antigen. ELISA was also performed using sera obtained at 6 weeks post-primary vaccination from the PBS/10% glycerol immunised control group.

Note: ELISA plates were coated with the ID-93 protein at a concentration of 1 μg/ml.

TABLE 3
ID-93 protein immunisation and GBS challenge experiment
Statistical analysis of Survival Times

| | Group | |
|---|---|---|
| | PBS + Alum | ID-93 + Alum |
| Survival | 22.37 | 29.37 |
| Times | 22.37 | 35.12 |
| (hours) | 15.37 | 32.62 |
| | 28.03 | 32.62 |
| | 29.53 | 37.12 |
| | 26.53 | 27.87 |
| Mean | 24.03 | 32.45 |
| sd | 5.16 | 3.45 |
| p value | | 0.01 |

p value refers to statistical significance when compared to unvaccinated controls.

Comment

ID-93 (RS-70)

Mice immunised with the ID-93-Alum vaccine exhibited significantly longer survival times when compared with the PBS-Alum control group.

(Statistical Significance was determined by the Mann-Whitney U test using a 95% confidence level (p>0.05).

Protein Vaccination—ELISA results for ID-93

Mice (6 per group) were immunised with two doses of the ID-93 vaccine with a four week interval. Mice were tail bled at 3 weeks and 6 weeks post primary vaccination to obtain sera. The Immunoglobulin G (IgG) titres to the vaccine protein component in sera were determined by enzyme-linked immunosorbent assay (ELISA), using the purified ID-93 protein as the coating antigen. Subsequent to optimisation, ELISA plates were coated with the purified ID-93 protein at a concentration of 1 μg/ml. Total IgG titres were measured against pre-immune serum (1/50 dilution). The results are shown in Table 4 and graphically in FIG. 10.

TABLE 4

| | Serum Group | | | |
|---|---|---|---|---|
| | | | PBS/10% glycerol | |
| | ID-93 + Alum(n = 6) | | (n = 6) (control) | |
| | Coating antigen | | | |
| | ID-93 | ID-93 | ID-93 | ID-93 |
| Bleed | 3 weeks | 6 weeks | 3 weeks | 6 weeks |
| Total IgG | 87196 | 3000000 | 39 | 100 |
| Titres | 99544 | 8000000 | 31 | 16 |
| (mouse 1-6) | 19620 | 2000000 | 31 | 79 |
| | 34724 | 10000000 | 59 | 48 |
| | 59990 | 10000000 | 24 | 328 |
| | 30041 | 4000000 | 13 | 40 |
| Average | 55186 | 6166667 | 33 | 102 |
| Standard | 32654 | 3600926 | 15 | 115 |
| error | | | | |

Protein Immunisation Data

ID-89 and ID-96

The ID-89 and ID-96 vaccines were composed of the target proteins in phosphate buffered saline/10% glycerol mixed with TitreMax Gold adjuvant (Sigma, Mo., USA) according to the manufacturers instructions. The ID-89 vaccine contained 25 μg of purified protein 50 μl of PBS/10% glycerol, mixed with 50 μl of TitreMax Gold. The ID-96 vaccine contained 12.5 μg of purified protein 50 μl of PBS/10% glycerol, mixed with 50 μl of TitreMax Gold. Groups of 6-8 week old CBA/ca mice (Harlan, UK) were immunised subcutaneously with the ID-89 and ID-96 vaccines and again 4 weeks later. A control group received a 100 μl dose PBS/10% glycerol with TitreMax Gold (1:1). Both groups consisted of 6 mice. Mice were tail bled at 3 weeks and 6 weeks post primary vaccination to obtain sera. The presence of total Immunoglobulin G (IgG) antibodies to the ID-65 and ID-83 protein in sera was determined by enzyme-linked immunosorbent assay (ELISA), using the purified protein as the coating antigen. ELISA was also performed using sera obtained at 3 weeks and 6 weeks post-primary vaccination from the PBS/10% glycerol immunised control group.

Note: ELISA plates were coated with the ID-89 or ID-96 proteins at a concentration of 1 μg/ml and 3 μg/ml respectively.

Protein Vaccination—ELISA results for ID-89 and ID-96

Mice (6 per group) were immunised with two doses of the ID-89 and ID-96 vaccines with a four week interval. Mice were tail bled at 3 weeks and 6 weeks post primary vaccination to obtain sera. The Immunoglobulin G (IgG) titres to the vaccine protein component in sera were determined by enzyme-linked immunosorbent assay (ELISA), using the purified ID-65 and ID-83 proteins as the coating antigen. Subsequent to optimisation, ELISA plates were coated with purified ID-89 and ID-96 protein at a concentration 1 ug/ml and 3 μg/ml respectively. Total IgG titres were measured against pre-immune serum (1/50 dilution). ELISA was also performed on both proteins using sera obtained at 3 weeks and 6 weeks post-primary vaccination from the PBS/10% glycerol immunised control group. Results are shown in tables 5a and 5b and graphically in FIG. 11.

TABLE 5a

| Serum | ID-89 + TitreMax Gold | | ID-96 + TitreMax Gold | |
|---|---|---|---|---|
| | (n = 6) | | (n = 6) | |
| Coating | ID-89 | | ID-96 | |
| antigen | | | | |
| Bleed | 3 weeks | 6 weeks | 3 weeks | 6 weeks |
| Total IgG | 146940 | 1000000 | 190371 | 10000000 |
| Titres | 89672 | 1000000 | 212505 | 10000000 |
| (mouse 1-6) | 173532 | 2000000 | 167613 | 5000000 |
| | 85161 | 751210 | 110378 | 5000000 |
| | 8895 | 551281 | 142614 | 1000000 |
| | 27880 | 2000000 | 191085 | 1000000 |
| Average | 10202 | 1217082 | 169094 | 5333333 |
| Standard | 51451 | 629364 | 37341 | 4033196 |
| Deviation | | | | |

TABLE 5B

| Serum | PBS/10% glycerol | | PBS/10% glycerol | |
|---|---|---|---|---|
| | (n = 6) | | (n = 6) | |
| Coating | ID-89 | | ID-96 | |
| protein | | | | |
| Bleed | 3 weeks | 6 weeks | 3 weeks | 6 weeks |
| Total | 3 | 7 | 33 | 31 |
| IgG | 8 | 18 | 77 | 62 |
| Titres | 29 | 31 | 77 | 1 |
| (mouse 1-6) | 34 | 4 | 52 | 29 |
| | 0 | 2 | 125 | 31 |
| | 5 | 1 | 113 | 0 |
| Average 13 | 11 | 80 | 26 | |
| Standard | 15 | 12 | 35 | 23 |
| deviation | | | | |

### EXAMPLE 4

Conservation and variability of candidate vaccine antigen genes among different isolates of Group B Streptococci

An initial Southern blot analysis was carried out to determine cross-serotype conservation of novel Group B Streptococcal genes isolated using the LEEP system unless stated otherwise. Analysing the serotype distribution of a target gene will also determine their potential use as antigen components in a GBS vaccine. The Group B Streptococcal strains whose DNA was analysed as part of this study are listed in APPENDIX III

Amplification and labelling of specific target genes as DNA probes for southern blot analysis.

Oligonucleotide primers were designed for each individual gene of interest derived using the LEEP system unless stated otherwise. The same primers already described in APPENDIX II were used to amplify corresponding gene-specific DNA probes. Specific gene targets were amplified by PCR using Vent DNA polymerase (NEB) according to the manufacturers instructions. Typical reactions were carried out in a 100 μl volume containing 50 ng of GBS template DNA, a one tenth volume of enzyme reaction buffer, 1 μM of each primer, 250 μM of each dNTP and 2 units of Vent DNA polymerase. A typical reaction contained an initial 2 minute denaturation at 95° C., followed by 35 cycles of denaturation at 95° C. for 30 seconds, annealing at the appropriate melting temperature for 30 seconds, and extension at 72° C. for 1 minute (1 minute per kilobase of DNA being amplified). The annealing temperature was determined by the lower melting temperature of the two oligonucleotide primers. The reaction was concluded with a final extension period of 10 minutes at 72° C.

All PCR amplified products were extracted once with phenol chloroform (2:1:1) and once with chloroform (1:1) and ethanol precipitated. Specific DNA fragments were isolated from agarose gels using the QIAquick Gel Extraction Kit (Qiagen). For use as DNA probes, purified amplified gene DNA fragments were labelled with digoxygenin using the DIG Nucleic Acid Labelling Kit (Boehringer Mannheim) according to the manufacturer's instructions.

Southern blot Hybridisation analysis of Group B Streptococcal genomic DNA

Genomic DNA had previously been isolated from all strains of Group B Streptococci which were investigated for conservation of LEEP-derived (unless stated otherwise) gene targets. Appropriate DNA concentrations were digested using either Hin DIII or Eco RI restriction enzymes (NEB) according to manufacturer instructions and analysed by agarose gel electrophoresis. Following agarose gel electrophoresis of DNA samples, the gel was denatured in 0.25M HCl for 20 minutes and DNA was transferred onto Hybond™ N⁺ membrane (Amersham) by overnight capillary blotting. The method is essentially as described in Sambrook et al. (1989) using Whatman 3MM wicks on a platform over a reservoir of 0.4M NaOH. After transfer, the filter was washed briefly in 2×SSC and stored at 4° C. in Saran wrap (Dow chemical company).

Filters were prehybridised, hybridised with the digoxygenin labelled DNA probes and washed using conditions recommended by Boehringer Mannheim when using their DIG Nucleic Acid Detection Kit. Filters were prehybridised at 68° C. for one hour in hybridisation buffer (1% w/v supplied blocking reagent, 5×SSC, 0.1% v/v N-lauryl sarcosine, 0.02% v/v sodium dodecyl sulphate[SDS]). The digoxygenin labelled DNA probe was denatured at 99.9° C. for 10 minutes before being added to the hybridisation buffer. Hybridisation was allowed to proceed overnight in a rotating Hybaid tube in a Hybaid Mini-hybridisation oven. Unbound probe was removed by washing the filter twice with 2×SSC-0.1% SDS for 5 minutes at room temperature. For increased stringency filters were then washed twice with 0.1×SSC-0.1% SDS for 15 minutes at 68° C. The DIG Nucleic Acid Detection Kit (Boehringer Mannheim) was used to immunologically detect specifically bound digoxygenin labelled DNA probes.

Results of Southern blot analysis

Unless otherwise stated, all genomic digests and their corresponding Southern blots followed an identical lane order as described in Table 6 below.

TABLE 6

| | Lane | | | | | | |
|---|---|---|---|---|---|---|---|
| | 1 | 2 | 3 | 4 | 5 | 6 | 7 |
| Strain | 1 kb | 515 | A909 | SB35 | H36B | 18RS21 | 1954/92 |
| | molecular | | | | | | |
| Serotype | Weight | Ia | Ia | Ib | Ib | II | II |
| | Marker | | | | | | |

| | Lane | | | | | | |
|---|---|---|---|---|---|---|---|
| | 8 | 9 | 10 | 11 | 12 | 13 | 14 |
| Strain | 118/158 | 97/0057 | BS30 | M781 | 97/0099 | 3139 | 1169-NT |
| Serotype | II | II | III | III | III | IV | V |

| | Lane | | | | | |
|---|---|---|---|---|---|---|
| | 15 | 16 | 17 | 18 | 19 | 20 |
| Strain | GBS 6 | 7271 | JM9 | Group | Streptococcus | 1 kb |
| | | | | A | pneumoniae | molecular |
| | | | | Strepo- | | |
| | | | | coccus | | |
| Serotype | VI | VII | VIII | — | 14 | Weight |
| | | | | | | Marker |

For comparative purposes, it was decided to analyse the serotype distribution of the GBS rib gene, which encodes the known protective immunogen Rib. Rib has previously been shown to be present in serotype III and some strains of serotype II but not in serotypes Ia or Ib (Stalhammar-Carlemalm et al., J. Exp. Med. 177: 1593-1603 (1993)).

Confirmation of this pattern would not only give increased confidence in interpreting subsequent results, it would also determine if a rib gene homologue was present in the remaining GBS serotypes being investigated here. Primers designed for the amplification of rib for use as a gene probe in Southern blot analysis are described in APPENDIX II.

TABLE 7
Lane order for FIG. 12 (rib gene Southern blot analysis)

| | Lane | | | | | | |
|---|---|---|---|---|---|---|---|
| | 1 | 2 | 3 | 4 | 5 | 6 | 7 |
| strain | 1 kb | 515 | A909 | SB35 | H36B | 18RS21 | 1954/92 |
| | molecular | | | | | | |
| serotype | Weight | Ia | Ia | Ib | Ib | II | II |
| | Marker | | | | | | |

| | Lane | | | | | | |
|---|---|---|---|---|---|---|---|
| | 8 | 9 | 10 | 11 | 12 | 13 | 14 |
| strain | 118/158 | 97/0057 | BM110 | BS30 | M781 | 97/0099 | 3139 |
| serotype | II | II | III | III | III | III | IV |

| | Lane | | | | | |
|---|---|---|---|---|---|---|
| | 15 | 16 | 17 | 18 | 19 | 20 |
| strain | 1169-NT | GBS 6 | 7271 | JM9 | Group | Streptococcus |
| | | | | | A | pneumoniae |
| | | | | | Strepo- | |
| | | | | | coccus | |
| serotype | V | VI | VII | VIII | — | 14 |

Rib (FIG. 12) Comment

The Southern blot analysis shown in FIG. 12 indicates that the rib gene is not conserved across all GBS serotypes. rib appears to be absent from all serotype Ia and Ib strains (lanes 2 to 5) and from strains 118/158 and 97/0057 of serotype II (lanes 8 and 9). However, rib would appear to present in strains 18RS21 and 1954/92 of serotype II (lanes 6 and 7) and in all strains of serotype III (lanes 10 to 13). This is in agreement with previously published data (Stalhammar-Carlemalm et al., 1993 [supra]). rib would also appear to be present in strains representing serotypes VII and VII (lanes 17 and 18) but was absent from strains representing serotypes IV, V and V (lanes 14 to 16) as well as the control strains (lanes 19 and 20). The rib gene probe did hybridise with lower intensity to genomic DNA fragments from strains representing serotypes Ia, Ib, IV, VI, VII and serotype II strains 118/158 and 97/0057. This may indicate the presence of a gene in these strains with a lower level of homology to rib. These hybridising DNA fragments may contain a homologue of the GBS bca gene encoding the Ca protein antigen which has been shown to be closely homologous to the Rib protein (Wastfelt et al., J. Biol. Chem. 271:18892-18897 (1996)). If this is the case, it would be in agreement with previous work which showed all strains of serotypes Ia, Ib, II and III to be positive for one the two proteins (Stalhammar-Carlemalm et al., 1993 [supra]). However, the apparent variable distribution of the rib gene amongst different GBS serotypes, makes it a less than ideal candidate for use in a GBS vaccine that is cross-protective against all serotypes.

ID-65 (FIG. 13) Comment

The Southern blot analysis described in FIG. 13 indicates that gene ID-65 is conserved across all GBS serotypes. The gene probe hybridised specifically to a Hin DIII-digested genomic DNA fragment of approximately 3.0 kb in DNA digests from all GBS representatives, and was absent from both the control strains (lanes 18 and 19). This would suggest that the ID-65 gene is conserved across all GBS serotypes (and strains) at both the gene and locus level. The ID-65 DNA probe also hybridised weakly to the 1.636 bp molecular weight marker (the 1 kb DNA ladder from NEB was used to estimate DNA fragment sizes in all Southern blot analyses).

ID-89 (FIG. 14) Comment

The Southern blot analysis described in FIG. 14 indicates that gene ID-89 may not be conserved across all GBS serotypes. A 4.0 kb HinDIII-digested genomic DNA fragment from 12 out of 16 GBS strains hybridised specifically to the ID-89 gene probe. In addition, a 3.25 kb HinDIII-digested genomic DNA fragment from the GBS strain Ib (SB35) [lane 4) also hybridised specifically with the ID-89 gene probe. However, the ID-89 gene probe did not hybridise to digested genomic DNA fragments from strains Ia (515) [lane 2], IV (3139) [lane 13] and V (1169-NT) [lane 14], suggesting that these strains do not possess a ID-89 gene homologue.

ID-93 (FIG. 15) Comment

The Southern blot analysis described in FIG. 15 indicates that gene ID-93 is conserved across all GBS serotypes. The gene probe hybridised specifically to a Hin DIII-digested genomic DNA fragment of approximately 3.25 kb in DNA digests from all GBS representatives, and was absent from both the control strains (lanes 18 and 19). This would suggest that the ID-93 gene is conserved across all GBS serotypes (and strains) at both the gene and locus level.

ID-96 (FIG. 16) Comment

The Southern blot analysis described in FIG. 16 indicates that gene ID-96 is conserved across all GBS serotypes. The gene probe hybridised specifically to a Eco RI-digested genomic DNA fragment of approximately 12.0 kb in DNA digests from all GBS representatives, and was absent from both the control strains (lanes 18 and 19). This would suggest that the ID-96 gene is conserved across all GBS serotypes (and strains) at both the gene and locus level.

[image]

[image]

[image]

[image]

[image]

1. A Group B Streptococcus polypeptide or protein having a sequence selected from those described in FIG. 1, or fragments or derivatives thereof.

2. Derivatives or variants of the proteins, polypeptides, and peptides as claimed in claim 1 which show at least 50% identity to those proteins, polypeptides and peptides claimed in claim 1.

3. A Group B Streptococcus polypeptide or protein, or derivative or variant thereof, as claimed in claim 1 or claim 2, which is isolated or recombinant.

4. A nucleic molecule comprising or consisting of a sequence which is:
(i) any of the DNA sequences set out in FIG. 1 herein or their RNA equivalents;
(ii) a sequence which is complementary to any of the sequences of (i);
(iii) a sequence which codes for the same protein or polypeptide, as those sequences of (i) or (ii);
(iv) a sequence which shows substantial identity with any of those of (i), (ii) and (iii); or
(v) a sequence which codes for a derivative, or fragment of a nucleic acid molecule shown in FIG. 1.

5. A vector comprising one or nucleic acid molecules as defined in claim 4.

6. A vector as claimed in claim 4 further comprising nucleic acid encoding any one or more of the following: promoters, enhancers, signal sequences, leader sequences, translation start and stop signals, DNA stability controlling regions, or a fusion partner.

7. The use of a vector as claimed in claim 5 or claim 6 in the transformation or transfection of a prokaryotic or eukaryotic host.

8. A host cell transformed with a vector as defined in claim 5 or claim 6..

9. A process for producing a Group B Streptococcus polypeptide or protein, or derivative or variant thereof, as claimed in claim 1 or claim 2, the process comprising expressing the polypeptide or protein in a host cell as claimed in claim 8.

10. An antibody, an affibody, or a derivative thereof which binds to one or more of the proteins, polypeptides, peptides, fragments or derivatives thereof, as defined in any one of claims 1 to 3.

11. An immunogenic composition comprising one or more of the proteins, polypeptides, peptides, fragments or derivatives thereof as defined in any one of claims 1 to 3.

12. An immunogenic composition as claimed in claim 11 wherein the proteins, polypeptides, peptides, or fragments or derivatives thereof include ID-65 or ID-83, ID-89, ID-93 or ID-96.

13. An immunogenic composition as claimed in claim 11 or claim 12 which is a vaccine.

14. An immunogenic composition comprising one or more of the nucleic acid sequences as defined in claim 4.

15. An immunogenic composition as claimed in claim 14 wherein the nucleic acid sequences include ID-65 or ID-66.

16. An immunogenic composition as claimed in claim 14 or claim 15 which is a vaccine.

17. Use of an immunogenic composition as defined in any one of claims 11 to 16 in the preparation of a medicament for the treatment or prophylaxis of Group B Streptococcus infection.

18. A method of detection of Group B Streptococcus which comprises the step of bringing into contact a sample to be tested with at least one antibody, affibody, or a derivative thereof, as defined in claim 10.

19. A method of detection of Group B Streptococcus which comprises the step of bringing into contact a sample to be tested with at least one protein, polypeptide, peptide, fragments or derivatives as defined in any one of claims 1 to 3.

20. A method of detection of Group B Streptococcus which comprises the step of bringing into contact a sample to be tested with at least one nucleic acid molecule as defined in claim 4.

21. A kit for the detection of Group B Streptococcus comprising at least one antibody, affibody, or derivatives thereof as defined in claim 10.

22. A kit for the detection of Group B Streptococcus comprising at least one Group B Streptococcus protein, polypeptide, peptide, fragment or derivative thereof as defined in any one of claims 1 to 3.

23. A kit for the detection of Group B Streptococcus comprising at least one nucleic acid molecule as defined in claim 4.

24. A method of determining whether a protein, polypeptide, peptide, fragment or derivative thereof as defined in any one of claims 1 to 3 represents a potential anti-microbial target which comprises inactivating said protein and determining whether Group B Streptococcus is still viable.
